(19) United States
(12) Patent Application Publication
(10) Pub. No.: US 2016/0318862 A1
(43) Pub. Date: Nov. 3, 2016

(54) SYNTHESIS OF DELTA 12-PGJ3 AND RELATED COMPOUNDS

(71) Applicant: WILLIAM MARSH RICE UNIVERSITY, Houston, TX (US)

(72) Inventors: Kyriacos C. NICOLAOU, Houston, TX (US); Philipp HERETSCH, Houston, TX (US); Christopher R.H. HALE, Houston, TX (US); Abdellatif El Marrouni EL GHZAOUI, Houston, TX (US); Kiran Kumar PULUKURI, Houston, TX (US); Ruocheng YU, Houston, TX (US); Charles GROVE, Houston, TX (US)

(73) Assignee: WILLIAM MARSH RICE UNIVERSITY, Houston, TX (US)

(21) Appl. No.: 15/024,427

(22) PCT Filed: Sep. 25, 2014
(86) PCT No.: PCT/US14/57426

§ 371 Date: Mar. 24, 2016

Related U.S. Application Data
(60) Provisional application No. 61/882,093, filed on Sep. 25, 2013.
(60) Provisional application No. 61/897,681, filed on Oct. 30, 2013.
(60) Provisional application No. 61/920,302, filed on Dec. 23, 2013.
(60) Provisional application No. 61/954,295, filed on Mar. 17, 2014.
(60) Provisional application No. 61/979,276, filed on Apr. 14, 2014.

(51) Int. Cl. C07C 405/00; C07D 277/24; C07D 257/04; C07C 45/61; C07C 49/743; C07C 49/647; C07D 333/32; C07D 295/116; C07D 313/00; C07D 277/30

(52) U.S. Cl. CPC C07C 405/00; C07D 313/00; C07D 277/24; C07D 257/04; C07D 277/30; C07C 405/0016; C07C 49/743; C07C 49/647; C07D 333/32; C07D 295/116; C07C 405/0041; C07C 45/61; C07C 2101/10; C07C 2103/90; C07C 2101/02; C07C 2102/38

(57) ABSTRACT

In one aspect, the present invention provides novel derivatives of Δ¹²-PGJ₃ and modular synthetic pathways to obtaining Δ¹²-PGJ₃ and derivatives thereof. In some aspects, the present derivatives of Δ¹²-PGJ₃ are useful as chemotherapeutic agents. The present disclosure also describes compositions of these derivatives as well as methods of use of the derivatives thereof.

This application claims the benefit of U.S. Provisional Application 61/882,093, filed on Sep. 25, 2013, U.S. Provisional Application 61/897,681, filed on Oct. 30, 2013, U.S. Provisional Application 61/920,302, filed on Dec. 23, 2013, U.S. Provisional Application 61/954,295, filed on Mar. 17, 2014, and U.S. Provisional Application 61/979,276, filed on Apr. 14, 2014, the entire contents of each of these applications are incorporated by reference herein.

### BACKGROUND OF THE INVENTION

1. Field of the Invention

This disclosure relates to the fields of medicine, pharmacology, chemistry and oncology. In particular, new compounds and methods of synthesis relating to prostaglandins are disclosed.

2. Related Art

Leukemia is a type of cancer of the blood or bone marrow characterized by an abnormal increase of immature white blood cells called “blasts.” Leukemia is a broad term covering a spectrum of diseases. In turn, it is part of the even broader group of diseases affecting the blood, bone marrow, and lymphoid system, which are all known as hematological neoplasms. Leukemia can affect people at any age. In 2000, approximately 256,000 children and adults around the world had developed some form of leukemia, and 209,000 have died from it. About 90% of all leukemias are diagnosed in adults.

Leukemia is a treatable disease. Most treatments involve chemotherapy, medical radiation therapy, hormone treatments, or bone marrow transplant. The rate of cure depends on the type of leukemia as well as the age of the patient. Children are more likely to be permanently cured than adults. Even when a complete cure is unlikely, most people with a chronic leukemia and many people with an acute leukemia can be successfully treated for years. Nonetheless, new and improved treatments for the disease would provide a greater chance of survival for some leukemia patients, and potentially increased survival for many others.

One of the particularly challenging aspects of treating leukemia is the presence of cancer stem cells. Leukemia stem cells are notoriously refractory to conventional drugs; consequently, their eradication is an important unrealized therapeutic goal. It was shown recently (Hegde, et al., 2011), that Δ¹²-PGJ₃ (1, FIG. 1), a naturally-occurring cyclopentenone prostaglandin derived biosynthetically from the dietary fish-oil omega-3 polyunsaturated fatty acid eicosapentaenoic acid 20:5(n−3), alleviates the development of leukemia in two well-studied murine models of leukemia. Intraperitoneal administration of Δ¹²-PGJ₃ to mice infected with Friend erythroleukemia virus or those expressing chronic myelogenous leukemia (CML) oncoprotein BCR-abl in the hematopoietic stem cell (HSC) pool completely restored normal hematological parameters, splenic histology, and enhanced the survival of such mice. More importantly, Δ¹²-PGJ₃ selectively targets and induces apoptosis of leukemia stem cells (LSCs) in mice spleen and bone marrow. The treatment completely eradicated LSCs in vivo as demonstrated by the inability of donor cells from treated mice to cause leukemia in secondary transplants. This appears to be the first example of a compound that eradicates leukemia stem cells and effectively cures CML in a mouse model, thereby prolonging life of the leukemic mice indefinitely. Given the potency of Δ¹²-PGJ₃ and the well-known refractoriness of LSC to currently used clinical agents, this natural product represents a highly interesting target for a chemical total synthesis, both, to provide sufficient quantities of so far only scarcely available Δ¹²-PGJ3 (1) and, furthermore, of analogs with enhanced and fine-tuned physico-chemical and pharmacological properties.

The structurally related 15-deoxy-Δ^{12,14}-PGJ₂ (3, FIG. 1) represents the most potent natural ligand reported to date for the peroxisome proliferator-activated receptor γ (PPARγ), a receptor that has been linked to non-insulin dependent diabetes mellitus (type II diabetes), obesity, hypertension, and atherosclerosis (Narumiya and Fukushima, 1986; Narumiya, et al., 1986; Narumiya, et al., 1987; Rocchi and Auwerx, 1999; Kersten, et al., 2000). Inhibition of the NF-κB-mediated transcription is another property of 3. On the other hand, Δ¹²-PGJ₂ (4, FIG. 1) exhibits strong antitumor effects by incorporating into tumor cells and transferring into nuclei, activating the gadd45 promoter independently of p53 (Ricote, et al., 1998; Jiang, et al., 1998; Rossi, et al., 2000; Sasaki and Fukushima, 1994) and inhibiting topoisomerase (Ohtani-Fujita, et al., 1998; Suzuki, et al., 1998). Among these targets, only 3 and 4 have been synthesized so far. In 2003, Sutton (Bickley, et al., 2003) accessed racemic 3 by Meinwald rearrangement of a norbornadiene and an asymmetric acetylation using enzymes in two stages to accomplish resolutions at the stereocenters on the ω-chain and on the cyclopentene ring. Later, 3 was synthesized (again as a racemate) by Brummond (Brummond, et al., 2004) through a Si-tethered allenic [2+2+1] cycloaddition. At the same time Kobayashi (Acharya and Kobayashi, et al., 2004; Acharya and Kobayashi, 2006) reported an approach to optically active PGJs 3 and 4. The former two syntheses by Sutton and Brummond seem to present little advantage over the latter method with respect to product selectivity, efficiency, and, in particular, diastereoselectivity. Furthermore, the reaction conditions would be hardly applicable to the synthesis of 1. Thus, improved synthetic methods are needed.

### SUMMARY OF THE INVENTION

Thus, in accordance with the present disclosure, there is provided a compound of the formula:

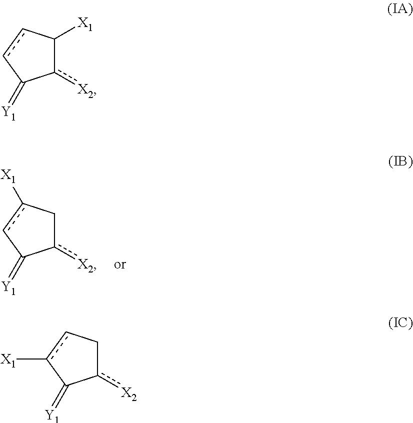

wherein: Y₁ is O, NR₁, or N—OR₁; wherein: R₁ is hydrogen, alkyl_{(C≦6)}, or substituted alkyl_{(C≦6)}; X₁ is hydrogen, alkyl_{(C≦8)}, substituted alkyl_{(C≦8)},

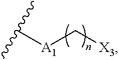

or taken together with X₂ as defined below; wherein: A₁ is alkanediyl_{(C≦8)}, alkenediyl_{(C≦8)}, alkynediyl_{(C≦8)}, arenediyl_{(C≦12)}, heteroarenediyl_{(C≦12)}, or a substituted version of any of these groups; n is 0, 1, 2, 3, 4, 5, or 6; X₃ is hydrogen, hydroxy, amino, cyano, or; alkyl_{(C≦12)}, alkenyl_{(C≦12)}, alkynyl_{(C≦12)}, aryl_{(C≦12)}, heteroaryl_{(C≦12)}, heterocycloalkyl_{(C≦12)}, alkoxy_{(C≦12)}, alkenyloxy_{(C≦12)}, alkynyloxy_{(C≦12)}, aryloxy_{(C≦12)}, heteroaryloxy_{(C≦12)}, heterocycloalkyloxy_{(C≦12)}, acyloxy_{(C≦12)}, alkylamino_{(C≦12)}, dialkylamino_{(C≦12)}, alkenylamino_{(C≦12)}, alkynylamino_{(C≦12)}, arylamino_{(C≦12)}, heteroarylamino_{(C≦12)}, heterocycloalkylamino_{(C≦12)}, amido_{(C≦12)}, or a substituted version of any of these groups; or —C(O)NR₂R₃, —C(O)R₂; or —Y₂—R₄; wherein: R₂ and R₃ are each independently hydrogen, hydroxy, alkyl_{(C≦8)}, aryl_{(C≦8)}, alkoxy_{(C≦8)}, alkylsulfonyl_{(C≦8)}, arylsulfonyl_{(C≦8)}, or a substituted version of any of the last five groups; or R₂ is -alkoxydiyl_{(C≦6)}-S(O)₂-aryl_{(C≦12)} or a substituted version of this group; Y₂ is alkanediyl_{(C≦12)}, substituted alkanediyl_{(C≦12)}; alkoxydiyl_{(C≦12)}, or substituted alkoxydiyl_{(C≦12)}; and R₄ is hydrogen, —C(O)NR₂R₃, or —C(O)R₂; wherein R₂ and R₃ are as defined above and X₂ is

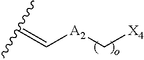

or taken together with X₁ as defined below; wherein: A₂ is alkanediyl_{(C≦8)}, alkenediyl_{(C≦8)}, alkynediyl_{(C≦8)} or a substituted version of any of these groups; or —CH₂CH(OR₄)—; wherein: R₄ is alkyl_{(C≦12)}; alkenyl_{(C≦12)}, alkynyl_{(C≦12)}, aryl_{(C≦12)}, heteroaryl_{(C≦12)}, acyl_{(C≦12)}, or a substituted version of any of these groups; X₄ is hydrogen, hydroxy, or alkyl_{(C≦12)}, alkenyl_{(C≦12)}, alkynyl_{(C≦12)}, aryl_{(C≦12)}, heteroaryl_{(C≦12)}, heterocycloalkyl_{(C≦12)}, aryloxy_{(C≦12)}, heteroaryloxy_{(C≦12)}, heterocycloalkoxy_{(C≦12)}, arylthio_{(C≦12)}, heteroarylthio_{(C≦12)}, heterocycloalkylthio_{(C≦12)}, arylsulfinyl_{(C≦12)}, heteroarylsulfinyl_{(C≦12)}, heterocycloalkylsulfinyl_{(C≦12)}, arylsulfonyl_{(C≦12)}, heteroarylsulfonyl_{(C≦12)}, heterocycloalkylsulfonyl_{(C≦12)}, or a substituted version of any of these groups; and o is 0, 1, 2, 3, 4, 5, or 6; wherein: X₁ and X₂ are taken together as shown in formula (II):

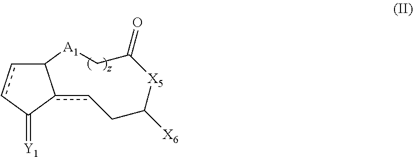

wherein: Y₁ is O, NH, or N—OR₁; wherein: R₁ is hydrogen, alkyl_{(C≦6)}, or substituted alkyl_{(C≦6)}; A₁ is alkanediyl_{(C≦8)}, alkenediyl_{(C≦8)}, alkynediyl_{(C≦8)}, arenediyl_{(C≦12)}, heteroarenediyl_{(C≦12)}, or a substituted version of any of these groups; z is 1, 2, 3, 4, 5, or 6; X₅ is CR₄R₅, O, NH, NR₆, or S; wherein: R₄, R₅, and R₆ are each independently H, alkyl_{(C≦8)}, aryl_{(C≦8)}, aralkyl_{(C≦8)}; or a substituted version of any of the last three groups; and X₆ is alkyl_{(C≦12)}; alkenyl_{(C≦12)}, alkynyl_{(C≦12)}, aryl_{(C≦12)}, heteroaryl_{(C≦12)} or a substituted version of any of these groups; provided that the compound does not have the formula:

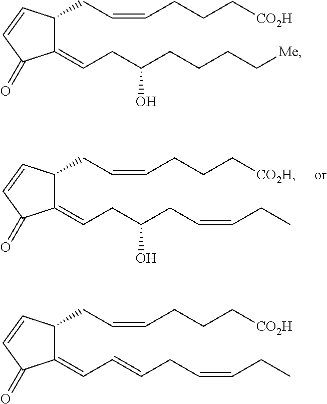 [SMILES: CCCCC[C@H](O)C/C=C1/C(=O)C=C[C@@H]1C/C=C\CCCC(=O)O ; CC/C=C\C[C@H](O)C/C=C1/C(=O)C=C[C@@H]1C/C=C\CCCC(=O)O ; "or" ; CC/C=C\C/C=C/C=C1/C(=O)C=C[C@@H]1C/C=C\CCCC(=O)O]

or a pharmaceutically acceptable salt thereof. In some embodiments, the compound is further defined by the formula:

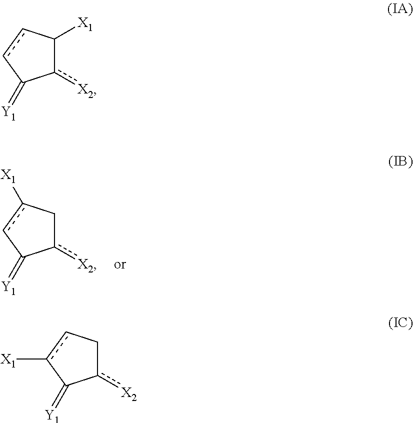

wherein: Y₁ is O, NR₁, or N—OR₁; wherein: R₁ is hydrogen, alkyl_{(C≦6)}, or substituted alkyl_{(C≦6)}; X₁ is hydrogen, alkyl_{(C≦8)}, substituted alkyl_{(C≦8)},

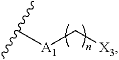

or taken together with X₂ as defined below; wherein: A₁ is alkanediyl_{(C≦8)}, alkenediyl_{(C≦8)}, alkynediyl_{(C≦8)}, arenediyl_{(C≦12)}, heteroarenediyl_{(C≦12)}, or a substituted version of any of these groups; n is 0, 1, 2, 3, 4, 5, or 6; X₃ is hydrogen, hydroxy, amino, cyano, or alkyl_{(C≦12)}, alkenyl_{(C≦12)}, alkynyl_{(C≦12)}, aryl_{(C≦12)}, heteroaryl_{(C≦12)}, heterocycloalkyl_{(C≦12)}, or a substituted version of any of these groups; or —C(O)NR₂R₃ or —C(O)R₂; wherein: R₂ and R₃ are each independently hydrogen, hydroxy, alkyl_{(C≦6)}, aryl_{(C≦8)}, alkoxy_{(C≦6)}, alkylsulfonyl_{(C≦8)}, arylsulfonyl_{(C≦8)}, or a substituted version of any of the last five groups; or R₂ is -alkoxydiyl_{(C≦6)}-S(O)₂-aryl_{(C≦12)} or a substituted version of this group; and X₂ is

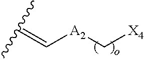

or taken together with X₁ as defined below; wherein: A₂ is alkanediyl_{(C≦8)}, alkenediyl_{(C≦8)}, alkynediyl_{(C≦8)} or a substituted version of any of these groups; X₄ is hydrogen, hydroxy, or alkyl_{(C≦12)}, alkenyl_{(C≦12)}, alkynyl_{(C≦12)}, aryl_{(C≦12)}, heteroaryl_{(C≦12)}, heterocycloalkyl_{(C≦12)}, aryloxy_{(C≦12)}, heteroaryloxy_{(C≦12)}, heterocycloalkoxy_{(C≦12)}, arylthio_{(C≦12)}, heteroarylthio_{(C≦12)}, heterocycloalkylthio_{(C≦12)}, arylsulfinyl_{(C≦12)}, heteroarylsulfinyl_{(C≦12)}, heterocycloalkylsulfinyl_{(C≦12)}, arylsulfonyl_{(C≦12)}, heteroarylsulfonyl_{(C≦12)}, heterocycloalkylsulfonyl_{(C≦12)}, or a substituted version of any of these groups; and o is 0, 1, 2, 3, 4, 5, or 6; wherein: X₁ and X₂ are taken together as shown in formula (II):

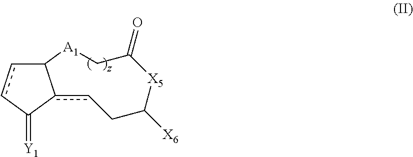

wherein: Y₁ is O, NH, or N—OR₁; wherein: R₁ is hydrogen, alkyl_{(C≦6)}, or substituted alkyl_{(C≦6)}; A₁ is alkanediyl_{(C≦8)}, alkenediyl_{(C≦8)}, alkynediyl_{(C≦8)}, arenediyl_{(C≦12)}, heteroarenediyl_{(C≦12)}, or a substituted version of any of these groups; z is 1, 2, 3, 4, 5, or 6; X₅ is CR₄R₅, O, NH, NR₆, or S; wherein: R₄, R₅, and R₆ are each independently H, alkyl_{(C≦8)}, aryl_{(C≦8)}, aralkyl_{(C≦8)}; or a substituted version of any of the last three groups; and X₆ is alkyl_{(C≦12)}; alkenyl_{(C≦12)}, alkynyl_{(C≦12)}, aryl_{(C≦12)}, heteroaryl_{(C≦12)} or a substituted version of any of these groups; provided that the compound does not have the formula:

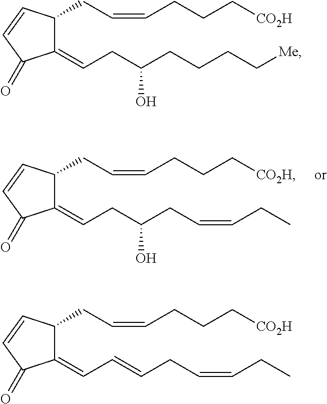 [SMILES: CCCCC[C@H](O)C/C=C1/C(=O)C=C[C@@H]1C/C=C\CCCC(=O)O ; CC/C=C\C[C@H](O)C/C=C1/C(=O)C=C[C@@H]1C/C=C\CCCC(=O)O ; "or" ; CC/C=C\C/C=C/C=C1/C(=O)C=C[C@@H]1C/C=C\CCCC(=O)O]

or a pharmaceutically acceptable salt thereof. In some embodiments, the compound is further defined as:

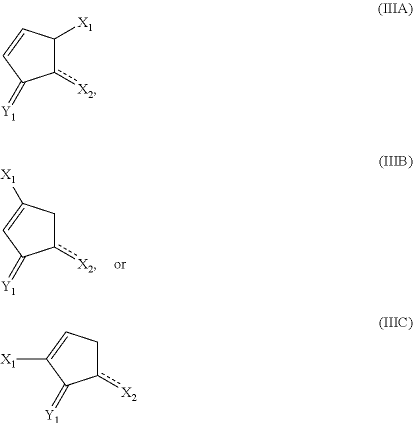

wherein: Y₁ is O or N—OR₁; wherein: R₁ is hydrogen or alkyl_{(C≦6)}; X₁ is hydrogen or

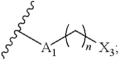

wherein: A₁ is alkanediyl_{(C≦8)}, alkenediyl_{(C≦8)}, alkynediyl_{(C≦8)}, or heteroarenediyl_{(C≦12)}; n is 0, 1, 2, 3, or 4; X₃ is hydrogen, hydroxy, alkyl_{(C≦6)}, heteroaryl_{(C≦8)}, or a substituted version of any of these groups; or —C(O)NR₂R₃ or —C(O)R₂; wherein: R₂ and R₃ are each independently hydrogen, alkyl_{(C≦6)}, aryl_{(C≦8)}, alkoxy_{(C≦6)}, or a substituted version of any of the last three groups; and X₂ is

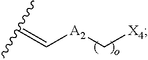

wherein: A₂ is alkanediyl_{(C≦8)}, alkenediyl_{(C≦8)}, or a substituted version of any of these groups; X₄ is hydrogen, hydroxy, or alkyl_{(C≦12)}, alkenyl_{(C≦12)}, alkynyl_{(C≦12)}, aryl_{(C≦12)}, heteroaryl_{(C≦12)}, heterocycloalkyl_{(C≦12)}, aryloxy_{(C≦12)}, heteroaryloxy_{(C≦12)}, heterocycloalkoxy_{(C≦12)}, arylthio_{(C≦12)}, heteroarylthio_{(C≦12)}, heterocycloalkylthio_{(C≦12)}, arylsulfonyl_{(C≦12)}, heteroarylsulfonyl_{(C≦12)}, heterocycloalkylsulfonyl_{(C≦12)}, or a substituted version of any of these groups; and o is 0, 1, 2, 3, 4, 5, or 6; wherein: X₁ and X₂ are taken together as shown in formula (IV):

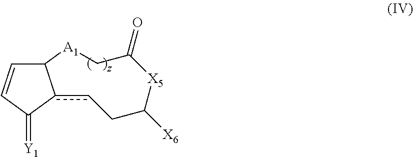

wherein: Y₁ is O, NH, or N—OR₁; wherein: R₁ is hydrogen, alkyl_{(C≦6)}, or substituted alkyl_{(C≦6)}; A₁ is alkanediyl_{(C≦8)}, alkenediyl_{(C≦8)}, alkynediyl_{(C≦8)}, arenediyl_{(C≦12)}, heteroarenediyl_{(C≦12)}, or a substituted version of any of these groups; z is 1, 2, 3, 4, 5, or 6; X₅ is CR₄R₅, O, NH, NR₆, or S; wherein: R₄, R₅, and R₆ are each independently H, alkyl_{(C≦8)}, aryl_{(C≦8)}, aralkyl_{(C≦8)}; or a substituted version of any of the last three groups; and X₆ is alkyl_{(C≦12)}; alkenyl_{(C≦12)}, alkynyl_{(C≦12)}, aryl_{(C≦12)}, heteroaryl_{(C≦12)} or a substituted version of any of these groups; provided that the compound does not have the formula:

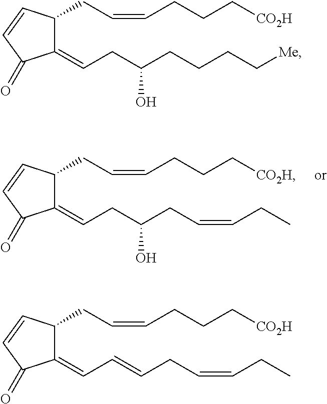 [SMILES: CCCCC[C@H](O)C/C=C1/C(=O)C=C[C@@H]1C/C=C\CCCC(=O)O ; CC/C=C\C[C@H](O)C/C=C1/C(=O)C=C[C@@H]1C/C=C\CCCC(=O)O ; "or" ; CC/C=C\C/C=C/C=C1/C(=O)C=C[C@@H]1C/C=C\CCCC(=O)O]

or a pharmaceutically acceptable salt thereof. In some embodiments, A₂ is not

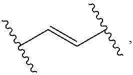 [SMILES: */C=C/*]

when X₁ is

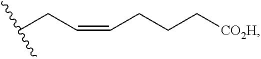 [SMILES: *C/C=C\CCCC(=O)O]

or that A₂ is not

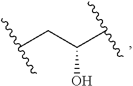 [SMILES: *C[C@H](*)O]

when X₁ is

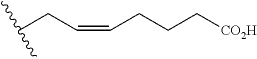 [SMILES: *C/C=C\CCCC(=O)O]

### and X₄ is

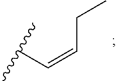 [SMILES: */C=C\CC]

or a pharmaceutically acceptable salt thereof. In some embodiments, the compound is further defined by formula IA. In some embodiments, the compound is further defined by formula IB. In some embodiments, the compound is further defined by formula IC. In some embodiments, Y₁ is O. In some embodiments, Y₁ is N—OH or N—OMe. In some embodiments, Y₁ is N—OMe. In some embodiments, X₁ is

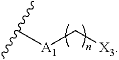

In some embodiments, A₁ is alkanediyl_{(C≦6)}. In some embodiments, A₁ is —CH₂CH₂—. In some embodiments, A₁ is alkenediyl_{(C≦6)}. In some embodiments, A₁ is —CH₂CH═CH—. In some embodiments, A₁ is akynediyl_{(C≦6)}. In some embodiments, A₁ is —CH₂C≡C—. In some embodiments, A₁ is heteroarenediyl_{(C≦6)}. In some embodiments, A₁ is

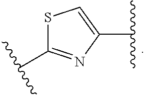 [SMILES: *c1csc(*)n1]

In some embodiments, n is 3 or 4. In some embodiments, n is 3. In some embodiments, n is 4. In some embodiments, n is 0. In some embodiments, X₃ is hydroxy. In some embodiments, X₃ is acyl_{(C≦6)} or substituted acyl_{(C≦6)}. In some embodiments, X₃ is —CO₂H or —CO₂Me. In some embodiments, X₃ is heteroaryl_{(C≦6)}. In some embodiments, X₃ is

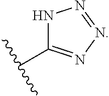 [SMILES: *c1nnn[nH]1]

In some embodiments, X₃ is aryloxy_{(C≦12)} or substituted aryloxy_{(C≦12)}. In some embodiments, X₃ is —OCH₂C₆H₄OMe. In some embodiments, X₃ is —C(O)NR₂R₃, wherein R₂ and R₃ are each independently hydrogen, hydroxy, alkyl_{(C≦8)}, aryl_{(C≦8)}, alkoxy_{(C≦8)}, alkylsulfonyl_{(C≦8)}, arylsulfonyl_{(C≦8)}, or a substituted version of any of the last five groups; or R₂ is alkanediyl_{(C≦6)}-S(O)₂-aryl_{(C≦12)}, alkoxydiyl_{(C≦6)}-S(O)₂-aryl_{(C≦12)}, or a substituted version of either group. In some embodiments, R₂ is alkyl_{(C≦8)} or substituted alkyl_{(C≦8)}. In some embodiments, R₂ is methyl. In some embodiments, R₂ is alkylsulfonyl_{(C≦8)} or substituted alkylsulfonyl_{(C≦8)}. In some embodiments, R₂ is —S(O)₂Me or —S(O)₂Et. In some embodiments, R₂ is arylsulfonyl_{(C≦8)} or substituted arylsulfonyl_{(C≦8)}. In some embodiments, R₂ is —S(O)₂Ph. In some embodiments, R₃ is hydrogen. In some embodiments, R₃ is alkyl_{(C≦8)} or substituted alkyl_{(C≦8)}. In some embodiments, R₃ is methyl. In some embodiments, R₃ is alkoxy_{(C≦8)} or substituted alkoxy_{(C≦8)}. In some embodiments, R₃ is methoxy. In some embodiments, X₃ is —C(O)R₂ wherein R₂ is hydroxy, alkoxy_{(C≦8)}, or substituted alkoxy_{(C≦8)}; or R₂ is alkanediyl_{(C≦6)}-S(O)₂-aryl_{(C≦12)}, -alkoxydiyl_{(C≦6)}-S(O)₂-aryl_{(C≦12)}, or a substituted version of either group. In some embodiments, R₂ is alkoxy_{(C≦8)} or substituted alkoxy_{(C≦8)}. In some embodiments, R₂ is methoxy, isopropoxy, or cyclopropoxy. In some embodiments, R₂ is -alkoxydiyl_{(C≦6)}-S(O)₂-aryl_{(C≦12)} or a substituted version thereof. In some embodiments, R₂ is —OCH₂CH₂—S(O)₂-Ph. In some embodiments, X₂ is

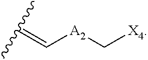

In some embodiments, A₂ is alkanediyl_{(C≦8)} or substituted alkanediyl_{(C≦8)}. In some embodiments, A₂ is —CH₂—, —CH₂CH₂—, —C(CH₂)₂CH₂—, or —C(CH₃)₂CH₂. In some embodiments, A₂ is —CH₂CH(OH)—, —C(CH₂)₂CH(OH)—, —C(CH₃)₂CH(OH)—, —CH₂CH(F)—, —C(CH₂)₂CH(F)—, or —C(CH₃)₂CH(F)—. In some embodiments, A₂ is alkenediyl_{(C≦8)} or substituted alkenediyl_{(C≦8)}. In some embodiments, A₂ is —CH═CH—, —C(CH₂)₂CH₂CH₂CH═CH—, or —C(CH₃)₂CH₂CH₂CH═CH—. In some embodiments, o is 0, 1, 2, 3, or 4. In some embodiments, o is 0, 1, 2, or 3. In some embodiments, o is 0. In some embodiments, o is 1 or 2. In some embodiments, X₄ is hydrogen. In some embodiments, X₄ is hydroxy. In some embodiments, X₄ is alkyl_{(C≦12)} is a fused cycloalkyl_{(C≦12)}. In some embodiments, X₄ is cubanyl or bicyclo[1.1.1]pentyl. In some embodiments, X₄ is alkenyl_{(C≦8)} or substituted alkenyl_{(C≦8)}. In some embodiments, X₄ is —CH═CHCH₂CH₃. In some embodiments, X₄ is —CH═CHCH₂CF₃. In some embodiments, X₄ is alkynyl_{(C≦8)}. In some embodiments, X₄ is —C≡CCH₂CH₃. In some embodiments, X₄ is aryl_{(C≦12)} or substituted aryl_{(C≦12)}. In some embodiments, X₄ is aryl_{(C≦12)}. In some embodiments, X₄ is phenyl. In some embodiments, X₄ is substituted aryl_{(C≦12)}. In some embodiments, X₄ is 2-methoxyphenyl, 3-methoxyphenyl, 4-methoxyphenyl, 2-fluorophenyl, 3-fluorophenyl, 4-fluorophenyl, 2-chlorophenyl, 3-chlorophenyl, 4-chlorophenyl, 2-trifluoromethylphenyl, 3-trifluoromethylphenyl, 4-trifluoro-methylphenyl, 2-dimethylaminophenyl, 3-dimethylaminophenyl, 4-dimethylaminophenyl, 2-methoxy-methylphenyl, 3-methoxymethylphenyl, 4-methoxymethylphenyl, 2-dimethylaminomethylphenyl, 3-dimethyl-aminomethylphenyl, or 4-dimethylaminomethylphenyl. In some embodiments, X₄ is heteroaryl_{(C≦12)} or substituted heteroaryl_{(C≦12)}. In some embodiments, X₄ is 2-thiazoyl, 4-thiazoyl, 2-thienyl, 3-thienyl, 2-oxazolyl, 3-oxazolyl, or

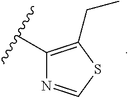 [SMILES: *c1ncsc1CC]

In some embodiments, X₄ is heterocycloalkyl_{(C≦12)} or substituted heterocycloalkyl_{(C≦12)}. In some embodiments, X₄ is N-morpholinyl. In some embodiments, X₄ is aryloxy_{(C≦12)} or substituted aryloxy_{(C≦12)}. In some embodiments, X₄ is aryloxy_{(C≦12)}. In some embodiments, X₄ is phenyloxy. In some embodiments, X₄ is substituted aryloxy_{(C≦2)}. In some embodiments, X₄ is 2-methoxyphenyoxy, 3-methoxyphenyloxy, 4-methoxyphenyloxy, 2-fluorophenyloxy, 3-fluorophenyloxy, 4-fluorophenyloxy, 2-chlorophenyloxy, 3-chlorophenyloxy, 4-chlorophenyloxy, 2-trifluoromethylphenyloxy, 3-trifluoromethylphenyloxy, 4-trifluoro-methylphenyloxy, 2-dimethylaminophenyloxy, 3-dimethylaminophenyloxy, 4-dimethylaminophenyloxy, 2-methoxymethylphenyloxy, 3-methoxymethylphenyloxy, 4-methoxymethylphenyloxy, 2-dimethylaminomethylphenyloxy, 3-dimethylaminomethylphenyloxy, or 4-dimethylaminomethylphenyloxy. In some embodiments, X₄ is heteroaryloxy_{(C≦12)} or substituted heteroaryloxy_{(C≦12)}. In some embodiments, X₄ is 2-thiazoyloxy, 4-thiazoyloxy, 2-thienyloxy, 3-thienyloxy, 2-oxazolyloxy, 3-oxazolyloxy, or

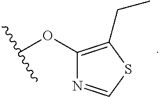 [SMILES: *Oc1ncsc1CC]

In some embodiments, X₄ is arylthio_{(C≦12)} or substituted arylthio_{(C≦12)}. In some embodiments, X₄ is arylthio_{(C≦12)}. In some embodiments, X₄ is phenylthio. In some embodiments, X₄ is substituted arylthio_{(C≦12)}. In some embodiments, X₄ is 2-methoxyphenylthio, 3-methoxyphenylthio, 4-methoxyphenylthio, 2-fluorophenylthio, 3-fluorophenylthio, 4-fluorophenylthio, 2-chlorophenylthio, 3-chlorophenylthio, 4-chlorophenylthio, 2-trifluoromethylphenylthio, 3-trifluoromethylphenylthio, 4-trifluoromethylphenylthio, 2-dimethylaminophenylthio, 3-dimethylaminophenylthio, 4-dimethylaminophenylthio, 2-methoxymethylphenylthio, 3-methoxymethylphenylthio, 4-methoxymethylphenylthio, 2-dimethylaminomethylphenylthio, 3-dimethylaminomethylphenylthio, or 4-dimethylaminomethylphenylthio. In some embodiments, X₄ is heteroarylthio_{(C≦12)} or substituted heteroarylthio_{(C≦12)}. In some embodiments, X₄ is 2-thiazoylthio, 4-thiazoylthio, 2-thienylthio, 3-thienylthio, 2-oxazolylthio, 3-oxazolylthio, or

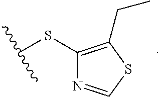 [SMILES: *Sc1ncsc1CC]

In some embodiments, X₄ is arylsulfonyl_{(C≦12)} or substituted arylsulfonyl_{(C≦12)}. In some embodiments, X₄ is arylsulfonyl_{(C≦12)}. In some embodiments, X₄ is phenylsulfonyl. In some embodiments, X₄ is substituted arylsulfonyl_{(C≦12)}. In some embodiments, X₄ is 2-methoxyphenylsulfonyl, 3-methoxyphenylsulfonyl, 4-methoxyphenylsulfonyl, 2-fluorophenylsulfonyl, 3-fluorophenylsulfonyl, 4-fluoro-phenylsulfonyl, 2-chlorophenylsulfonyl, 3-chlorophenylsulfonyl, 4-chlorophenylsulfonyl, 2-trifluoromethyl-phenylsulfonyl, 3-trifluoromethylphenylsulfonyl, 4-trifluoromethylphenylsulfonyl, 2-dimethylaminophenyl-sulfonyl, 3-dimethylaminophenylsulfonyl, 4-dimethylaminophenylsulfonyl, 2-methoxymethyl-phenylsulfonyl, 3-methoxymethylphenylsulfonyl, 4-methoxymethylphenylsulfonyl, 2-dimethylamino-methylphenylsulfonyl, 3-dimethylaminomethylphenylsulfonyl, or 4-dimethylaminomethylphenylsulfonyl. In some embodiments, X₄ is heteroarylsulfonyl_{(C≦12)} or substituted heteroarylsulfonyl_{(C≦12)}. In some embodiments, X₄ is 2-thiazoylsulfonyl, 4-thiazoylsulfonyl, 2-thienylsulfonyl, 3-thienylsulfonyl, 2-oxazolylsulfonyl, 3-oxazolylsulfonyl, or

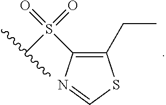 [SMILES: *S(=O)(=O)c1ncsc1CC]

In some embodiments, X₁ and X₂ are taken together as defined by the formula:

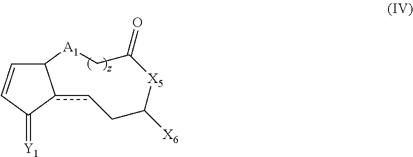

wherein: Y₁ is O, NH, or N—OR₁; wherein: R₁ is hydrogen, alkyl_{(C≦6)}, or substituted alkyl_{(C≦6)}; A₁ is alkanediyl_{(C≦8)}, alkenediyl_{(C≦8)}, alkynediyl_{(C≦8)}, arenediyl_{(C≦12)}, heteroarenediyl_{(C≦12)}, or a substituted version of any of these groups; z is 1, 2, 3, 4, 5, or 6; X₅ is CR₄R₅, O, NH, NR₆, or S; wherein: R₄, R₅, and R₆ are each independently H, alkyl_{(C≦8)}, aryl_{(C≦8)}, aralkyl_{(C≦8)}; or a substituted version of any of the last three groups; and X₆ is alkyl_{(C≦12)}; alkenyl_{(C≦12)}, alkynyl_{(C≦12)}, aryl_{(C≦12)}, heteroaryl_{(C≦12)} or a substituted version of any of these groups. In some embodiments, A₁ is alkenediyl_{(C≦6)}. In some embodiments, A₁ is —CH₂C═CH—. In some embodiments, z is 1, 2, 3, or 4. In some embodiments, z is 3 or 4. In some embodiments, z is 3. In some embodiments, X₅ is O. In some embodiments, X₆ is alkenyl_{(C≦12)}. In some embodiments, X₆ is —CH₂—CH═CH—CH₂CH₃. In some embodiments, the compound is further defined as:

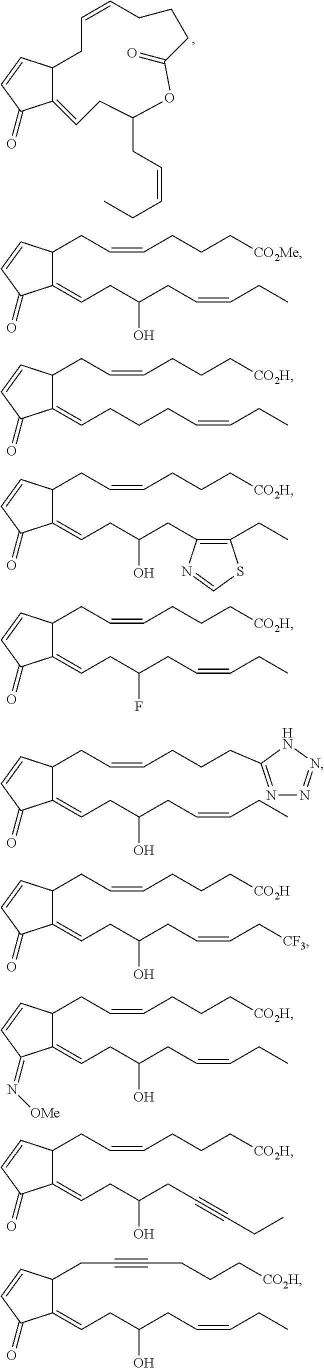

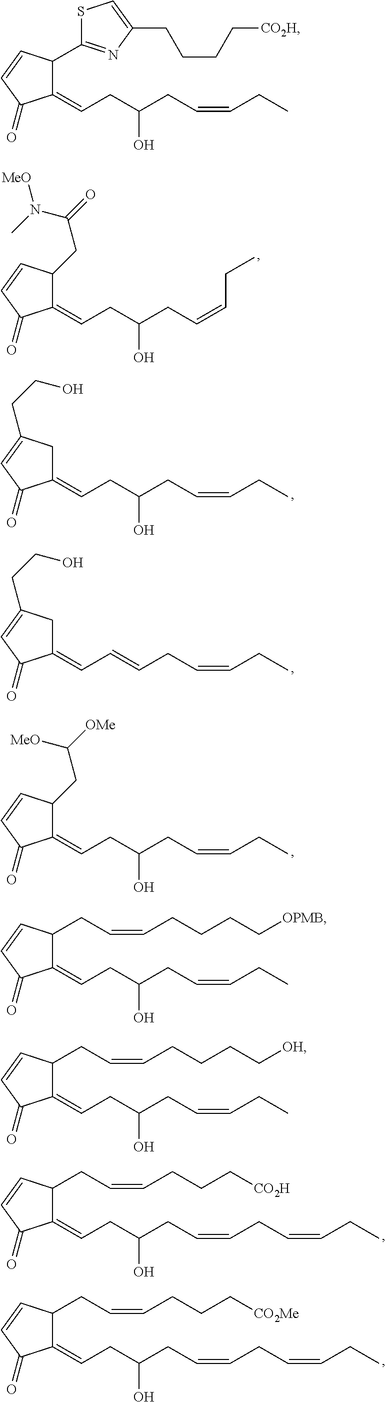

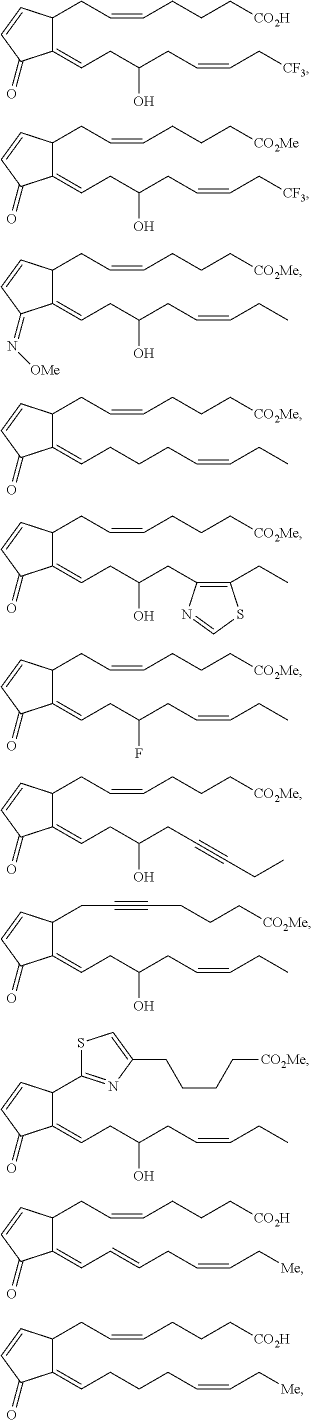

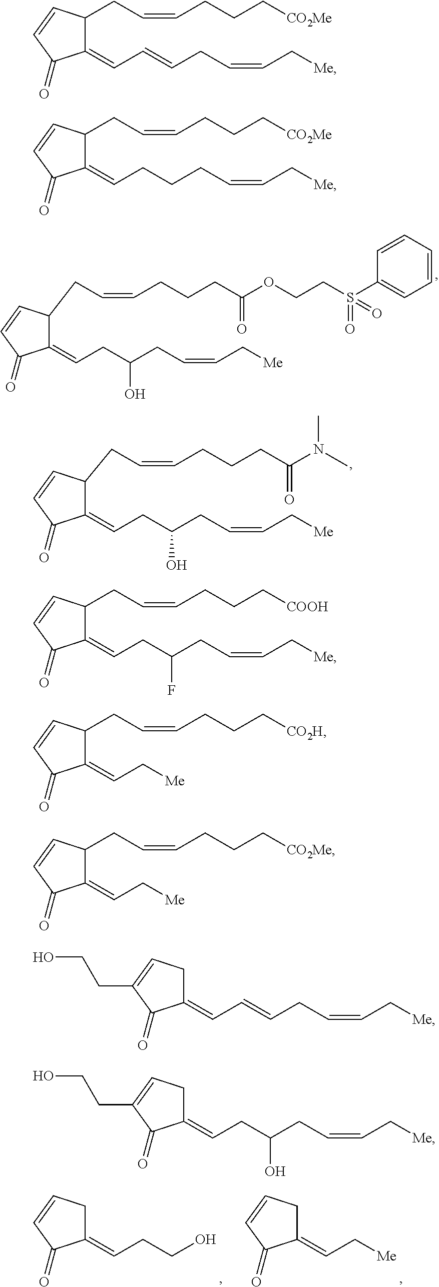

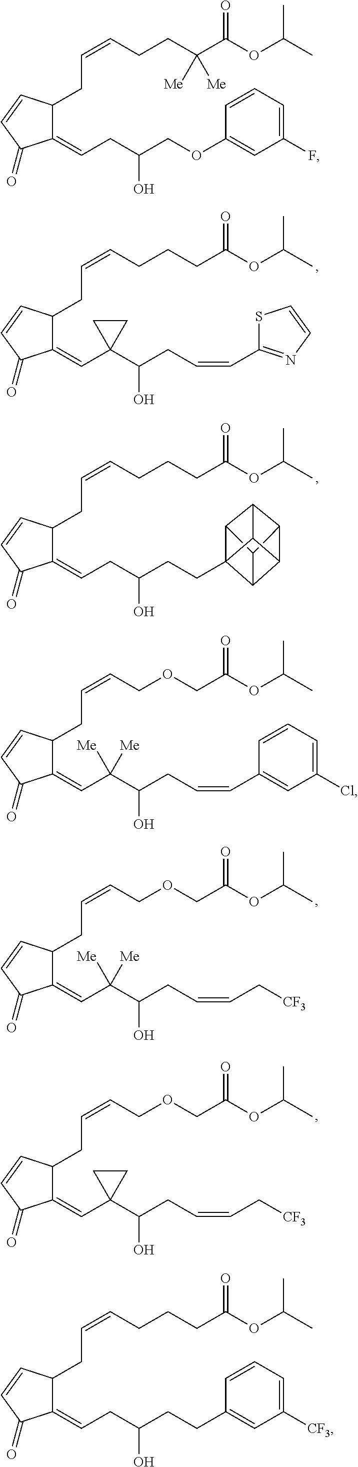

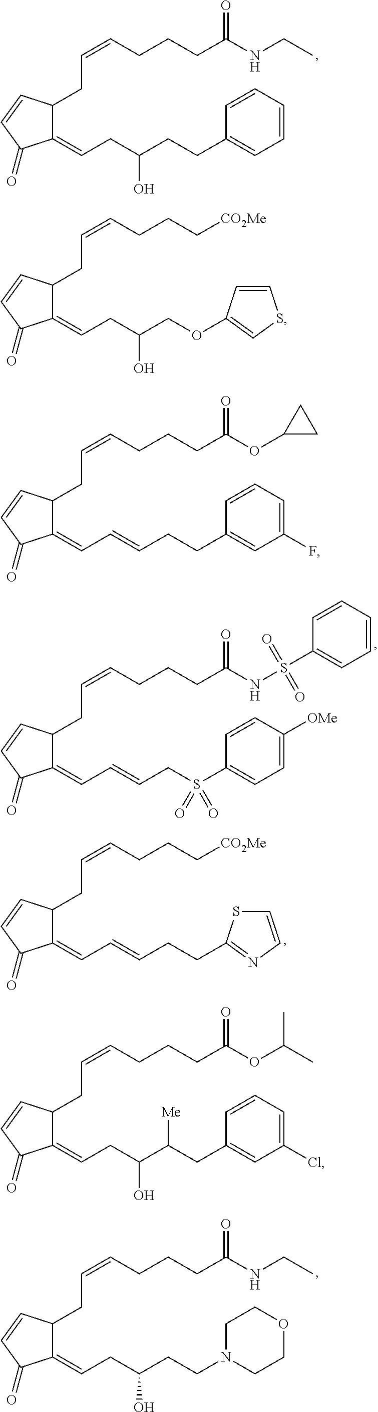

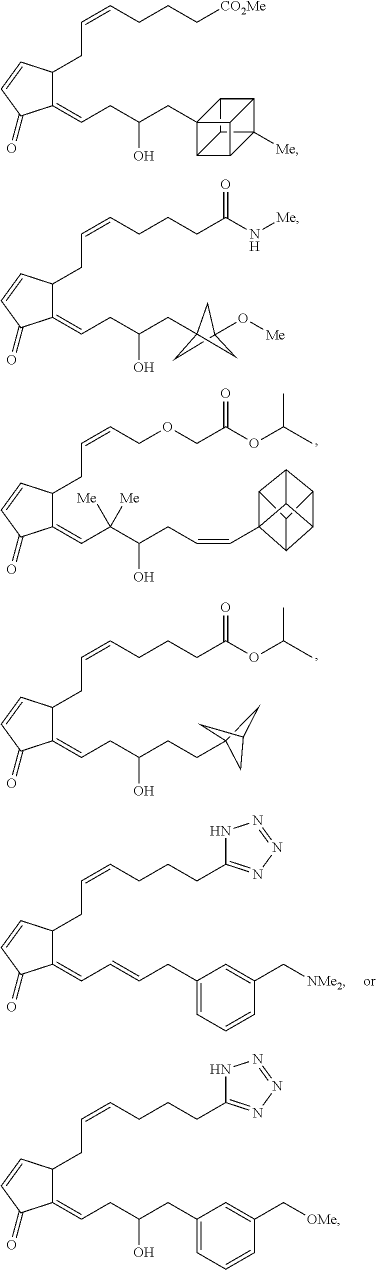

or an optical isomer or pharmaceutically acceptable salt thereof. In some embodiments, the compound further defined as:

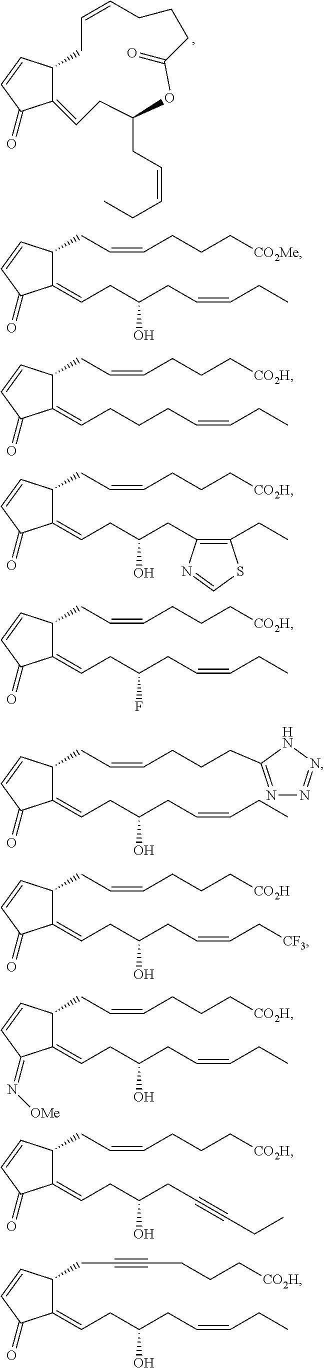

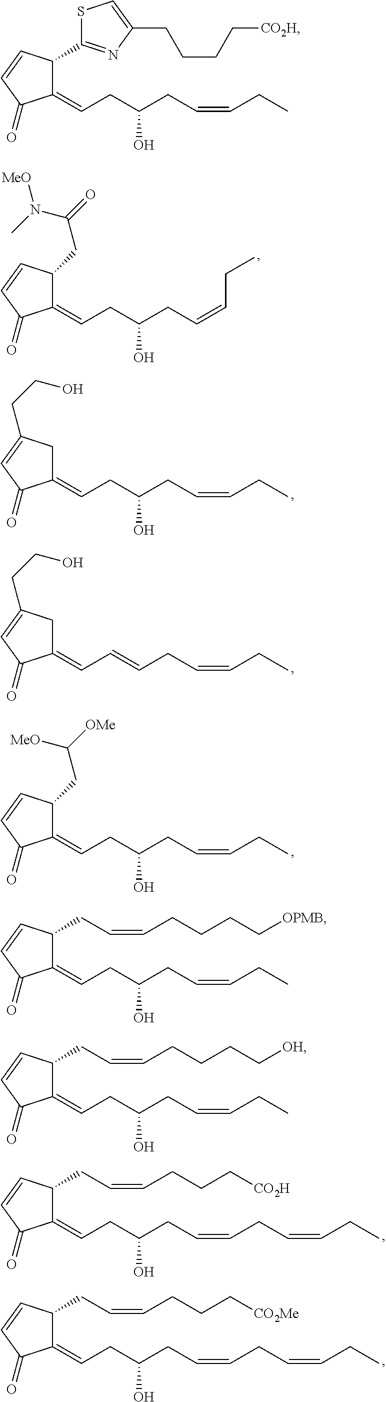

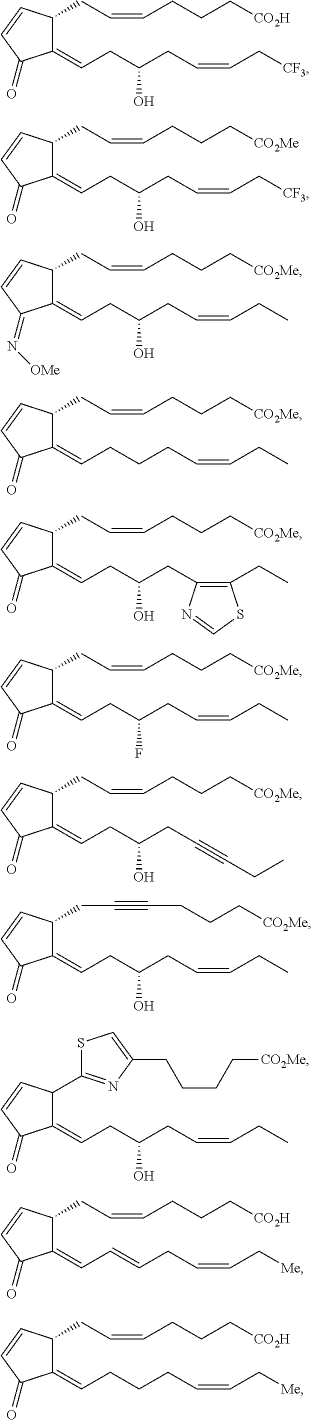

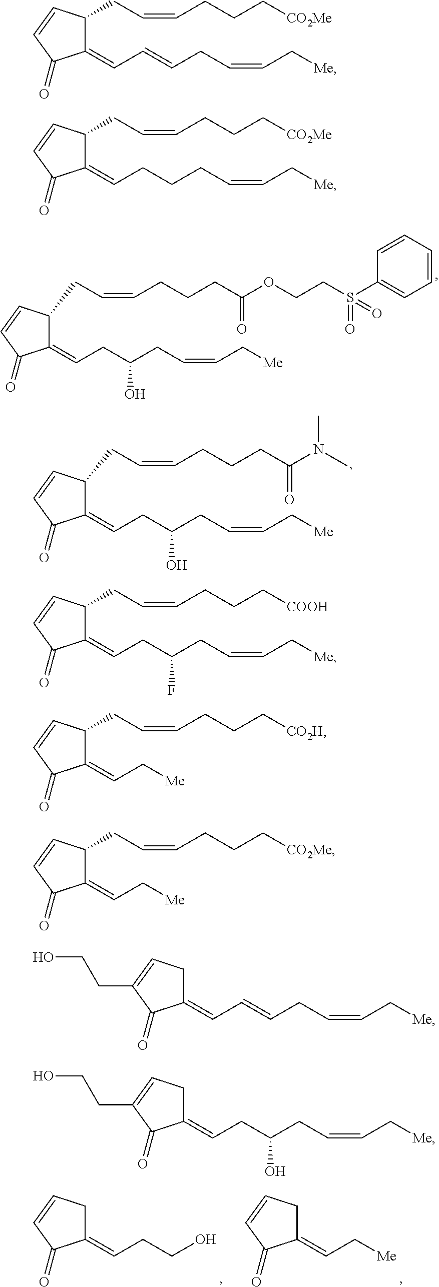

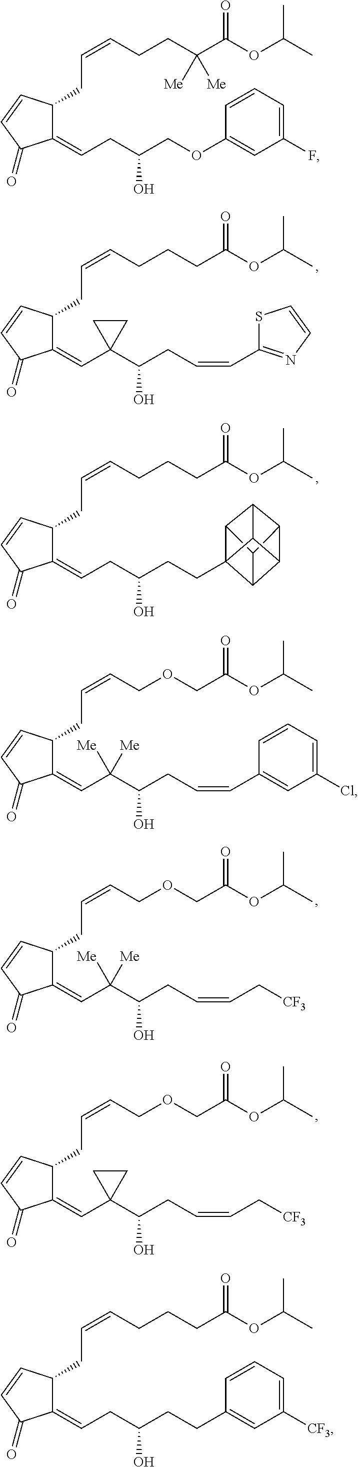

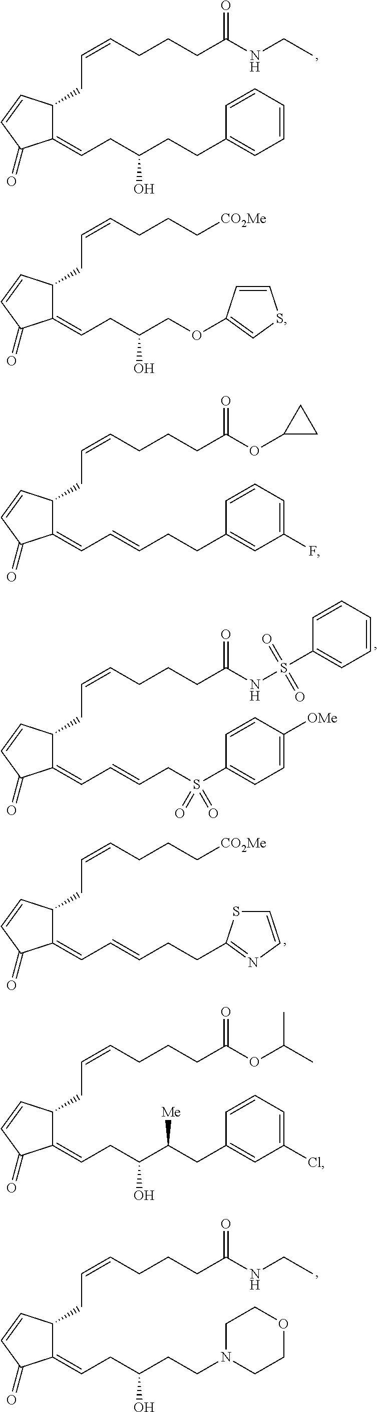

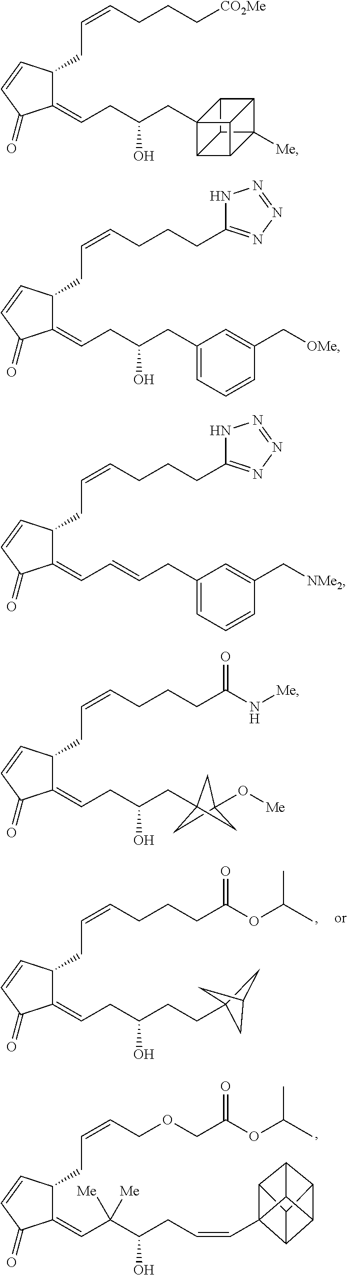

or a pharmaceutically acceptable salt thereof.

In another aspect, the present invention provides a pharmaceutical composition comprising a compound of the present invention and an excipient. In some embodiments, the composition is formulated for oral, intraadiposal, intraarterial, intraarticular, intracranial, intradermal, intralesional, intramuscular, intranasal, intraocular, intrapericardial, intraperitoneal, intrapleural, intraprostatical, intrarectal, intrathecal, intratracheal, intratumoral, intraumbilical, intravaginal, intravenous, intravesicularl, intravitreal, liposomal, local, mucosal, parenteral, rectal, subconjunctival, subcutaneous, sublingual, topical, transbuccal, transdermal, vaginal, via a catheter, via a lavage, via continuous infusion, via infusion, via inhalation, via injection, via local delivery, or via localized perfusion administration. In some embodiments, the composition is formulated for oral, topical, intraarterial, intraperitoneal, or intravenous administration. In some embodiments, the composition is formulated for oral administration. In some embodiments, the composition is formulated as a hard capsule, a soft capsule, a tablet, a syrup, a suspension, an emulsion, a solution, a solid dispersion, a wafer, or an elixir. In other embodiments, the composition is formulated for intraperitoneal administration. In other embodiments, the composition is formulated for intravenous administration. In some embodiments, the composition further comprises an agent which improves the solubility of the compound.

In yet another aspect, the present invention provides a method of treating a disease or disorder in a patient in need thereof comprising administering to the patient a pharmaceutically effective amount of a compound or composition of the present disclosure described herein. In some embodiments, the disease is cancer. In some embodiments, the cancer is of the bladder, blood, bone, brain, breast, central nervous system, cervix, colon, endometrium, esophagus, gall bladder, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. In some embodiments, the cancer is a carcinoma, sarcoma, leukemia, melanoma, mesothelioma, multiple myeloma, or seminoma. In some embodiments, the cancer is leukemia. In some embodiments, the leukemia is acute myelogenous leukemia, acute lymphocytic leukemia, chronic lymphocyctic leukemia, chronic myelogenous leukemia, hairy cell leukemia, T-cell prolymphocytic leukemia, large granular lymphocytic leukemia, or adult T-cell leukemia. In some embodiments, the leukemia is chronic myelogenous leukemia. In some embodiments, the leukemia produces leukemia stem cells. In some embodiments, the treatment comprises inducing apoptosis in leukemia stem cells. In some embodiments, the method comprises administering a second therapeutic agent or modality. In some embodiments, the method comprises administering a second chemotherapeutic agent. In some embodiments, the second chemotherapeutic agent is imatinib. In some embodiments, the second therapeutic agent or modality is surgery, radiotherapy, or immunotherapy. In some embodiments, the patient is a mammal. In some embodiments, the patient is a human. In some embodiments, the patient is resistant to imitanib.

In yet another aspect, the present invention provides a method of treating a disease or disorder associated with the peroxisome proliferator-activator receptor γ (PPARγ) in a patient in need thereof comprising administering to the patient a pharmaceutically effective amount of a compound or composition of the present disclosure described herein. Some non-limiting examples of diseases or disorders associated with PPAR γ are cancer and inflammatory conditions.

In another aspect, the present invention provides a method of treating leukemia by inducing apoptosis in leukemia stem cells in a patient in need thereof comprising administering to the patient a pharmaceutically effective amount of a compound or composition of the present disclosure described herein.

In yet another aspect, the present invention provides a method of preparing a compound of the formula:

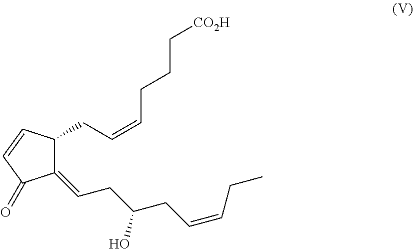 [SMILES: CC/C=C\C[C@H](O)C/C=C1/C(=O)C=C[C@@H]1C/C=C\CCCC(=O)O]

wherein: the method comprises the steps of a) reacting a compound of the formula:

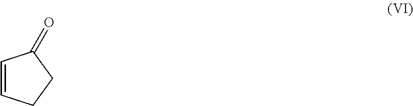

to form a compound of the formula:

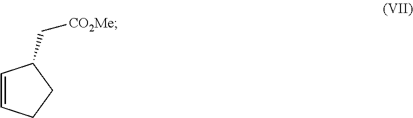

b) oxidizing the compound of formula VII to form a compound of the formula:

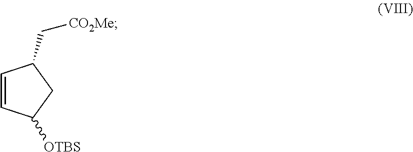 [SMILES: COC(=O)C[C@H]1C=CC(O[Si](C)(C)C(C)(C)C)C1]

c) reacting the compound of formula VIII to form a compound of the formula:

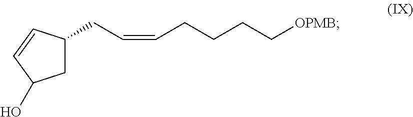

d) oxidizing the compound of formula IX to form a compound of the formula:

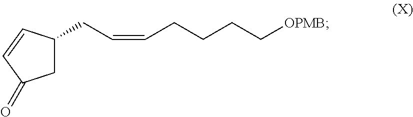

e) reacting the compound of formula X with a compound of the formula:

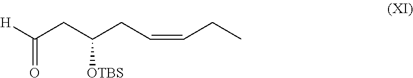 [SMILES: CC/C=C\C[C@@H](CC=O)O[Si](C)(C)C(C)(C)C]

to form a compound of the formula:

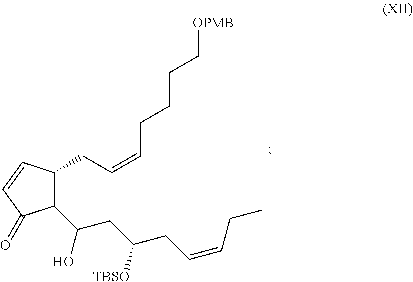

f) dehydrating the compound of formula XII to form a compound of the formula:

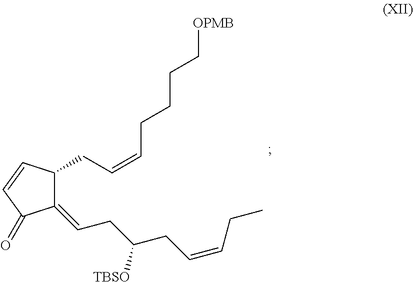

and
g) oxidizing the compound of formula XIII to give a compound of the formula:

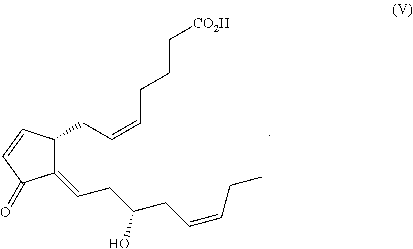 [SMILES: CC/C=C\C[C@H](O)C/C=C1/C(=O)C=C[C@@H]1C/C=C\CCCC(=O)O]

In some embodiments, step a) further comprises the following steps:
a₁) reducing a compound of the chemical formula comprising adding the compound with a reducing agent:

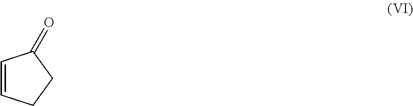

to give a compound of the formula:

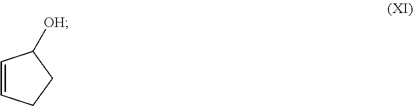

a₂) acylating the compound of formula XI to give a compound of the formula:

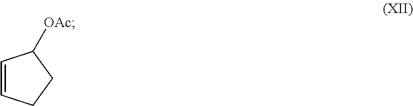

a₃) reacting the compound of formula XII with dimethyl malonate, a ligand and a metal salt to form a compound of the formula:

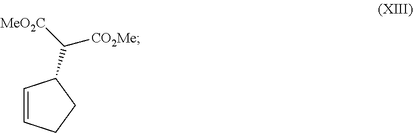 [SMILES: COC(=O)C(C(=O)OC)[C@H]1C=CCC1]

and
a₄) reacting with heat and potassium iodide the compound of formula XIII to form a compound of the formula:

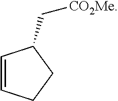

In some embodiments, the reducing agent is sodium borohydride or lithium aluminum hydride. In some embodiments, the reducing agent is lithium aluminum hydride. In some embodiments, about 0.5 equivalents of lithium aluminum hydride are used in step a₁). In some embodiments, step a₁) is reacted for a time period from about 5 to about 90 minutes. In some embodiments, the time period is about 10 minutes. In some embodiments, step a₁) is reacted at room temperature. In some embodiments, the acylation in step a₂) comprises reacting the compound of formula XI with acetic anhydride to form an acetyl ester. In some embodiments, about 2 equivalents of acetic anhydride is used in step a₂). In some embodiments, the acylation in step a₂) further comprises using one or more nitrogenous base. In some embodiments, the nitrogenous base is 4,4-dimethylaminopyridine, 1,8-diazabicyclo[5.4.0]undec-7-ene, diisopropylethylamine, or triethylamine. In some embodiments, the nitrogenous base is 4,4-dimethylaminopyridine and triethylamine. In some embodiments, about 0.1 equivalents of 4,4-dimethylaminopyridine are used in the reaction in step b). In some embodiments, about 2.5 equivalents of triethylamine are used in the reaction in step a₂). In some embodiments, the reaction in step a₂) proceeds from about 12 hours to about 24 hours. In some embodiments, the reaction in step a₂) proceeds for about 18 hours. In some embodiments, the total yield for steps a₁) and a₂) is greater than 50%. In some embodiments, the total yield is greater than 65%. In some embodiments, step a₃) further comprises a base. In some embodiments, the base is a metal carbonate. In some embodiments, the base is cesium carbonate. In some embodiments, about 3.0 equivalents of base are used in step a₃). In some embodiments, the ligand has the formula:

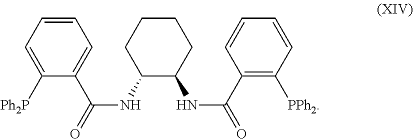

In some embodiments, about 0.015 equivalents of the ligand are used in step a₃). In some embodiments, the metal salt is a palladium salt. In some embodiments, the palladium salt is [(η-C₃H₅)₂PdCl]₂. In some embodiments, 0.005 equivalents of the metal salt are used. In some embodiments, the method further comprises allowing the reaction to proceed for about 3 hours. In some embodiments, step a₃) gives a yield greater than 50%. In some embodiments, step a₃) gives a yield greater than 70%. In some embodiments, step a₃) gives an enantiomeric excess of greater than 90%. In some embodiments, step a₃) gives an enantiomeric excess of greater than 95%. In some embodiments, step a₃) gives an enantiomeric excess equal to or greater than 97%. In some embodiments, step a₄) further comprises a solvent. In some embodiments, the solvent used in step a₄) is a mixture of water and 1,3-dimethyl-2-imidazolidinone. In some embodiments, the mixture of water to 1,3-dimethyl-2-imidazolidinone is from about 1 to 5 to about 1 to 20. In some embodiments, the mixture of water to 1,3-dimethyl-2-imidazolidinone is about 1 to 10. In some embodiments, step a₄) further comprises heating to a temperature heated from about 120° C. to about 150° C. In some embodiments, the temperature is about 130° C. In some embodiments, step a₄) gives a yield greater than 75%. In some embodiments, step a₄) gives a yield greater than 90%. In some embodiments, step a₄) further comprises reacting the reaction mixture for a time period of about 6 hours to about 24 hours. In some embodiments, the time period is about 12 hours. In some embodiments, step b) further comprises the following steps: b₁) oxidizing the compound of formula VII to form a compound of the formula:

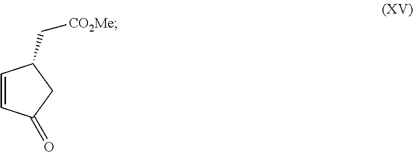 [SMILES: COC(=O)C[C@H]1C=CC(=O)C1]

b₂) reducing the compound of formula XV to give a compound of the formula:

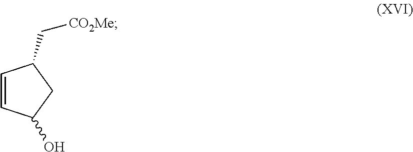 [SMILES: COC(=O)C[C@H]1C=CC(O)C1]

and
b₃) silylating the compound of formula XVI to give a compound of the formula:

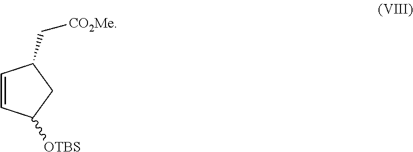 [SMILES: COC(=O)C[C@H]1C=CC(O[Si](C)(C)C(C)(C)C)C1]

In some embodiments, the oxidation of step b₁) further comprises uses a peroxide as an oxidant. In some embodiments, the peroxide is an alkyl hydroperoxide_{(C≦12)}. In some embodiments, the peroxide is tert-butyl hydroperoxide. In some embodiments, the oxidation of step b₁ comprises adding about 5 equivalents based upon the amount of formula VII used of tert-butyl hydroperoxide. In some embodiments, step b₁) further comprises a transition metal catalyst and a base. In some embodiments, the transition metal catalyst is Rh₂(cap)₄. In some embodiments, the oxidation of step b₁ comprises adding 0.001 equivalents based upon the amount of formula VII used of Rh₂(cap)₄. In some embodiments, the base is a metal carbonate. In some embodiments, the base is potassium carbonate. In some embodiments, the oxidation of step b₁ comprises adding about 0.5 equivalents based upon the amount of formula VII used of base. In some embodiments, the reaction in step b₁ is run under an oxygen atmosphere. In some embodiments, step b₁ comprises first admixing the transition metal catalyst and the base with the alkylperoxide_{(C≦12)} and allowing the mixture to react for about 1.5 hours and then a second addition of the same amount of transition metal catalyst and alkylperoxide_{(C≦12)} is added and reacted for about an additional 1.5 hours. In some embodiments, the yield of step b₁ is greater than 40%. In some embodiments, reduction of step b₂ further comprises using a metal hydride as a reducing agent. In some embodiments, the metal hydride is sodium borohydride. In some embodiments, the reduction of step b₂ comprises adding about 1 equivalent based upon the amount of formula XV used of the reducing agent. In some embodiments, the reduction of step b₂ further comprises using a solvent. In some embodiments, the solvent is an alcohol_{(C≦6)}. In some embodiments, the alcohol_{(C≦6)} is methanol. In some embodiments, the reduction of step b₂ further comprises cooling the reaction to a temperature from about −78° C. to about 0° C. In some embodiments, the temperature is about −30° C. In some embodiments, the reduction of step b₂ further comprises reacting the compound of the formula XV and the metal hydride for a time period from about 1 minute to about 1 hour. In some embodiments, the time period is about 10 minutes. In some embodiments, the reduction of step b₂ further comprises adding a metal salt to the reaction. In some embodiments, the metal salt is CeCl₃ or a hydrate thereof. In some embodiments, the reduction of step b₂ comprises adding about 1 equivalent based upon the amount of formula XV used of the metal salt. In some embodiments, the silylation of step b₃ comprises adding a silylating agent and a base. In some embodiments, the silylating agent is t-butyldimehtylsilyl chloride. In some embodiments, silyation of step b₃ comprises adding about 1.5 equivalents based upon the amount of formula XVI used of the silylating agent. In some embodiments, the base is an imidazole. In some embodiments, the silylation of step b₃ comprises adding about 3.0 equivalents based upon the amount of formula XVI used of the base. In some embodiments, the silylation of step b₃ further comprises using a solvent. In some embodiments, the solvent is a haloalkane_{(C≦6)}. In some embodiments, the solvent is CH₂Cl₂. In some embodiments, the silylation of step b₃ further comprises cooling the reaction to a temperature from about −30° C. to about 50° C. In some embodiments, the temperature is about 0° C. In some embodiments, the temperature is about 25° C. In some embodiments, the temperature is allowed to warm from about 0° C. to about 25° C. In some embodiments, the silylation of step b₃ further comprises reacting the compound of formula XVI and the silylating agent for a time period from about 5 minutes to about 1 hour. In some embodiments, the time period is about 15 minutes. In some embodiments, the method further comprising beginning to measure the time after the reaction has warmed to about 25° C. In some embodiments, step c) comprises the following steps: c₁) reducing a compound of the formula:

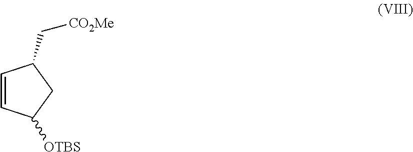 [SMILES: COC(=O)C[C@H]1C=CC(O[Si](C)(C)C(C)(C)C)C1]

to form a compound of the formula:

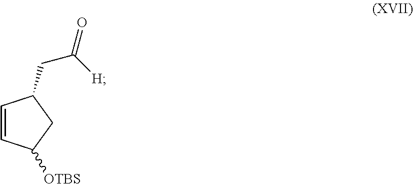

c₂) reacting the compound of formula (XVII) with a compound of the formula:

IPh₃P(CH₂)₅OPMB (XVIII)

to form a compound of the formula:

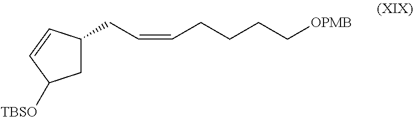

c₃) deprotecting the compound of formula XIX to give a compound of the formula:

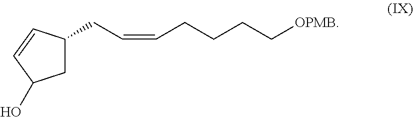

In some embodiments, the reduction in step c₁ further comprises adding a reducing agent. In some embodiments, the reducing agent is a metal hydride. In some embodiments, the reducing agent is DIBAl-H. In some embodiments, the reduction in step c₁ comprises adding about 1.1 equivalents of the reducing agent. In some embodiments, the reduction in step c₁ further comprises a solvent. In some embodiments, the solvent is a haloalkane_{(C≦6)}. In some embodiments, the solvent is CH₂Cl₂. In some embodiments, reduction in step c₁ further comprises cooling the reaction to a temperature from about −100° C. to about 0° C. In some embodiments, the reduction in step c₁ is reacted at about −78° C. In some embodiments, the reduction in step c₁ comprises reacting the compound of formula VIII and the reducing agent for a time period from about 15 minutes to about 2 hours. In some embodiments, the time period is about 45 minutes. In some embodiments, the reaction in step c₂ further comprises a base. In some embodiments, the base is an alkyllithium_{(C≦12)}, a metal amide_{(C≦12)}, or a metal silylamide_{(C≦12)}. In some embodiments, the base is sodium bis(trimethylsilyl)amide. In some embodiments, the reduction in step c₂ comprises adding about 2 equivalents of the base. In some embodiments, the reduction in step c₂ comprises adding about 1.5 equivalents of the compound of formula XVII. In some embodiments, the reduction in step c₂ cooling the reaction to a temperature from about −100° C. to about 50° C. In some embodiments, the temperature is about −78° C. In some embodiments, the method further comprising allowing the reaction to warm to about room temperature after 1 hour at about −78° C. In some embodiments, the reaction in step c₂ comprises reacting the compounds of formulas XVII and XVII for a time period from about 2 hours to about 12 hours. In some embodiments, the time period is about 6 hours. In some embodiments, the reaction in step c₂ further comprises a solvent. In some embodiments, the solvent is an ether_{(C≦12)}. In some embodiments, the ether is tetrahydrofuran. In some embodiments, the yield of steps c₁ and c₂ is greater than 70%. In some embodiments, the yield is greater than 90%. In some embodiments, deprotection of step c₃ further comprises adding a fluoride source. In some embodiments, the fluoride source is tetrabutylammonium fluoride. In some embodiments, the deprotection of step c₃ comprise adding about 1.2 equivalents of the fluoride source. In some embodiments, the deprotection further comprises a solvent. In some embodiments, the solvent is an organic solvent. In some embodiments, the solvent is an ether_{(C≦12)}. In some embodiments, the solvent is tetrahydrofuran. In some embodiments, the reaction in step c₃ is reacted for a time period from about 2 hours to about 12 hours. In some embodiments, the time period is about 5 hours. In some embodiments, the deprotection of step c₃ further comprise cooling the reaction to a temperature from about −30° C. to about 25° C. In some embodiments, the temperature is about 0° C. In some embodiments, the method further comprises warming the deprotection to room temperature after addition of the fluoride source. In some embodiments, the deprotection of step c₃ has a yield of greater than 80%. In some embodiments, the yield of step c₃ is greater than 90%. In some embodiments, the oxidation of step d) further comprises adding an oxidizing agent. In some embodiments, the oxidizing agent is pyridinium chlorochromate. In some embodiments, the oxidation of step d) comprises adding about 2 equivalents of the oxidizing agent. In some embodiments, the oxidation of step d) further comprises a solvent. In some embodiments, the solvent is an organic solvent. In some embodiments, the solvent is dichloromethane. In some embodiments, the oxidation of step d) is reacted at about room temperature. In some embodiments, the oxidation of step d) is reacted a time period from about 30 minutes to about 8 hours. In some embodiments, the time period is about 3 hours. In some embodiments, the oxidation of step d) has a yield of greater than 75%. In some embodiments, the yield is greater than 85%. In some embodiments, the yield is greater than 90%. In some embodiments, the reaction of step e) further comprises adding a base. In some embodiments, the base is a metal amide_{(C≦12)} or alkyllithium_{(C≦12)}. In some embodiments, the base is lithium diisopropylamide. In some embodiments, the reaction of step e) comprises adding about 2 equivalents of base. In some embodiments, the reaction of step e) comprises adding about 1.2 equivalents of the compound of formula XI. In some embodiments, the reaction of step e) comprises cooling the reaction to a temperature from about −100° C. to about −30° C. In some embodiments, the reaction of step e) reacted at about −78° C. In some embodiments, the reaction of step e) further comprises a solvent. In some embodiments, the solvent is an ether_{(C≦12)}. In some embodiments, the solvent is tetrahydrofuran. In some embodiments, the reaction of step e) is reacted for a time period from about 5 minutes to about 4 hours. In some embodiments, the time period is about 30 minutes. In some embodiments, the reaction of step e) has a yield greater than about 50%. In some embodiments, the yield is greater than 75%. In some embodiments, the reaction of step 1) further comprises the following steps: f₁) reacting a compound of formula VIII to form a compound of the formula:

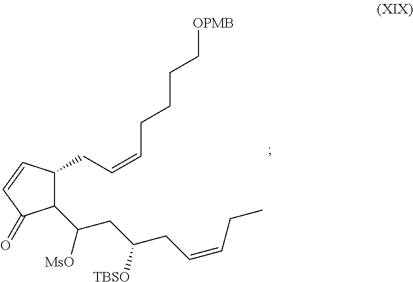

and
f₂) reacting a compound of formula XIX to form a compound of the formula:

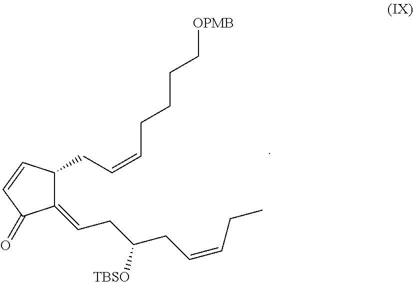

In some embodiments, the reaction of step f₁ further comprises reacting the compound of formula VIII with a group which enhances the ability of the hydroxyl group to be eliminated. In some embodiments, the group which enhances the ability of the hydroxyl group to be eliminated is methanesulfonyl chloride. In some embodiments, the method of step f₁ comprising adding about 5 equivalents of methanesulfonyl chloride. In some embodiments, the reaction of step f₁ further comprises a base. In some embodiments, the base is triethylamine. In some embodiments, the reaction of step f₁ comprises adding about 10 equivalents of base. In some embodiments, the reaction of f₁ further comprises a solvent. In some embodiments, the solvent is an organic solvent. In some embodiments, the solvent is a haloalkane_{(C≦12)} or an ether_{(C≦12)}. In some embodiments, the solvent is dichloromethane. In some embodiments, the reaction of step f₁ is reacted at a temperature from about −30° C. to about 25° C. In some embodiments, the temperature is about 0° C. In some embodiments, the reaction of step f₁ is reacted for a time period from about 1 minute to about 1 hour. In some embodiments, the time period is about 5 minutes. In some embodiments, the reaction of step f₂ further comprises adding Al₂O₃ as a dehydrating agent. In some embodiments, the reaction of step f₂ comprises adding from about 20 to about 25 equivalents of Al₂O₃. In some embodiments, the reaction of step f₂ further comprises a solvent. In some embodiments, the solvent is an organic solvent. In some embodiments, the solvent is a haloalkane_{(C≦12)} or an ether_{(C≦12)}. In some embodiments, the solvent is dichloromethane. In some embodiments, the reaction of step f₂ is reacted at a temperature from about 0° C. to about 50° C. In some embodiments, the temperature is about 25° C. In some embodiments, the temperature is about room temperature. In some embodiments, the reaction of step f₂ is reacted for a time period from about 4 hours to about 24 hours. In some embodiments, the time period is about 8 hours. In some embodiments, the reaction of step 1) has a yield greater than about 50%. In some embodiments, the yield is greater than 60%. In some embodiments, the oxidation of step g) further comprises the following steps:
g₁) deprotecting the compound of the formula IX to form a compound of the formula:

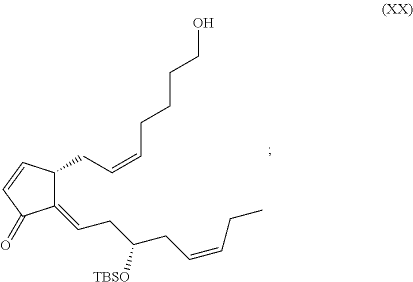 [SMILES: CC/C=C\C[C@@H](C/C=C1/C(=O)C=C[C@@H]1C/C=C\CCCCO)O[Si](C)(C)C(C)(C)C]

g₂) oxidizing the compound of formula XX with an oxidizing agent to give a compound of the formula:

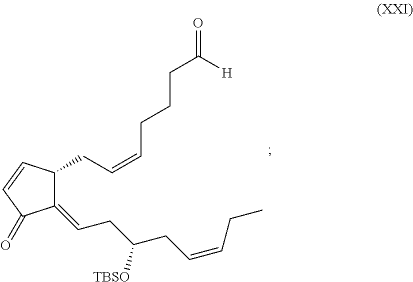 [SMILES: CC/C=C\C[C@@H](C/C=C1/C(=O)C=C[C@@H]1C/C=C\CCCC=O)O[Si](C)(C)C(C)(C)C]

g₃) oxidizing the compound of formula XXI with an oxidizing agent to give a compound of the formula:

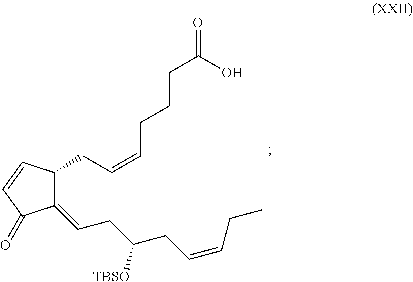 [SMILES: CC/C=C\C[C@@H](C/C=C1/C(=O)C=C[C@@H]1C/C=C\CCCC(=O)O)O[Si](C)(C)C(C)(C)C]

and
g₄) deprotecting the compound of formula XXII to give a compound of the formula:

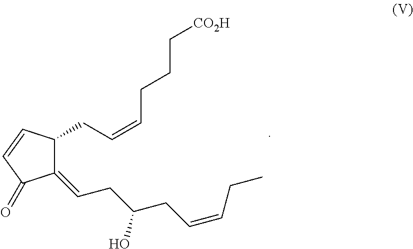 [SMILES: CC/C=C\C[C@H](O)C/C=C1/C(=O)C=C[C@@H]1C/C=C\CCCC(=O)O]

In some embodiments, the deprotection reaction of step g₁ further comprises using 2,3-dichloro-5,6-dicyano-1,4-benzoquinone (DDQ). In some embodiments, the deprotection reaction of step g₁ comprises adding about 1.5 equivalents of DDQ. In some embodiments, the deprotection of step g₁ further comprises a solvent. In some embodiments, the solvent is an organic solvent, water, or a mixture thereof. In some embodiments, the organic solvent is haloalkane_{(C≦12)}. In some embodiments, the haloalkane_{(C≦12)} is dichloromethane. In some embodiments, the solvent is a mixture of dichloromethane and water. In some embodiments, the mixture of dichloromethane to water is about 16 to 1. In some embodiments, the deprotection reaction of step g₁ is reacted at a temperature from about −30° C. to about 25° C. In some embodiments, the temperature is about 0° C. In some embodiments, the deprotection reaction of step g₁ is reacted for a time period from about 15 minutes to about 4 hours. In some embodiments, the time period is about 45 minutes. In some embodiments, the oxidizing agent of step g₂ is pyridinium chlorochromate. In some embodiments, the oxidation of step g₂ comprises adding about 2 equivalents of oxidizing agent. In some embodiments, the oxidation of step g₂ further comprises a solvent. In some embodiments, the solvent is an organic solvent. In some embodiments, the solvent is a haloalkane_{(C≦12)}. In some embodiments, the solvent is dichloromethane. In some embodiments, the oxidation of step g₂ is reacted for a time period from about 30 minutes to about 4 hours. In some embodiments, the time period is about 2 hours. In some embodiments, the oxidation of step g₂ is reacted at a temperature from about 0° C. to about 35° C. In some embodiments, the temperature is about 25° C. In some embodiments, the temperature is about room temperature. In some embodiments, the oxidizing agent of step g₃ is sodium chlorite. In some embodiments, the oxidation of step g₂ comprises adding about 1.5 equivalents of the oxidizing agent. In some embodiments, the oxidation of step g₃ further comprises sodium dihydrogen phosphate. In some embodiments, the oxidation of step g₃ comprises adding about 1.5 equivalents of sodium dihydrogen phosphate. In some embodiments, the oxidation of step g₃ further comprises 2-methyl-2-butene. In some embodiments, the oxidation of step g₃ comprises adding about 10 equivalents of 2-methyl-2-butene. In some embodiments, the oxidation of step g₃ further comprises a solvent. In some embodiments, the solvent is an organic solvent, water, or a mixture thereof. In some embodiments, the solvent is a water and organic mixture. In some embodiments, the organic solvent is an alcohol_{(C≦12)}. In some embodiments, the alcohol is tert-butanol. In some embodiments, the solvent is a mixture of tert-butanol and water. In some embodiments, the ratio of tert-butanol to water is about 4 to 3. In some embodiments, the oxidation of step g₃ is reacted at a temperature from about 0° C. to about 35° C. In some embodiments, the temperature is about 25° C. In some embodiments, the temperature is about room temperature. In some embodiments, the oxidation of step g₃ is reacted for a time period from about 10 minutes to about 2 hours. In some embodiments, the time period is about 30 minutes. In some embodiments, the reactions of steps g₁ to g₃ result in a yield of greater than about 60%. In some embodiments, the yield is greater than 70%. In some embodiments, the deprotection of step g₄ comprises adding an acid to the reaction. In some embodiments, the acid is aqueous hydrofluoric acid. In some embodiments, the acid is 50% aqueous hydrofluoric acid. In some embodiments, the deprotection of step g₄ comprises adding about 50 equivalents of acid. In some embodiments, the deprotection of step g₄ further comprises a solvent. In some embodiments, the solvent is an organic solvent. In some embodiments, the solvent is acetonitrile. In some embodiments, the deprotection of step g₄ is reacted at a temperature from about −30° C. to about 25° C. In some embodiments, the temperature is about 0° C. In some embodiments, the deprotection of step g₄ is reacted for a time period from about 30 minutes to 2 hours. In some embodiments, the time period is about 45 minutes. In some embodiments, the yield of the deprotection of step g₄ is greater than 80%. In some embodiments, the yield is greater than 90%. In some embodiments, the method further comprising step h) wherein a compound of the formula V is reacted to form a compound of the formula:

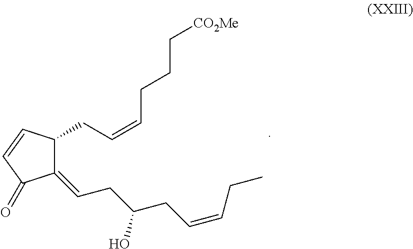 [SMILES: CC/C=C\C[C@H](O)C/C=C1/C(=O)C=C[C@@H]1C/C=C\CCCC(=O)OC]

In some embodiments, the reaction of step h) further comprises adding a methylating agent to the reaction. In some embodiments, the methylating agent is trimethylsilyldiazomethane. In some embodiments, the reaction of step h) comprises adding about 1.5 equivalents of methylating agent. In some embodiments, the reaction further comprises a solvent. In some embodiments, the solvent is an organic solvent. In some embodiments, the solvent is a mixture of organic solvents. In some embodiments, the mixture of organic solvents comprises benzene and methanol. In some embodiments, the ratio of benzene to methanol is 3 to 2.

In some embodiments, the reaction of step h) is reacted for a time period from about 10 minutes to about 2 hours. In some embodiments, the time period is about 30 minutes. In some embodiments, the reaction of step h) is reacted at a temperature from about 0° C. to about 35° C. In some embodiments, the temperature is about 25° C. In some embodiments, the temperature is about room temperature. In some embodiments, the reaction of step h) has a yield greater than about 80%. In some embodiments, the yield is greater than about 90%. In some embodiments, the method further comprising step h) wherein a compound of the formula V is reacted to form a compound of the formula:

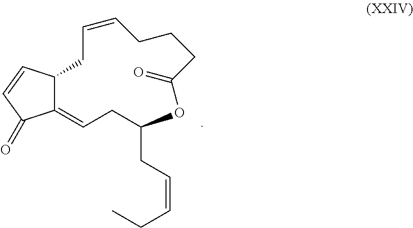 [SMILES: CC/C=C\C[C@H]1C/C=C2/C(=O)C=C[C@@H]2C/C=C\CCCC(=O)O1]

In some embodiments, the reaction of step h) further comprises adding an activating agent to the reaction. In some embodiments, the activating agent is 2-methyl-6-nitrobenzoic anhydride. In some embodiments, the reaction of step h) comprises adding about 1.4 equivalents of activating agent. In some embodiments, the reaction of step h) further comprises adding 4,4-dimethylaminopyridine. In some embodiments, the reaction of step h) further comprises adding about 6 equivalents of 4,4-dimethylaminopyridine. In some embodiments, the reaction further comprises a solvent. In some embodiments, the solvent is an organic solvent. In some embodiments, the solvent is haloalkane_{(C≦12)}. In some embodiments, the solvent is dichloromethane. In some embodiments, the reaction of step h) is reacted for a time period from about 12 hours to about 36 hours. In some embodiments, the reaction of step h) comprises adding the compound of formula V dropwise over 15 hours. In some embodiments, the reaction of step h) comprises further reacting the compound of formula V for about 2 hours after the compound has finished added. In some embodiments, the time period is about 30 minutes. In some embodiments, the reaction of step h) is reacted at a temperature from about 0° C. to about 35° C. In some embodiments, the temperature is about 25° C. In some embodiments, the temperature is about room temperature. In some embodiments, the reaction of step h) has a yield greater than about 80%. In some embodiments, the yield is greater than about 90%. In some embodiments, the compound of formula VII is prepared by a method comprising the following steps:

a) reacting a compound of the formula:

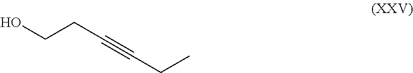

with a compound of the formula:

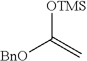

to form a compound of the formula:

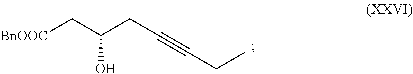

and
b) reducing a compound of formula X to form a compound of the formula:

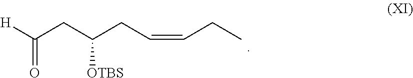 [SMILES: CC/C=C\C[C@@H](CC=O)O[Si](C)(C)C(C)(C)C]

In some embodiments, step a) is further comprised by the steps of
a₂₋₁) oxidizing a compound of the formula:

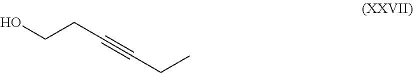

to form a compound of the formula:

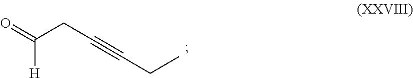

and
a₂₋₂) reacting a compound of the formula

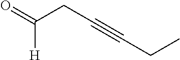

with a compound of the formula:

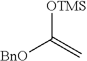

to form a compound of the formula:

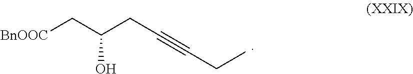

In some embodiments, the oxidation of step a₂₋₁ further comprises an oxidizing agent. In some embodiments, oxidizing agent is 1,1,1-triacetoxy-1,1-dihydro-1,2-benziodoxol-3(1H)-one. In some embodiments, the oxidation of step a₂₋₁ comprises adding about 1.3 equivalents of the oxidizing agent. In some embodiments, the oxidation of step a₂₋₁ further comprises a solvent. In some embodiments, the solvent is an organic solvent. In some embodiments, the solvent is a haloalkane_{(C≦12)}. In some embodiments, the solvent is dichloromethane. In some embodiments, the oxidation of step a₂₋₁ is reacted at a temperature from about −35° C. to about 35° C. In some embodiments, the temperature is about 0° C. In some embodiments, after five minutes at 0° C., the reaction is warmed to about room temperature. In some embodiments, the oxidation reaction of step a₂₋₁ is reacted for a time period from about 30 minutes to about 4 hours. In some embodiments, the time period is about 1.5 hours. In some embodiments, the oxidation reaction of step a₂₋₁ has a yield of greater than 80%. In some embodiments, the yield is greater than 90%. In some embodiments, the reaction in step a₂₋₂ further comprises a transition metal catalyst. In some embodiments, the transition metal catalyst contains a titanium metal ion. In some embodiments, the transition metal catalyst is prepared by reacting about 2 equivalents of 2′-{[3-bromo-5-(t-butyl)benzylidene]amino}-[(R)-1,1′-binaphthalen]-2-ol with about 1 equivalent of titanium tetraisopropoxide. In some embodiments, the reaction is stirred at room temperature for about 1 hour. In some embodiments, the method further comprising adding about 1 equivalent of 3,5-di-t-buyl-salicylic acid to reaction. In some embodiments, the reaction is stirred for about 1 hour after the addition of the 3,5-di-t-butyl-salicylic acid. In some embodiments, the transition metal catalyst is prepared at about room temperature. In some embodiments, the transition metal catalyst is prepared in a solvent. In some embodiments, the solvent is toluene. In some embodiments, the reaction of step a₂₋₂ comprises adding about 2 equivalents of

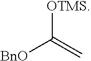

In some embodiments, the reaction is reacted at a temperature from about −100° C. to about 0° C. In some embodiments, the temperature is about −78° C. In some embodiments, the reaction is allowed to warm over about an hour to a temperature of about −15° C. In some embodiments, the reaction of step a₂₋₂ is reacted for a time period from about 2 hours to about 8 hours. In some embodiments, the reaction of step a₂₋₂ is reacted for about 4 hours at a temperature of about −15° C. In some embodiments, the reaction in step a₂₋₂ further comprises an acidic workup. In some embodiments, the acidic workup comprises quenching the reaction with a saturated solution of weak acid solution and is extracted with an ether_{(C≦12)}. In some embodiments, the method further comprising concentrating the ether_{(C≦12)} to form a crude solid of an intermediate. In some embodiments, the method further comprising dissolving the intermediate into a solution with tetrahydrofuran and tetrabutylammonium fluoride. In some embodiments, the solution is stirred for a time period from about 10 minutes to about 2 hours. In some embodiments, the time period is about 30 minutes. In some embodiments, the reaction is reacted at a temperature from about 0° C. to about 35° C. In some embodiments, the temperature is about 25° C. In some embodiments, the temperature is about room temperature. In some embodiments, the reaction comprises adding about four equivalents of tetrabutylammonium fluoride. In some embodiments, the reaction results in a yield greater than 50%. In some embodiments, the yield is greater than 70%. In some embodiments, the reaction product has an enantiomeric excess of greater than 80%. In some embodiments, the enantiomeric excess of the reaction is greater than 90%. In some embodiments, the enantiomeric excess of the reaction is at least 95%. In some embodiments, step b) further comprises the following steps:
B₂₋₁) protecting the compound of formula XXVI to form a compound of the formula:

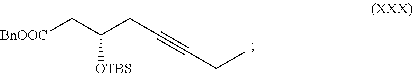

B₂₋₂) reducing the compound of step BO to form a compound of the formula:

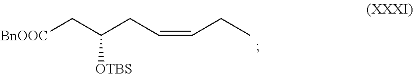

B₂₋₃) reducing the compound of step B₂₋₂) to form a compound of the formula:

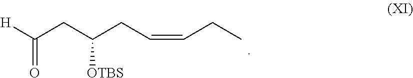 [SMILES: CC/C=C\C[C@@H](CC=O)O[Si](C)(C)C(C)(C)C]

In some embodiments, the protection of step b₂₋₁ further comprises reacting the compound of formula XXVI with tributylsilyl chloride. In some embodiments, the protection of step b₂₋₁ comprises adding about 2 equivalents of tributylsilyl chloride. In some embodiments, step b₂₋₁ further comprises adding a base to the reaction. In some embodiments, the base is imidazole. In some embodiments, step b₂₋₁ comprises adding about 3 equivalents of base. In some embodiments, the protection reaction of step b₂₋₁ further comprises a solvent. In some embodiments, the solvent is an organic solvent. In some embodiments, the solvent is dichloromethane. In some embodiments, the protection reaction of step b₂₋₁ reacts for a time period from about 1 hour to 6 hours. In some embodiments, the protection reaction reacts for about 3 hours. In some embodiments, the protection reaction of step b₂₋₁ reacts at a temperature from about 0° C. to about 35° C. In some embodiments, the temperature is about 25° C. In some embodiments, the temperature is about room temperature. In some embodiments, the protection reaction of step b₂₋₁ results in a yield of greater than 75%. In some embodiments, the yield is greater than 85%. In some embodiments, the reduction of step b₂₋₂ further comprises reacting the compound of formula XXVI with a reducing agent. In some embodiments, the reducing agent is hydrogen gas (H₂). In some embodiments, the reduction of step b₂₋₂ comprises adding about an atmosphere of H₂ gas. In some embodiments, step b₂₋₂ further comprises adding a catalyst to the reaction. In some embodiments, the catalyst is Lindlar's catalyst. In some embodiments, step b₂₋₂ comprises adding about 0.1 equivalents of catalyst. In some embodiments, step b₂₋₂ further comprises adding quinoline to the reaction. In some embodiments, step b₂₋₂ comprises adding about 1 equivalents of quinoline. In some embodiments, the reduction of step b₂₋₂ further comprises a solvent. In some embodiments, the solvent is an organic solvent. In some embodiments, the solvent is ethyl acetate. In some embodiments, the reduction of step b₂₋₂ reacts for a time period from about 5 minutes to 2 hours. In some embodiments, the time period is about 30 minutes. In some embodiments, the reduction of step b₂₋₂ reacts at a temperature from about 0° C. to about 35° C. In some embodiments, the temperature is about 25° C. In some embodiments, the temperature is about room temperature. In some embodiments, the reduction of step b₂₋₂ results in a yield of greater than 75%. In some embodiments, the yield is greater than 85%. In some embodiments, the reduction of step b₂₋₃ further comprises reacting the compound of formula XXVI with a reducing agent. In some embodiments, the reducing agent is a metal hydride. In some embodiments, the metal hydride is DIBAl-H. In some embodiments, the reduction of step b₂₋₃ comprises adding about 1.3 equivalents of reducing agent. In some embodiments, the reduction of step b₂₋₃ further comprises a solvent. In some embodiments, the solvent is an organic solvent. In some embodiments, the solvent is a haloalkane_{(C≦12)}. In some embodiments, the solvent is dichloromethane. In some embodiments, the reduction of step b₂₋₃ reacts for a time period from about 45 minutes to 4 hours. In some embodiments, the time period is about 1 hour. In some embodiments, the reduction of step b₂₋₃ reacts at a temperature from about −100° C. to about 35° C. In some embodiments, the temperature is about −78° C. In some embodiments, the reduction of step b₂₋₃ is allowed to warmed to −25° C. over 1 hour. In some embodiments, the reduction of step b₂₋₃ results in a yield of greater than 75%. In some embodiments, the yield is greater than 85%.

In another aspect, the present invention provides a method of preparing a compound comprised by reacting a base with a compound of the formula:

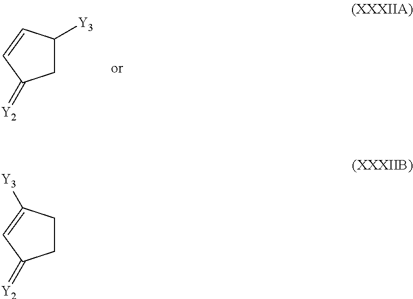

wherein: Y₂ is O, S, NH, or NA₁ wherein: A₁ is alkyl_{(C≦6)}, alkoxy_{(C≦6)}, or a substituted version of any of these groups; Y₃ is alkyl_{(C≦18)}, alkenyl_{(C≦18)}, alkynyl_{(C≦18)}, aryl_{(C≦18)}, aralkyl_{(C≦18)}, heteroaryl_{(C≦18)}, heteroaralkyl_{(C≦18)}, heterocycloalkyl_{(C≦18)}, or a substituted version of any these groups; —X₁-A₂-R₁; wherein: X₁ is alkanediyl_{(C≦8)}, alkenediyl_{(C≦8)}, alkynediyl_{(C≦8)}, arenediyl_{(C≦12)}, heteroarenediyl_{(C≦12)}, or a substituted version of any of these groups; A₂ is a covalent bond, O, S, S(O), S(O)₂, NH, or NR₂; wherein R₂ is alkyl_{(C≦6)} or substituted alkyl_{(C≦6)}; and R₁ is alkyl_{(C≦12)}, alkenyl_{(C≦12)}, alkynyl_{(C≦12)}, aryl_{(C≦12)}, aralkyl_{(C≦12)}, heteroaryl_{(C≦12)}, heteroaralkyl_{(C≦12)}, heterocycloalkyl_{(C≦12)}, acyl_{(C≦12)}, or a substituted version of any of these groups; and then adding a compound of the formula:

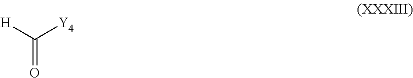

wherein: Y₄ is alkyl_{(C≦18)}, alkenyl_{(C≦18)}, alkynyl_{(C≦18)}, aryl_{(C≦18)}, aralkyl_{(C≦18)}, heteroaryl_{(C≦18)}, heteroaralkyl_{(C≦18)}, heterocycloalkyl_{(C≦18)}, or a substituted version of any these groups; or —X₂-A₃-R₃; wherein: X₂ is alkanediyl_{(C≦8)}, alkenediyl_{(C≦8)}, alkynediyl_{(C≦8)}, arenediyl_{(C≦12)}, heteroarenediyl_{(C≦12)}, or a substituted version of any of these groups; A₃ is a covalent bond, O, S, S(O), S(O)₂, NH, or NR₄; wherein R₄ is alkyl_{(C≦6)} or substituted alkyl_{(C≦6)}; and R₃ is alkyl_{(C≦12)}, alkenyl_{(C≦12)}, alkynyl_{(C≦12)}, aryl_{(C≦12)}, aralkyl_{(C≦12)}, heteroaryl_{(C≦12)}, heteroaralkyl_{(C≦12)}, heterocycloalkyl_{(C≦12)}, acyl_{(C≦12)}, or a substituted version of any of these groups; to form a compound of the formula:

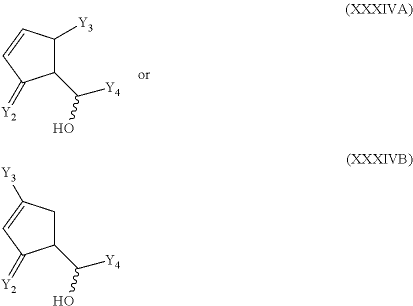

wherein Y₂, Y₃, and Y₄ are as defined above. In some embodiments, Y₂ is O. In some embodiments, Y₃ is alkyl_{(C≦18)}, alkenyl_{(C≦18)}, alkynyl_{(C≦18)}, heteroaryl_{(C≦18)}, heteroaralkyl_{(C≦18)}, or a substituted version of any of these groups. In some embodiments, Y₃ is alkenyl_{(C≦18)} or substituted alkenyl_{(C≦18)}. In other embodiments, Y₃ is alkynyl_{(C≦18)} or substituted alkynyl_{(C≦18)}. In other embodiments, Y₃ is heteroaryl_{(C≦18)} or substituted heteroaryl_{(C≦18)}. In other embodiments, Y₃ is heteroaralkyl_{(C≦18)} or substituted heteroaralkyl_{(C≦18)}. In some embodiments, Y₄ is alkyl_{(C≦18)}, alkenyl_{(C≦18)}, alkynyl_{(C≦18)}, heteroaryl_{(C≦18)}, heteroaralkyl_{(C≦18)}, or a substituted version of any these groups. In some embodiments, Y₄ is alkenyl_{(C≦18)} or substituted alkenyl_{(C≦18)}. In some embodiments, Y₄ is alkynyl_{(C≦18)} or substituted alkynyl_{(C≦18)}. In some embodiments, Y₄ is heteroaralkyl_{(C≦18)} or substituted heteroaralkyl_{(C≦18)}. In some embodiments, the base is selected from a metal amide, an alkyllithium, or a metal hydride. In some embodiments, the base is lithium diisopropyl amide. In some embodiments, the method comprises adding from about 1.5 to about 3 equivalents of base. In some embodiments, the method comprises adding about 2 equivalents of base. In some embodiments, the method comprises a solvent. In some embodiments, the solvent is an organic solvent. In some embodiments, the solvent is haloalkane_{(C≦12)} or ether_{(C≦12)}. In some embodiments, the solvent is tetrahydrofuran. In some embodiments, the method is reacted at a temperature from about −100° C. to about 25° C. In some embodiments, the temperature is about 0° C. In some embodiments, the method is cooled from about 0° C. to about −78° C. after about 20 minutes. In some embodiments, the base is added before the compound of formula XXXIII. In some embodiments, the base is added and allowed to react with the compound XXXII for a time period from about 10 minutes to about 1 hour. In some embodiments, the time period is about 20 minutes. In some embodiments, the reaction is allowed to react a time period from about 10 minutes to about 1 hour after the addition of the compound of formula XXXIII. In some embodiments, the time period is about 30 minutes. In some embodiments, the reaction has a yield of greater than 50%. In some embodiments, the yield is greater than 70%. In some embodiments, the yield is greater than 75%.

In yet another aspect, the present invention provides a method of preparing a compound of the formula:

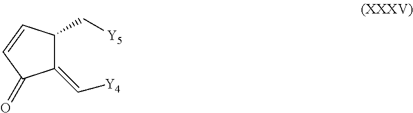

wherein: Y₄ is alkyl_{(C≦18)}, alkenyl_{(C≦18)}, alkynyl_{(C≦18)}, aryl_{(C≦18)}, aralkyl_{(C≦8)}, heteroaryl_{(C≦18)}, heterocycloalkyl_{(C≦8)}, or a substituted version of any these groups; Y₅ is hydrogen, hydroxy, amino, cyano, or; alkyl_{(C≦12)}, alkenyl_{(C≦12)}, alkynyl_{(C≦12)}, aryl_{(C≦12)}, heteroaryl_{(C≦12)}, heterocycloalkyl_{(C≦12)}, alkoxy_{(C≦12)}, alkenyloxy_{(C≦12)}, alkynyloxy_{(C≦12)}, aryloxy_{(C≦12)}, heteroaryloxy_{(C≦12)}, heterocycloalkyloxy_{(C≦12)}, acyloxy_{(C≦12)}, alkylamino_{(C≦12)}, dialkylamino_{(C≦12)}, alkenylamino_{(C≦12)}, alkynylamino_{(C≦12)}, arylamino_{(C≦12)}, heteroarylamino_{(C≦12)}, heterocycloalkylamino_{(C≦12)}, amido_{(C≦12)}, or a substituted version of any of these groups; or —C(O)NR₂R₃ or —C(O)R₂; wherein: R₂ and R₃ are each independently hydrogen, hydroxy, alkyl_{(C≦6)}, aryl_{(C≦8)}, alkoxy_{(C≦6)}, or a substituted version of any of the last three groups; and wherein: the method comprises the steps of a) reacting a compound of the formula:

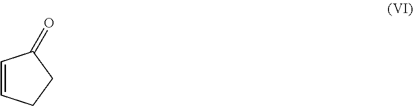

to form a compound of the formula:

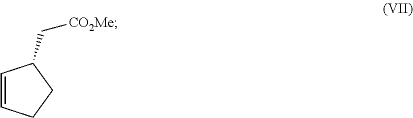

b) reacting the compound of formula VII to form a compound of the formula:

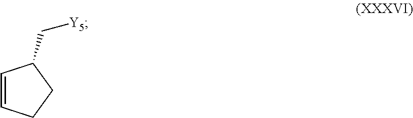

wherein: Y₅ is as defined above; c) oxidizing the compound of formula XXXVI to form a compound of the formula:

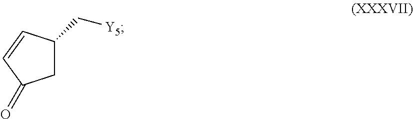

wherein: Y₅ is as defined above; d) reacting the compound of formula XXXVII with a compound of the formula:

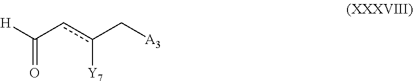

wherein: Y₇ is hydrogen, amino, hydroxy, mercapto, —OR₈, —SR₉, or —NR₁₀R₁₁; wherein: R₈ is alkyl_{(C≦12)}, aralkyl_{(C≦12)}, acyl_{(C≦12)}, a substituted version of any of these three groups, or a hydroxy protecting group; R₉ is alkyl_{(C≦12)}, aralkyl_{(C≦12)}, acyl_{(C≦12)}, a substituted version of any of these three groups, or a thiol protecting group; and R₁₀ and R₁₁ are each independently, alkyl_{(C≦12)}, aralkyl_{(C≦12)}, acyl_{(C≦12)}, a substituted version of any of these three groups, or a monovalent amino protecting group; or R₁₀ and R₁₁ are taken together to form a divalent amino protecting group; and A₃ is alkyl_{(C≦13)}, alkenyl_{(C≦13)}, alkynyl_{(C≦13)}, aralkyl_{(C≦13)}, heteroaralkyl_{(C≦13)}, or a substituted version of any of these groups; to form a compound of the formula:

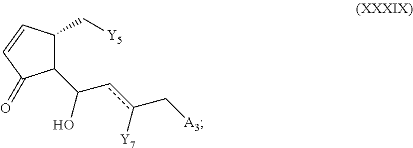

wherein: Y₅, Y₇, and A₃ are as defined above; e) dehydrating the compound of formula XXXIX to form a compound of the formula:

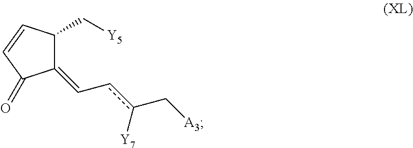

wherein: Y₅, Y₇, and A₃ are as defined above; and f) deprotecting the compound of formula XL to give a compound of the formula:

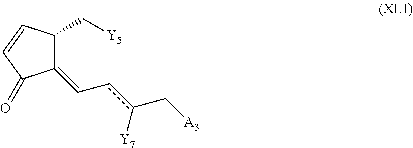

wherein: Y₅, Y₇, and A₃ are as defined above. In some embodiments, the method of preparing a compound of the formula:

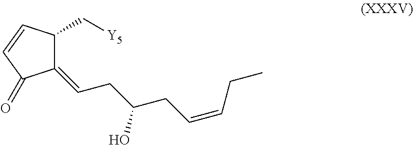

wherein: Y₅ is hydrogen, hydroxy, amino, cyano, or; alkyl_{(C≦12)}, alkenyl_{(C≦12)}, alkynyl_{(C≦12)}, aryl_{(C≦12)}, heteroaryl_{(C≦12)}, heterocycloalkyl_{(C≦12)}, alkoxy_{(C≦12)}, alkenyloxy_{(C≦12)}, alkynyloxy_{(C≦12)}, aryloxy_{(C≦12)}, heteroaryloxy_{(C≦12)}, heterocycloalkyloxy_{(C≦12)}, acyloxy_{(C≦12)}, alkylamino_{(C≦12)}, dialkylamino_{(C≦12)}, alkenylamino_{(C≦12)}, alkynylamino_{(C≦12)}, arylamino_{(C≦12)}, heteroarylamino_{(C≦12)}, heterocycloalkylamino_{(C≦12)}, amido_{(C≦12)}, or a substituted version of any of these groups; or —C(O)NR₂R₃ or —C(O)R₂; wherein: R₂ and R₃ are each independently hydrogen, hydroxy, alkyl_{(C≦6)}, aryl_{(C≦8)}, alkoxy_{(C≦6)}, or a substituted version of any of the last three groups; and wherein: the method comprises the steps of
a) reacting a compound of the formula:

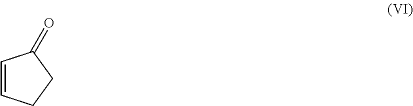

to form a compound of the formula:

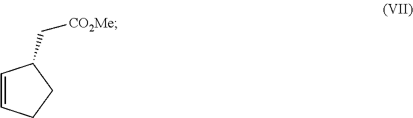

b) reacting the compound of formula VII to form a compound of the formula:

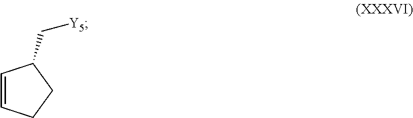

wherein: Y₅ is as defined above; c) oxidizing the compound of formula XXXVI to form a compound of the formula:

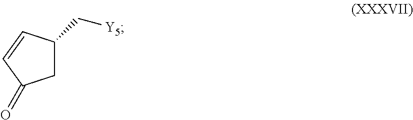

wherein: Y₅ is as defined above; d) reacting the compound of formula XXXVII with a compound of the formula:

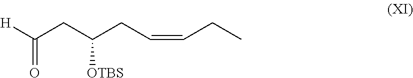 [SMILES: CC/C=C\C[C@@H](CC=O)O[Si](C)(C)C(C)(C)C]

to form a compound of the formula:

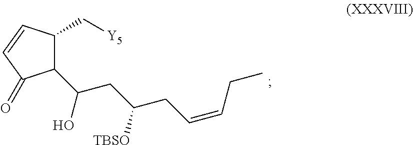

wherein: Y₅ is as defined above; e) dehydrating the compound of formula XXXVIII to form a compound of the formula:

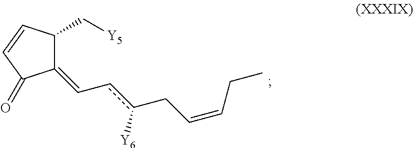

wherein: Y₅ is as defined above; and Y₆ is —OH, —OTBS, or —H; and f) deprotecting the compound of formula XXXIX to give a compound of the formula:

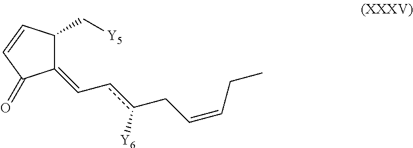

wherein: Y₅ is as defined above; and Y₆ is —OH or —H. In some embodiments, Y₅ is substituted alkyl_{(C≦6)}. In some embodiments, Y₅ is —CH₂OH. In other embodiments, Y₅ is —CH₂OTBS. In other embodiments, Y₅ is —C(O)NR₂R₃. In some embodiments, R₂ is alkyl_{(C≦6)}. In some embodiments, R₂ is —CH₃. In some embodiments, R₃ is alkoxy_{(C≦6)}. In some embodiments, R₃ is —OMe. In other embodiments, Y₅ is —C(O)N(OCH₃)CH₃. In some embodiments, Y₆ is —H. In some embodiments, Y₆ is —OH. In some embodiments, step a) further comprises the following steps:
a₁) reducing a compound of the chemical formula comprising adding the compound with a reducing agent:

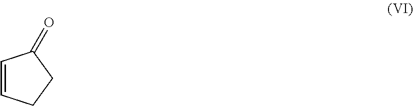

to give a compound of the formula:

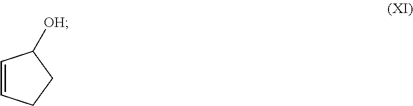

a₂) acylating the compound of formula XI to give a compound of the formula:

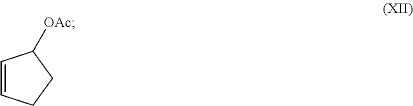

a₃) reacting the compound of formula XII with dimethyl malonate, a ligand and a metal salt to form a compound of the formula:

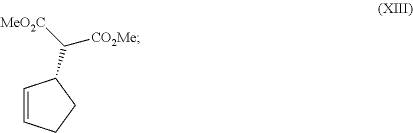 [SMILES: COC(=O)C(C(=O)OC)[C@H]1C=CCC1]

and
a₄) reacting with heat and potassium iodide the compound of formula XIII to form a compound of the formula:

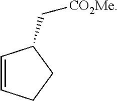

In some embodiments, the reducing agent is sodium borohydride or lithium aluminum hydride. In some embodiments, the reducing agent is lithium aluminum hydride. In some embodiments, about 0.5 equivalents of lithium aluminum hydride are used in step a₁). In some embodiments, step a₁) is reacted for a time period from about 5 to about 90 minutes. In some embodiments, the time period is 10 minutes. In some embodiments, step a₁) is reacted at room temperature. In some embodiments, the acylation in step a₂) comprises reacting the compound of formula XI with acetic anhydride to form an acetyl ester. In some embodiments, about 2 equivalents of acetic anhydride are used in step a₂). In some embodiments, the acylation in step a₂) further comprises using one or more nitrogenous base. In some embodiments, the nitrogenous base is 4,4-dimethylaminopyridine, 1,8-diazabicyclo[5.4.0]undec-7-ene, diisopropylethylamine, or triethylamine. In some embodiments, the nitrogenous base is 4,4-dimethylaminopyridine and/or triethylamine. In some embodiments, about 0.1 equivalents of 4,4-dimethylaminopyridine are used in the reaction in step a₂). In some embodiments, about 2.5 equivalents of triethylamine are used in the reaction in step a₂). In some embodiments, the reaction in step a₂) proceeds from about 12 hours to about 24 hours. In some embodiments, the reaction in step a₂) proceeds for about 18 hours. In some embodiments, the total yield for steps a₁) and a₂) is greater than 50%. In some embodiments, the total yield is greater than 65%. In some embodiments, step a₃) further comprises a base. In some embodiments, the base is a metal carbonate. In some embodiments, the base is cesium carbonate. In some embodiments, about 3.0 equivalents of base are used in step a₃). In some embodiments, the ligand has the formula:

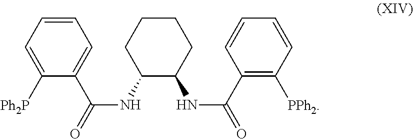

In some embodiments, about 0.015 equivalents of the ligand are used in step a₃). In some embodiments, the metal salt is a palladium salt. In some embodiments, the palladium salt is [(η-C₃H₅)₂PdCl]₂. In some embodiments, 0.005 equivalents of the metal salt are used. In some embodiments, the method further comprises allowing the reaction of step a₃) to proceed for about 3 hours. In some embodiments, step a₃) gives a yield greater than 50%. In some embodiments, step a₃) gives a yield greater than 70%. In some embodiments, step a₃) gives an enantiomeric excess of greater than 90%. In some embodiments, step a₃) gives an enantiomeric excess of greater than 95%. In some embodiments, step a₃) gives an enantiomeric excess equal to or greater than 97%. In some embodiments, step a₄) further comprises a solvent. In some embodiments, the solvent used in step a₄) is a mixture of water and 1,3-dimethyl-2-imidazolidinone. In some embodiments, the mixture of water to 1,3-dimethyl-2-imidazolidinone is from about 1 to 5 to about 1 to 20. In some embodiments, the mixture of water to 1,3-dimethyl-2-imidazolidinone is about 1 to 10. In some embodiments, step a₄) further comprises heating to a temperature heated from about 120° C. to about 150° C. In some embodiments, the temperature is about 130° C. In some embodiments, step a₄) gives a yield greater than 75%. In some embodiments, step a₄) gives a yield greater than 90%. In some embodiments, step a₄) further comprises reacting the reaction mixture for a time period of about 6 hours to about 24 hours. In some embodiments, the time period is about 12 hours. In some embodiments, the compound of formula XXXVI is further defined as:

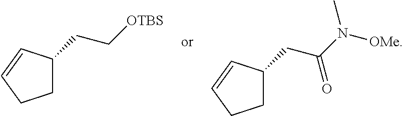

In some embodiments, the compound of formula XXXVI is further defined as:

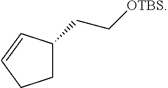 [SMILES: CC(C)(C)[Si](C)(C)OCC[C@H]1C=CCC1]

In some embodiments, the reaction of step b further comprises reacting the compound of formula XXXVI with a reducing agent. In some embodiments, the reducing agent is a metal hydride. In some embodiments, the reducing agent is diisobutylaluminum hydride. In some embodiments, reducing of step b comprises adding about 2.5 equivalents of reducing agent. In some embodiments, the reaction of step b further comprises a solvent. In some embodiments, the solvent is an organic solvent. In some embodiments, the solvent is a haloalkane_{(C≦12)}. In some embodiments, the solvent is dichloromethane. In some embodiments, the reaction of step b reacts for a time period from about 1 minute to about 1 hour. In some embodiments, the time period is about 10 minutes. In some embodiments, the reaction of step b reacts at a temperature from about −100° C. to about |70° C. In some embodiments, the temperature is about −78° C. In some embodiments, the reaction of step b further comprises allowing the solution to warm from about −78° C. to about 25° C. In some embodiments, the reaction of step b further comprises allowing the reduction to occur for about 10 minutes at about 25° C. In some embodiments, the reaction of step b results in a yield of greater than 80%. In some embodiments, the yield is greater than 90%. In some embodiments, the reaction of step b further comprises reacting the product with an alkylsilyl_{(C≦12)} chloride and a base. In some embodiments, the alkylsilyl_{(C≦12)} chloride is tert-butyldimethylsilyl chloride. In some embodiments, the base is imidazole. In some embodiments, the compound of formula XXXVI is further defined as:

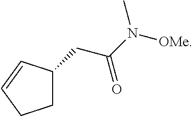

In some embodiments, the reaction of step b further comprises reacting the compound of formula XXXVI with a N,O-dimethylhydroxylamine hydrochloride. In some embodiments, the reaction of step b comprises adding about 2.0 equivalents of the N,O-dimethylhydroxylamine hydrochloride. In some embodiments, the reaction of step b further comprises adding a trialkyl_{(C≦12)} aluminum reagent. In some embodiments, the trialkyl_{(C≦12)} aluminum reagent is trimethyl aluminum. In some embodiments, reaction of step b comprises adding about 2.0 equivalents of the trialkyl_{(C≦12)} aluminum reagent. In some embodiments, the reaction of step b further comprises a solvent. In some embodiments, the solvent is an organic solvent. In some embodiments, the solvent is a haloalkane_{(C≦12)}. In some embodiments, the solvent is dichloromethane. In some embodiments, the reaction of step b reacts for a time period from about 40 minutes to about 4 hours. In some embodiments, the time period is about 45 minutes. In some embodiments, the reaction of step b reacts at a temperature from about −100° C. to about −70° C. In some embodiments, the temperature is about −78° C. In some embodiments, the reaction of step b further comprises allowing the solution to warm from about −78° C. to about 25° C. In some embodiments, the reaction of step b further comprises allowing the reduction to occur for about 45 minutes at about 25° C. In some embodiments, the reaction of step b results in a yield of greater than 80%. In some embodiments, the yield is greater than 90%. In some embodiments, the compound of formula XXXVI is further defined as:

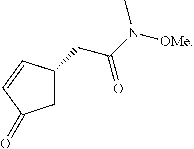

In some embodiments, the oxidation of step c further comprises adding a metal salt. In some embodiments, the metal salt is a rhodium salt. In some embodiments, the metal salt is dirhodium tetracaprolactamate. In some embodiments, the oxidation of step c comprises adding about 0.005 equivalents of metal salt. In some embodiments, the oxidation of step c further comprises adding a base. In some embodiments, the base is a metal carbonate. In some embodiments, the metal salt is K₂CO₃. In some embodiments, the oxidation of step c comprises adding about 0.5 equivalents of base. In some embodiments, the oxidation of step c further comprises an oxygen atmosphere. In some embodiments, the oxygen atmosphere comprises a pressure from about 0.1 atmospheres to about 10 atmospheres. In some embodiments, the oxidation of step c further comprises reacting the compound of formula XXXVI with an oxidizing agent. In some embodiments, the oxidizing agent is a peroxide. In some embodiments, the oxidizing agent is tert-butyl hydroperoxide. In some embodiments, oxidation of step c comprises adding about 5 equivalents of oxidizing agent. In some embodiments, the oxidation of step c comprises adding the metal salt and the base and then the oxidizing agent. In some embodiments, the oxidation of step c further comprises a solvent. In some embodiments, the solvent is an organic solvent. In some embodiments, the solvent is a haloalkane_{(C≦12)}. In some embodiments, the solvent is dichloromethane. In some embodiments, the oxidation of step c reacts for a time period from about 1 hour to about 6 hours. In some embodiments, the time period is about 1.5 hours. In some embodiments, the oxidation of step c reacts at a temperature from about 0° C. to about 30° C. In some embodiments, the temperature is about 25° C. In some embodiments, the oxidation of step c further comprises adding a second portion of the metal salt and the oxidizing agent. In some embodiments, the second portion of the metal salt is about 0.005 equivalents. In some embodiments, the second portion of the oxidizing agent is about 5 equivalents. In some embodiments, the oxidation of step c further comprises allowing the reduction to occur for a time period from about 1 hour to about 3 hours after the addition of the second portion of the metal salt and the oxidizing agent. In some embodiments, the time period is about 1.5 hours. In some embodiments, the oxidation of step c results in a yield of greater than 50%. In some embodiments, the yield is greater than 60%. In some embodiments, the compound of formula XXXVI is further defined as:

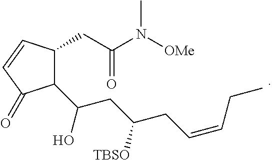 [SMILES: CC/C=C\C[C@@H](CC(O)C1C(=O)C=C[C@@H]1CC(=O)N(C)OC)O[Si](C)(C)C(C)(C)C]

In some embodiments, the reaction of step d further comprises adding a base. In some embodiments, the base is lithium diisopropylamide. In some embodiments, the reaction of step d comprises adding about 1.95 equivalents of base. In some embodiments, the reaction of step d comprises adding the base and the compound of formula XXXVI and then adding the compound of formula XXXVIII. In some embodiments, the reaction of step d comprises adding about 1.2 equivalents of the compound of formula XXXVIII. In some embodiments, the reaction of step d further comprises a solvent. In some embodiments, the solvent is an organic solvent. In some embodiments, the solvent is an ether_{(C≦12)}. In some embodiments, the solvent is tetrahydrofuran. In some embodiments, the reaction of step d reacts for a time period from about 15 minutes to about 3 hours. In some embodiments, the time period is about 20 minutes. In some embodiments, the reaction of step d reacts at a temperature from about −100° C. to about −70° C. In some embodiments, the temperature is about −78° C. In some embodiments, the reaction of step d further comprises allowing the reduction to occur for a time period from about 15 minutes to about 60 minutes after the addition of the compound of formula XI. In some embodiments, the time period is about 30 minutes. In some embodiments, the reaction of step d results in a yield of greater than 50%. In some embodiments, the yield is greater than 65%. In some embodiments, the reaction of step d further comprises reacting the compound of formula XLII with a group that which enhances the ability of the hydroxyl group to be eliminated. In some embodiments, the group that which enhances the ability of the hydroxyl group to be eliminated is an agent which enhances the leaving ability of a hydroxyl group. In some embodiments, the group that which enhances the ability of the hydroxyl group to be eliminated is methanesulfonyl chloride. In some embodiments, the reaction of compound of formula XLII with an activating agent comprises adding 5 equivalents of the group that which enhances the ability of the hydroxyl group to be eliminated. In some embodiments, the reaction of compound of formula XLII with the group that which enhances the ability of the hydroxyl group to be eliminated further comprises a base. In some embodiments, the base is a triethylamine. In some embodiments, the reaction of compound of formula XLII with the group that which enhances the ability of the hydroxyl group to be eliminated further comprises a solvent. In some embodiments, the solvent is an organic solvent. In some embodiments, the solvent is haloalkane_{(C≦12)}. In some embodiments, the solvent is dichloromethane. In some embodiments, the reaction of a compound of formula XLII with the group that which enhances the ability of the hydroxyl group to be eliminated comprises reacting the compound and the group that which enhances the ability of the hydroxyl group to be eliminated for a time period from about 1 minute to about 30 minutes. In some embodiments, the time period is about 5 minutes. In some embodiments, the reaction of a compound of formula XLII with the group that which enhances the ability of the hydroxyl group to be eliminated reacts at a temperature from about −30° C. to about 30° C. In some embodiments, the temperature is about 0° C. In some embodiments, the reaction of a compound of formula XXXVI with the group that which enhances the ability of the hydroxyl group to be eliminated results in a yield of greater than 80%. In some embodiments, the yield is greater than 90%. In some embodiments, the compound of formula XLIII is further defined as:

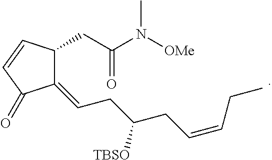 [SMILES: CC/C=C\C[C@@H](C/C=C1/C(=O)C=C[C@@H]1CC(=O)N(C)OC)O[Si](C)(C)C(C)(C)C]

In some embodiments, the dehydration of step e further comprises reacting the compound with Al₂O₃. In some embodiments, dehydration of step e comprises adding about 7 equivalents of Al₂O₃. In some embodiments, the dehydration of step e further comprises adding about a second and third portion of Al₂O₃ at 2 and 4 hours, respectively. In some embodiments, the second and third portion of Al₂O₃ comprise adding about 7 equivalents of Al₂O₃. In some embodiments, the Al₂O₃ is activated by heating to about 400° C. under vacuum for about 5 minutes. In some embodiments, the reaction of step b further comprises a solvent. In some embodiments, the solvent is an organic solvent. In some embodiments, the solvent is a haloalkane_{(C≦12)}. In some embodiments, the solvent is dichloromethane. In some embodiments, the reaction of step b reacts for a time period from about 10 minutes to about 10 hours. In some embodiments, the time period is about 30 minutes. In some embodiments, the time period is about 8 hours. In some embodiments, the reaction of step b reacts at a temperature from about 0° C. to about 35° C. In some embodiments, the temperature is about 25° C. In some embodiments, the reaction of step b results in a yield of greater than 75%. In some embodiments, the yield is greater than 85%. In some embodiments, the compound of formula XLIII is further defined as:

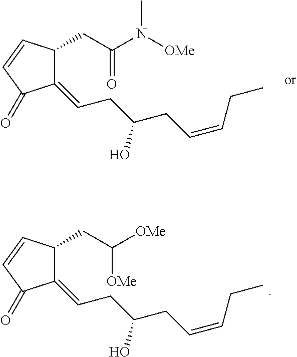 [SMILES: CC/C=C\C[C@H](O)C/C=C1/C(=O)C=C[C@@H]1CC(=O)N(C)OC ; CC/C=C\C[C@H](O)C/C=C1/C(=O)C=C[C@@H]1CC(OC)OC]

In some embodiments, the deprotection of step f further comprises reacting the compound of formula XLIII with a fluoride source. In some embodiments, the fluoride source is hydrofluoric acid. In some embodiments, the hydrofluoric acid is a 50% aqueous hydrofluoric acid solution. In some embodiments, dehydration of step f comprises adding about 50 equivalents of HF. In some embodiments, the dehydration of step f further comprises a solvent. In some embodiments, the solvent is an aqueous and organic solvent mixture. In some embodiments, the solvent is an alkane_{(C≦12)} substituted with —CN. In some embodiments, the solvent is acetonitrile and water mixture. In some embodiments, the dehydration of step f reacts for a time period from about 30 minutes to about 4 hours. In some embodiments, the time period is about 1 hour. In some embodiments, the dehydration of step f reacts at a temperature from about −30° C. to about 30° C. In some embodiments, the temperature is about 0° C. In some embodiments, the dehydration of step f results in a yield of greater than 90%. In some embodiments, the yield is greater than 95%. In some embodiments, the yield is greater than 98%.

In yet another aspect, the present invention provides a method of preparing a compound of the formula:

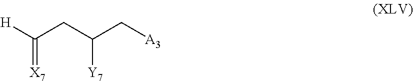

wherein: X₇ is O, S, or NR₇; wherein: R₇ is hydrogen, alkyl_{(C≦12)}, substituted alkyl_{(C≦12)}, or an amine protecting group; Y₇ is hydrogen, amino, hydroxy, mercapto, —OR₈, —SR₉, or —NR₁₀R₁₁; wherein: R₈ is alkyl_{(C≦12)}, aralkyl_{(C≦12)}, acyl_{(C≦12)}, a substituted version of any of these three groups, or a hydroxy protecting group; R₉ is alkyl_{(C≦12)}, aralkyl_{(C≦12)}, acyl_{(C≦12)}, a substituted version of any of these three groups, or a thiol protecting group; and R₁₀ and R₁₁ are each independently, alkyl_{(C≦12)}, aralkyl_{(C≦12)}, acyl_{(C≦12)}, a substituted version of any of these three groups, or a monovalent amino protecting group; or R₁₀ and R₁₁ are taken together to form a divalent amino protecting group; and A₃ is alkyl_{(C≦13)}, alkenyl_{(C≦13)}, alkynyl_{(C≦13)}, aralkyl_{(C≦13)}, heteroaralkyl_{(C≦13)}, or a substituted version of any of these groups; comprising: a) reacting a compound of the formula:

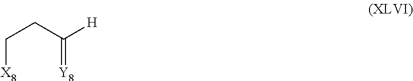

wherein: X₈ is amino, hydroxy, mercapto, —OR₁₂, —SR₁₃, or —NR₁₄R₁₅; wherein: R₁₂ is alkyl_{(C≦12)}, aralkyl_{(C≦12)}, acyl_{(C≦12)}, a substituted version of any of these three groups, or a hydroxy protecting group; R₁₃ is alkyl_{(C≦12)}, aralkyl_{(C≦12)}, acyl_{(C≦12)}, a substituted version of any of these three groups, or a thiol protecting group; and R₁₄ and R₁₅ are each independently, alkyl_{(C≦12)}, aralkyl_{(C≦12)}, acyl_{(C≦12)}, a substituted version of any of these three groups, or a monovalent amino protecting group; or R₁₄ and R₁₅ are taken together to form a divalent amino protecting group; Y₈ is O, S, or NR₁₆; wherein: R₁₆ is hydrogen, alkyl_{(C≦12)}, substituted alkyl_{(C≦12)}, or an amine protecting group; with a compound of the formula:

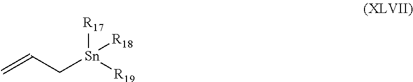

wherein: R₁₇, R₁₈, and R₁₉ are each independently alkyl_{(C≦12)}, alkenyl_{(C≦12)}, alkynyl_{(C≦12)}, aryl_{(C≦12)}, aralkyl_{(C≦12)}, heteroaryl_{(C≦12)}, heterocycloalkyl_{(C≦12)}, or a substituted version of any of these groups; to form a compound of the formula:

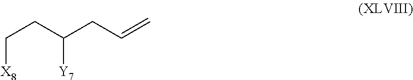

wherein X₈ and Y₇ are as defined above; b) reacting the compound of formula XLVIII with ozone to form a compound of the formula:

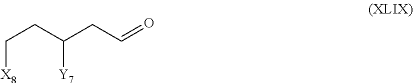

wherein X₈ and Y₇ are as defined above; c) reacting the compound of formula XLIX with a haloalkane_{(C≦12)}, haloalkene_{(C≦12)}, haloalkyne_{C≦12)}, haloaralkane_{C≦12)}, haloheteroaralkane_{(C≦12)}, or a substituted version of any of these groups to form a compound of the formula:

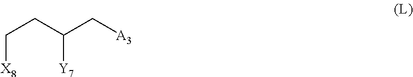

wherein X₈ and Y₇ are as defined above; and d) oxidizing the compound of formula L in the presence of an oxidizing agent to form the compound of formula XLV. In some embodiments, X₇ is O. In some embodiments, Y₇ is hydroxy. In some embodiments, Y₇ is —OR₈ wherein R₈ is alkyl_{(C≦12)}, aralkyl_{(C≦12)}, acyl_{(C≦12)}, a substituted version of any of these three groups, or a hydroxy protecting group. In some embodiments, Y₇ is —OR₈ wherein R₈ is a hydroxy protecting group. In some embodiments, the hydroxy protecting group is an alkylsilyl_{(C≦12)}. In some embodiments, the alkylsilyl_{(C≦12)} is tert-butyldimethylsilyl. In some embodiments, X₈ is hydroxy. In some embodiments, X₈ is —OR₁₂ wherein R₁₂ is alkyl_{(C≦12)}, aralkyl_{(C≦12)}, acyl_{(C≦12)}, a substituted version of any of these three groups, or a hydroxy protecting group. In some embodiments, R₁₂ is hydroxy protecting group. In some embodiments, the hydroxy protecting group is an alkylsilyl_{(C≦12)}. In some embodiments, the alkylsilyl_{(C≦12)} is tert-butyldimethylsilyl. In some embodiments, Y₈ is O. In some embodiments, R₁₇ is alkyl_{(C≦12)}. In some embodiments, R₁₈ is alkyl_{(C≦12)}. In some embodiments, R₁₉ is alkyl_{(C≦12)}. In some embodiments, R₁₇, R₁₈, and R₁₉ are alkyl_{(C≦12)}. In some embodiments, R₁₇, R₁₈, and R₁₉ are butyl. In some embodiments, A₃ is alkenyl_{(C≦13)}, substituted alkenyl_{(C≦13)}, alkynyl_{(C≦13)}, or substituted alkynyl_{(C≦13)}. In some embodiments, A₃ is alkenyl_{(C≦13)}. In some embodiments, A₃ is —CH═CHCH₂CH═CHCH₂CH₃. In some embodiments, A₃ is substituted alkenyl_{(C≦13)}. In some embodiments, A₃ is —CH═CHCH₂CF₃. In some embodiments, A₃ is alkynyl_{(C≦13)}. In some embodiments, A₃ is —C≡CCH₂CH₃. In some embodiments, the reaction of step a) further comprises a solvent. In some embodiments, the solvent is an organic solvent. In some embodiments, the solvent is an aryl_{(C≦12)} or aralkyl_{(C≦12)}. In some embodiments, the solvent is toluene. In some embodiments, the reaction of step a) comprises heating the reaction to a temperature from about −85° C. to about 0° C. In some embodiments, the temperature is about −20° C. In some embodiments, the reaction of step a) further comprises adding an enantiomer of a binaphthyl compound. In some embodiments, the binaphthyl compound is BINOL. In some embodiments, the enantiomer of a binaphthyl compound is a (S)-BINOL. In some embodiments, the reaction of step a) comprises adding about 0.01 to about 0.2 equivalents relative to the compound of formula XLVI of the enantiomer of a binaphthyl compound. In some embodiments, about 0.05 equivalents of the enantiomer of a binapthyl compound are added to the reaction. In some embodiments, the reaction of step a) further comprises adding a titanium compound. In some embodiments, the titanium compound is titanium(IV) tetraalkoxide. In some embodiments, the titanium compound is Ti(OiPr)₄. In some embodiments, the reaction of step a) comprises adding from about 0.01 to about 0.1 equivalents of a titanium compound relative to the compound of formula XLVI. In some embodiments, the reaction of step a) comprises adding from about 1.0 to about 3.0 equivalents of the compound of formula XLVII relative to the compound of formula XLVI. In some embodiments, the reaction comprises adding about 1.5 equivalents of the compound of formula XLVII relative to the compound of formula XLVI. In some embodiments, the reaction of step a) comprises reacting the compound of formula XLVII with the compound of formula XLVI for a time period from about 96 hours to about 180 hours. In some embodiments, the time period is about 139 hours. In some embodiments, the reaction of step a) produces a yield of a compound of formula XLVIII of greater than 30%. In some embodiments, the yield is greater than 40%. In some embodiments, the reaction of step a) produces a yield based on recovered starting material of a compound of formula XLVIII of greater than 50%. In some embodiments, the yield based upon recovered starting material is greater than 60%. In some embodiments, the reaction of step a) produces the compound of formula XLVIII in an enantiomeric excess (ee) of greater than 90%. In some embodiments, the ee is greater than 95%. In some embodiments, the reaction of step a) further comprises a second step comprising reacting the compound of formula XLVIII with a protecting agent. In some embodiments, the protecting agent is an alkylsilyl_{(C≦12)} halide. In some embodiments, the protecting agent is tert-butylsilyl chloride. In some embodiments, the second step comprises adding from about 1.0 to about 3.0 equivalents of the protecting agent relative to the compound of formula IV. In some embodiments, the second step comprises adding about 1.4 equivalents of the protecting agent relative to the compound of formula XLVIII. In some embodiments, the second step further comprises adding a base. In some embodiments, the base is a nitrogenous base. In some embodiments, the base is imidazole. In some embodiments, the second step comprises adding from about 2 to about 5 equivalents of the base relative to the compound of formula XLVIII. In some embodiments, the second step comprises adding about 3.1 equivalents of the base relative to the compound of formula XLVIII. In some embodiments, the second step comprises reacting the compound of formula XLVIII in a solvent. In some embodiments, the solvent is an organic solvent. In some embodiments, the solvent is a haloalkane_{(C≦12)}. In some embodiments, the solvent is dichloromethane. In some embodiments, the second step comprises heating the reaction to a temperature from about 5° C. to about 40° C. In some embodiments, the temperature is about 25° C. In some embodiments, the second step comprises reacting for a time period from about 1 hour to about 4 hours. In some embodiments, the time period is 90 minutes. In some embodiments, the yield of the second step is greater than about 75%.

In some embodiments, the yield is greater than 85%. In some embodiments, the reaction of step b) further comprises a solvent. In some embodiments, the solvent is an organic solvent. In some embodiments, the solvent is haloalkane_{(C≦12)}. In some embodiments, the haloalkane_{(C≦12)} is dichloromethane. In some embodiments, the reaction of step b) further comprises adding ozone until the solvent turns blue in color. In some embodiments, the method further comprises bubbling nitrogen through the solvent until the blue color disappears. In some embodiments, the reaction of step b) further comprises heating to a temperature from about −100° C. to about −20° C. In some embodiments, the temperature is about −78° C. In some embodiments, the reaction of step b) further comprises allowing the reaction to warm to a temperature from about 0° C. to about 35° C. after the disappearance of the blue color. In some embodiments, the temperature is about 25° C. In some embodiments, the reaction of step b) comprises reacting for a time period from about 1 hour to about 6 hours. In some embodiments, the time period is about 3 hours. In some embodiments, the time period starts after the disappearance of the blue color. In some embodiments, the reaction of step b) further comprises a base. In some embodiments, the base is a metal bicarbonate or carbonate. In some embodiments, the base is a metal bicarbonate. In some embodiments, the base is sodium bicarbonate. In some embodiments, the reaction further comprises adding from about 1 mg to about 250 mg of base per gram of the compound of formula XLVIII. In some embodiments, the reaction of step b) further comprises adding a phosphine_{(C≦24)}. In some embodiments, the phosphine_{(C≦24)} is triphenylphosphine. In some embodiments, the phosphine is added after the disappearance of the blue color. In some embodiments, the method comprises adding from about 1.1 to about 4 equivalents of the phosphine relative to the compound of formula XLVIII to the reaction. In some embodiments, the method comprises adding about 2 equivalents of the phosphine relative to the compound of formula XLVIII. In some embodiments, the reaction of step b) comprises a yield of greater than 85%. In some embodiments, the yield is greater than 90%. In some embodiments, the yield is greater than 95%. In some embodiments, the reaction of step c) further comprises reacting the haloalkane_{(C≦12)}, haloalkene_{(C≦12)}, haloalkyne_{C≦12)}, haloaralkane_{C≦12)}, haloheteroaralkane_{(C≦12)}, or the substituted version of any of these groups with a phosphine_{(C≦24)}. In some embodiments, the phosphine_{(C≦24)} was triphenylphosphine. In some embodiments, the reaction further comprises adding from about 1 to about 4 equivalents of phosphine relative to the compound of formula XLIX to the reaction. In some embodiments, the reaction further comprises adding about 2 equivalents of phosphine relative to the compound of formula XLIX to the reaction. In some embodiments, the reaction further comprises adding from about 1 to about 4 equivalents of the haloalkane_{(C≦12)}, haloalkene_{(C≦12)}, haloalkyne_{C≦12)}, haloaralkane_{C≦12)}, haloheteroaralkane_{(C≦12)}, or the substituted version of any of these groups relative to the compound of formula XLIX to the reaction. In some embodiments, the reaction further comprises adding about 2 equivalents of the haloalkane_{(C≦12)}, haloalkene_{(C≦12)}, haloalkyne_{(C≦12)}, haloaralkane_{C≦12)}, haloheteroaralkane_{(C≦12)}, or the substituted version of any of these groups relative to the compound of the formula XLIX to the reaction. In some embodiments, the reaction further comprises a base. In some embodiments, the base is a non-nucleophilic base. In some embodiments, the base is a metal bis(trimethylsilyl)amine. In some embodiments, the base is NaHMDS. In some embodiments, the reaction further comprises a solvent. In some embodiments, the solvent is an organic solvent. In some embodiments, the solvent is an ether_{(C≦12)}. In some embodiments, the ether_{(C≦12)} is tetrahydrofuran. In some embodiments, the reaction further comprises reacting at a temperature from about −20° C. to about 20° C. In some embodiments, the temperature is about 0° C. In some embodiments, the reaction is reacted for a first time period from about 10 minutes to about 2 hours. In some embodiments, the first time period is about 30 minutes. In some embodiments, the reaction further comprises reducing the temperature to a temperature from about −100° C. to about −20° C. In some embodiments, the temperature is about −78° C. In some embodiments, the reaction comprises adding the compound of formula XLIX to the reaction after the first time period. In some embodiments, the compound of formula XLIX is added dropwise. In some embodiments, the reaction reacts for a second time period from about 10 minutes to about 2 hours. In some embodiments, the second time period is about 30 minutes. In some embodiments, the reaction is warmed to a temperature from about 10° C. to about 35° C. after the second time period. In some embodiments, the temperature is about 25° C. In some embodiments, the reaction reacts for a third time period from about 1 hour to about 6 hours. In some embodiments, the third time period is about 2 hours. In some embodiments, the reaction is quenched by adding a saturated aqueous NH₄Cl solution after the third time period. In some embodiments, quenching the reaction further comprises stirring for a fourth time period from about 5 minutes to about 1 hour. In some embodiments, the fourth time period is about 20 minutes. In some embodiments, the reaction of step c) has a yield between about 1% and about 100%. In some embodiments, the yield is greater than about 60%. In some embodiments, the yield is greater than about 85%. In some embodiments, the reaction of step c) has a Z/E ratio between about 1:15 and about 15:1. In some embodiments, the Z/E ratio is about 1:1 and 15:1. In some embodiments, the reaction of step c) further comprises deprotecting X₈ comprising adding pyridinium tribromide. In some embodiments, the deprotection further comprises a solvent. In some embodiments, the solvent is an organic solvent. In some embodiments, the solvent is an alcohol_{(C≦12)}. In some embodiments, the alcohol_{(C≦12)} is methanol. In some embodiments, the deprotection comprises adding from about 0.01 to about 0.25 equivalents of pyridinium tribromide relative to the compound of the formula L. In some embodiments, the deprotection comprises adding about 0.05 equivalents of pyridinium tribromide relative to the compound of the formula L. In some embodiments, the deprotection further comprises reacting at a temperature from about −25° C. to about 0° C. In some embodiments, the temperature is about −10° C. In some embodiments, the deprotection further comprises reacting for a time period from about 2 hours to about 10 hours. In some embodiments, the time period is about 5 hours. In some embodiments, the deprotection further comprises quenching the reaction with water. In some embodiments, the quenching further comprises warming the reaction to a temperature from about 5° C. to about 35° C. In some embodiments, the temperature is about 25° C. In some embodiments, the oxidation of step d) further comprises a solvent. In some embodiments, the solvent is an organic solvent. In some embodiments, the solvent is a haloalkane_{(C≦12)}. In some embodiments, the haloalkane_{(C≦12)} is dichloromethane. In some embodiments, the oxidizing agent is Dess-Martin periodinane. In some embodiments, the oxidation of step d) further comprises cooling the reaction to a temperature from about −20° C. to about 10° C. In some embodiments, the temperature is 0° C. In some embodiments, the oxidation of step d) further comprises adding from about 1 equivalent to about 4 equivalents of oxidizing agent relative to the compound of formula L. In some embodiments, the method further comprises adding about 1.5 equivalents of the oxidizing agent relative to the compound of formula L. In some embodiments, the oxidation of step d) further comprises oxidizing the compound of formula L for a first time period from about 5 minutes to about 2 hours. In some embodiments, the first time period is about 30 minutes. In some embodiments, the method further comprises allowing the oxidation to warm to a temperature from about 10° C. to about 35° C. after the first time period. In some embodiments, the temperature is about 25° C. In some embodiments, the method further comprises oxidizing for a second time period from about 15 minutes to about 4 hours. In some embodiments, the second time period is from about 60 to about 90 minutes. In some embodiments, oxidation further comprises oxidizing for a total time period from about 20 minutes to about 6 hours. In some embodiments, the total time period is from about 90 minutes to 2 hours. In some embodiments, the oxidation of step d) further comprises quenching the reaction with an aqueous solution. In some embodiments, the aqueous solution contains a metal bicarbonate. In some embodiments, the metal bicarbonate is sodium bicarbonate. In some embodiments, the aqueous solution contains a metal thiosulfate. In some embodiments, the metal thiosulfate is sodium thiosulfate. In some embodiments, the aqueous solution contains a mixture of sodium bicarbonate and sodium thiosulfate. In some embodiments, the mixture is a 1:1 mixture of sodium bicarbonate to sodium thiosulfate. In some embodiments, the method further comprises quenching for a time period from about 5 minutes to about 1 hour. In some embodiments, the time period is about 20 minutes. In some embodiments, the oxidation of step d) has a yield from about 1% to about 100%. In some embodiments, the yield is greater than about 70%. In some embodiments, the yield is greater than 85%. In some embodiments, the reaction of step c) further comprises reacting the haloalkane_{(C≦12)} or substituted haloalkane_{(C≦12)} with a phosphine_{(C≦24)}. In some embodiments, the haloalkane is CBr₄. In some embodiments, the phosphine_{(C≦24)} was triphenylphosphine. In some embodiments, the reaction further comprises adding from about 1 to about 8 equivalents of phosphine relative to the compound of formula XLIX to the reaction. In some embodiments, the reaction further comprises adding about 4 equivalents of phosphine relative to the compound of formula XLIX to the reaction. In some embodiments, the reaction further comprises adding from about 1 to about 4 equivalents of the haloalkane_{(C≦12)} or substituted haloalkane_{(C≦12)} relative to the compound of formula XLIX to the reaction. In some embodiments, the reaction further comprises adding about 2 equivalents of the haloalkane_{(C≦12)} or substituted haloalkane_{(C≦12)} relative to the compound of the formula XLIX to the reaction. In some embodiments, the reaction further comprises a first solvent. In some embodiments, the first solvent is an organic solvent. In some embodiments, the first solvent is a haloalkane_{(C≦12)}. In some embodiments, the haloalkane_{(C≦12)} is dichloromethane. In some embodiments, the reaction further comprises reacting at a temperature from about −20° C. to about 20° C. In some embodiments, the temperature is about 0° C. In some embodiments, the reaction is reacted for a first time period from about 1 minute to about 1 hour. In some embodiments, the first time period is about 10 minutes. In some embodiments, the reaction further comprises warming the reaction to a temperature from about 10° C. to about 35° C. In some embodiments, the temperature is about 25° C. In some embodiments, the reaction comprises adding the compound of formula XLIX to the reaction after the first time period. In some embodiments, the compound of formula XLIX is added dropwise. In some embodiments, the reaction reacts for a second time period from about 10 minutes to about 2 hours. In some embodiments, the second time period is about 30 minutes. In some embodiments, the reaction further comprises adding a second solvent. In some embodiments, the second solvent is an organic solvent. In some embodiments, the second solvent is an alkane_{(C≦12)}. In some embodiments, the alkane_{(C≦12)} is hexanes. In some embodiments, the reaction further comprises filtering the solvent through diatomaceous earth after the reaction. In some embodiments, the diatomaceous earth is Celite®. In some embodiments, the reaction of step c) has a yield between about 1% and about 100%. In some embodiments, the yield is greater than about 60%. In some embodiments, the yield is greater than about 85%. In some embodiments, the reaction of step c) has a Z/E ratio between about 1:15 and about 15:1. In some embodiments, the Z/E ratio is about 1:1 and 15:1. In some embodiments, the method further comprises reacting the product of the reaction of the haloalkane_{(C≦12)}, phosphine_{(C≦24)}, and the compound of formula XLIX with a base. In some embodiments, the base is a strong base. In some embodiments, the base is an organolithium reagent. In some embodiments, the organolithium reagent is n-butyl lithium. In some embodiments, the reaction further comprises a solvent. In some embodiments, the solvent is an organic solvent. In some embodiments, the solvent is an ether_{(C≦12)}. In some embodiments, the ether_{(C≦12)} is tetrahydrofuran. In some embodiments, the reaction further comprises a temperature from about −100° C. to about −20° C. In some embodiments, the temperature is about −78° C. In some embodiments, the reaction further comprises adding from about 1.25 equivalents to about 5 equivalents of base relative to the compound of formula XLIX to the reaction. In some embodiments, the method further comprises adding about 3 equivalents of base relative to the compound of formula XLIX to the reaction. In some embodiments, the reaction further comprises warming the reaction to a temperature from about −20° C. to about 20° C. over a first time period from about 10 minutes to about 2 hours. In some embodiments, the temperature is about 0° C. In some embodiments, the first time period is about 30 minutes. In some embodiments, the reaction further comprises reacting for a second time period from about 10 minutes to about 2 hours. In some embodiments, the second time period is about 30 minutes. In some embodiments, the reaction further comprises adding a second haloalkane_{(C≦12)}, haloalkene_{(C≦12)}, haloalkyne_{C≦12)}, haloaralkane_{C≦12)}, haloheteroaralkane_{(C≦12)}, or a substituted version of any of these groups. In some embodiments, the second haloalkane_{(C≦12)} is ethyliodide. In some embodiments, the reaction further comprises cooling the solvent to a temperature from about −100° C. to about −20° C. before the addition of the second haloalkane_{(C≦12)}, haloalkene_{(C≦12)}, haloalkyne_{C≦12)}, haloaralkane_{C≦12)}, haloheteroaralkane_{(C≦12)}, or a substituted version of any of these groups. In some embodiments, the reaction comprises adding from about 5 equivalents to about 20 equivalents of the haloalkane_{(C≦12)}, haloalkene_{(C≦12)}, haloalkyne_{C≦12)}, haloaralkane_{C≦12)}, haloheteroaralkane_{(C≦12)}, or a substituted version of any of these groups relative to the compound of formula XLIX to the reaction. In some embodiments, the reaction comprises adding about 10 equivalents of the haloalkane_{(C≦12)}, haloalkene_{(C≦12)}, haloalkyne_{C≦12)}, haloaralkane_{C≦12)}, haloheteroaralkane_{(C≦12)}, or a substituted version of any of these groups relative to the compound of formula XLIX to the reaction. In some embodiments, the method further comprises after the addition of the haloalkane_{(C≦12)}, haloalkene_{(C≦12)}, haloalkyne_{C≦12)}, haloaralkane_{C≦12)}, haloheteroaralkane_{(C≦12)}, or a substituted version of any of these groups to the reaction allowing the reaction to warm to a temperature from about 10° C. to about 40° C. In some embodiments, the temperature is about 25° C. In some embodiments, the reaction is allowed to proceed for a time period from about 1 hour to about 48 hours. In some embodiments, the time period is from about 3 hours to about 24 hours. In some embodiments, the time period is about 18 hours. In some embodiments, the reaction is allowed to precede for a time period along enough that the starting material is not present through a spectroscopic method. In some embodiments, the spectroscopic method is ¹H NMR. In some embodiments, the method further comprises quenching the reaction by adding a saturated ammonium solution. In some embodiments, the saturated ammonium solution is ammonium chloride. In some embodiments, the reaction has a yield between about 1% and about 100%. In some embodiments, the yield is greater than 50%. In some embodiments, the yield is greater than 65%. In some embodiments, the reaction of step c) further comprises deprotecting X₈ comprising adding pyridinium tribromide. In some embodiments, the deprotection further comprises a solvent. In some embodiments, the solvent is an organic solvent. In some embodiments, the solvent is an alcohol_{(C≦12)}. In some embodiments, the alcohol_{(C≦12)} is methanol. In some embodiments, the deprotection comprises adding from about 0.01 to about 0.25 equivalents of pyridinium tribromide relative to the compound of the formula L. In some embodiments, the deprotection comprises adding about 0.05 equivalents of pyridinium tribromide relative to the compound of the formula L. In some embodiments, the deprotection further comprises reacting at a temperature from about −25° C. to about 0° C. In some embodiments, the temperature is about −10° C. In some embodiments, the deprotection further comprises reacting for a time period from about 2 hours to about 10 hours. In some embodiments, the time period is about 5 hours. In some embodiments, the deprotection further comprises quenching the reaction with water. In some embodiments, the quenching further comprises warming the reaction to a temperature from about 5° C. to about 35° C. In some embodiments, the temperature is about 25° C. In some embodiments, the method further comprises reacting the product of the reaction of the haloalkane_{(C≦12)}, phosphine_{(C≦24)}, and the compound of formula XLIX with a haloalkyne_{C≦12)} or substituted haloalkyne_{(C≦12)}, a base, and a metal. In some embodiments, the base is a metal carbonate. In some embodiments, the base is a potassium carbonate, sodium carbonate, or lithium carbonate. In some embodiments, the base is potassium carbonate. In some embodiments, the metal is a metal salt. In some embodiments, the metal salt is a copper(I) salt. In some embodiments, the metal salt is CuI. In some embodiments, the haloalkyne_{(C≦12)} is a haloalkyne_{(C≦8)}. In some embodiments, the haloalkyne_{(C≦8)} is 1-bromo-2-pentyne. In some embodiments, the method further comprises adding an iodide salt. In some embodiments, the iodide salt is sodium iodide, potassium iodide, lithium iodide, magnesium iodide, or calcium iodide. In some embodiments, the reaction further comprises a solvent. In some embodiments, the solvent is an organic solvent. In some embodiments, the solvent is an amide_{(C≦12)}. In some embodiments, the amide_{(C≦12)} is NA-dimethylformamide. In some embodiments, the reaction further comprises a temperature from about 10° C. to about 40° C. In some embodiments, the temperature is about 25° C. In some embodiments, the reaction further comprises adding from about 1.0 equivalents to about 3.0 equivalents of haloalkyne_{(C12)} or substituted haloalkyne_{(C≦12)} relative to the compound of formula XLIX to the reaction. In some embodiments, the method further comprises adding about 1.2 equivalents of haloalkyne_{(C12)} or substituted haloalkyne_{(C≦12)} relative to the compound of formula XLIX to the reaction. In some embodiments, the reaction further comprises adding from about 1 equivalents to about 3 equivalents of base relative to the compound of formula XLIX to the reaction. In some embodiments, the method further comprises adding about 1.3 equivalents of base relative to the compound of formula XLIX to the reaction. In some embodiments, the reaction further comprises adding from about 1 equivalents to about 3 equivalents of metal relative to the compound of formula XLIX to the reaction. In some embodiments, the method further comprises adding about 1.3 equivalents of metal relative to the compound of formula XLIX to the reaction. In some embodiments, the reaction further comprises adding from about 1 equivalents to about 3 equivalents of iodide salt relative to the compound of formula V to the reaction. In some embodiments, the method further comprises adding about 1.3 equivalents of iodide salt relative to the compound of formula XLIX to the reaction. In some embodiments, the reaction is vigorously stirred. In some embodiments, the reaction is carried out in the dark. In some embodiments, the reaction is allowed to proceed for a time period from about 1 hour to about 36 hours. In some embodiments, the time period is from about 3 hours to about 24 hours. In some embodiments, the time period is about 15 hours. In some embodiments, the method further comprises diluting the reaction with an ether_{(C≦12)} and filtering through diatomaceous earth. In some embodiments, the ether_{(C≦12)} is diethyl ether. In some embodiments, the diatomaceous earth is Celite®. In some embodiments, the reaction has a yield between about 1% and about 100%. In some embodiments, the yield is greater than 60%. In some embodiments, the yield is greater than 75%. In some embodiments, the reaction further comprises reducing the product of the reaction in the presence of a metal suspension. In some embodiments, the reaction further comprises a solvent. In some embodiments, the solvent is an organic solvent. In some embodiments, the solvent is an alcohol_{(C≦12)}. In some embodiments, the alcohol_{(C≦12)} is ethanol. In some embodiments, the metal suspension is a transition metal. In some embodiments, the metal suspension comprises a metal salt. In some embodiments, the metal salt is a nickel(II) salt. In some embodiments, the nickel(II) salt is nickel(II) diacetate. In some embodiments, the nickel(II) salt is a tetrahydrate. In some embodiments, the method further comprises adding a reducing agent. In some embodiments, the reducing agent is a metal borohydride. In some embodiments, the metal hydride is sodium borohydride, lithium borohyride, and potassium borohyride. In some embodiments, the method further comprises adding H₂ gas. In some embodiments, the reaction further comprises adding 1,2-diaminoethane after the addition of H₂ gas. In some embodiments, the reaction comprises adding from about 2 equivalents to about 5 equivalents of the 1,2-diaminoethane relative to the compound of the formula XLIX to the reaction. In some embodiments, the reaction comprises 3.6 equivalents of the 1,2-diaminoethane relative to the compound of the formula XLIX to the reaction. In some embodiments, the reaction comprises adding from about 0.25 equivalents to about 1.25 equivalents of the reducing agent relative to the compound of the formula XLIX to the reaction. In some embodiments, the reaction comprises adding about 0.77 equivalents of the reducing agent relative to the compound of the formula XLIX to the reaction. In some embodiments, the reaction comprises adding from about 0.1 equivalents to about 0.75 equivalents of the metal salt relative to the compound of the formula XLIX to the reaction. In some embodiments, the reaction comprises adding about 0.32 equivalents of the metal salt relative to the compound of the formula XLIX to the reaction. In some embodiments, the reaction comprises reacting a temperature from about 10° C. to about 40° C. In some embodiments, the temperature is about 25° C. In some embodiments, the reaction comprises reacting for a time period from about 12 hours to about 36 hours. In some embodiments, the time period is about 18 hours. In some embodiments, the reaction comprises running the reaction in the dark. In some embodiments, the reaction further comprises a yield from about 1% to about 100%. In some embodiments, the yield is greater than 50%. In some embodiments, the yield is greater than 75%.

In some embodiments, the method of preparing an intermediate or compound as described in the present invention wherein any of the steps further comprise a purification step or performing a purification. In some embodiments, the purification step is chromatography or an extraction. In some embodiments, the chromatrography is high pressure liquid chromatography or flash chromatography. In some embodiments, the extraction is an organic/aqueous extraction.

In some embodiments, the method further comprises reacting the compound of the formula XLI with a methylating agent. In some embodiments, the methylating agent is trimethylsilyl diazomethane. In some embodiments, the method comprises using from about 1 equivalent of the methylating agent to about 4 equivalents of methylating agent relative to the compound of the formula XLI. In some embodiments, the method comprises adding about 1.5 equivalents of the methylating agent relative to the compound of the formula XLI. In some embodiments, the method comprises reacting for a time period from about 15 minutes to about 3 hours. In some embodiments, the time period is about 30 minutes. In some embodiments, the method further comprises a solvent. In some embodiments, the solvent is an organic solvent. In some embodiments, the solvent is a mixture of two or more organic solvents. In some embodiments, the solvent is a first organic solvent and a second organic solvent. In some embodiments, the first organic solvent is an arene_{(C≦12)} or aralkane_{(C≦12)}. In some embodiments, the first organic solvent is benzene. In some embodiments, the second organic solvent is an alcohol_{(C≦12)}. In some embodiments, the second organic solvent is methanol. In some embodiments, first organic solvent and second organic solvent have a ratio of first organic solvent to second organic solvent from about 6:1 to about 1:6. In some embodiments, the ratio of first organic solvent to second organic solvent is 3:2. In some embodiments, the method comprises reacting the compounds at a temperature form about 10° C. to about 40° C. In some embodiments, the temperature is about 25° C. In some embodiments, the reaction has a yield from about 1% to about 100%. In some embodiments, the yield is greater than 50%. In some embodiments, the yield is greater than 70%.

In yet another aspect, the present invention provides a method of preparing a compound of the formula:

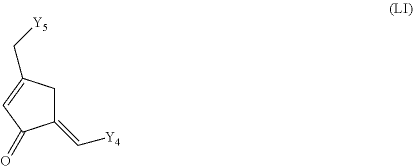

wherein: Y₄ is alkyl_{(C≦18)}, alkenyl_{(C≦18)}, alkynyl_{(C≦18)}, aryl_{(C≦18)}, aralkyl_{(C≦18)}, heteroaryl_{(C≦18)}, heterocycloalkyl_{(C≦18)}, or a substituted version of any these groups; Y₅ is hydrogen, hydroxy, amino, cyano, or; alkyl_{(C≦18)}, alkenyl_{(C≦18)}, alkynyl_{(C≦18)}, aryl_{(C≦8)}, heteroaryl_{(C≦8)}, heterocycloalkyl_{(C≦18)}, alkoxy_{(C≦18)}, alkenyloxy_{(C≦8)}, alkynyloxy_{(C≦18)}, aryloxy_{(C≦18)}, heteroaryloxy_{(C≦18)}, heterocycloalkyloxy_{(C≦18)}, acyloxy_{(C≦18)}, alkylamino_{(C≦8)}, alkylamino_{(C≦18)}, alkenylamino_{(C≦8)}, alkynylamino_{(C≦18)}, arylamino_{(C≦18)}, heteroarylamino_{(C≦8)}, heterocycloalkylamino_{(C≦8)}, amido_{(C≦18)}, or a substituted version of any of these groups; or —C(O)NR₂R₃ or —C(O)R₂; wherein: R₂ and R₃ are each independently hydrogen, hydroxy, alkyl_{(C≦6)}, aryl_{(C≦8)}, alkoxy_{(C≦6)}, or a substituted version of any of the last three groups; and wherein: the method comprises the steps of a) oxidizing the compound of formula XXXVI to form a compound of the formula:

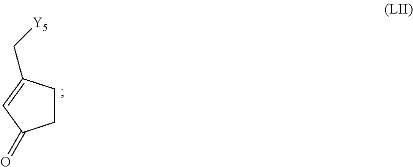

wherein: Y₅ is as defined above; b) reacting the compound of formula LII with a compound of the formula:

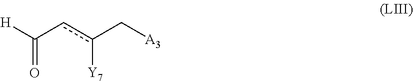

wherein: Y₇ is hydrogen, amino, hydroxy, mercapto, —OR₈, —SR₉, or —NR₁₀R₁₁; wherein: R₈ is alkyl_{(C≦12)}, aralkyl_{(C≦12)}, acyl_{(C≦12)}, a substituted version of any of these three groups, or a hydroxy protecting group; R₉ is alkyl_{(C≦12)}, aralkyl_{(C≦12)}, acyl_{(C≦12)}, a substituted version of any of these three groups, or a thiol protecting group; and R₁₀ and R₁₁ are each independently, alkyl_{(C≦12)}, aralkyl_{(C≦12)}, acyl_{(C≦12)}, a substituted version of any of these three groups, or a monovalent amino protecting group; or R₁₀ and R₁₁ are taken together to form a divalent amino protecting group; and A₃ is alkyl_{(C≦13)}, alkenyl_{(C≦13)}, alkynyl_{(C≦13)}, aralkyl_{(C≦13)}, heteroaralkyl_{(C≦13)}, or a substituted version of any of these groups; to form a compound of the formula:

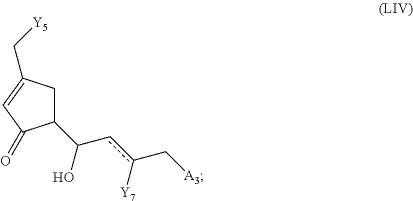

wherein: Y₅, Y₇, and A₃ are as defined above; c) dehydrating the compound of formula LIV to form a compound of the formula:

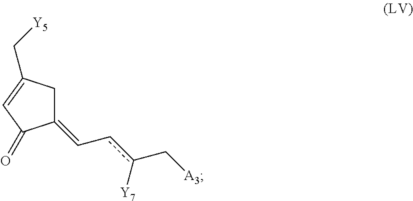

wherein: Y₅, Y₇, and A₃ are as defined above; and d) deprotecting the compound of formula LV to give a compound of the formula:

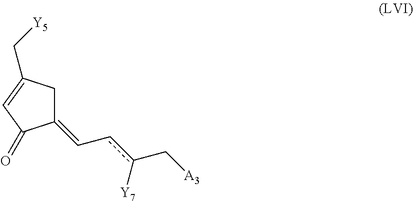

wherein: Y₅, Y₇, and A₃ are as defined above. In some embodiments, the compound is further defined as:

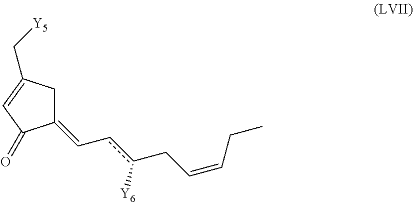

wherein: Y₅ is hydrogen, hydroxy, amino, cyano, or; alkyl_{(C≦12)}, alkenyl_{(C≦12)}, alkynyl_{(C≦12)}, aryl_{(C≦12)}, heteroaryl_{(C≦12)}, heterocycloalkyl_{(C≦12)}, alkoxy_{(C≦12)}, alkenyloxy_{(C≦12)}, alkynyloxy_{(C≦12)}, aryloxy_{(C≦12)}, heteroaryloxy_{(C≦12)}, heterocycloalkyloxy_{(C≦12)}, acyloxy_{(C≦12)}, alkylamino_{(C≦12)}, dialkylamino_{(C≦12)}, alkenylamino_{(C≦12)}, alkynylamino_{(C≦12)}, arylamino_{(C≦12)}, hetero arylamino_{(C≦12)}, heterocycloalkylamino_{(C≦12)}, amido_{(C≦12)}, or a substituted version of any of these groups; or —C(O)NR₂R₃ or —C(O)R₂; wherein: R₂ and R₃ are each independently hydrogen, hydroxy, alkyl_{(C≦6)}, aryl_{(C≦8)}, alkoxy_{(C≦6)}, or a substituted version of any of the last three groups; and Y₆ is —OH, —OTBS, or —H; wherein: the method comprises the steps of a) oxidizing the compound of formula XXXVI to form a compound of the formula:

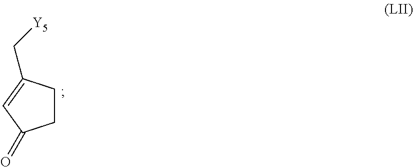

wherein: Y₅ is as defined above; b) reacting the compound of formula LII with a compound of the formula:

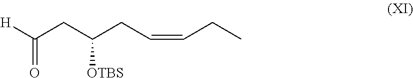 [SMILES: CC/C=C\C[C@@H](CC=O)O[Si](C)(C)C(C)(C)C]

to form a compound of the formula:

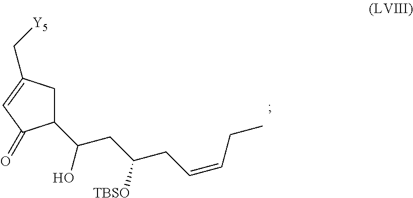

wherein: Y₅ is as defined above; c) dehydrating the compound of formula LVIII to form a compound of the formula:

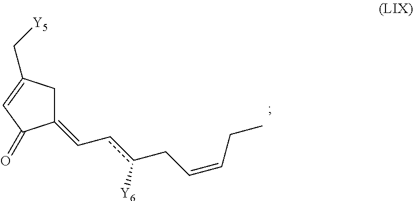

wherein: Y₅ is as defined above; and Y₆ is —OH, —OTBS, or —H; and d) deprotecting the compound of formula LIX to give a compound of the formula:

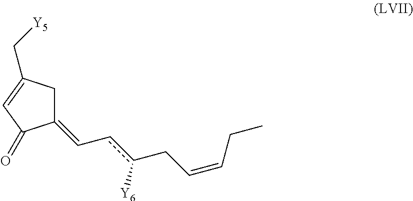

wherein: Y₅ is as defined above; and Y₆ is —OH or —H. In some embodiments, Y₅ is substituted alkyl_{(C≦8)}. In some embodiments, Y₅ is —CH₂OH. In some embodiments, Y₅ is —CH₂OTBS. In some embodiments, Y₅ is —C(O)NR₂R₃. In some embodiments, R₂ is alkyl_{(C≦6)}. In some embodiments, R₂ is —CH₃. In some embodiments, R₃ is alkoxy_{(C≦6)}. In some embodiments, R₃ is —OMe. In some embodiments, Y₅ is —C(O)N(OCH₃)CH₃. In some embodiments, Y₆ is —H. In some embodiments, Y₆ is —OH. In some embodiments, the oxidation of step a further comprises adding a metal salt. In some embodiments, the metal salt is a rhodium salt. In some embodiments, the metal salt is dirhodium tetracaprolactamate. In some embodiments, the oxidation of step a comprises adding about 0.005 equivalents of metal salt. In some embodiments, the oxidation of step a further comprises adding a base. In some embodiments, the base is a metal carbonate. In some embodiments, the metal salt is K₂CO₃. In some embodiments, the oxidation of step a comprises adding about 0.5 equivalents of base. In some embodiments, the oxidation of step a further comprises an oxygen atmosphere. In some embodiments, the oxygen atmosphere comprises a pressure from about 0.1 atmospheres to about 10 atmospheres. In some embodiments, the oxidation of step a further comprises reacting the compound of formula XXXVI with a oxidizing agent. In some embodiments, the oxidizing agent is a peroxide. In some embodiments, the oxidizing agent is tert-butyl hydroperoxide. In some embodiments, oxidation of step a comprises adding about 5 equivalents of oxidizing agent. In some embodiments, the oxidation of step a comprises adding the metal salt and the base and then the oxidizing agent. In some embodiments, the oxidation of step a further comprises a solvent. In some embodiments, the solvent is an organic solvent. In some embodiments, the solvent is a haloalkane_{(C≦12)}. In some embodiments, the solvent is dichloromethane. In some embodiments, the oxidation of step a reacts for a time period from about 1 hour to about 6 hours. In some embodiments, the time period is about 1.5 hours. In some embodiments, the oxidation of step a reacts at a temperature from about 0° C. to about 30° C. In some embodiments, the temperature is about 25° C. In some embodiments, the oxidation of step a further comprises adding a second portion of the metal salt and the oxidizing agent. In some embodiments, the second portion of the metal salt is about 0.005 equivalents. In some embodiments, the second portion of the oxidizing agent is about 5 equivalents. In some embodiments, the oxidation of step a further comprises allowing the oxidation to occur for a time period from about 1 hour to about 3 hours after the addition of the second portion of the metal salt and the oxidizing agent. In some embodiments, the time period is about 1.5 hours. In some embodiments, the oxidation of step a results in a yield of greater than 50%. In some embodiments, the yield is greater than 60%. In some embodiments, the compound of formula LIV is further defined as:

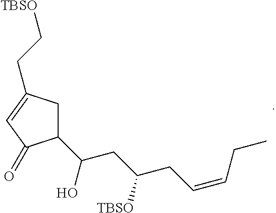 [SMILES: CC/C=C\C[C@@H](CC(O)C1CC(CCO[Si](C)(C)C(C)(C)C)=CC1=O)O[Si](C)(C)C(C)(C)C]

In some embodiments, the reaction of step b further comprises adding a base. In some embodiments, the base is lithium diisopropylamide. In some embodiments, the reaction of step b comprises adding about 1.95 equivalents of base. In some embodiments, the reaction of step b comprises adding the base and the compound of formula LII and then adding the compound of formula LIII. In some embodiments, the reaction of step b comprises adding about 1.2 equivalents of the compound of formula LIII. In some embodiments, the reaction of step b further comprises a solvent. In some embodiments, the solvent is an organic solvent. In some embodiments, the solvent is an ether_{(C≦12)}. In some embodiments, the solvent is tetrahydrofuran. In some embodiments, the reaction of step b reacts for a time period from about 15 minutes to about 3 hours. In some embodiments, the time period is about 20 minutes. In some embodiments, the reaction of step b reacts at a temperature from about −100° C. to about −70° C. In some embodiments, the temperature is about −78° C. In some embodiments, the reaction of step b further comprises allowing the reduction to occur for a time period from about 15 minutes to about 60 minutes after the addition of the compound of formula LIII. In some embodiments, the time period is about 30 minutes. In some embodiments, the reaction of step b results in a yield of greater than 50%. In some embodiments, the yield is greater than 65%. In some embodiments, the reaction of step b further comprises reacting the compound of formula LII with a group which enhances the ability of the hydroxyl group to be eliminated to form a compound containing a leaving group. In some embodiments, the group which enhances the ability of the hydroxyl group to be eliminated is an agent which enhances the leaving ability of a hydroxyl group. In some embodiments, the group which enhances the ability of the hydroxyl group to be eliminated is methanesulfonyl chloride. In some embodiments, the reaction of compound of formula LII with the group which enhances the ability of the hydroxyl group to be eliminated comprises adding 5 equivalents of methanesulfonyl chloride. In some embodiments, the reaction of compound of formula LII with the group which enhances the ability of the hydroxyl group to be eliminated further comprises a base. In some embodiments, the base is a triethylamine. In some embodiments, the reaction of compound of formula LII with the group which enhances the ability of the hydroxyl group to be eliminated further comprises a solvent. In some embodiments, the solvent is an organic solvent. In some embodiments, the solvent is haloalkane_{(C≦12)}. In some embodiments, the solvent is dichloromethane. In some embodiments, the reaction of a compound of formula LII with the group which enhances the ability of the hydroxyl group to be eliminated comprises reacting the compound for a time period from about 1 minute to about 30 minutes. In some embodiments, the time period is about 5 minutes. In some embodiments, the reaction of a compound of formula LII with the group which enhances the ability of the hydroxyl group to be eliminated reacts at a temperature from about −30° C. to about 30° C. In some embodiments, the temperature is about 0° C. In some embodiments, the reaction of a compound of formula LII with the group which enhances the ability of the hydroxyl group to be eliminated results in a yield of greater than 80%. In some embodiments, the yield is greater than 90%. In some embodiments, the compound of formula LIV is further defined as:

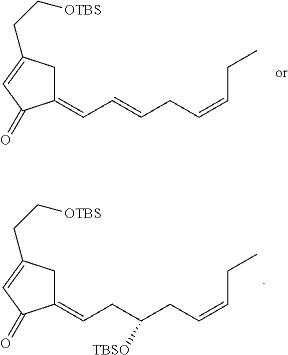 [SMILES: CC/C=C\C/C=C/C=C1\CC(CCO[Si](C)(C)C(C)(C)C)=CC1=O ; CC/C=C\C[C@@H](C/C=C1\CC(CCO[Si](C)(C)C(C)(C)C)=CC1=O)O[Si](C)(C)C(C)(C)C]

In some embodiments, the dehydration of step c further comprises reacting the compound containing a leaving group with Al₂O₃. In some embodiments, the dehydration of step c comprises adding about 7 equivalents of Al₂O₃. In some embodiments, the dehydration of step c further comprises adding about a second and third portion of Al₂O₃ at 2 and 4 hours, respectively. In some embodiments, the second and third portions of Al₂O₃ comprise adding about 7 equivalents of Al₂O₃. In some embodiments, the Al₂O₃ is activated by heating to about 400° C. under vacuum for about 5 minutes. In some embodiments, the reaction of step c further comprises a solvent. In some embodiments, the solvent is an organic solvent. In some embodiments, the solvent is a haloalkane_{(C≦12)}. In some embodiments, the solvent is dichloromethane. In some embodiments, the reaction of step c reacts for a time period from about 45 minutes to about 16 hours. In some embodiments, the time period is about 8 hours. In some embodiments, the reaction of step c reacts at a temperature from about 0° C. to about 35° C. In some embodiments, the temperature is about 25° C. In some embodiments, the temperature is room temperature. In some embodiments, the reaction of step c results in a yield of greater than 75%. In some embodiments, the yield is greater than 85%. In some embodiments, the compound of formula LV is further defined as:

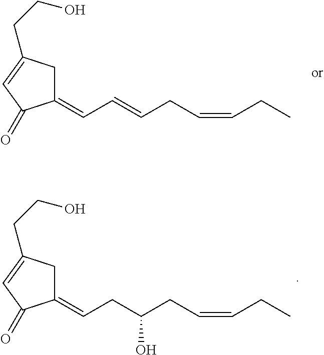 [SMILES: CC/C=C\C/C=C/C=C1\CC(CCO)=CC1=O ; CC/C=C\C[C@H](O)C/C=C1\CC(CCO)=CC1=O]

In some embodiments, the deprotection of step d further comprises reacting the compound of formula XL with a fluoride source. In some embodiments, the fluoride source is hydrofluoric acid. In some embodiments, the hydrofluoric acid is a 50% aqueous hydrofluoric acid solution. In some embodiments, the deprotection of step d comprises adding about 50 equivalents of HF. In some embodiments, the deprotection of step d further comprises a solvent. In some embodiments, the solvent is an aqueous and organic solvent mixture. In some embodiments, the organic solvent is an alkane_{(C≦12)} substituted with —CN. In some embodiments, the solvent is acetonitrile and water mixture. In some embodiments, the deprotection of step d reacts for a time period from about 30 minutes to about 4 hours. In some embodiments, the time period is about 1 hour. In some embodiments, the deprotection of step d reacts at a temperature from about −30° C. to about 30° C. In some embodiments, the temperature is about 0° C. In some embodiments, the deprotection of step d results in a yield of greater than 90%. In some embodiments, the yield is greater than 95%. In some embodiments, the yield is greater than 98%.
In yet another aspect, the present disclosure provides a method of preparing an enone of the formula:

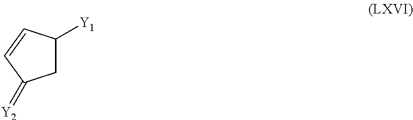

wherein: Y₁ is alkyl_{(C≦18)}, alkenyl_{(C≦18)}, alkynyl_{(C≦18)}, aralkyl_{(C≦18)}, heteroaralkyl_{(C≦18)}, or a substituted version of any of these groups; and Y₂ is O, S, and NR₁, wherein R₁ is hydrogen, alkyl_{(C≦6)}, or substituted alkyl_{(C≦6)}; comprising A) reacting a compound of the formula:

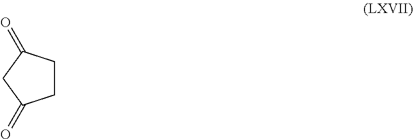

with an acid and a compound of the formula:

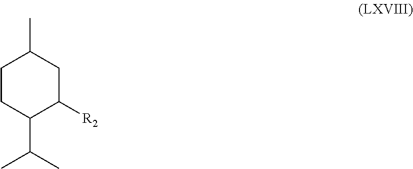

wherein: R₂ is hydroxy, mercapto, or —NHR₁ wherein R₁ is as defined above; to form a compound of the formula:

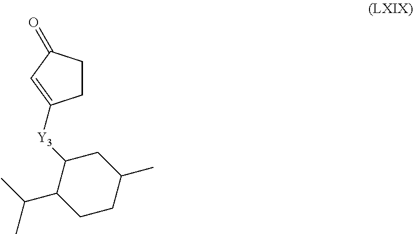

wherein: Y₃ is —O—, —S—, or —NR₁—, wherein R₁ is as defined above; B) reacting the compound of formula LXIX with a base and a compound of formula X₁—Y₁, wherein X₁ is halo or a group which enhances the ability of the hydroxyl group to be eliminated and Y₁ is as defined above; to form a compound of the formula:

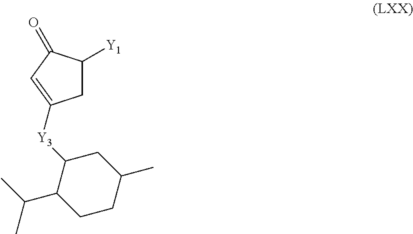

wherein: Y₁ and Y₃ are as defined above; and C) reducing the compound of formula LXX with a reducing agent to form a compound of formula LXVI. In some embodiments, the method further comprises epimerizing an enantiomer produced in step B) to a desired enantiomer with a base. In some embodiments, the base is an alkoxide base. In some embodiments, the base is potassium tert-butoxide. In some embodiments, the method further comprises an organic solvent. In some embodiments, the organic solvent is tetrahydrofuran. In some embodiments, the acid of step A) is a strong acid with a pKₐ in water of less than 0. In some embodiments, the acid is p-toluenesulfonic acid. In some embodiments, step A) further comprises an organic solvent. In some embodiments, the organic solvent is benzene. In some embodiments, the base of step B) is a strong non-nucleophilic base. In some embodiments, the base is dialkyl_{(C≦12)} amide. In some embodiments, the base is lithium diisopropylamide. In some embodiments, the reaction of step B) further comprises adding 1,3-dimethyl-2-imidazolidone. In some embodiments, the reaction of step B) further comprises adding the base and the compound of formula LXIX before adding X₁—Y₁. In some embodiments, the reaction of step B) further comprises an organic solvent. In some embodiments, the organic solvent is tetrahydrofuran. In some embodiments, the reducing agent of step C) is an aluminum hydride reagent. In some embodiments, the reducing agent is DIBAL-H. In some embodiments, the reduction of step C) further comprises an organic solvent. In some embodiments, the organic solvent is diethyl ether. In some embodiments, the method results in a yield from the three steps of greater than 25%. In some embodiments, the yield is greater than 40%. In some embodiments, the method further comprises a method of preparing a compound of the formula: X₁—Y₁, wherein the formula is further defined as:

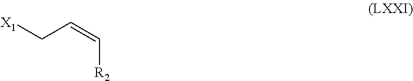

wherein: X₁ is halo or a group which enhances the ability of the hydroxyl group to be eliminated; and R₂ is alkyl_{(C≦15)}, alkenyl_{(C≦15)}, alkynyl_{(C≦15)}, aralkyl_{(C≦15)}, heteroaralkyl_{(C≦15)}, or a substituted version of any of these groups; and the method comprises A) reacting a compound of the formula:

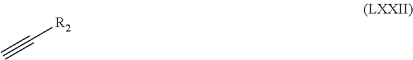

with paraformaldehyde in the presence of a base to form a compound of the formula:

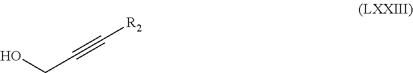

wherein: R₂ is as defined above; B) reducing the compound of formula LXXIII with a reducing agent to produce a compound of formula:

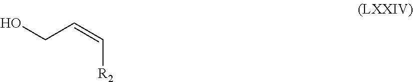

wherein: R₂ is as defined above; and C) reacting the compound of formula LXXIV with a leaving group agent or a halogenating agent to form a compound of LXXI. In some embodiments, X₁ is halo. In some embodiments, the base of step A) is a strong non-nucleophilic base. In some embodiments, the base is an alkyl_{(C≦12)} lithium. In some embodiments, the base is n-butyllithium. In some embodiments, paraformaldehyde is a compound of the formula: HO(CH₂O)ₙH wherein n is 1-250. In some embodiments, n is 8 to 100. In some embodiments, the reaction of step A) comprises an organic solvent. In some embodiments, the organic solvent is tetrahydrofuran. In some embodiments, the reducing agent of step B) comprises a transition metal, a ligand, a borohydride, and hydrogen gas. In some embodiments, the transition metal is nickel(II) acetate. In some embodiments, the transition metal is nickel(II) acetate tetrahydrate. In some embodiments, the ligand is ethylenediamine. In some embodiments, the borohydride is sodium borohydride. In some embodiments, the reduction of step B) further comprises an organic solvent. In some embodiments, the organic solvent is ethanol. In some embodiments, the reaction of step C) comprises a halogenating agent. In some embodiments, the halogenating agent is carbon tetrabromide and triphenyl phosphine. In some embodiments, the reaction of step C) further comprises an organic solvent. In some embodiments, the organic solvent is acetonitrile.

In another aspect, the present disclosure provides a method of preparing an enone of the formula:

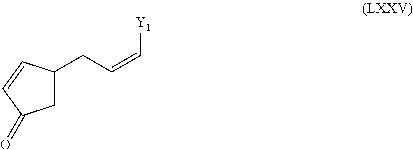

wherein: Y₁ is alkyl_{(C≦15)}, alkenyl_{(C≦15)}, alkynyl_{(C≦15)}, aralkyl_{(C≦15)}, heteroaralkyl_{(C≦15)}, or a substituted version of any of these groups; comprising: A) reacting a compound of the formula:

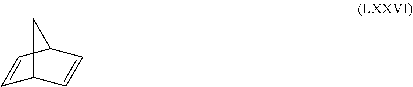

with an oxidizing agent to form a compound of the formula:

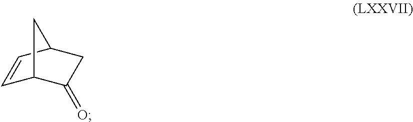

B) reacting the compound of formula LXXVII with a peroxide and a base to form a compound of the formula:

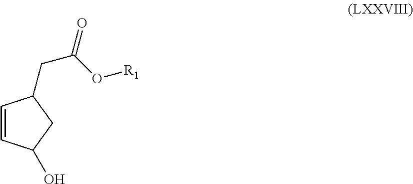

wherein: R₁ is alkyl_{(C≦12)} or substituted alkyl_{(C≦12)}; C) reducing the compound of formula LXXVIII with a reducing agent to form a compound of the formula;

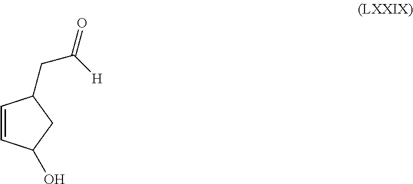

D) reacting the compound of formula LXXIX with a strong base, triphenylphosphine, and a haloalkane_{(C≦16)}, haloalkene_{(C≦16)}, haloalkyne_{(C≦16)}, haloaralkane_{(C≦16)}, haloheteroaralkane_{(C≦16)}, or a substituted version of any of these groups to form a compound of formula:

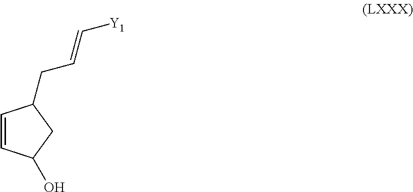

E) oxidizing the compound of formula LXXX with an oxidizing agent to form a compound of formula LXXV. In some embodiments, the oxidation of step A) comprises an oxidation with trichlorosilane followed by addition of a fluoride source and a peroxide. In some embodiments, the oxidation with trichlorosilane further comprises (S)-2-(diphenylphosphino)-2′-methoxy-1,1′-binaphthyl and a palladium salt. In some embodiments, the palladium salt is [η³-C₃H₅PdCl]₂. In some embodiments, the fluoride source is an inorganic fluoride. In some embodiments, the fluoride source is potassium fluoride. In some embodiments, the peroxide is hydrogen peroxide. In some embodiments, the oxidation further comprises an organic solvent. In some embodiments, the organic solvent is a mixture of two or more organic solvents. In some embodiments, the organic solvent is a mixture of tetrahydrofuran and methanol. In other embodiments, the solvent is dichloromethane. In some embodiments, the oxidation of step A) further comprises an oxidation with oxalyl chloride, dimethyl sulfoxide, and a base after the oxidation with trichlorosilane. In some embodiments, the base is a nitrogenous base. In some embodiments, the base is triethylamine. In some embodiments, the peroxide of step B) is triphenylmethyl peroxide. In some embodiments, the base of step B) is a strong non-nucleophilic base. In some embodiments, the base is an alkyl_{(C≦12)} lithium. In some embodiments, the base is n-butyllithium. In some embodiments, the reaction of step B) further comprises a second base. In some embodiments, the second base is an alkoxide_{(C≦12)} or substituted alkoxide_{(C≦12)}. In some embodiments, the second base is methoxide. In some embodiments, the reducing agent of step C) is an aluminum hydride. In some embodiments, the aluminum hydride is DIBAL-H. In some embodiments, the strong base of step D) is a strong non-nucleophilic base. In some embodiments, the strong base is NaHMDS. In some embodiments, the haloalkane_{(C≦16)} is ICH₂CH₂CH₂CH₂CH₂OC₆H₄OCH₃. In some embodiments, the reaction of step D) comprises an organic solvent. In some embodiments, the organic solvent is tetrahydrofuran. In some embodiments, the oxidizing agent of step E) is a chromate reagent. In some embodiments, the oxidizing agent is pyridinium chlorochromate. In some embodiments, the oxidation of step E) comprises an organic solvent. In some embodiments, the organic solvent is dichloromethane.
In yet another aspect, the present disclosure provides a method of preparing an aldehyde of the formula:

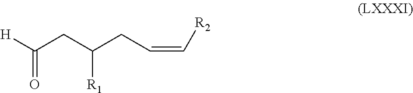

wherein: R₁ is halo, hydroxy, or —OX₁ wherein X₁ is a hydroxy protecting group; and R₂ is alkyl_{(C≦12)}, alkenyl_{(C≦12)}, alkynyl_{(C≦12)}, aralkyl_{(C≦12)}, heteroaralkyl_{(C≦12)}, or a substituted version of any of these groups; comprising: A) reducing a compound of the formula:

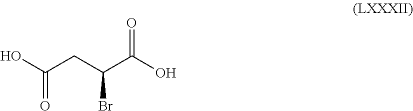

with a reducing agent to form a compound of the formula:

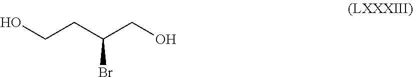

B) reacting a compound of the formula LXXXIII with a base to form a compound of the formula:

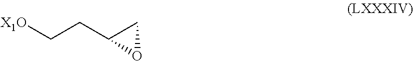

wherein: X₁ is hydrogen or a hydroxy protecting group; C) homologating the compound of formula LXXXIV to form a compound of the formula:

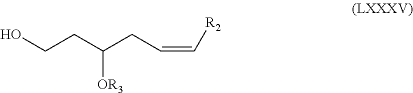

D) oxidizing the compound of formula LXXXV with an oxidizing agent to form a compound of LXXXI. In some embodiments, the reducing agent of step A) is a borohydride reagent. In some embodiments, reducing agent is boron trihydride dimethylsulfide. In some embodiments, step A) further comprises an organic solvent. In some embodiments, the organic solvent is tetrahydrofuran. In some embodiments, the base of step B) comprises a strong non-nucleophilic base. In some embodiments, the base is metal hydride. In some embodiments, the base is sodium hydride. In some embodiments, step B) further comprises adding a hydroxy group protecting agent. In some embodiments, the hydroxy group protecting agent is p-methoxybenzyl bromide. In some embodiments, the step B) further comprises tetrabutylammonium iodide. In some embodiments, step B) further comprises an organic solvent. In some embodiments, the organic solvent is tetrahydrofuran. In some embodiments, the homologation of step C) comprises: A) reacting a compound of formula LXXXIV with MgBr—CH═CHX₂ wherein X₂ is alkyl_{(C≦12)}, alkenyl_{(C≦12)}, alkynyl_{(C≦12)}, aralkyl_{(C≦12)}, heteroaralkyl_{(C≦12)}, or a substituted version of any of these groups to form a compound of the formula:

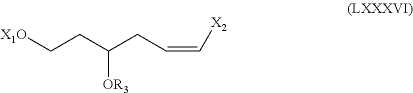

wherein: R₃ is hydrogen and X₁ and X₂ are as defined above; B) protecting the compound of formula LXXXVI with a hydroxy protecting agent and a base to form a compound of the formula:

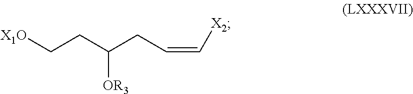

wherein: X₁ and X₂ are as defined above and R₃ is a hydroxy protecting group. In some embodiments, the homologation of step C) further comprises A) reacting the compound of formula LXXXVII with an oxidizing agent to form a compound of the formula:

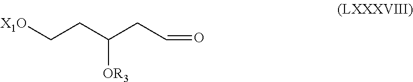

wherein: X₁ and R₃ is as defined above; and; B) reacting the compound of formula LXXXVIII with phosphine, a haloalkane_{(C≦11)}, haloalkene_{(C≦11)}, haloalkyne_{(C≦11)}, haloaralkane_{(C≦11)}, haloheteroaralkane_{(C≦11)}, or a substituted version of any of these groups, and a base to form a compound of the formula:

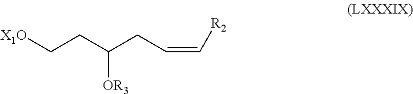

wherein: X₁, R₂, and R₃ are as defined above. In some embodiments, the oxidizing agent of step A) is an osmium compound. In some embodiments, the oxidizing agent is osmium tetraoxide. In some embodiments, the oxidizing agent further comprises a second oxidizing agent. In some embodiments, the second oxidizing agent is a hypervalent iodide compound. In some embodiments, the second oxidizing agent is sodium periodate. In some embodiments, the oxidation of step A) further comprises an organic solvent. In some embodiments, the organic solvent is a mixture. In some embodiments, the organic solvent is a mixture of tetrahydrofuran and water. In some embodiments, the base of step B) is a strong non-nucleophilic base. In some embodiments, the base is NaHMDS. In some embodiments, the phosphine is triphenyl phosphine. In some embodiments, the reaction of step B) further comprises an organic solvent. In some embodiments, the organic solvent is tetrahydrofuran. In some embodiments, the reaction of step A) further comprises an organic solvent. In some embodiments, the organic solvent is tetrahydrofuran. In some embodiments, the hydroxy protecting agent of step B) is a alkylsilyl_{(C≦18)} halide. In some embodiments, the hydroxy protecting agent is tri-tert-butylsilyl chloride. In some embodiments, the base of step B) is a nitrogenous base. In some embodiments, the base is imidazole. In some embodiments, the protection of step B) further comprises an organic solvent. In some embodiments, the organic solvent is dichloromethane. In some embodiments, the homologation of step C) further comprises deprotecting the compound of a formula LXXXIX with an oxidizing agent. In some embodiments, the oxidizing agent is 2,3-dichloro-5,6-dicyano-para-benzoquinone. In some embodiments, the deprotection further comprises a solvent. In some embodiments, the organic solvent is a mixture. In some embodiments, the organic solvent is a mixture of dichloromethane and pH 7.0 aqueous buffer. In some embodiments, the homologation of step C) comprises: A) reacting the compound of formula LXXXV with a terminal alkyne_{(C≦14)}, a Lewis base, and a base to produce a compound of the formula:

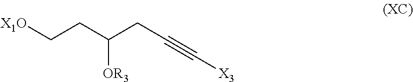

wherein: X₁ is as defined above, R₃ is hydrogen, and X₃ is the terminal alkyne_{(C≦12)}; B) protecting the compound of the formula XC with a hydroxy protecting agent and a base to form a compound of the formula:

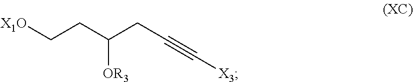

and
wherein: X₁ and X₃ are as defined above and R₃ is a hydroxy protecting group; and C) reducing the compound of the formula XC with a reducing agent to form a compound of the formula LXXXV. In some embodiments, the Lewis base is a boron compound. In some embodiments, the Lewis base is trifluoroborate etherate. In some embodiments, the base is a strong base. In some embodiments, the base is an alkyl_{(C≦12)} lithium. In some embodiments, the base is n-butyl lithium. In some embodiments, the reaction of step A) further comprises an organic solvent. In some embodiments, the organic solvent is tetrahydrofuran. In some embodiments, the hydroxy protecting agent of step B) is a silylating agent. In some embodiments, the hydroxy protecting agent is tri-tert-butylsilyl chloride. In some embodiments, the base of step B) is a nitrogenous base. In some embodiments, the base is imidazole. In some embodiments, the protection of step B) further comprises an organic solvent. In some embodiments, the organic solvent is dichloromethane. In some embodiments, the reducing agent of step C) comprises a transition metal, a ligand, a borohydride, and hydrogen gas. In some embodiments, the transition metal is nickel(II) acetate. In some embodiments, the transition metal is nickel(II) acetate tetrahydrate. In some embodiments, the ligand is ethylenediamine. In some embodiments, the borohydride is sodium borohydride. In some embodiments, the reduction of step C) further comprises an organic solvent. In some embodiments, the organic solvent is ethanol. In some embodiments, the oxidizing agent of step D) is a hypervalent iodide compound. In some embodiments, the oxidizing agent is 1,1,1-triacetoxy-1,1-dihydro-1,2-benziodoxol-3(1H)-one. In some embodiments, the oxidation of step D) further comprises an organic solvent. In some embodiments, the organic solvent is dichloromethane.

It is contemplated that any method or composition described herein can be implemented with respect to any other method or composition described herein. For example, an aldehyde synthesized by one method may be used in the preparation of a final compound according to a different method.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed.

FIG. 1—Molecular structures of Δ¹²-PGJ₃, Δ¹²-PGJ₃ methyl ester, 15-deoxy-Δ^{12,14}-PGJ₂ and Δ¹²-PGJ₂.

FIG. 2—Retrosynthetic analysis for Δ¹²-PGJ₃.

FIG. 3—Retrosynthetic analysis for alternative synthesis pathways for Δ¹²-PGJ₃.

### DESCRIPTION OF ILLUSTRATIVE EMBODIMENTS

AML is one of the most common types of leukemia in adults. Unfortunately, the five year relative survival rates for AML are the lowest when compared to other forms of leukemia. AML is a stem cell disease where LSCs occupy the apex of the disease hierarchy. LSCs can self renew and generate non-stem cell progeny that make up the bulk of the leukemia cells. Although chemotherapy agents can effectively target bulk leukemia cells, LSCs have active mechanisms to avoid killing by these drugs. As a consequence, failure to eliminate LSCs results in relapse of the disease. Because of this property, specific targeting of LSCs is essential for successful treatment.

Although the need for new anti-LSC based therapies is well recognized, the identification of mechanism-based drugs to target LSCs has been lacking. Clearly new approaches are needed. Described herein are compositions and methods relating to an ω-3-derived fatty acid, Δ¹²-PGJ₃, which was previously reported to effectively eradicate LSCs in two mouse models of chronic leukemia. In experiments reported in U.S. Patent Publication 2013/0005737, Δ¹²-PGJ₃ was shown to effectively target AML LSCs by inducing apoptosis in murine models of AML and in human AML leukemia samples. In contrast, PGJ₃ has no effect on normal hematopoietic stem cells or the differentiation of hematopoietic progenitors. Δ¹²-PGJ₃ acts by inducing the expression of p53 in LSCs and leukemia cells. High-level expression of p53 in LSCs is incompatible with self renewal and leads to apoptosis.

The present disclosure provides new synthetic methods for Δ¹²-PGJ₃ and also permits the production of new Δ¹²-PGJ₃ derivatives. These and other aspects of the disclosure are described in greater detail below.

### I. Δ¹²-PGJ₃ PROSTAGLANDINS, DERIVATIVES, AND FORMULATIONS THEREOF

In one aspect, the present invention provides compounds of the formula:

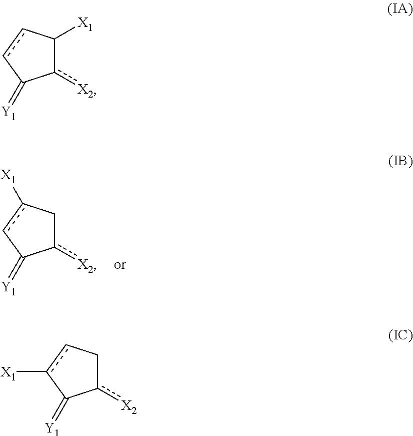

wherein: Y₁ is O, NR₁, or N—OR₁; wherein: R₁ is hydrogen, alkyl_{(C≦6)}, or substituted alkyl_{(C≦6)}; X₁ is hydrogen, alkyl_{(C≦8)}, substituted alkyl_{(C≦8)},

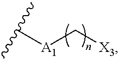

or taken together with X₂ as defined below; wherein: A₁ is alkanediyl_{(C≦8)}, alkenediyl_{(C≦8)}, alkynediyl_{(C≦8)}, arenediyl_{(C≦12)}, heteroarenediyl_{(C≦12)}, or a substituted version of any of these groups; n is 0, 1, 2, 3, 4, 5, or 6; X₃ is hydrogen, hydroxy, amino, cyano, or; alkyl_{(C≦12)}, alkenyl_{(C≦12)}, alkynyl_{(C≦12)}, aryl_{(C≦12)}, heteroaryl_{(C≦12)}, heterocycloalkyl_{(C≦12)}, alkoxy_{(C≦12)}, alkenyloxy_{(C≦12)}, alkynyloxy_{(C≦12)}, aryloxy_{(C≦12)}, heteroaryloxy_{(C≦12)}, heterocycloalkyloxy_{(C≦12)}, acyloxy_{(C≦12)}, alkylamino_{(C≦12)}, dialkylamino_{(C≦12)}, alkenylamino_{(C≦12)}, alkynylamino_{(C≦12)}, arylamino_{(C≦12)}, heteroarylamino_{(C≦12)}, heterocycloalkylamino_{(C≦12)}, amido_{(C≦12)}, or a substituted version of any of these groups; or —C(O)NR₂R₃, —C(O)R₂; or —Y₂—R₄; wherein: R₂ and R₃ are each independently hydrogen, hydroxy, alkyl_{(C≦8)}, aryl_{(C≦8)}, alkoxy_{(C≦8)}, alkylsulfonyl_{(C≦8)}, arylsulfonyl_{(C≦8)}, or a substituted version of any of the last five groups; or R₂ is -alkoxydiyl_{(C≦6)}-S(O)₂-aryl_{(C≦12)} or a substituted version of this group; Y₂ is alkanediyl_{(C≦12)}, substituted alkanediyl_{(C≦12)}; alkoxydiyl_{(C≦12)}, or substituted alkoxydiyl_{(C≦12)}; and R₄ is hydrogen, —C(O)NR₂R₃, or —C(O)R₂; wherein R₂ and R₃ are as defined above and X₂ is

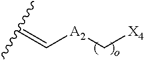

or taken together with X₁ as defined below; wherein: A₂ is alkanediyl_{(C≦8)}, alkenediyl_{(C≦8)}, alkynediyl_{(C≦8)} or a substituted version of any of these groups; or —CH₂CH(OR₄)—; wherein: R₄ is alkyl_{(C≦12)}; alkenyl_{(C≦12)}, alkynyl_{(C≦12)}, aryl_{(C≦12)}, heteroaryl_{(C≦12)}, acyl_{(C≦12)}, or a substituted version of any of these groups; X₄ is hydrogen, hydroxy, or alkyl_{(C≦12)}, alkenyl_{(C≦12)}, alkynyl_{(C≦12)}, aryl_{(C≦12)}, heteroaryl_{(C≦12)}, heterocycloalkyl_{(C≦12)}, aryloxy_{(C≦12)}, heteroaryloxy_{(C≦12)}, heterocycloalkoxy_{(C≦12)}, arylthio_{(C≦12)}, heteroarylthio_{(C≦12)}, heterocycloalkylthio_{(C≦12)}, arylsulfinyl_{(C≦12)}, heteroarylsulfinyl_{(C≦12)}, heterocycloalkylsulfinyl_{(C≦12)}, arylsulfonyl_{(C≦12)}, heteroarylsulfonyl_{(C≦12)}, heterocycloalkylsulfonyl_{(C≦12)}, or a substituted version of any of these groups; and o is 0, 1, 2, 3, 4, 5, or 6; wherein: X₁ and X₂ are taken together as shown in formula (II):

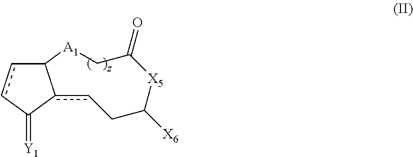

wherein: Y₁ is O, NH, or N—OR₁; wherein: R₁ is hydrogen, alkyl_{(C≦6)}, or substituted alkyl_{(C≦6)}; A₁ is alkanediyl_{(C≦8)}, alkenediyl_{(C≦8)}, alkynediyl_{(C≦8)}, arenediyl_{(C≦12)}, heteroarenediyl_{(C≦12)}, or a substituted version of any of these groups; z is 1, 2, 3, 4, 5, or 6; X₅ is CR₄R₅, O, NH, NR₆, or S; wherein: R₄, R₅, and R₆ are each independently H, alkyl_{(C≦8)}, aryl_{(C≦8)}, aralkyl_{(C≦8)}; or a substituted version of any of the last three groups; and X₆ is alkyl_{(C≦12)}; alkenyl_{(C≦12)}, alkynyl_{(C≦12)}, aryl_{(C≦12)}, heteroaryl_{(C≦12)} or a substituted version of any of these groups; provided that the compound does not have the formula:

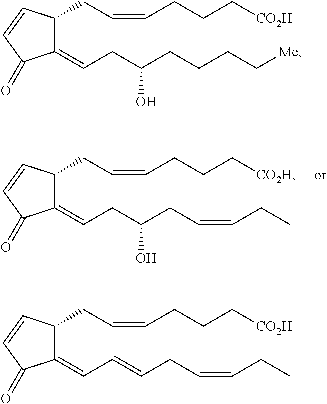 [SMILES: CCCCC[C@H](O)C/C=C1/C(=O)C=C[C@@H]1C/C=C\CCCC(=O)O ; CC/C=C\C[C@H](O)C/C=C1/C(=O)C=C[C@@H]1C/C=C\CCCC(=O)O ; "or" ; CC/C=C\C/C=C/C=C1/C(=O)C=C[C@@H]1C/C=C\CCCC(=O)O]

or a pharmaceutically acceptable salt thereof. In some embodiments, the compounds of the formula:

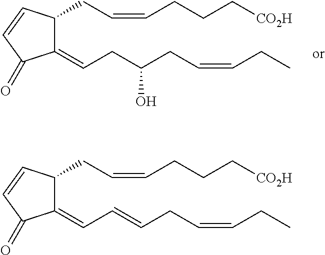 [SMILES: CC/C=C\C[C@H](O)C/C=C1/C(=O)C=C[C@@H]1C/C=C\CCCC(=O)O ; CC/C=C\C/C=C/C=C1/C(=O)C=C[C@@H]1C/C=C\CCCC(=O)O]

are specifically excluded from the compounds, compositions, or methods. In other embodiments, the compounds of the formula:

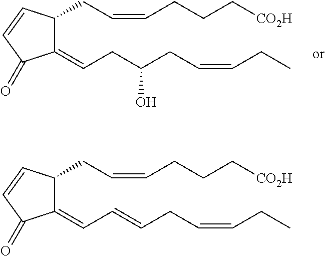 [SMILES: CC/C=C\C[C@H](O)C/C=C1/C(=O)C=C[C@@H]1C/C=C\CCCC(=O)O ; CC/C=C\C/C=C/C=C1/C(=O)C=C[C@@H]1C/C=C\CCCC(=O)O]

are specifically included from the compounds, compositions, or methods.

The compounds provided by the present disclosure are shown, for example, above in the summary of the invention section and in the claims below. They may be made using the methods outlined in the Examples section. Δ¹²-PGJ₃ can be synthesized according to the methods described, for example, in the Examples section below. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

The Δ¹²-PGJ₃ and derivatives thereof of the invention may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the Δ¹²-PGJ₃ and derivatives thereof of the present invention can have the S or the R configuration.

Chemical formulas used to represent compounds of the invention will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

Compounds of the invention may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the Δ¹²-PGJ₃ and derivatives thereof of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

Compounds of the present invention may also exist in prodrug form. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

B. Formulations

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

Formulations for oral use include tablets containing the active ingredient(s) (e.g., Δ¹²PGJ₃ or a derivative thereof) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

### II. LEUKEMIA

Clinically and pathologically, leukemia is subdivided into a variety of large groups. The first division is between its acute and chronic forms. Acute leukemia is characterized by a rapid increase in the number of immature blood cells. Crowding due to such cells makes the bone marrow unable to produce healthy blood cells. Immediate treatment is required in acute leukemia due to the rapid progression and accumulation of the malignant cells, which then spill over into the bloodstream and spread to other organs of the body. Acute forms of leukemia are the most common forms of leukemia in children. Chronic leukemia is characterized by the excessive build up of relatively mature, but still abnormal, white blood cells. Typically taking months or years to progress, the cells are produced at a much higher rate than normal, resulting in many abnormal white blood cells. Whereas acute leukemia must be treated immediately, chronic forms are sometimes monitored for some time before treatment to ensure maximum effectiveness of therapy. Chronic leukemia mostly occurs in older people, but can theoretically occur in any age group.

Additionally, the diseases are subdivided according to which kind of blood cell is affected. This split divides leukemias into lymphoblastic or lymphocytic leukemias and myeloid or myelogenous leukemias. In lymphoblastic or lymphocytic leukemias, the cancerous change takes place in a type of marrow cell that normally goes on to form lymphocytes, which are infection-fighting immune system cells. Most lymphocytic leukemias involve a specific subtype of lymphocyte, the B cell. In myeloid or myelogenous leukemias, the cancerous change takes place in a type of marrow cell that normally goes on to form red blood cells, some other types of white cells, and platelets.

Combining these two classifications provides a total of four main categories. Within each of these four main categories, there are typically several subcategories. Finally, some rarer types are usually considered to be outside of this classification scheme:
Acute lymphoblastic leukemia (ALL) is the most common type of leukemia in young children. This disease also affects adults, especially those age 65 and older. Standard treatments involve chemotherapy and radiotherapy. The survival rates vary by age: 85% in children and 50% in adults. Subtypes include precursor B acute lymphoblastic leukemia, precursor T acute lymphoblastic leukemia, Burkitt's leukemia, and acute biphenotypic leukemia.
Chronic lymphocytic leukemia (CLL) most often affects adults over the age of 55. It sometimes occurs in younger adults, but it almost never affects children. Two-thirds of affected people are men. The five-year survival rate is 75%. It is incurable, but there are many effective treatments. One subtype is B-cell prolymphocytic leukemia, a more aggressive disease.
Acute myelogenous leukemia (AML) occurs more commonly in adults than in children, and more commonly in men than women. AML is treated with chemotherapy. The five-year survival rate is 40%, except for acute promyelocytic leukemia (APL), which is over 90%. Subtypes of AML include acute promyelocytic leukemia (APL), acute myeloblastic leukemia, and acute megakaryoblastic leukemia.
Chronic myelogenous leukemia (CML) occurs mainly in adults; a very small number of children also develop this disease. Treatment is with imatinib (Gleevec in United States, Glivec in Europe) or other drugs. The five-year survival rate is 90%. One subtype is chronic monocytic leukemia.
Hairy cell leukemia (HCL) is sometimes considered a subset of chronic lymphocytic leukemia, but does not fit neatly into this pattern. About 80% of affected people are adult men. No cases in children have been reported. HCL is incurable, but easily treatable. Survival is 96% to 100% at ten years. T-cell prolymphocytic leukemia (T-PLL) is a very rare and aggressive leukemia affecting adults; somewhat more men than women are diagnosed with this disease. Despite its overall rarity, it is also the most common type of mature T cell leukemia; nearly all other leukemias involve B cells. It is difficult to treat, and the median survival is measured in months.
Large granular lymphocytic leukemia may involve either T-cells or NK cells; like hairy cell leukemia, which involves solely B cells, it is a rare and indolent (not aggressive) leukemia.
   Adult T-cell leukemia is caused by human T-lymphotropic virus (HTLV), a virus similar to HIV. Like HIV, HTLV infects CD4+ T-cells and replicates within them; however, unlike HIV, it does not destroy them. Instead, HTLV “immortalizes” the infected T-cells, giving them the ability to proliferate abnormally. Human T cell lymphotropic virus types I and II (HTLV-VII) are endemic in certain areas of the world.

### III. THERAPIES

A. Pharmaceutical Formulations and Routes of Administration

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. Such routes include oral, nasal, buccal, rectal, vaginal or topical route. Alternatively, administration may be by orthotopic, intradermal, subcutaneous, intramuscular, intratumoral, intraperitoneal, or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

B. Methods of Treatment

Cancer, known medically as a malignant neoplasm, is a broad group of diseases involving unregulated cell growth. In cancer, cells divide and grow uncontrollably, forming malignant tumors, and invading nearby parts of the body. The cancer may also spread to more distant parts of the body through the lymphatic system or bloodstream. Not all tumors are cancerous; benign tumors do not invade neighboring tissues and do not spread throughout the body. There are over 200 different known cancers that affect humans.

The causes of cancer are diverse, complex, and only partially understood. Many things are known to increase the risk of cancer, including tobacco use, dietary factors, certain infections, exposure to radiation, lack of physical activity, obesity, and environmental pollutants. These factors can directly damage genes or combine with existing genetic faults within cells to cause cancerous mutations. Approximately 5-10% of cancers can be traced directly to inherited genetic defects. Many cancers could be prevented by not smoking, eating more vegetables, fruits and whole grains, eating less meat and refined carbohydrates, maintaining a healthy weight, exercising, minimizing sunlight exposure, and being vaccinated against some infectious diseases.

In particular, the compositions disclosed herein find use in treating leukemia in a subject (e.g., a human subject). Examples of leukemias that can be treated using the compositions include Acute Myelogenous Leukemia (AML), CML, Acute Lymphocytic Leukemia (ALL) and Chronic Lymphocytic Leukemia (CLL). In one embodiment, a composition includes a therapeutically effective amount of Δ¹²-PGJ₃, or a derivative thereof (a first anti-cancer drug), for inhibiting leukemia stem cell (LSC) growth in a subject having LSCs, and a pharmaceutically acceptable carrier Inhibiting LSC growth includes inducing death (killing of) of the cancer cells, and/or inducing differentiation of the cancer cells (promoting a more differentiated phenotype, e.g., causing differentiation of LSCs into terminally differentiated cells). Any suitable form of Δ¹²-PGJ₃ or derivative thereof can be used (e.g., synthesized, isolated). Δ¹²-PGJ₃ derivatives that may find particular use in the compositions and methods described herein are those that induce apoptosis or differentiation of LSCs (e.g., Δ¹²-PGJ₃ lactone). In such embodiments, when administered to a subject, the composition induces apoptosis of LSCs. The composition can further include one or more additional anti-cancer drugs (e.g., a second anti-cancer drug). Examples of additional anti-cancer drugs include imatinib, nilotinib, dasafanib, new generation BCR-ABL inhibitors, and standard chemotherapy drugs such as cytarabine or doxorubicin or similar classes of drugs. In one embodiment, a combination therapy including imatinib or a new generation BCR-ABL inhibitor and Δ¹²-PGJ₃ may be particularly therapeutic.

The compositions described above are preferably administered to a mammal (e.g., rodent, human, non-human primates, canine, bovine, ovine, equine, feline, etc.) in an effective amount, that is, an amount capable of producing a desirable result in a treated subject (e.g., inhibiting growth of LSCs and/or inducing death of LSCs in the subject). Toxicity and therapeutic efficacy of the compositions utilized in methods of the invention can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms of the cancer and other drugs being administered concurrently. A composition as described herein is typically administered at a dosage that induces death of LSCs (e.g., induces apoptosis of LSCs), as assayed by identifying a reduction in hematological parameters (Complete blood count (CBC)), or cancer cell growth or proliferation. In the experiments described herein, the amount of Δ¹²-PGJ₃ used to eradicate LSCs was calculated to be 0.6 micrograms/day/gram mouse for 7 days. Generally, the dose is in mg/Kg subject/day=μg/g subject/day. In a typical embodiment, a dose in the range of about 0.025 to about 0.05 mg/Kg/day is administered. Such a dose is typically administered once a day for a few weeks.

The therapeutic methods of the invention (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, marker (as defined herein), family history, and the like).

In one embodiment, the invention provides a method of monitoring treatment progress. The method includes the step of determining a level of changes in hematological parameters and LSC analysis with cell surface proteins as diagnostic markers (which can include, for example, but are not limited to CD34, CD38, CD90, and CD117) or diagnostic measurement (e.g., screen, assay) in a subject suffering from or susceptible to a disorder or symptoms thereof associated with cancer (e.g., leukemia) in which the subject has been administered a therapeutic amount of a composition as described herein. The level of marker determined in the method can be compared to known levels of marker in either healthy normal controls or in other afflicted patients to establish the subject's disease status. In preferred embodiments, a second level of marker in the subject is determined at a time point later than the determination of the first level, and the two levels are compared to monitor the course of disease or the efficacy of the therapy. In certain preferred embodiments, a pre-treatment level of marker in the subject is determined prior to beginning treatment according to the methods described herein; this pre-treatment level of marker can then be compared to the level of marker in the subject after the treatment commences, to determine the efficacy of the treatment.

C. Combination Therapies

It is very common in the field of cancer therapy to combine therapeutic modalities. The following is a general discussion of therapies that may be used in conjunction with the therapies of the present disclosure.

To treat cancers using the methods and compositions of the present invention, one would generally contact a tumor cell or subject with a peptide and at least one other therapy. These therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the peptide and the other includes the other agent.

Alternatively, the peptide may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the peptide or the other therapy will be desired. Various combinations may be employed, where a compound of the present disclosure is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. The following is a general discussion of cancer therapies that may be used in combination with the peptides of the present disclosure.

1. Chemotherapy

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma I and calicheamicin omega I; dynemicin, including dynemicin A, uncialamycin, and derivatives thereof; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, famesyl-protein tansferase inhibitors, transplatinum, 5-fluorouracil, vincristin, vinblastin and methotrexate and pharmaceutically acceptable salts, acids or derivatives of any of the above.

In particular, tyrosine kinase inhibitors are a class of agent that can be used in combination with the compounds of the present application. For example, Imatinib is a tyrosine-kinase inhibitor used in the treatment of multiple cancers, most notably Philadelphia chromosome-positive (Ph⁺) chronic myelogenous leukemia (CML). Like all tyrosine-kinase inhibitors, imatinib works by preventing a tyrosine kinase enzyme, in this case BCR-Abl, from phosphorylating subsequent proteins and initiating the signaling cascade necessary for cancer development, thus preventing the growth of cancer cells and leading to their death by apoptosis. Because the BCR-Abl tyrosine kinase enzyme exists only in cancer cells and not in healthy cells, imatinib works as a form of targeted therapy only cancer cells are killed through the drug's action. In this regard, imatinib was one of the first cancer therapies to show the potential for such targeted action, and is often cited as a paradigm for research in cancer therapeutics.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present invention may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used with the present invention. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1β, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

It also should be pointed out that any of the foregoing therapies may prove useful by themselves in treating cancer.

### IV. SYNTHETIC METHODS

In some aspects, the compounds of this invention can be synthesized using the methods of organic chemistry as described in this application. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein

1. Process Scale-Up

The synthetic methods described herein can be further modified and optimized for preparative, pilot- or large-scale production, either batch of continuous, using the principles and techniques of process chemistry as applied by a person skilled in the art. Such principles and techniques are taught, for example, in Practical Process Research & Development (2000), which is incorporated by reference herein. The synthetic method described herein could be used to produce preparative scale quantities of Δ¹²-PGJ₃ and derivatives thereof.

2. Chemical Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; and “sulfinyl” means —S(O)—.

In the context of chemical formulas, the symbol “—” means a single bond, “═” means a double bond, and “≡” means triple bond. The symbol “- - - -” represents an optional bond, which if present is either single or double. The symbol “ [symbol] ” represents a single bond or a double bond. Thus, for example, the formula

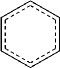

includes

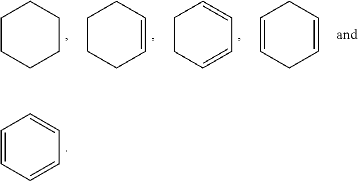

And it is understood that no one such ring atom forms part of more than one double bond. Furthermore, it is noted that the covalent bond symbol “—”, when connecting one or two stereogenic atoms, does not indicate any preferred stereochemistry. Instead, it cover all stereoisomers as well as mixtures thereof. The symbol “ [symbol] ”, when drawn perpendicularly across a bond

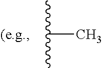

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. The bond orders described above are not limiting when one of the atoms connected by the bond is a metal atom (M). In such cases, it is understood that the actual bonding may comprise significant multiple bonding and/or ionic character. Therefore, unless indicated otherwise, the formulas M-C, M=C, M- - - -C, and M [symbol] C, each refers to a bond of any and type and order between a metal atom and a carbon atom. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

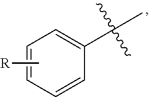

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

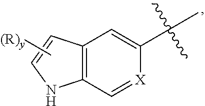

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the groups and classes below, the following parenthetical subscripts further define the group/class as follows: “(Cn)” defines the exact number (n) of carbon atoms in the group/class. “(C≦n)” defines the maximum number (n) of carbon atoms that can be in the group/class, with the minimum number as small as possible for the group in question, e.g., it is understood that the minimum number of carbon atoms in the group “alkenyl_{(C≦8)}” or the class “alkene_{(C≦8)}” is two. For example, “alkoxy_{(C≦10)}” designates those alkoxy groups having from 1 to 10 carbon atoms. (Cn-n′) defines both the minimum (n) and maximum number (n′) of carbon atoms in the group. Similarly, “alkyl_{(C2-10)}” designates those alkyl groups having from 2 to 10 carbon atoms.

The term “saturated” as used herein means the compound or group so modified has no carbon-carbon double and no carbon-carbon triple bonds, except as noted below. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded.

The term “aliphatic” when used without the “substituted” modifier signifies that the compound/group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single bonds (alkanes/alkyl), or unsaturated, with one or more double bonds (alkenes/alkenyl) or with one or more triple bonds (alkynes/alkynyl).

The term “alkyl” when used without the “substituted” modifier refers to a monovalent saturated aliphatic group with a carbon atom as the point of attachment, a linear or branched, cyclo, cyclic or acyclic structure, and no atoms other than carbon and hydrogen. Thus, as used herein cycloalkyl is a subset of alkyl, with the carbon atom that forms the point of attachment also being a member of one or more non-aromatic ring structures wherein the cycloalkyl group consists of no atoms other than carbon and hydrogen. As used herein, the term does not preclude the presence of one or more alkyl groups (carbon number limitation permitting) attached to the ring or ring system. The groups —CH₃ (Me), —CH₂CH₃ (Et), —CH₂CH₂CH₃ (n-Pr or propyl), —CH(CH₃)₂ (i-Pr, ⁱPr or isopropyl), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), —CH₂C(CH₃)₃ (neo-pentyl), cyclobutyl, cyclopentyl, cyclohexyl, and cyclohexylmethyl are non-limiting examples of alkyl groups. The term “alkanediyl” when used without the “substituted” modifier refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, —CH₂CH₂CH₂—, and

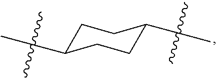 [SMILES: *C1CCC(*)CC1]

are non-limiting examples of alkanediyl groups. The term “alkylidene” when used without the “substituted” modifier refers to the divalent group ═CRR′ in which R and R′ are independently hydrogen, alkyl, or R and R′ are taken together to represent an alkanediyl having at least two carbon atoms. Non-limiting examples of alkylidene groups include: ═CH₂, ═CH(CH₂CH₃), and ═C(CH₃)₂. An “alkane” refers to the compound H—R, wherein R is alkyl as this term is defined above. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —OSi(CH₃)₂C(CH₃)₃, —OSi(CH₃)₃, —OSi(CH₂CH₃)₂, —OSi(CH(CH₃)₂)₃, —OSi(C₆H₅)₂C(CH₃)₃, —C(O)CH₃, —NHCH₃, —NHCH₂CH₃, —N(CH₃)₂, —C(O)NH₂, —OC(O)CH₃, —S(O)₂NH₂, or —OX₁ or —NX₂X₃, wherein X₁ is a hydroxy protecting group, X₂ is a monovalent amine protecting group or is taken together with X₃ and is a divalent amine protecting group and X₃ is hydrogen or is taken together with X₂ and is a divalent amine protecting group. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which one or more hydrogen atoms has been substituted with a halo group and no other atoms aside from carbon, hydrogen and halogen are present. The group, —CH₂Cl is a non-limiting example of a haloalkyl. The term “fluoroalkyl” is a subset of substituted alkyl, in which one or more hydrogen has been substituted with a fluoro group and no other atoms aside from carbon, hydrogen and fluorine are present. The groups, —CH₂F, —CF₃, and —CH₂CF₃ are non-limiting examples of fluoroalkyl groups. The term “fused cycloalkyl” is a subset of alkyl in which the alkyl group is a cycloalkyl and is defined as a monovalent saturated aliphatic group with a carbon atom as the point of attachment, said carbon atom forming part of a structure containing 2 or more fused rings wherein at least one of the rings is non-aromatic and at least 2 or more bridgehead carbon atoms, no carbon-carbon double or triple bonds, and no atoms other than carbon and hydrogen. Non-limiting examples of fused cycloalkyl groups include:

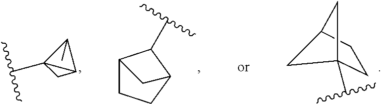

The term “alkenyl” when used without the “substituted” modifier refers to an monovalent unsaturated aliphatic group with a carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, cyclo, cyclic or acyclic structure, at least one nonaromatic carbon-carbon double bond, no carbon-carbon triple bonds, and no atoms other than carbon and hydrogen. The groups, —CH═CH—, —CH═C(CH₃)CH₂—, —CH═CHCH₂—, and

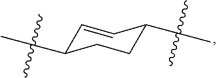 [SMILES: *C1C=CC(*)CC1]

are non-limiting examples of alkenediyl groups. It is noted that while the alkenediyl group is aliphatic, once connected at both ends, this group is not precluded from forming part of an aromatic structure. The terms “alkene” or “olefin” are synonymous and refer to a compound having the formula H—R, wherein R is alkenyl as this term is defined above. A “terminal alkene” refers to an alkene having just one carbon-carbon double bond, wherein that bond forms a vinyl group at one end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —OSi(CH₃)₂C(CH₃)₃, —OSi(CH₃)₃, —OSi(CH₂CH₃)₂, —OSi(CH(CH₃)₂)₃, —OSi(C₆H₅)₂C(CH₃)₃, —C(O)CH₃, —NHCH₃, —NHCH₂CH₃, —N(CH₃)₂, —C(O)NH₂, —OC(O)CH₃, —S(O)₂NH₂, or —OX₁ or —NX₂X₃, wherein X₁ is a hydroxy protecting group, X₂ is a monovalent amine protecting group or is taken together with X₃ and is a divalent amine protecting group and X₃ is hydrogen or is taken together with X₂ and is a divalent amine protecting group. The groups, —CH═CHF, —CH═CHCl and —CH═CHBr, are non-limiting examples of substituted alkenyl groups. The term “haloalkenyl” is a subset of substituted alkenyl, in which one or more hydrogen atoms has been substituted with a halo group and no other atoms aside from carbon, hydrogen and halogen are present.

The term “alkynyl” when used without the “substituted” modifier refers to an monovalent unsaturated aliphatic group with a carbon atom as the point of attachment, a linear or branched, cyclo, cyclic or acyclic structure, at least one carbon-carbon triple bond, and no atoms other than carbon and hydrogen. As used herein, the term alkynyl does not preclude the presence of one or more non-aromatic carbon-carbon double bonds. The groups, —C≡CH, —C≡CCH₃, and —CH₂C≡CCH₃, are non-limiting examples of alkynyl groups. An “alkyne” refers to the compound H—R, wherein R is alkynyl. When any of these terms are used with the “substituted” modifier one or more hydrogen atom has been independently replaced by When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —OSi(CH₃)₂C(CH₃)₃, —OSi(CH₃)₃, —OSi(CH₂CH₃)₂, —OSi(CH(CH₃)₂)₃, —OSi(C₆H₅)₂C(CH₃)₃, —C(O)CH₃, —NHCH₃, —NHCH₂CH₃, —N(CH₃)₂, —C(O)NH₂, —OC(O)CH₃, —S(O)₂NH₂, or —OX₁ or —NX₂X₃, wherein X₁ is a hydroxy protecting group, X₂ is a monovalent amine protecting group or is taken together with X₃ and is a divalent amine protecting group and X₃ is hydrogen or is taken together with X₂ and is a divalent amine protecting group. The term “haloalkynyl” is a subset of substituted alkynyl, in which one or more hydrogen atoms has been substituted with a halo group and no other atoms aside from carbon, hydrogen and halogen are present.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

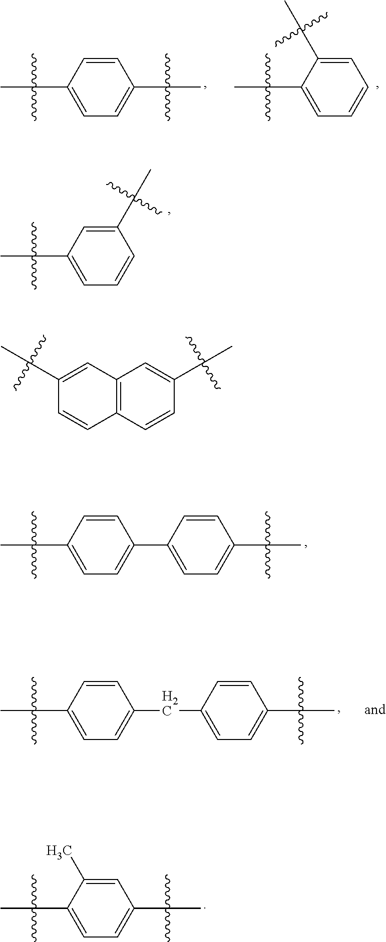

An “arene” refers to the compound H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —OSi(CH₃)₂C(CH₃)₃, —OSi(CH₃)₃, —OSi(CH₂CH₃)₂, —OSi(CH(CH₃)₂)₃, —OSi(C₆H₅)₂C(CH₃)₃, —C(O)CH₃, —NHCH₃, —NHCH₂CH₃, —N(CH₃)₂, —C(O)NH₂, —OC(O)CH₃, —S(O)₂NH₂, or —OX₁ or —NX₂X₃, wherein X₁ is a hydroxy protecting group, X₂ is a monovalent amine protecting group or is taken together with X₃ and is a divalent amine protecting group and X₃ is hydrogen or is taken together with X₂ and is a divalent amine protecting group. The term “haloaryl” is a subset of substituted aryl, in which one or more hydrogen atoms has been substituted with a halo group and no other atoms aside from carbon, hydrogen and halogen are present.

The term “aralkyl” when used without the “substituted” modifier refers to the monovalent group -alkanediyl-aryl, in which the terms alkanediyl and aryl are each used in a manner consistent with the definitions provided above. Non-limiting examples of aralkyls are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —OSi(CH₃)₂C(CH₃)₃, —OSi(CH₃)₃, —OSi(CH₂CH₃)₂, —OSi(CH(CH₃)₂)₃, —OSi(C₆H₅)₂C(CH₃)₃, —C(O)CH₃, —NHCH₃, —NHCH₂CH₃, —N(CH₃)₂, —C(O)NH₂, —OC(O)CH₃, —S(O)₂NH₂, or —OX₁ or —NX₂X₃, wherein X₁ is a hydroxy protecting group, X₂ is a monovalent amine protecting group or is taken together with X₃ and is a divalent amine protecting group and X₃ is hydrogen or is taken together with X₂ and is a divalent amine protecting group. Non-limiting examples of substituted aralkyls are: (3-chlorophenyl)-methyl, and 2-chloro-2-phenyl-eth-1-yl. The term “haloaralkyl” is a subset of substituted aralkyl, in which one or more hydrogen atoms has been substituted with a halo group and no other atoms aside from carbon, hydrogen and halogen are present.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl, pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, said atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl, aryl, and/or aralkyl groups (carbon number limitation permitting) attached to the aromatic ring or aromatic ring system. Non-limiting examples of heteroarenediyl groups include:

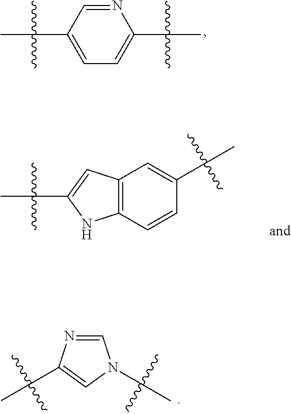 [SMILES: *c1ccc(*)nc1 ; *c1ccc2[nH]c(*)cc2c1 ; *c1cn(*)cn1]

A “heteroarene” refers to the compound H—R, wherein R is heteroaryl. Pyridine and quinoline are non-limiting examples of heteroarenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —OSi(CH₃)₂C(CH₃)₃, —OSi(CH₃)₃, —OSi(CH₂CH₃)₂, —OSi(CH(CH₃)₂)₃, —OSi(C₆H₅)₂C(CH₃)₃, —C(O)CH₃, —NHCH₃, —NHCH₂CH₃, —N(CH₃)₂, —C(O)NH₂, —OC(O)CH₃, —S(O)₂NH₂, or —OX₁ or —NX₂X₃, wherein X₁ is a hydroxy protecting group, X₂ is a monovalent amine protecting group or is taken together with X₃ and is a divalent amine protecting group and X₃ is hydrogen or is taken together with X₂ and is a divalent amine protecting group. The term “haloheteroaryl” is a subset of substituted heteroaryl, in which one or more hydrogen atoms has been substituted with a halo group and no other atoms aside from carbon, hydrogen and halogen as well as the heteroatom(s) selected from oxygen, nitrogen, and sulfur.

The term “heteroaralkyl” when used without the “substituted” modifier refers to the monovalent group -alkanediyl-heteroaryl, in which the terms alkanediyl and heteroaryl are each used in a manner consistent with the definitions provided above. Non-limiting examples of heteroaralkyls are: pyridiylmethyl and 3-thienylethyl. When the term heteroaralkyl is used with the “substituted” modifier one or more hydrogen atom from the alkanediyl and/or the heteroaryl group has been independently replaced by —OH, —F, —Cl, —Br, —I, —NH₂, —NO₂, —CO₂H, —CO₂CH₃, —CN, —SH, —OCH₃, —OCH₂CH₃, —OSi(CH₃)₂C(CH₃)₃, —OSi(CH₃)₃, —OSi(CH₂CH₃)₂, —OSi(CH(CH₃)₂)₃, —OSi(C₆H₅)₂C(CH₃)₃, —C(O)CH₃, —NHCH₃, —NHCH₂CH₃, —N(CH₃)₂, —C(O)NH₂, —OC(O)CH₃, —S(O)₂NH₂, or —OX₁ or —NX₂X₃, wherein X₁ is a hydroxy protecting group, X₂ is a monovalent amine protecting group or is taken together with X₃ and is a divalent amine protecting group and X₃ is hydrogen or is taken together with X₂ and is a divalent amine protecting group. Non-limiting examples of substituted aralkyls are: 3-chloropyridylmethyl and 1-quinolyl-3-hydroxy-butyl. The term “haloheteroaralkyl” is a subset of substituted heteroaralkyl, in which one or more hydrogen atoms has been substituted with a halo group and no other atoms aside from carbon, hydrogen and halogen as well as the heteroatom(s) selected from oxygen, nitrogen, and sulfur.

The term “heterocycloalkyl” when used without the “substituted” modifier refers to a monovalent non-aromatic group with a carbon atom or nitrogen atom as the point of attachment, said carbon atom or nitrogen atom forming part of one or more non-aromatic ring structures wherein at least one of the ring atoms is nitrogen, oxygen or sulfur, and wherein the heterocycloalkyl group consists of no atoms other than carbon, hydrogen, nitrogen, oxygen and sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. The term “heterocycloalkanediyl” when used without the “substituted” modifier refers to an divalent cyclic group, with two carbon atoms, two nitrogen atoms, or one carbon atom and one nitrogen atom as the two points of attachment, said atoms forming part of one or more ring structure(s) wherein at least one of the ring atoms is nitrogen, oxygen or sulfur, and wherein the divalent group consists of no atoms other than carbon, hydrogen, nitrogen, oxygen and sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkanediyl groups include:

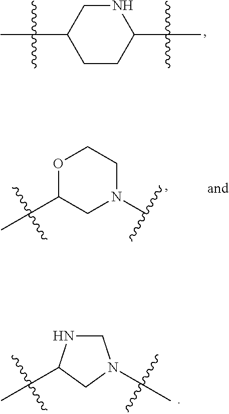 [SMILES: *C1CCC(*)NC1 ; *C1CN(*)CCO1 ; *C1CN(*)CN1]

When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —OSi(CH₃)₂C(CH₃)₃, —OSi(CH₃)₃, —OSi(CH₂CH₃)₂, —OSi(CH(CH₃)₂)₃, —OSi(C₆H₅)₂C(CH₃)₃, —C(O)CH₃, —NHCH₃, —NHCH₂CH₃, —N(CH₃)₂, —C(O)NH₂, —OC(O)CH₃, —S(O)₂NH₂, or —OX₁ or —NX₂X₃, wherein X₁ is a hydroxy protecting group, X₂ is a monovalent amine protecting group or is taken together with X₃ and is a divalent amine protecting group and X₃ is hydrogen or is taken together with X₂ and is a divalent amine protecting group. The term “haloheterocycloalkyl” is a subset of substituted alkyl, in which one or more hydrogen atoms has been substituted with a halo group and no other atoms aside from carbon, hydrogen and halogen are present.

The term “acyl” when used without the “substituted” modifier refers to the group —C(O)R, in which R is a hydrogen, alkyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —OSi(CH₃)₂C(CH₃)₃, —OSi(CH₃)₃, —OSi(CH₂CH₃)₂, —OSi(CH(CH₃)₂)₃, —OSi(C₆H₅)₂C(CH₃)₃, —C(O)CH₃, —NHCH₃, —NHCH₂CH₃, —N(CH₃)₂, —C(O)NH₂, —OC(O)CH₃, —S(O)₂NH₂, or —OX₁ or —NX₂X₃, wherein X₁ is a hydroxy protecting group, X₂ is a monovalent amine protecting group or is taken together with X₃ and is a divalent amine protecting group and X₃ is hydrogen or is taken together with X₂ and is a divalent amine protecting group. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The term “haloacyl” is a subset of substituted acyl, in which one or more hydrogen atoms has been substituted with a halo group and no other atoms aside from carbon, hydrogen, oxygen, and halogen are present.

The term “alkoxy” when used without the “substituted” modifier refers to the group —OR, in which R is an alkyl, as that term is defined above. Non-limiting examples of alkoxy groups include: —OCH₃ (methoxy), —OCH₂CH₃ (ethoxy), —OCH₂CH₂CH₃, —OCH(CH₃)₂ (isopropoxy), —O(CH₃)₃ (tert-butoxy), —OCH(CH₂)₂, —O-cyclopentyl, and —O-cyclohexyl. The terms “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is alkenyl, alkynyl, aryl, aralkyl, heteroaryl, heterocycloalkyl, and acyl, respectively. The term “alkoxydiyl” refers to the divalent group —O-alkanediyl-, —O-alkanediyl-O—, or -alkanediyl-O-alkanediyl-. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —OSi(CH₃)₂C(CH₃)₃, —OSi(CH₃)₃, —OSi(CH₂CH₃)₂, —OSi(CH(CH₃)₂)₃, —OSi(C₆H₅)₂C(CH₃)₃, —C(O)CH₃, —NHCH₃, —NHCH₂CH₃, —N(CH₃)₂, —C(O)NH₂, —OC(O)CH₃, —S(O)₂NH₂, or —OX₁ or —NX₂X₃, wherein X₁ is a hydroxy protecting group, X₂ is a monovalent amine protecting group or is taken together with X₃ and is a divalent amine protecting group and X₃ is hydrogen or is taken together with X₂ and is a divalent amine protecting group.

The term “haloalkoxy” is a subset of substituted alkoxy, in which one or more hydrogen atoms has been substituted with a halo group and no other atoms aside from carbon, hydrogen, oxygen, and halogen are present.

The term “alkylamino” when used without the “substituted” modifier refers to the group —NHR, in which R is an alkyl, as that term is defined above. Non-limiting examples of alkylamino groups include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂, —N(CH₃)(CH₂CH₃), and N-pyrrolidinyl. The terms “alkoxyamino”, “alkenylamino”, “alkynylamino”, “arylamino”, “aralkylamino”, “heteroarylamino”, “heterocycloalkylamino” and “alkylsulfonylamino” when used without the “substituted” modifier, refers to groups, defined as —NHR, in which R is alkoxy, alkenyl, alkynyl, aryl, aralkyl, heteroaryl, heterocycloalkyl, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. The term “alkylaminodiyl” refers to the divalent group —NH-alkanediyl-, —NH-alkanediyl-NH—, or -alkanediyl-NH-alkanediyl-. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —OSi(CH₃)₂C(CH₃)₃, —OSi(CH₃)₃, —OSi(CH₂CH₃)₂, —OSi(CH(CH₃)₂)₃, —OSi(C₆H₅)₂C(CH₃)₃, —C(O)CH₃, —NHCH₃, —NHCH₂CH₃, —N(CH₃)₂, —C(O)NH₂, —OC(O)CH₃, —S(O)₂NH₂, or —OX₁ or —NX₂X₃, wherein X₁ is a hydroxy protecting group, X₂ is a monovalent amine protecting group or is taken together with X₃ and is a divalent amine protecting group and X₃ is hydrogen or is taken together with X₂ and is a divalent amine protecting group. The groups —NHC(O)OCH₃ and —NHC(O)NHCH₃ are non-limiting examples of substituted amido groups. The term “haloamido” or “haloalkylamino” or “halodialkylamino” is a subset of substituted amido, alkylamino, or dialkylamino in which one or more hydrogen atoms has been substituted with a halo group and no other atoms aside from carbon, hydrogen, amino, and halogen are present.

The terms “alkylsulfonyl” and “alkylsulfinyl” when used without the “substituted” modifier refers to the groups —S(O)₂R and —S(O)R, respectively, in which R is an alkyl, as that term is defined above. The terms “alkenylsulfonyl”, “alkynylsulfonyl”, “arylsulfonyl”, “aralkylsulfonyl”, “heteroarylsulfonyl”, and “heterocycloalkylsulfonyl” are defined in an analogous manner. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —OSi(CH₃)₂C(CH₃)₃, —OSi(CH₃)₃, —OSi(CH₂CH₃)₂, —OSi(CH(CH₃)₂)₃, —OSi(C₆H₅)₂C(CH₃)₃, —C(O)CH₃, —NHCH₃, —NHCH₂CH₃, —N(CH₃)₂, —C(O)NH₂, —OC(O)CH₃, —S(O)₂NH₂, or —OX₁ or —NX₂X₃, wherein X₁ is a hydroxy protecting group, X₂ is a monovalent amine protecting group or is taken together with X₃ and is a divalent amine protecting group and X₃ is hydrogen or is taken together with X₂ and is a divalent amine protecting group. The term “haloalkylsulfonyl” is a subset of substituted alkylsulfonyl, in which one or more hydrogen atoms has been substituted with a halo group and no other atoms aside from carbon, hydrogen, oxygen, sulfur, and halogen are present.

The term “alkylsilyl” when used without the “substituted” modifier refers to a monovalent group, defined as —SiH₂R, —SiHRR′, or —SiRR′R″, in which R, R′ and R″ can be the same or different alkyl groups, or any combination of two of R, R′ and R″ can be taken together to represent an alkanediyl. The groups, —SiH₂CH₃, —SiH(CH₃)₂, —Si(CH₃)₃ and —Si(CH₃)₂C(CH₃)₃, are non-limiting examples of unsubstituted alkylsilyl groups. The term “substituted alkylsilyl” refers —SiH₂R, —SiHRR′, or —SiRR′R″, in which at least one of R, R′ and R″ is a substituted alkyl or two of R, R′ and R″ can be taken together to represent a substituted alkanediyl. When more than one of R, R′ and R″ is a substituted alkyl, they can be the same or different. Any of R, R′ and R″ that are not either substituted alkyl or substituted alkanediyl, can be either alkyl, either the same or different, or can be taken together to represent a alkanediyl with two or more saturated carbon atoms, at least two of which are attached to the silicon atom. The term “arylsilyl” or “aralkylsilyl” refers to the group as defined above where at least one of R, R′, or R″ is an aryl or aralkyl group as those groups are defined above. The term “haloalkylsilyl” is a subset of substituted alkylsilyl, in which one or more hydrogen atoms has been substituted with a halo group and no other atoms aside from carbon, hydrogen, silicon, and halogen are present.

A “base” in the context of this application is a compound which has a lone pair of electron. Non-limiting examples of a base can include triethylamine, a metal hydroxide, metal hydride, or a metal alkane. An alkyllithium or organolithium is a compound of the formula alkyl_{(C≦12)}-Li. A nitrogenous base is an alkylamine, dialkylamine, trialkylamine, nitrogen containing heterocycloalkane or heteroarene wherein the base can accept a proton to form a positively charged species. For example, but not limited to, a nitrogenous base could be 4,4-dimethylpyridine, pyridine, 1,8-diazabicyclo[5.4.0]undec-7-ene, diisopropylethylamine, or triethylamine.

A “reducing agent” in the context of this application is a compound which causes the reduction of a compound through the donation of an electron. Some non-limiting examples of reducing agents are sodium borohydride, lithium aluminum hydride, diisobutylaluminum hydride, hydrogen gas, or metal hydride.

An “oxidizing agent” in the context of this application is a compound which causes the oxidation of a compound by accepting an electron. Some non-limiting examples of oxidizing agent are oxygen gas, hypervalent iodide compound such as Dess-Martin periodinate, an oxygen radical compound such as TEMPO or TEMPACE, peroxides, chlorite, hypochlorite, or a chromium compound such as pyridinium chlorochromate or hydrochromic acid.

A “metal” in the context of this application is a transition metal or a metal of groups I or II. In some embodiments, a metal is lithium, sodium, or potassium.

A “methylating agent” in the context of this application is a reagent which reacts to generate a methyl group on a reactive functional group. In some non-limiting examples of methylating agents including trimethylsilyl diazomethane or methyl halides.

A “fluoride source” in the context of this application is a reagent which generates or contains a fluoride ion. Some non-limiting examples include hydrofluoric acid, metal fluoride, or tetrabutylammonium fluoride.

An “alkylaluminium” in the context of this application is a reagent which contains one, two, three, or four alkyl groups as that group is defined above to a central aluminum atom. Some non-limiting examples of alkylaluminiums are trimethylaluminum or tetramethylaluminium.

A “silylating agent” in the context of this application is a reagent which contains an alkylsilyl, arylsilyl, or aralkylsilyl group bound to a halogen, mesylate, tosylate or other leaving group. Some non-limiting examples of silylating agents are t-butyldimehtylsilyl chloride (TBSCl) or trimethylsilyl chloride (TMSCl) which can be used to produce hydroxyl groups protected with the t-butyldimethylsilyl (TBS) or trimethylsilyl (TMS) group.

A “group which enhances the ability of the hydroxyl group to be eliminated” or a “leaving group” in the context of this application is a reagent which converts the hydroxyl group into a group which has the ability to be displaced from the molecule through nucleophilic attack. This reagent make the hydroxyl group a better leaving group by stabilizing the charge on the oxygen when the atom bears a negative charge. The reagent makes the hydroxyl group more susceptible to a nucleophilic attack and displacement. An “agent which enhances the ability of the hydroxyl group to be eliminated” or a “leaving group agent” comprises the “group” plus some form of leaving group such a halide or a substituted alkylsulfonyl such that when reacted with a hydroxyl group the hydroxyl group is converted into a group which enhances the ability of the hydroxyl group to be eliminated. An “agent which enhances the ability of the hydroxyl group to be eliminated” or a “leaving group agent” can be used to add a “group which enhances the ability of the hydroxyl group to be eliminated” or a “leaving group” to a compound or formula. Some non-limiting examples include methanesulfonyl chloride, p-toluenesulfonyl chloride, or fluoro derivatives of these compounds. Additionally, the group could be a halogen atom especially a bromide or iodide. In some aspects, the “agent which enhances the ability of the hydroxyl group to be eliminated” or a “leaving group agent” could be a “halogenating agent” which introduces a halogen atom into the molecule. Some non-limiting examples of “halogenating agents” include phosphorus tribromide, carbon tetrabromide with triphenyl phosphine, potassium iodide, or thionyl chloride.

An “activating agent” in the context of this application is a reagent which enhances the reactivity of the compound. In some aspects, the activating agent is a compound which reacts with the group —C(O)OH to enhance its ability to react with an alcohol or amine to form an ester or amide. Some non-limiting examples of activating agents include carbonyl diimidazole, dicyclohexylcarbodiimide, 2-methyl-6-nitrobenzoic anhydride, or a benzotriazole phosphonium reagent such as BOP and PyBOP.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≦15%, more preferably ≦10%, even more preferably ≦5%, or most preferably ≦1% of another stereoisomer(s).

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

All reactions were carried out under an argon atmosphere with dry solvents under anhydrous conditions, unless otherwise noted. Dry tetrahydrofuran (THF), toluene, diethyl ether (Et₂O), acetonitrile (MeCN), methylene chloride (CH₂Cl₂), triethylamine (Et₃N), diisopropylamine, and pyridine were obtained by passing commercially available pre-dried, oxygen-free formulations through activated alumina columns. Yields refer to chromatographically and spectroscopically (¹H NMR) homogeneous materials, unless otherwise stated. Reagents were purchased at the highest commercial quality and used without further purification, unless otherwise stated. Reactions were monitored by thin-layer chromatography (TLC) carried out on 0.25 mm E Merck silica gel plates (60F₂₅₄) using UV light as visualizing agent and an aqueous or ethanolic solution of phosphomolybdic acid and cerium sulfate or an aqueous solution of potassium permanganate and heat as developing agents. Acros Organics silica gel (60, particle size 0.0354.07 mm) was used for flash column chromatography. Preparative thin-layer chromatography (PTLC) separations were carried out on 0.25 mm E Merck silica gel plates (60F₂₅₄). NMR spectra were recorded on a Bruker 400 MHz, a Bruker Avance III 500 MHz, and a Bruker Avance III HD 600 MHz instrument equipped with a 5 mm DCH cryoprobe and calibrated using residual undeuterated solvent for ¹H NMR [δ_{H}=7.26 (CHCl₃) and 7.16 ppm (C₆D₅H)] and ¹³C deuterated solvent for ¹³C NMR [δ_{C}=77.16 (CDCl₃) and 128.06 ppm (C₆D₆)] as an internal reference at 298 K. The following abbreviations were used to explain the multiplicities: s=singlet, d=doublet, t=triplet, q=quartet, p=quintuplet, m=multiplet, b=broad, and ap=apparent. IR spectra were recorded on a Perkin-Elmer Spectrum 100 FT-IR spectrometer. High-resolution mass spectra (HRMS) were recorded on an Ion Trap-Time of Flight Mass Spectrometer (Shimadzu, Columbia, Md.) operated with an ESI source interface. Optical rotations were measured on a Schmidt+Haensch Polartronic M100 polarimeter at 589.44 nm using 100 mm cells and the solvent and concentration indicated. UV-Vis spectra were measured using a Varian Cary 5000 UV-Vis-NIR spectrophotometer.

### Example 2

### Experimental Results

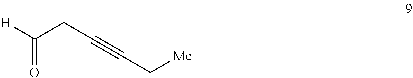

### Hex-3-ynal (9)

To a stirred solution of 3-hexyn-1-ol (491 mg; 5.00 mmol; 1.0 equiv.) in CH₂Cl₂ (50 mL) was added Dess-Martin's periodinane (2.76 g; 6.50 mmol; 1.3 equiv.) at 0° C. in one portion. After 5 min at this temperature the reaction mixture was warmed to 25° C. and stirred for 1.5 h. The suspension formed was then cooled to −15° C. and diluted with pentane (50 mL), stirred for an additional 5 min and then quickly filtered through a pad of SiO₂, pre-soaked with pentane:Et₂O (4/1; pre-cooled to −50° C.), and washed with pentane:Et₂O (4/1; 200 mL; pre-cooled to −50° C.). The clear solution so-obtained was carefully concentrated at 25° C. (minimum pressure: 200 mbar) to give pure title compound (457 mg; 4.75 mmol; 95%) which was immediately dissolved in Et₂O (10 mL) and used in the next step. All analytical data were identical to those reported in the literature (Wavrin and Viala, 2002).

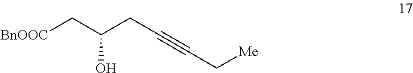

### β-Hydroxyester 17

To a stirred solution of 2′-{[3-bromo-5-(t-butyl)benzylidene]amino}-[(R)-1,1′-binaphthalen]-2-ol (Carreira, et al., 1994) (288 mg; 0.55 mmol; 0.11 equiv.) in toluene (40 mL) was added titanium tetraisopropoxide (74 μL; 0.25 mmol; 0.05 equiv.) dropwise at 25° C. The orange solution turned red and stirring was continued at the same temperature for 1 h, after which a solution of 3,5-di-t-butyl-salicylic acid (75 mg; 0.30 mmol; 0.06 equiv.; azeotropically dried by concentrating twice from toluene) in toluene (5 mL) was added dropwise. Stirring was continued for 1 h, all volatiles were evaporated under exclusion of moisture and air (final vacuum: 1 mbar), and the red solid so-obtained was dissolved in Et₂O (20 mL) and cooled to −78° C. To this solution were added sequentially, dropwise benzyl acetate-trimethylsilyl acetal (Kiyooka, et al., 2010) (2.22 g; 10.0 mmol; 2.0 equiv.) and a solution of aldehyde 9 (457 mg; 4.75 mmol) in Et₂O (10 mL) from the previous step. The reaction mixture was warmed to −15° C. over the course of 1 h and stirred at that temperature for 4 h. The reaction mixture was then quenched with sat. aq. NaHCO₃ solution (20 mL), the phases were separated, and the aq. layer was extracted with Et₂O (3×20 mL). The combined organic extracts were washed with sat. brine (20 mL), dried (Na₂SO₄), filtered, and concentrated. The crude intermediate was then dissolved in THF (10 mL) and tetrabutylammonium fluoride (1 M in THF; 20.0 mL; 20.0 mmol; 4.0 equiv.) was added dropwise with stirring at 25° C. After stirring for 30 min, the reaction mixture was partitioned between Et₂O (50 mL) and aq. HCl (1 M; 20 mL), the organic layer was washed with sat. aq. NaHCO₃ solution (20 mL) and sat. brine (20 mL), dried (Na₂SO₄), filtered, and concentrated. Flash column chromatography (SiO₂; hexane:EtOAc, 20:1→10:1) yielded slightly contaminated title compound (ca. 90% purity; 930 mg; 3.42 mmol; 72%; ≧95% ee by Mosher ester analysis of pure title compound) as a colorless oil. An analytically pure sample was obtained by repeated flash column chromatography (SiO₂; C₆H₆:MeOH, 100:1).

17: R_{f}=0.35 (hexane:EtOAc, 5:1); [α]_{D}²⁵=+24.0° (c=1.0 in CHCl₃); IR (film): νₘₐₓ 3368, 2928, 2856, 1737, 1461, 1253, 1152, 1103 cm⁻¹; ¹H-NMR (500 MHz, C₆D₆) δ 7.22-7.02 (m, 5H), 4.93 (d, J=3.4 Hz, 2H), 4.35 (d, J=5.3 Hz, 1H), 4.12 (ddddd, J=8.5, 6.7, 5.6, 4.6, 3.9 Hz, 1H), 2.85 (d, J=4.7 Hz, 1H), 2.51 (dd, J=16.2, 3.9 Hz, 1H), 2.44 (dd, J=16.2, 8.5 Hz, 1H), 2.31 (ddt, J=16.5, 5.6, 2.4 Hz, 1H), 2.25 (ddt, J=16.4, 6.7, 2.4 Hz, 1H), 1.92 (dt, J=7.5, 2.4 Hz, 1H), 1.89 (dt, J=7.5, 2.4 Hz, 1H), 0.90 (t, J=7.5 Hz, 3H) ppm; ¹³C-NMR (125 MHz, C₆D₆) δ 172.07, 136.40, 128.71, 128.54, 127.82, 84.62, 75.55, 67.32, 66.40, 40.66, 27.26, 14.30, 12.67 ppm; HR-MS (ESI-TOF): calcd for C₁₅H₁₉O₃ [M+H]⁺: 247.1329. found: 247.1327.

Full Mosher-analysis of β-hydroxyester 17:

### (R)-MTPA ester 17a

To a stirred solution of β-hydroxyester 17 (10.0 mg; 40.6 μmol; 1.0 equiv.) in CH₂Cl₂ (0.5 mL) was added pyridine (33 μL; 406.0 μmol; 10 equiv.) and (S)-(+)-Mosher chloride (23 μL; 121.8 μmol; 3.0 equiv.) at 25° C. After stirring for 3 h at the same temperature, the reaction mixture was diluted with CH₂Cl₂ (5 mL), quenched with sat. aq. NH₄Cl-solution (5 mL), the phases were separated, and the aq. layer was extracted with CH₂Cl₂ (2×5 mL). The combined organic extracts were dried (Na₂SO₄), filtered, and concentrated. Preparative thin layer chromatography (SiO₂; hexane:EtOAc, 10:1) yielded pure title compound (12.2 mg; 26.4 μmol; 65%) as a colorless oil.

17a: R_{f}=0.28 (hexane:EtOAc, 10:1); IR (film): νₘₐₓ 2920, 2850, 1749, 1454, 1269, 1168, 1017 cm⁻¹; ¹H-NMR (500 MHz, C₆D₆) δ 7.84-7.77 (m, 1H), 7.14-6.99 (m, 9H), 5.63 (dtd, J=8.8, 5.4, 4.2 Hz, 1H), 4.85 (d, J=12.3 Hz, 1H), 4.82 (d, J=12.3 Hz, 1H), 3.52 (q, J=1.2 Hz, 3H), 2.59 (dd, J=16.6, 8.8 Hz, 1H), 2.43 (ddt, J=17.0, 5.4, 2.4 Hz, 1H), 2.28 (dd, J=16.6, 4.3 Hz, 1H), 2.26 (ddt, J=17.1, 5.4, 2.2 Hz, 1H), 1.85 (qt, J=7.5, 2.4 Hz, 2H), 0.86 (t, J=7.5 Hz, 3H) ppm; ¹³C-NMR (125 MHz, C₆D₆) δ 169.20, 165.84, 136.16, 133.08, 129.66, 128.71, 128.68, 128.54, 128.49, 128.44, 124.25 (q, J=288.6 Hz), 85.26, 85.16 (d, J=26.8 Hz), 73.86, 71.47, 66.62, 55.60 (q, J=1.5 Hz), 37.70, 23.95, 14.03, 12.53 ppm; ¹⁹F-NMR (471 MHz, C₆D₆) δ −70.63 ppm; HRMS (ESI-TOF): calcd for C₂₅H₂₅F₃O₅Na [M+Na]⁺: 485.1546. found: 485.1531.

### (S)-MTPA ester 17b

The title compound was prepared in the same manner and on the same scale as above using (R)-(−)-Mosher chloride. Preparative thin layer chromatography (SiO₂; hexane:EtOAc, 10:1) yielded pure title compound (11.1 mg; 24.0 μmol; 59%) as a colorless oil.

17b: R_{f}=0.28 (hexane:EtOAc, 10:1); ¹H-NMR (500 MHz, C₆D₆) δ 7.73 (d, J=7.7 Hz, 1H), 7.15-7.01 (m, 9H), 5.61 (dddd, J=9.2, 6.4, 4.9, 3.7 Hz, 1H), 4.89 (s, 2H), 3.44 (q, J=1.2 Hz, 3H), 2.60 (dd, J=16.7, 9.3 Hz, 1H), 2.43 (dd, J=16.7, 3.7 Hz, 1H), 2.35 (ddt, J=16.8, 6.4, 2.4 Hz, 1H), 2.26 (ddt, J=16.8, 4.8, 2.3 Hz, 1H), 1.79 (qt, J=7.5, 2.4 Hz, 2H), 0.83 (t, J=7.5 Hz, 3H) ppm; ¹³C-NMR (125 MHz, C₆D₆) δ 169.62, 166.09, 136.12, 133.05, 129.64, 128.74, 128.70, 128.55, 128.49, 128.44, 124.21 (q, J=288.6 Hz), 85.27 (q, J=27.5 Hz), 85.13, 73.44, 71.44, 66.68, 55.59 (q, J=1.5 Hz), 37.68, 23.71, 14.02, 12.52 ppm; ¹⁹F-NMR (471 MHz, C₆D₆) δ −70.66 ppm.

| | Chemical shift of | Chemical shift of | Δ(S)-MTPA − |
|---|---|---|---|
| Proton | (S)-MTPA derivative | (R)-MTPA derivative | (R)-MTPA |
| 1′ | 4.89 | 4.85 | +0.04 |
| 2a | 2.60 | 2.59 | +0.01 |
| 2b | 2.43 | 2.28 | +0.15 |
| 4a | 2.35 | 2.43 | −0.08 |
| 4b | 2.26 | 2.26 | ±0.00 |
| 7 | 1.79 | 1.85 | −0.06 |
| 8 | 0.83 | 0.86 | −0.03 |

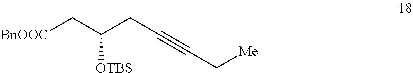

### t-Butyldimethylsilyl-alcohol 18

To a stirred solution of β-hydroxyester 17 (ca. 90% purity; 890 mg; 3.24 mmol; 1.0 equiv.) in CH₂Cl₂ (15 mL) at 25° C. were added sequentially imidazole (662 mg; 9.72 mmol; 3.0 equiv.) and t-butyldimethylsilyl chloride (977 mg; 6.48 mmol; 2.0 equiv.). The reaction mixture was stirred for 3 h at that temperature, quenched with sat. aq. NH₄Cl solution (15 mL), the phases were separated and the aq. layer was extracted with CH₂Cl₂ (15 mL). The combined organic extracts were washed with H₂O (10 mL), dried (MgSO₄), filtered, and concentrated. Flash column chromatography (SiO₂; hexane:EtOAc, 20:1) yielded pure title compound (1.03 g; 2.85 mmol; 88%) as a colorless oil.

18: R_{f}=0.67 (hexane:EtOAc, 10:1); [α]_{D}²⁵=+29.5° (c=1.0 in C₆H₆); IR (film): νₘₐₓ 2929, 2856, 1738, 1462, 1254, 1155, 1101 cm⁻¹; ¹H-NMR (500 MHz, C₆D₆) δ 7.23-7.18 (m, 2H), 7.12-7.03 (m, 3H), 5.01 (d, J=6.7 Hz, 2H), 4.39 (dddt, J=8.9, 7.9, 3.8, 2.7 Hz, 1H), 2.68 (ddd, J=15.2, 4.0, 1.5 Hz, 1H), 2.60 (ddd, J=15.2, 8.1, 1.5 Hz, 1H), 2.36 (ddddd, J=18.4, 16.4, 14.2, 6.2, 2.1 Hz, 2H), 1.95 (dddd, J=9.5, 7.5, 5.4, 2.0 Hz, 2H), 0.97-0.92 (m, 12H), 0.09 (s, 3H), 0.08 (s, 3H) ppm; ¹³C-NMR (125 MHz, C₆D₆) δ 171.05, 136.64, 128.69, 128.63, 127.71, 84.27, 76.11, 69.22, 66.27, 42.32, 28.32, 26.02, 18.25, 14.33, 12.76, −4.49, −4.73 ppm; HR-MS (ESI-TOF): calcd for C₂₁H₃₃O₃Si [M+H]⁺: 361.2193. found: 361.2190.

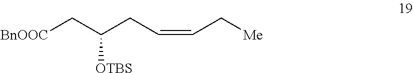

### Alkene 19

To a stirred suspension of Lindlar-catalyst (5% Pd on CaCO₃, Pb poisoned; 370 mg; 0.18 mmol; 0.1 equiv.) in EtOAc (10 mL) were sequentially added a solution of t-butyldimethylsilyl-alcohol 18 (642 mg; 1.78 mmol; 1.0 equiv.) in EtOAc (10 mL) and quinolone (210 μL; 1.78 mmol; 1.0 equiv.) at 25° C. The suspension was degassed and purged with Ar three times, then the atmosphere was changed to H₂ (balloon). After vigorous stirring for 30 min at the same temperature, the reaction mixture was filtered through Celite®, washed with EtOAc, and concentrated. Flash column chromatography (SiO₂; hexane:EtOAc, 20:1) yielded pure title compound (639 mg; 1.76 mmol; 99%) as a colorless oil.

19: R_{f}=0.63 (hexane:EtOAc, 10:1); [α]_{D}²⁵=+28.3° (c=1.0 in C₆H₆); IR (film): νₘₐₓ 2957, 2929, 2856, 1737, 1462, 1253, 1158, 1087 cm⁻¹; ¹H-NMR (500 MHz, C₆D₆) δ 7.25-7.20 (m, 2H), 7.14-7.02 (m, 3H), 5.48-5.33 (m, 2H), 5.03 (d, J=2.5 Hz, 2H), 4.29 (ddt, J=7.8, 6.6, 4.9 Hz, 1H), 2.49 (dd, J=15.0, 7.8 Hz, 1H), 2.38 (dd, J=15.0, 4.6 Hz, 1H), 2.33-2.19 (m, 2H), 1.96-1.87 (m, 2H), 0.97 (s, 9H), 0.86 (t, J=7.5 Hz, 3H), 0.11 (s, 3H), 0.10 (s, 3H) ppm; ¹³C-NMR (125 MHz, C₆D₆) δ 171.20, 136.68, 134.32, 128.72, 128.70, 128.31, 124.53, 69.87, 66.25, 42.35, 35.69, 26.07, 21.05, 18.27, 14.38, 4.33, 4.64 ppm; HR-MS (ESI-TOF): calcd for C₂₁H₃₅O₃Si [M+H]⁺: 363.2350. found: 363.2341.

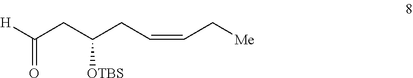

### Aldehyde 8

To a stirred solution of alkene 19 (639 mg; 1.76 mmol; 1.0 equiv.) in CH₂Cl₂ (30 mL) was added dropwise diisobutylaluminum hydride (1 M in CH₂Cl₂; 2.29 mL; 2.29 mmol; 1.3 equiv.) at −78° C. The reaction mixture was gradually warmed to −25° C. over the course of 1 h and then quenched with MeOH (3 mL), diluted with CH₂Cl₂ (70 mL) and warmed to 25° C. Under vigorous stirring, sat. aq. Rochelle-salt solution (Na/KC₄H₄O₆.4H₂O; 50 mL) was added, and the resulting mixture was stirred for an additional 1 h at the same temperature before the phases were separated and the aq. layer was extracted with CH₂Cl₂ (2×20 mL). The combined organic extracts were washed with H₂O (20 mL), dried (Na₂SO₄), filtered, and concentrated. Flash column chromatography (SiO₂; hexane:EtOAc, 20:1→15:1) yielded pure title compound (402 mg; 1.57 mmol; 89%) as a colorless oil.

R_{f}=0.70 (hexane:EtOAc, 10:1); [α]_{D}²⁵=+16.3° (c=1.0 in C₆H₆); IR (film): νₘₐₓ 2958, 2930, 2857, 1727, 1463, 1254, 1089 cm⁻¹; ¹H-NMR (500 MHz, C₆D₆) δ 9.51 (dd, J=2.7, 1.6 Hz, 1H), 5.48-5.39 (m, 1H), 5.29 (dtt, J=10.7, 7.5, 1.6 Hz, 1H), 4.04 (tdd, J=7.0, 5.3, 4.5 Hz, 1H), 2.25 (ddd, J=16.0, 7.2, 2.7 Hz, 1H), 2.21-2.06 (m, 3H), 1.96-1.86 (m, 2H), 0.93 (s, 9H), 0.88 (t, J=7.5 Hz, 3H), 0.03 (s, 3H), 0.02 (s, 3H) ppm; ¹³C-NMR (125 MHz, C₆D₆) δ 200.10, 134.41, 124.32, 68.25, 50.72, 35.80, 25.99, 21.06, 18.20, 14.34, −4.31, −4.68 ppm; HR-MS (ESI-TOF): calcd for C₁₄H₂₉O₂Si [M+H]⁺: 257.1931. found: 257.1941.

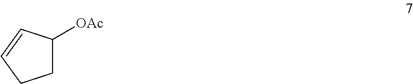

### Cyclopent-2-en-1-yl acetate (7)

To a stirred solution of LiAlH₄ (1.90 g; 50.0 mmol; 0.5 equiv.) in Et₂O (150 mL) at 25° C. was added dropwise a solution of 2-cyclopentenone (8.21 g; 100.0 mmol; 1.0 equiv.) in Et₂O (50 mL). After stirring for 10 min at the same temperature the reaction mixture was cooled to 0° C. and carefully quenched with H₂O (10 mL) and then aq. HCl (1 M; 250 mL). The phases were separated, the aq. layer was extracted with Et₂O (3×100 mL), and the combined organic extracts were washed sequentially with sat. aq. NaHCO₃-solution (100 mL) and sat. brine (50 mL). Drying (Na₂SO₄), filtration, and careful concentration (minimum pressure 150 mbar) gave crude allylic alcohol (7.80 g; 92.7 mmol; 93%) as a racemate which was used in the next step without purification. To a stirred solution of the crude allylic alcohol obtained above (7.80 g; 92.7 mmol) in CH₂Cl₂ (75 mL) were added sequentially, at 0° C., Et₃N (34.8 mL; 250.0 mmol; 2.5 equiv.), 4-dimethylaminopyridine (1.22 g; 10.0 mmol; 0.1 equiv.), and Ac₂O (18.9 mL; 200.0 mmol; 2.0 equiv.). The reaction mixture was warmed to 25° C. and stirred for 18 h. The brown solution was then partitioned between CH₂Cl₂ (200 mL) and aq. HCl (200 mL), the organic layer was washed with sat. aq. NaHCO₃-solution (100 mL), dried (MgSO₄), filtered, and carefully concentrated (minimum pressure 150 mbar). Flash column chromatography (SiO₂; pentane:Et₂O, 20:1) yielded the slightly contaminated, racemic title compound (ca. 90% purity; 9.41 g; 67.1 mmol; 72%; 67% for two steps) as a colorless oil.

7: R_{f}=0.85 (hexane:EtOAc, 2:1). All analytical data were identical to those reported in the literature (By Jacquet et al., 2010)

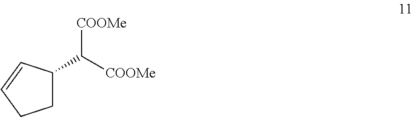 [SMILES: COC(=O)C(C(=O)OC)[C@H]1C=CCC1]

### (R)-Dimethyl-2-(cyclopent-2-en-1-yl)malonate (11)

To a stirred suspension of (S,S)-DACH-phenyl Trost ligand ((S,S)-1,2-diaminocyclohexane-N,N′-bis(2′-diphenylphosphinobenzoyl); 600 mg; 0.87 mmol; 0.015 equiv.), allylpalladium(II) chloride dimer (106 mg; 0.29 mmol; 0.005 equiv.) and Cs2CO3 (56.7 g; 174.0 mmol; 3.0 equiv.) in CH₂Cl₂ (100 mL) was added cyclopent-2-en-1-yl acetate (7) (ca. 90% purity; 8.10 g; 58.0 mmol; 1.0 equiv.) dropwise at 25° C. The yellow suspension was stirred for 15 min and dimethyl malonate (19.9 mL; 174.0 mmol; 3.0 equiv.) was added dropwise over the course of 20 min After stirring for an additional 45 min at the same temperature, the reaction mixture was partitioned between EtOAc (300 mL) and H₂O (150 mL), the aq. layer was extracted with EtOAc (2×100 mL), and the combined organic extracts were washed with sat. brine (100 mL), dried (MgSO4), filtered, and carefully concentrated (minimum pressure 100 mbar). Flash column chromatography (SiO₂; pentane:Et₂O, 10:1) yielded pure title compound (8.16 g; 41.2 mmol; 71%) as a colorless oil.

11: R_{f}=0.58 (hexane:Et₂O, 6:1); [α]_{D}²⁵=+87.3° (c=1.11 in CHCl₃), ca. 97% ee, lit. (Miyazaki, et al., 2007): [α]_{D}²⁵=−86° (c=1.11 in CHCl₃) for the other enantiomer of 96% ee. All other analytical data were identical to those reported in the literature (Miyazaki, et al., 2007).

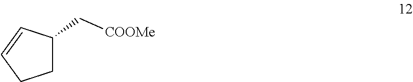

### (S)-Methyl-2-(cyclopent-2-en-1-yl)acetate (12)

To a stirred solution of (R)-dimethyl-2-(cyclopent-2-en-1-yl)malonate (11) (8.00 g; 40.3 mmol; 1.0 equiv.) in a mixture of 1,3-dimethyl-2-imidazolidinone:H₂O (10:1; 154 mL) at 25° C. was added KI (53.52 g; 322.4 mmol; 8.0 equiv.) in one portion and the reaction mixture was heated under reflux (130° C.) for 12 h. After cooling to 25° C., the reaction mixture was extracted with hexane (4×100 mL), the combined organic extracts were dried (MgSO₄), filtered, and carefully concentrated (minimum pressure 200 mbar). Flash column chromatography (SiO₂; pentane:Et₂O, 30:1) yielded pure title compound (5.31 g; 37.9 mmol; 94%) as a colorless oil.

12: R_{f}=0.63 (hexane:EtOAc, 7:1). All analytical data were identical to those reported in the literature (Miyazaki, et al., 2007).

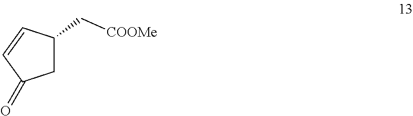 [SMILES: COC(=O)C[C@H]1C=CC(=O)C1]

### (S)-Methyl-2-(4-oxocyclopent-2-en-1-yl)acetate (13)

To a stirred solution of (S)-methyl-2-(cyclopent-2-en-1-yl)acetate (12) (2.00 g; 14.27 mmol; 1.0 equiv.) in CH₂Cl₂ (50 mL) under an atmosphere of oxygen (balloon) at 25° C. were added sequentially K₂CO₃ (987 mg; 7.14 mmol; 0.5 equiv.) and dirhodium tetracaprolactamate (47 mg; 0.071 mmol; 0.005 equiv.). To the resulting light-blue suspension was then added t-butyl hydroperoxide (5.5 M in decane; 12.9 mL; 71.35 mmol; 5.0 equiv.) dropwise at the same temperature. The resulting purple suspension was stirred for 1.5 h (oxygen-evolution), and another portion of dirhodium tetracaprolactamate (47 mg; 0.071 mmol; 0.005 equiv.) and t-butylhydroperoxide (5.5 M in decane; 12.9 mL; 71.35 mmol; 5.0 equiv.) was added. Stirring was continued for another 1.5 h (oxygen-evolution), the mixture was filtered through a plug of SiO₂, washed with CH₂Cl₂:Et₂O, 1:1, and then carefully concentrated (minimum pressure: 100 mbar). Flash column chromatography (SiO₂; pentane:Et₂O, 4:1→2:1) yielded pure title compound (1.06 g; 6.85 mmol; 48%) as a colorless oil.

13: R_{f}=0.35 (hexane:EtOAc, 1:1); [α]_{D}²⁵=+53.0° (c=1.1 in CHCl₃); IR (film): νₘₐₓ 2955, 1733, 1711, 1588, 1438, 1365, 1271, 1182 cm⁻¹; ¹H-NMR (500 MHz, CDCl₃) δ 7.60 (dd, J=5.7, 2.5 Hz, 1H), 6.13 (dd, J=5.7, 2.1 Hz, 1H), 3.64 (s, 3H), 3.31 (dtq, J=8.1, 6.8, 2.3 Hz, 1H), 2.58 (dd, J=18.9, 6.5 Hz, 1H), 2.50 (dd, J=16.1, 6.9 Hz, 1H), 2.42 (dd, J=16.1, 8.1 Hz, 1H), 1.99 (dd, J=18.9, 2.4 Hz, 1H) ppm; ¹³C-NMR (125 MHz, CDCl₃) δ 208.70, 171.78, 166.37, 134.56, 51.86, 40.67, 38.44, 37.51 ppm; HR-MS (ESI-TOF): calcd for C₈H₁₁O₃ [M+H]⁺: 155.0703. found: 155.0696.

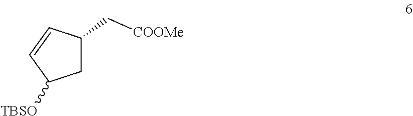 [SMILES: COC(=O)C[C@H]1C=CC(O[Si](C)(C)C(C)(C)C)C1]

### t-Butyldimethylsilyl-alcohol 6

To a stirred solution of (S)-methyl-2-(4-oxocyclopent-2-en-1-yl)acetate (13) (1.68 g; 10.89 mmol; 1.0 equiv.) in MeOH (100 mL) at 25° C. was added CeCl₃.7H₂O (4.00 g; 10.89 mmol; 1.0 equiv.). After dissolution of the reagent, the reaction mixture was cooled to −30° C. and NaBH₄ (412 mg; 10.89 mmol; 1.0 equiv.) was added in one portion. After stirring for 10 min at this temperature, the clear solution was quenched carefully with H₂O (200 mL), warmed to 25° C., and extracted with CH₂Cl₂ (2×100 mL). The combined organic extracts were washed with H₂O (2×50 mL), dried (Na₂SO₄), filtered, and carefully concentrated (minimum pressure: 80 mbar) to yield crude allylic alcohol as a mixture of diastereoisomers (ca. 3:1; 1.60 g; 10.24 mmol; 94%) as a colorless oil which was taken to the next step without purification. To a stirred solution of the crude allylic alcohols (1.60 g; 10.24 mmol) obtained above in CH₂Cl₂ (40 mL) at 0° C. were sequentially added imidazole (2.09 g; 30.72 mmol; 3.0 equiv.) and t-butyldimethylsilyl chloride (2.32 g; 15.36 mmol; 1.5 equiv.). After warming the reaction mixture to 25° C., stirring was continued for 15 min. The suspension formed was then quenched with sat. aq. NH₄Cl solution (40 mL), the phases were separated, and the aq. layer was extracted with CH₂Cl₂ (2×40 mL). The combined organic extracts were washed with H₂O (40 mL), dried (Na₂SO₄), filtered, and concentrated. Flash column chromatography (SiO₂; pentane:Et₂O, 20:1→40:1) yielded pure title compound as a mixture of diastereoisomers (ca. 3:1; 2.22 g; 8.19 mmol; 80%; 75% for two steps) as a colorless oil which was taken to the next step without further purification.

6: R_{f}=0.60 (hexane:EtOAc, 6:1); [α]_{D}²⁵=−13.0° (c=1.3 in CHCl₃); IR (film): νₘₐₓ: 2954, 2930, 2887, 2857, 1739, 1436, 1369, 1250, 1156, 1082 cm⁻¹; ¹H-NMR (500 MHz, CDCl₃, only major isomer) δ 5.83-5.75 (m, 1H), 5.75-5.69 (m, 1H), 4.87-4.77 (m, 1H), 3.66 (s, 3H), 2.98-2.86 (m, 1H), 2.51-2.41 (m, 2H), 2.36 (ddd, J=15.8, 8.2, 1.3 Hz, 1H), 1.29 (dddd, J=13.0, 6.3, 5.4, 1.1 Hz, 1H), 0.87 (d, J=1.3 Hz, 9H), 0.05 (d, J=1.2 Hz, 6H) ppm; ¹³C-NMR (125 MHz, CDCl₃, only major isomer) δ 173.23, 135.73, 135.08, 77.36, 51.60, 40.88, 40.70, 40.43, 26.04, 18.31, −4.51 ppm; HR-MS (ESI-TOF): calcd for C₁₄H₂₆O₃SiNa [M+Na]⁺: 293.1543. found: 293.1544.

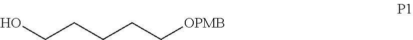

### 5-[(4-Methoxybenzyl)oxy]pentan-1-ol P1

To a stirred suspension of NaH (60% in mineral oil; 5.36 g; 134.0 mmol; 1.0 equiv.) in benzene (250 mL) at 25° C. was added dropwise a solution of 1,5-pentanediol (42.1 mL; 402.0 mmol; 3.0 equiv.) in benzene (50 mL). The resulting mixture was heated under reflux (80° C.) for 5 h, cooled to 25° C., and para-methoxybenzyl chloride (18.2 mL; 134.0 mmol; 1.0 equiv.) was added dropwise. The mixture was heated under reflux (80° C.) for 12 h, allowed to cool to 25° C. and quenched with H₂O (300 mL). The phases were separated, the organic layer was dried (Na₂SO₄), filtered, and concentrated. Flash column chromatography (SiO₂; hexane:EtOAc, 1:1) yielded pure title compound (26.4 g; 117.7 mmol; 88%) as a colorless oil.

P1: R_{f}=0.42 (hexane:EtOAc, 1:1). All analytical data were identical to those reported in the literature (Smith, et al., 2005).

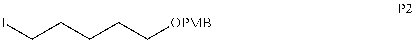

### 1-{[(5-Iodopentyl)oxy]methyl}-4-methoxybenzene P2

To a stirred solution of 5-[(4-methoxybenzyl)oxy]pentan-1-ol P1 (26.4 g; 117.7 mmol; 1.0 equiv.) in THF (400 mL) at 0° C. were added sequentially triphenylphosphine (43.2 g; 164.8 mmol; 1.4 equiv.), imidazole (16.0 g; 235.4 mmol; 2.0 equiv.), and a solution of iodine (38.8 g; 153.0 mmol; 1.3 equiv.) in THF (100 mL). After stirring for 1 h at this temperature, the reaction mixture was quenched with sat. aq. Na₂S₂O₃ solution (300 mL), the phases were separated, and the organic layer was washed with sat. brine (150 mL), dried (MgSO₄), filtered, and concentrated. Flash column chromatography (SiO₂; hexane:EtOAc, 9:1) yielded pure title compound (35.0 g; 104.8 mmol; 89%) as a colorless oil.

P2: R_{f}=0.91 (hexane:EtOAc, 1:1). All analytical data were identical to those reported in the literature (Smith, et al., 2005).

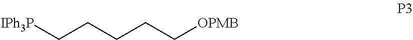

### {5-[(4-Methoxybenzyl)oxy]pentyl}triphenyl-phosphonium iodide P3

To a stirred solution of 1-{[(5-iodopentyl)oxy]methyl}-4-methoxybenzene P2 (35.0 g; 104.8 mmol; 1.0 equiv.) in toluene (350 mL) at 25° C. was added triphenylphosphine (137.4 g; 524.0 mmol; 5.0 equiv.). The reaction mixture was heated under reflux (110° C.) for 18 h, allowed to cool to 25° C., and the toluene layer was decanted from the solidified crude product. Flash column chromatography (SiO₂; hexane:acetone, 2:3→4:1:1) yielded pure title compound (59.6 g; 99.9 mmol; 95%) as a colorless amorphous solid.

P3: R_{f}=0.40 (hexane:acetone, 2:3); IR (film): νₘₐₓ 3052, 3006, 2934, 2860, 2795, 1705, 1611, 1586, 1511, 1436, 1244, 1111, 1027 cm⁻¹; ¹H-NMR (500 MHz, DMSO-d₆) δ 7.95-7.72 (m, 15H), 7.18 (d, J=8.6 Hz, 2H), 6.87 (d, J=8.6 Hz, 2H), 4.31 (s, 2H), 3.73 (s, 3H), 3.65-3.52 (m, 2H), 3.36-3.28 (m, 2H), 1.58-1.48 (m, 6H) ppm; ¹³C-NMR (125 MHz, DMSO-d₆) δ 158.58, 134.89 (d, J=3.0 Hz), 133.57 (d, J=10.1 Hz), 130.44, 130.23 (d, J=12.4 Hz), 129.07, 118.55 (d, J=85.6 Hz), 113.56, 71.48, 68.85, 55.08, 28.27, 26.86 (d, J=17.2 Hz), 21.60 (d, J=3.8 Hz), 20.13 (d, J=49.8 Hz) ppm; ³¹P-NMR (162 MHz, DMSO-d₆) δ 24.99 ppm; HR-MS (ESI-TOF): calcd for C₃₁H₃₄O₂P [M-I]⁺: 469.2291. found: 469.2295.

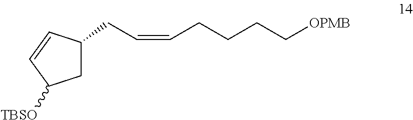

### Diene 14

To a stirred solution of t-butyldimethylsilyl-alcohol 6 (300 mg; 1.11 mmol; 1.0 equiv.) in CH₂Cl₂ (4.5 mL) at −78° C. was dropwise added diisobutylaluminum hydride (1 M in CH₂Cl₂; 1.22 mL; 1.22 mmol; 1.1 equiv.). After stirring for 45 min, the clear solution was quenched with H₂O (0.4 mL; 22.2 mmol; 20 equiv.), diluted with Et₂O (10 mL) and warmed to 25° C. Under vigorous stirring NaF (466 mg; 11.1 mmol; 10 equiv.) was added and the mixture was stirred for an additional 30 min before it was filtered through Celite, washed with Et₂O, and concentrated. Flash column chromatography (SiO₂; pentane:Et₂O, 10:1) yielded the corresponding aldehyde as a mixture of diastereoisomers (ca. 3:1; 260 mg; 1.08 mmol; 97%) as a colorless oil which was taken to the next step without further purification.

To a stirred solution of {5-[(4-methoxybenzyl)oxy]pentyl}triphenyl-phosphonium iodide P3 (975 mg; 1.63 mmol; 1.5 equiv.) in THF (10 mL) at 0° C. was added dropwise sodium bis(trimethylsilyl)amide (1 M in THF; 2.20 mL; 2.20 mmol; 2.0 equiv.). After stirring for 30 min at this temperature, the resulting bright orange solution was cooled to −78° C. and the aldehyde obtained above (260 mg; 1.08 mmol; 1.0 equiv.) in THF (1.5 mL) was added dropwise. After stirring at this temperature for 1 h, the reaction mixture was warmed to 25° C. over the course of 2 h and stirred for an additional 3 h. The reaction mixture was then quenched with sat. aq. NH₄Cl solution (20 mL) and extracted with hexane (4×20 mL). The combined organic extracts were dried (Na₂SO₄), filtered, and concentrated. Flash column chromatography (SiO₂; pentane:Et₂O, 25:1→8:1) yielded pure title compound as a mixture of diastereoisomers (ca. 3:1; 441 mg; 1.02 mmol; 95%; 92% for two steps) as a colorless oil which was taken to the next step without further purification.

14: R_{f}=0.72 (hexane:EtOAc, 10:1); [α]_{D}²⁵=+12.3° (c=1.0 in C₆H₆); IR (film): νmax 3056, 3004, 2929, 2855, 1613, 1586, 1513, 1247, 1098, 1080, 1039 cm⁻¹; ¹H-NMR (500 MHz, CDCl₃, only major isomer) δ 7.26 (d, J=8.8 Hz, 2H), 6.88 (d, J=8.6 Hz, 2H), 5.79 (ddd, J=5.6, 1.9, 1.5 Hz, 1H), 5.70 (ddt, J=5.7, 4.0, 2.0 Hz, 1H), 5.42-5.38 (m, 2H), 4.82 (ddt, J=7.3, 5.7, 1.6 Hz, 1H), 4.43 (s, 2H), 3.80 (s, 3H), 3.43 (t, J=6.6 Hz, 2H), 2.55-2.48 (m, 1H), 2.36 (dt, J=13.0, 7.4 Hz, 1H), 2.22-1.98 (m, 4H), 1.67-1.53 (m, 2H), 1.48-1.36 (m, 2H), 1.27 (ddd, J=13.0, 6.6, 5.7 Hz, 1H), 0.90 (s, 9H), 0.07 (s, 3H), 0.07 (s, 3H) ppm; ¹³C-NMR (125 MHz, CDCl₃, only major isomer) δ 159.20, 136.85, 134.30, 130.85, 130.69, 129.36, 128.20, 113.86, 77.71, 72.65, 70.16, 55.41, 44.46, 40.76, 34.00, 29.54, 27.29, 26.44, 26.13, 18.41, −4.40, −4.43 ppm; HR-MS (ESI-TOF): calcd for C₂₆H₄₂O₃SiNa [M+Na]⁺: 453.2795. found: 453.2795.

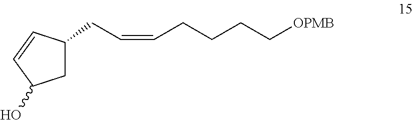

### Allylic Alcohol 15

To a stirred solution of diene 14 (750 mg; 1.74 mmol; 1.0 equiv.) in THF (20 mL) at 0° C. was added dropwise tetrabutylammonium fluoride (1 M in THF; 2.10 mL; 2.10 mmol; 1.2 equiv.). After warming the reaction mixture to 25° C., stirring was continued for 5 h. The brown solution was then quenched with sat. aq. NH₄Cl solution (50 mL) and diluted with EtOAc (50 mL). The phases were separated, the aq. layer was extracted with EtOAc (3×50 mL), and the combined organic extracts were washed with sat. brine (20 mL), dried (MgSO₄), filtered, and concentrated. Flash column chromatography (SiO₂; hexane:EtOAc, 5:2) yielded pure title compound as a mixture of diastereoisomers (ca. 3:1; 500 mg; 1.58 mmol; 91%) as a colorless oil which was taken to the next step without further purification.

15: R_{f}=0.46 (hexane:EtOAc, 2:1); [α]_{D}²⁵=+33.0° (c=1.0 in C₆H₆); IR (film): νmax 3394, 3051, 3003, 2932, 2855, 1612, 1586, 1512, 1246, 1097, 1035 cm⁻¹; ¹H-NMR (500 MHz, CDCl₃, only major isomer) δ 7.26 (d, J=8.8 Hz, 2H), 6.87 (d, J=8.6 Hz, 2H), 5.86 (ddd, J=5.6, 2.1, 1.3 Hz, 1H), 5.79 (dt, J=5.6, 2.0 Hz, 1H), 5.49-5.32 (m, 2H), 4.76 (qd, J=2.7, 1.4 Hz, 1H), 4.43 (s, 2H), 3.80 (s, 3H), 3.43 (t, J=6.5 Hz, 2H), 2.69-2.60 (m, 1H), 2.44 (ddd, J=13.7, 8.0, 7.5 Hz, 1H), 2.22-1.98 (m, 4H), 1.61 (dddd, J=10.8, 8.8, 6.8, 5.1 Hz, 2H), 1.42 (dddd, J=11.9, 7.6, 4.6, 3.2 Hz, 2H), 1.26 (ddd, J=13.7, 5.3, 4.6 Hz, 1H) ppm; ¹³C-NMR (125 MHz, CDCl₃, only major isomer) δ 159.19, 138.35, 133.55, 131.34, 130.82, 129.37, 127.71, 113.86, 77.36, 72.63, 70.04, 55.40, 44.57, 39.81, 33.69, 29.46, 27.22, 26.36 ppm; HR-MS (ESI-TOF): calcd for C₂₀H₂₈O₃Na [M+Na]⁺: 339.1931. found: 339.1926.

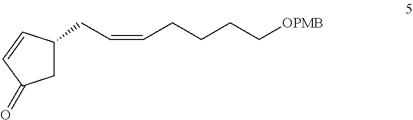

### Enone 5

To a vigorously stirred solution of allylic alcohol 15 (500 mg; 1.58 mmol; 1.0 equiv.) in CH₂Cl₂ (15 mL) at 25° C. was added in one portion pyridinium chlorochromate (680 mg; 3.16 mmol; 2.0 equiv.). After stirring for 2 h, the reaction mixture was diluted with Et₂O (25 mL), filtered through Celite®, washed with Et₂O, and concentrated. Flash column chromatography (SiO₂; hexane:EtOAc, 3:1) yielded slightly contaminated title compound (ca. 85% purity; 544 mg; 1.47 mmol; 93%) as a colorless oil. This material was used in the next step without further purification. An analytically pure sample was obtained by repeated flash column chromatography (SiO₂; CH₂Cl₂:Et₂O, 15:1).

5: R_{f}=0.57 (hexane:EtOAc, 2:1); [α]_{D}²⁵=+90.5° (c=1.0 in C₆H₆); IR (film): νₘₐₓ 3006, 2932, 2855, 1710, 1662, 1612, 1586, 1512, 1246, 1097, 1034 cm⁻¹; ¹H-NMR (500 MHz, CDCl3) δ 7.61 (dd, J=5.6, 2.5 Hz, 1H), 7.25 (d, J=8.9 Hz, 2H), 6.87 (d, J=8.6 Hz, 2H), 6.16 (dd, J=5.6, 2.0 Hz, 1H), 5.53-5.46 (m, 1H), 5.38-5.31 (m, 1H), 4.42 (s, 2H), 3.80 (s, 3H), 3.43 (t, J=6.5 Hz, 2H), 2.99 (dddt, J=11.6, 6.8, 4.5, 2.2 Hz, 1H), 2.50 (dd, J=18.9, 6.4 Hz, 1H), 2.32-2.24 (m, 1H), 2.23-2.15 (m, 1H), 2.07-1.97 (m, 3H), 1.65-1.56 (m, 2H), 1.46-1.39 (m, 2H) ppm; ¹³C-NMR (125 MHz, CDCl₃) δ 210.00, 168.13, 159.21, 134.21, 132.56, 130.77, 129.35, 125.84, 113.85, 72.68, 69.98, 55.39, 41.52, 40.61, 32.03, 29.49, 27.25, 26.30 ppm; HR-MS (ESI-TOF): calcd for C₂₀H₂₆O₃Na [M+Na]⁺: 337.1774. found: 337.1776.

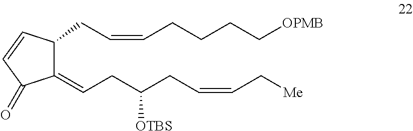

### Dienone 22

To a stirred solution of diisopropylamine (372 μL; 2.64 mmol; 2.2 equiv.) in THF (12 mL) at 0° C. was dropwise added n-butyl lithium (2.5 M in hexanes; 960 μL; 2.40 mmol; 2.0 equiv.). After stirring for 20 min at this temperature, the clear solution was cooled to −78° C. and a solution of enone 5 (ca. 85% purity; 443 mg; 1.20 mmol; 1.0 equiv.) in THF (8 mL) was added dropwise. After stirring the resulting slightly yellow solution for an additional 20 min at this temperature, a solution of aldehyde 8 (372 mg; 1.44 mmol; 1.2 equiv.) in THF (8 mL) was added dropwise and stirring at this temperature was continued for an additional 30 min. The reaction mixture was then quenched with sat. aq. NH₄Cl solution (75 mL), diluted with EtOAc (75 mL), and allowed to warm to 25° C. The phases were separated, the aq. layer was extracted with EtOAc (2×75 mL), and the combined organic extracts were washed with sat. brine (50 mL), dried (Na₂SO₄), filtered, and concentrated. The crude aldol product 20 was filtered through a short column (SiO₂; hexane:EtOAc, 3:1) to obtain a mixture of diastereoisomers (ca. 3:1; 540 mg; 0.95 mmol; 79%) as a colorless oil which was taken to the next step without further purification.

To a stirred solution of aldol product 20 (540 mg; 0.95 mmol) in CH₂Cl₂ (12 mL) at 0° C. was added Et₃N (1.32 mL; 9.48 mmol; 10 equiv.), and then, slowly and dropwise, methanesulfonyl chloride (366 μL; 4.74 mmol; 5.0 equiv.). After stirring for 5 min at this temperature, the reaction mixture was quenched with sat. aq. NaHCO₃ solution (50 mL), diluted with CH₂Cl₂ (50 mL), and allowed to warm to 25° C. The phases were separated, the aq. layer was extracted with CH₂Cl₂ (2×50 mL), and the combined organic layers were washed with H₂O (20 mL), dried (Na₂SO₄), filtered, and concentrated. The crude mesylate 21 was filtered through a short column (SiO₂; hexane:EtOAc, 3:1) to obtain a mixture of diastereoisomers (ca. 3:1; 540 mg; 0.83 mmol; 88%) as a colorless oil which was taken to the next step without further purification.

To a vigorously stirred solution of mesylate 21 (540 mg; 0.83 mmol) in CH₂Cl₂ (18 mL) at 25° C. was added Al₂O₃ (600 mg; 5.85 mmol; 7.0 equiv.). After 2 h and 4 h time intervals two more portions of Al₂O₃ (2×600 mg; 2×5.85 mmol; 2×7.0 equiv.) were added and vigorous stirring was continued for a total of 8 h.

The resulting suspension was then filtered through Celite, washed with EtOAc, and the solution obtained was concentrated. Flash column chromatography (SiO₂; hexane:EtOAc, 7:1) yielded pure title compound (324 mg; 0.58 mmol; 70%; 49% for three steps) as a colorless oil.

22: R_{f}=0.68 (hexane:EtOAc, 3:1); [α]_{D}²⁵=+125.0° (c=1.0 in C₆H₆); IR (film): νₘₐₓ 2930, 2856, 1704, 1656, 1613, 1584, 1513, 1462, 1247, 1095 cm⁻¹; ¹H-NMR (500 MHz, CDCl₃) δ 7.48 (ddd, J=6.0, 2.6, 1.0 Hz, 1H), 7.25 (d, J=8.9 Hz, 2H), 6.87 (d, J=8.7 Hz, 2H), 6.59 (ddt, J=8.1, 7.0, 1.3 Hz, 1H), 6.31 (dd, J=6.0, 1.8 Hz, 1H), 5.51-5.43 (m, 2H), 5.40-5.29 (m, 2H), 4.42 (s, 2H), 3.88 (p, J=6.0 Hz, 1H), 3.79 (s, 3H), 3.46-3.41 (m, 1H), 3.42 (t, J=6.5 Hz, 2H), 2.61 (dddd, J=12.6, 6.5, 3.1, 1.4 Hz, 1H), 2.42 (ddd, J=6.9, 6.0, 2.5 Hz, 2H), 2.30-2.19 (m, 2H), 2.19-2.10 (m, 1H), 1.99 (tdd, J=6.7, 4.6, 3.5 Hz, 4H), 1.58 (ddt, J=9.7, 8.5, 6.3 Hz, 2H), 1.41 (tdd, J=10.3, 7.0, 5.5 Hz, 2H), 0.93 (t, J=7.5 Hz, 3H), 0.87 (s, 9H), 0.05 (s, 3H), 0.04 (s, 3H) ppm; ¹³C-NMR (125 MHz, CDCl₃) δ 196.43, 161.76, 159.20, 138.89, 134.93, 134.10, 132.59, 132.56, 130.76, 129.32, 125.31, 124.39, 113.84, 72.66, 71.64, 70.01, 55.37, 43.54, 36.89, 35.29, 30.70, 29.52, 27.28, 26.31, 25.96, 20.87, 18.18, 14.32, −4.44, −4.45 ppm; HR-MS (ESI-TOF): calcd for C₃₄H₅₂O₄SiNa [M+Na]⁺: 575.3572. found: 575.3511.

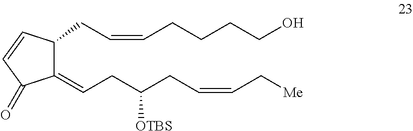 [SMILES: CC/C=C\C[C@@H](C/C=C1/C(=O)C=C[C@@H]1C/C=C\CCCCO)O[Si](C)(C)C(C)(C)C]

### Hydroxy dienone 23

To a vigorously stirred solution of dienone 22 (300 mg; 0.54 mmol; 1.0 equiv.) in a mixture of CH₂Cl₂:H₂O (16:1; 11 mL) at 0° C. was added in one portion 2,3-dichloro-5,6-dicyano-1,4-benzoquinone (187 mg; 0.81 mmol; 1.5 equiv.). After stirring at this temperature for 45 min, the reaction mixture was diluted with Et₂O (30 mL), filtered through Celite®, washed with Et₂O, and concentrated to a volume of ca. 1 mL (not to dryness!). Flash column chromatography (SiO₂; hexane:EtOAc, 3:1→2:1) yielded pure title compound (204 mg; 0.47 mmol; 87%) as a colorless oil.

23: R_{f}⁼0.38 (hexane:EtOAc, 2:1); [α]_{D}²⁵=+146.7° (c=1.0 in C₆H₆); IR (film): νₘₐₓ 3423, 2930, 2857, 1702, 1654, 1580, 1462, 1361, 1253, 1068 cm⁻¹; ¹H-NMR (500 MHz, CDCl₃) δ 7.50 (ddd, J=6.0, 2.6, 1.0 Hz, 1H), 6.59 (ddt, J=8.3, 7.1, 1.3 Hz, 1H), 6.32 (dd, J=6.0, 1.8 Hz, 1H), 5.53-5.43 (m, 2H), 5.40-5.32 (m, 2H), 3.89 (p, J=6.0 Hz, 1H), 3.63 (t, J=6.5 Hz, 2H), 3.44 (ddq, J=8.9, 4.2, 2.1 Hz, 1H), 2.63 (dddd, J=14.5, 6.4, 4.3, 1.5 Hz, 1H), 2.46-2.40 (m, 2H), 2.29-2.12 (m, 3H), 2.06-1.96 (m, 4H), 1.59-1.52 (m, 2H), 1.45-1.36 (m, 2H), 0.93 (t, J=7.5 Hz, 3H), 0.87 (s, 9H), 0.05 (s, 3H), 0.05 (s, 3H) ppm; ¹³C-NMR (125 MHz, CDCl₃) δ 196.47, 161.75, 138.93, 134.99, 134.15, 132.65, 132.48, 125.47, 124.39, 71.69, 62.87, 43.59, 36.96, 35.30, 32.45, 30.76, 27.22, 25.98, 25.85, 20.89, 18.22, 14.34, −4.42, −4.43 ppm; HR-MS (ESI-TOF): calcd for C₂₆H₄₄O₃ SiNa [M+Na]⁺: 455.2952. found: 455.2944.

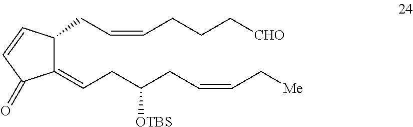 [SMILES: CC/C=C\C[C@@H](C/C=C1/C(=O)C=C[C@@H]1C/C=C\CCCC=O)O[Si](C)(C)C(C)(C)C]

### Aldehyde Dienone 24

To a vigorously stirred solution of hydroxy dienone 23 (200 mg; 0.46 mmol; 1.0 equiv.) in CH₂Cl₂ (5 mL) at 25° C. was added in one portion pyridinium chlorochromate (200 mg; 0.93 mmol; 2.0 equiv.). After stirring for 2 h, the reaction mixture was diluted with Et₂O (30 mL), filtered through Celite, washed with Et₂O, and concentrated to a volume of ca. 1 mL (not to dryness!). Flash column chromatography (SiO₂; hexane:EtOAc, 7:1→5:1) yielded pure title compound (181 mg; 0.42 mmol; 91%) as a colorless oil.

24: R_{f}⁼0.43 (hexane:EtOAc, 3:1); [α]_{D}²⁵=+155.0° (c=1.0 in C₆H₆); IR (film): νₘₐₓ 2955, 2930, 2856, 1724, 1703, 1655, 1581, 1462, 1361, 1254, 1086 cm⁻¹; ¹H-NMR (500 MHz, CDCl₃) δ 9.75 (t, J=1.6 Hz, 1H), 7.48 (ddd, J=6.0, 2.6, 1.0 Hz, 1H), 6.59 (ddt, J=8.3, 7.0, 1.3 Hz, 1H), 6.32 (dd, J=6.0, 1.8 Hz, 1H), 5.51-5.40 (m, 2H), 5.41-5.32 (m, 2H), 3.88 (p, J=6.0 Hz, 1H), 3.45 (ddt, J=8.6, 4.0, 2.1 Hz, 1H), 2.64-2.57 (m, 1H), 2.44-2.38 (m, 4H), 2.29-2.11 (m, 3H), 2.06-1.95 (m, 4H), 1.67 (p, J=7.4 Hz, 2H), 0.93 (t, J=7.5 Hz, 3H), 0.86 (s, 9H), 0.05 (s, 3H), 0.04 (s, 3H) ppm; ¹³C-NMR (125 MHz, CDCl₃) δ 202.26, 196.33, 161.49, 138.77, 135.09, 134.13, 132.72, 131.35, 126.38, 124.36, 71.63, 43.40, 43.39, 36.90, 35.30, 30.64, 26.73, 25.96, 21.95, 20.88, 18.20, 14.33, −4.43 ppm; HR-MS (ESI-TOF): calcd for C₂₆H₄₂O₃SiNa [M+Na]⁺: 453.2795. found: 453.2784.

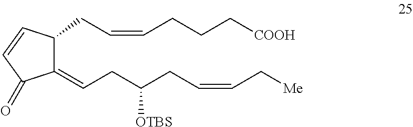 [SMILES: CC/C=C\C[C@@H](C/C=C1/C(=O)C=C[C@@H]1C/C=C\CCCC(=O)O)O[Si](C)(C)C(C)(C)C]

### Δ¹²-PGJ₃-14-t-butyldimethylsilyl-ether (25)

To a vigorously stirred solution of aldehyde dienone 24 (180 mg; 0.42 mmol; 1.0 equiv.) in t-BuOH (6 mL) at 25° C. were dropwise added sequentially 2-methyl-2-butene (470 μL; 4.18 mmol, 10 equiv.), a solution of NaH₂PO₄ (98 mg; 0.63 mmol; 1.5 equiv.) in H₂O (2.25 mL) and a solution of NaClO₂ (80%; 71 mg; 0.63 μmol; 1.5 equiv.) in H₂O (2.25 mL). After stirring for 30 min, the reaction mixture was diluted with a solution of NaH₂PO₄ (4.0 g) in H₂O (80 mL) and extracted with EtOAc (5×80 mL). The combined organic extracts were washed with sat. brine (50 mL), dried (Na₂SO₄), filtered, and concentrated to a volume of ca. 1 mL (not to dryness!). Flash column chromatography (SiO₂; CH₂Cl₂:EtOH, 20:1) yielded pure title compound (178 mg; 0.40 mmol; 95%) as a colorless oil.

25: R_{f}=0.57 (CH₂Cl₂:EtOH, 10:1); [α]_{D}²⁵=+140.5° (c=1.0 in C₆H₆); IR (film): νₘₐₓ 3089, 2956, 2929, 2856, 1705, 1654, 1580, 1462, 1251, 1086 cm⁻¹; ¹H-NMR (500 MHz, CDCl₃) δ 7.49 (ddd, J=6.0, 2.6, 1.0 Hz, 1H), 6.63-6.57 (m, 1H), 6.33 (dd, J=6.0, 1.8 Hz, 1H), 5.51-5.42 (m, 2H), 5.42-5.32 (m, 2H), 3.88 (p, J=6.0 Hz, 1H), 3.44 (dt, J=7.4, 2.2 Hz, 1H), 2.67-2.58 (m, 1H), 2.45-2.40 (m, 2H), 2.33 (t, J=7.4 Hz, 2H), 2.29-2.10 (m, 3H), 2.05 (q, J=7.4 Hz, 2H), 2.03-1.95 (m, 2H), 1.68 (p, J=7.3 Hz, 2H), 0.92 (t, J=7.5 Hz, 3H), 0.87 (s, 9H), 0.05 (s, 3H), 0.04 (s, 3H) ppm; ¹³C-NMR (125 MHz, CDCl₃) δ 196.55, 179.08, 161.75, 138.82, 135.02, 134.16, 132.84, 131.37, 126.34, 124.34, 71.73, 43.50, 36.92, 35.26, 33.48, 30.66, 26.71, 25.96, 24.57, 20.88, 18.21, 14.32, −4.44 ppm; HR-MS (ESI-TOF): calcd for C₂₆H₄₂O₄SiNa [M+Na]⁺: 469.2745. found: 469.2728.

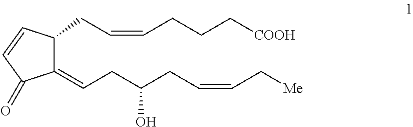 [SMILES: CC/C=C\C[C@H](O)C/C=C1/C(=O)C=C[C@@H]1C/C=C\CCCC(=O)O]

### Δ¹²-PGJ₃ (1)

To a stirred solution of Δ¹²-PGJ₃-14-t-butyldimethylsilyl-ether (25) (178 mg; 0.40 mmol; 1.0 equiv.) in MeCN (3.5 mL) at 0° C. was dropwise added a solution of HF (50% aq.; 700 μL; ca. 17.8 mmol; ca. 50 equiv.) in MeCN (3.5 mL). After stirring for 45 min at this temperature, the reaction mixture was quenched with sat. brine (30 mL) and extracted with EtOAc (5×50 mL). The combined organic extracts were dried (Na₂SO₄), filtered, and concentrated to a volume of ca. 1 mL (not to dryness!). Flash column chromatography (SiO₂; CH₂Cl₂:EtOH, 15:1) yielded pure title compound (123 mg; 0.37 mmol; 92%) as a colorless oil.

1: R_{f}=0.56 (CH₂Cl₂:EtOH, 10:1); [α]_{D}²⁵=+129.0° (c=0.5 in C₆H₆); UV(EtOH): λₘₐₓ (log ε) 249 (4.35) nm; IR (film): νₘₐₓ 3406, 3010, 2960, 2929, 2872, 1698, 1646, 1578, 1406, 1236, 1046 cm⁻¹; ¹H-NMR (600 MHz, CDCl₃) δ 7.55 (ddd, J=6.0, 2.6, 1.0 Hz, 1H), 6.63-6.57 (m, 1H), 6.35 (dd, J=6.0, 1.8 Hz, 1H), 5.62-5.55 (m, 1H), 5.52-5.33 (m, 3H), 3.88 (p, J=6.4 Hz, 1H), 3.47 (ddd, J=9.6, 4.4, 2.2 Hz, 1H), 2.74-2.66 (m, 1H), 2.57 (dt, J=15.1, 6.7 Hz, 1H), 2.48 (ddd, J=14.9, 8.4, 6.4 Hz, 1H), 2.34 (t, J=7.0 Hz, 2H), 2.31-2.27 (m, 2H), 2.16-2.01 (m, 5H), 1.68 (p, J=7.0 Hz, 2H), 0.95 (t, J=7.5 Hz, 3H) ppm; ¹³C-NMR (150 MHz, CDCl₃) δ 196.61, 177.79, 161.98, 139.65, 135.85, 135.01, 131.72, 131.67, 126.16, 123.73, 70.92, 43.77, 36.48, 34.63, 33.19, 30.53, 26.62, 24.58, 20.88, 14.34 ppm; HR-MS (ESI-TOF): calcd for C₂₀H₂₈O₄Na [M+Na]+: 355.1880. found: 355.1889.

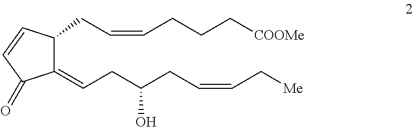 [SMILES: CC/C=C\C[C@H](O)C/C=C1/C(=O)C=C[C@@H]1C/C=C\CCCC(=O)OC]

### Δ¹²-PGJ₃ methyl ester (2)

To a stirred solution of Δ¹²-PGJ₃ (1) (5.0 mg; 15.0 μmol; 1.0 equiv.) in C₆H₆:MeOH (3:2; 0.5 mL) at 25° C. was dropwise added a solution of trimethylsilyl diazomethane (2 M in Et₂O; 12 μL; 22.5 μmol; 1.5 equiv.) (yellow color persists). After stirring for 30 min, the reaction mixture was concentrated. Flash column chromatography (SiO₂; hexane:EtOAc: 2:1→3:2) yielded pure title compound (4.9 mg; 14.0 μmol; 93%) as a colorless oil.

2: R_{f}=0.53 (hexane:EtOAc, 1:1); [α]_{D}²⁵=+206.3° (c=0.4 in C₆H₆); IR (film): νₘₐₓ 3446, 3009, 2956, 2927, 2872, 2855, 1736, 1700, 1651, 1579, 1436, 1208, 1049 cm⁻¹; ¹H-NMR (500 MHz, CDCl₃) δ 7.51 (ddd, J=6.0, 2.7, 1.0 Hz, 1H), 6.66-6.61 (m, 1H), 6.34 (dd, J=6.0, 1.8 Hz, 1H), 5.64-5.56 (m, 1H), 5.50-5.43 (m, 1H), 5.41-5.32 (m, 2H), 3.84 (p, J=6.4 Hz, 1H), 3.66 (s, 3H), 3.50 (ddd, J=6.6, 3.9, 2.0 Hz, 1H), 2.68-2.60 (m, 1H), 2.57-2.43 (m, 2H), 2.33-2.18 (m, 4H), 2.11-2.01 (m, 4H), 1.98 (br, 1H), 1.67 (dt, J=14.7, 7.4 Hz, 3H), 0.96 (t, J=7.5 Hz, 3H) ppm; ¹³C-NMR (125 MHz, CDCl₃) δ 196.50, 174.17, 161.81, 139.49, 135.87, 135.08, 131.79, 131.67, 125.95, 123.85, 70.64, 51.73, 43.45, 36.66, 35.00, 33.48, 30.41, 26.83, 24.77, 20.90, 14.37 ppm; HR-MS (ESI-TOF): calcd for C₂₁H₃₀O₄Na [M+Na]⁺: 369.2036. found: 369.2046.

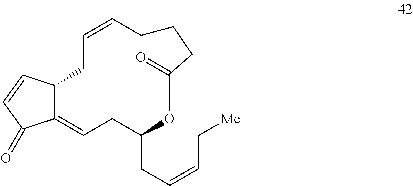 [SMILES: CC/C=C\C[C@H]1C/C=C2/C(=O)C=C[C@@H]2C/C=C\CCCC(=O)O1]

### Δ¹²-PGJ₃ lactone (42)

To a stirred solution of 2-methyl-6-nitrobenzoic anhydride (14.5 mg; 42.0 μmol; 1.4 equiv.) and 4-dimethylaminopyridine (22 mg; 180.0 μmol; 6.0 equiv.) in CH₂Cl₂ (20 mL) was added a solution of Δ¹²-PGJ₃ (1) (10.0 mg; 30.0 μmol; 1.0 equiv.) in CH₂Cl₂ (10 mL) at 25° C. dropwise via syringe pump over 15 h. After stirring for an additional 2 h, the reaction mixture was washed sequentially with sat. aq. NaHCO3 solution (10 mL), aq. HCl (0.2 M; 10 mL), and sat. brine (10 mL). The organic layer was dried (Na₂SO₄), filtered, and concentrated. Flash column chromatography (SiO₂; hexane:EtOAc: 3:1) yielded pure title compound (7.0 mg; 22.2 μmol; 74%) as a colorless oil.

42: R_{f}=0.35 (hexane:EtOAc, 3:1); [α]_{D}²⁵=+65.2° (c=0.6 in C₆H₆); IR (film): νₘₐₓ 3010, 2961, 2927, 2855, 1727, 1704, 1655, 1581, 1456, 1440, 1239, 1151, 1024 cm⁻¹; ¹H-NMR (600 MHz, CDCl₃) δ 7.52 (dd, J=6.1, 2.6 Hz, 1H), 6.51-6.46 (m, 1H), 6.38 (dd, J=6.1, 1.9 Hz, 1H), 5.59-5.52 (m, 1H), 5.40-5.31 (m, 2H), 5.19 (ddtd, J=25.5, 9.5, 6.1, 2.4 Hz, 2H), 3.73-3.68 (m, 1H), 2.81 (ddd, J=14.7, 10.0, 5.1 Hz, 1H), 2.75-2.67 (m, 1H), 2.54-2.37 (m, 4H), 2.31 (ddd, J=15.5, 9.6, 2.8 Hz, 1H), 2.24 (ddd, J=15.6, 8.7, 2.6 Hz, 1H), 2.10-2.03 (m, 2H), 1.96 (ddt, J=13.8, 8.0, 5.7 Hz, 1H), 1.71-1.59 (m, 2H), 1.55-1.46 (m, 1H), 0.98 (t, J=7.5 Hz, 3H) ppm; 13C-NMR (150 MHz, CDCl₃) δ 196.30, 173.08, 161.07, 140.08, 135.52, 135.47, 131.96, 131.61, 125.08, 122.76, 72.97, 43.28, 33.73, 32.88, 32.09, 28.37, 25.89, 24.50, 20.87, 14.30 ppm; HR-MS (ESI-TOF): calcd for C₂₀H₂₇O₃ [M+H]⁺: 315.1955. found: 315.1955.

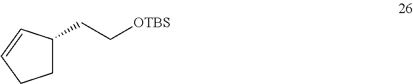 [SMILES: CC(C)(C)[Si](C)(C)OCC[C@H]1C=CCC1]

### (S)-t-Butyl[2-(cyclopent-2-en-1-yl)ethoxy]dimethylsilane (26)

To a stirred solution of (R)-dimethyl-2-(cyclopent-2-en-1-yl)malonate (11) (5.61 g; 40.0 mmol; 1.0 equiv.) in CH₂Cl₂ (200 mL) at −78° C. was dropwise added diisobutylaluminum hydride (1 M in CH₂Cl₂; 100.0 mL; 100.0 mmol; 2.5 equiv.). The reaction mixture was allowed to warm to 25° C. and stirring was continued for 10 min at this temperature. The so-obtained clear solution was quenched carefully with sat. aq. Rochelle-salt solution (Na/KC₄H₄O₆.4H₂O; 300 mL), stirred vigorously for 1 h, and the phases were separated. The aq. layer was extracted with CH₂Cl₂ (3×100 mL). The combined organic extracts were washed with H₂O (100 mL), dried (Na₂SO₄), filtered, and carefully concentrated (minimum pressure: 100 mbar) to yield the corresponding crude alcohol (4.26 g; 38.0 mmol; 95%) as a colorless oil which was taken to the next step without purification.

To a stirred solution of the crude alcohol (4.26 g; 38.0 mmol) in CH₂Cl₂ (100 mL) at 0° C. were sequentially added imidazole (6.47 g; 95.0 mmol; 2.5 equiv.) and t-butyldimethylsilyl chloride (7.45 g; 49.4 mmol; 1.3 equiv.). After stirring at this temperature for 15 min, the resulting suspension was quenched with sat. aq. NH₄Cl solution (100 mL), the phases were separated, and the aq. layer was extracted with CH₂Cl₂ (2×100 mL). The combined organic extracts were washed with H₂O (100 mL), dried (MgSO₄), filtered, and concentrated. Flash column chromatography (SiO₂; pentane:Et₂O, 50:1) yielded pure title compound (7.61 g; 33.6 mmol; 88%; 84% for two steps) as a colorless oil.

26: R_{f}=0.95 (hexane:EtOAc, 10:1); [α]_{D}²⁵=+56.8° (c=1.0 in CHCl₃); IR (film): νₘₐₓ: 2952, 2938, 2899, 2856, 1472, 1462, 1253, 1099 cm⁻¹; ¹H-NMR (500 MHz, CDCl₃) δ 5.71 (dq, J=5.7, 2.1 Hz, 1H), 5.68 (dq, J=5.8, 2.0 Hz, 1H), 3.70-3.62 (m, 2H), 2.74 (ddtt, J=10.7, 8.5, 4.4, 2.2 Hz, 1H), 2.38-2.22 (m, 2H), 2.10-1.99 (m, 1H), 1.65 (ddt, J=13.2, 7.2, 6.6 Hz, 1H), 1.54-1.45 (m, 1H), 1.45-1.37 (m, 1H), 0.90 (s, 9H), 0.05 (s, 6H) ppm; ¹³C-NMR (125 MHz, CDCl₃) δ 135.23, 130.36, 62.27, 42.32, 39.23, 32.06, 30.04, 26.14, 18.52, −5.12 ppm; HR-MS (ESI-TOF): calcd for C₁₃H₂₇OSi [M+H]⁺: 227.1826. found: 227.1833.

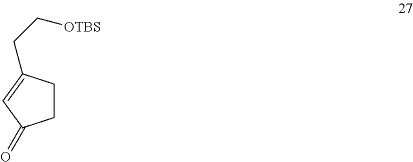 [SMILES: CC(C)(C)[Si](C)(C)OCCC1=CC(=O)CC1]

### (R)-4-{2-[(t-Butyldimethylsilyl)oxy]ethyl}cyclopent-2-enone (27)

To a stirred solution of (S)-t-butyl[2-(cyclopent-2-en-1-yl)ethoxy]dimethylsilane (26) (3.43 g; 14.27 mmol; 1.0 equiv.) in CH₂Cl₂ (50 mL) at 25° C. under an atmosphere of oxygen (balloon) were added sequentially K₂CO₃ (987 mg; 7.14 mmol; 0.5 equiv.) and dirhodium tetracaprolactamate (47 mg; 0.071 mmol; 0.005 equiv.). To the resulting light-blue suspension was then dropwise added t-butyl hydroperoxide (5.5 M in decane; 12.9 mL; 71.35 mmol; 5.0 equiv.). The resulting purple suspension was stirred for 1.5 h (oxygen-evolution), and another portion of dirhodium tetracaprolactamate (47 mg; 0.071 mmol; 0.005 equiv.) and t-butyl hydroperoxide (5.5 M in decane; 12.9 mL; 71.35 mmol; 5.0 equiv.) were added. Stirring was continued for another 1.5 h (oxygen-evolution), the mixture was filtered through a plug of SiO₂, washed with CH₂Cl₂:Et₂O, 1:1, and then carefully concentrated (minimum pressure: 50 mbar). Flash column chromatography (SiO₂; hexane:EtOAc, 5:1) yielded pure title compound (2.09 g; 8.70 mmol; 61%) as a colorless oil.

27: R_{f}=0.44 (hexane:EtOAc, 2:1); [α]_{D}²⁵=+0.3° (c=1.0 in C₆H₆); IR (film): νmax 2954, 2929, 2857, 1708, 1673, 1616, 1472, 1254, 1092 cm⁻¹; ¹H-NMR (500 MHz, C₆D₆) δ 7.16 (s, 1H), 5.90 (tt, J=1.8, 1.3 Hz, 1H), 3.44 (t, J=6.4 Hz, 2H), 2.08 (tdd, J=6.3, 0.4 Hz, 1H), 2.05-1.99 (m, 1H), 1.90 (dtd, J=5.2, 1.9, 0.9 Hz, 2H), 0.99-0.93 (m, 1H), 0.92 (s, 9H), −0.02 (s, 6H) ppm; ¹³C-NMR (125 MHz, C₆D₆) δ 208.11, 178.15, 130.80, 60.81, 36.56, 35.17, 31.66, 26.00, 18.35, −5.34 ppm; HR-MS (ESI-TOF): calcd for C₁₃H₂₅O₂Si [M+H]+: 241.1618. found: 241.1622.

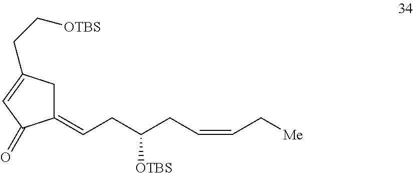 [SMILES: CC/C=C\C[C@@H](C/C=C1\CC(CCO[Si](C)(C)C(C)(C)C)=CC1=O)O[Si](C)(C)C(C)(C)C]

### Dienone 34

To a stirred solution of diisopropylamine (56 μL; 0.400 mmol; 2.0 equiv.) in THF (2 mL) at 0° C. was dropwise added n-butyl lithium (1.6 M in hexanes; 244 μL; 0.390 mmol; 1.95 equiv.). After stirring for 20 min at this temperature, the clear solution was cooled to −78° C. and a solution of (R)-4-{2-[(tbutyldimethylsilyl)oxy]ethyl}cyclopent-2-enone (27) (50 mg; 0.200 mmol; 1.0 equiv.) in THF (2 mL) was added dropwise. After stirring the resulting slightly yellow solution for an additional 20 min at this temperature, a solution of aldehyde 8 (62 mg; 0.240 mmol; 1.2 equiv.) in THF (2 mL) was added dropwise and stirring at this temperature was continued for an additional 30 min. The reaction mixture was then quenched with sat. aq. NH₄Cl solution (10 mL), diluted with EtOAc (10 mL), and allowed to warm to 25° C. The phases were separated, the aq. Layer was extracted with EtOAc (2×10 mL), and the combined organic extracts were washed with sat. brine (10 mL), dried (Na₂SO₄), filtered, and concentrated. The crude aldol product 28 was filtered through a short column (SiO₂; hexane:EtOAc, 5:1) to obtain a mixture of diastereoisomers (ca. 3:1; 70 mg; 0.140 mmol; 70%) as a colorless oil which was taken to the next step without further purification.

To a stirred solution of aldol product 28 (70 mg; 0.140 mmol) in CH₂Cl₂ (2 mL) at 0° C. was added Et₃N (194 μL; 1.400 mmol; 10 equiv.), and then, slowly and dropwise, methanesulfonyl chloride (54 μL; 0.700 mmol; 5.0 equiv.). After stirring for 5 min at this temperature, the reaction mixture was quenched with sat. aq. NaHCO₃ solution (10 mL), diluted with CH₂Cl₂ (10 mL), and allowed to warm to 25° C. The phases were separated, the aq. layer was extracted with CH₂Cl₂ (2×10 mL), and the combined organic layers were washed with H₂O (5 mL), dried (Na₂SO₄), filtered, and concentrated. The crude mesylate 29 was filtered through a short column (SiO₂; hexane:EtOAc, 4:1) to obtain a mixture of diastereoisomers (ca. 3:1; 74 mg; 0.129 mmol; 92%) as a colorless oil which was taken to the next step without further purification.

To a vigorously stirred solution of mesylate 29 (74 mg; 0.129 mmol) in CH₂Cl₂ (2 mL) at 25° C. was added Al₂O₃ (92 mg; 0.903 mmol; 7.0 equiv.). After 2 h and 4 h time intervals two more portions of Al₂O₃ (2×92 mg; 2×0.903 mmol; 2×7.0 equiv.) were added and vigorous stirring was continued for a total of 8 h. The resulting suspension was then filtered through Celite, washed with EtOAc, and the solution obtained was concentrated. Flash column chromatography (SiO₂; hexane:EtOAc, 10:1) yielded pure title compound (55 mg; 0.115 mmol; 89%; 57% for three steps) as a colorless oil.

34: R_{f}=0.73 (hexane:EtOAc, 3:1); [α]_{D}²⁵=+16.3° (c=1.0 in C₆H₆); IR (film): νₘₐₓ 2955, 2929, 2886, 2856, 1704, 1663, 1611, 1472, 1361, 1254, 1091 cm⁻¹; ¹H-NMR (500 MHz, C₆D₆) δ 7.16 (m, 1H), 6.85-6.75 (m, 1H), 6.17-6.11 (m, 1H), 5.55-5.39 (m, 2H), 3.80-3.69 (m, 1H), 3.48 (td, J=6.3, 0.5 Hz, 1H), 2.90-2.72 (m, 2H), 2.32-2.14 (m, 6H), 1.99 (ddt, J=7.9, 7.3, 6.3 Hz, 2H), 0.97 (s, 9H), 0.93 (s, 9H), 0.92 (t, J=7.5 Hz, 3H), 0.07 (d, J=0.4 Hz, 3H), 0.06 (d, J=0.4 Hz, 3H), −0.01 (s, 6H) ppm; ¹³C-NMR (125 MHz, C₆D₆) δ 194.49, 170.62, 137.55, 133.92, 132.72, 129.85, 125.06, 71.94, 60.98, 37.60, 36.36, 35.84, 35.42, 26.08, 26.03, 21.15, 18.36, 18.27, 14.44, −4.38, −4.39, −5.32 ppm; HR-MS (ESI-TOF): calcd for C₂₇H₅₁O₃Si₂ [M+H]⁺: 479.3371. found: 479.3375.

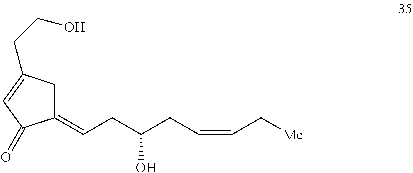 [SMILES: CC/C=C\C[C@H](O)C/C=C1\CC(CCO)=CC1=O]

### Δ¹²-PGJ₃ analog 35

To a stirred solution of dienone 34 (30.0 mg; 62.6 μmol; 1.0 equiv.) in MeCN (0.6 mL) at 0° C. was dropwise added a solution of HF (50% aq.; 124 μL; ca. 3.1 mmol; ca. 50 equiv.) in MeCN (0.6 mL). After stirring for 1 h at this temperature, the reaction mixture was quenched with sat. aq. NaHCO₃ solution (10 mL), and extracted with EtOAc (3×10 mL). The combined organic extracts were washed with sat. brine (5 mL), dried (Na₂SO₄), filtered, and concentrated to a volume of ca. 0.1 mL (not to dryness!). Flash column chromatography (SiO₂; EtOAc) yielded pure title compound (15.5 mg; 62.0 μmol; 99%) as a colorless oil.

35: R_{f}=0.25 (EtOAc); [α]_{D}²⁵=+9.8° (c=1.0 in C₆H₆); IR (film): νₘₐₓ 3372, 2960, 2930, 1692, 1648, 1603, 1413, 1334, 1275, 1048 cm⁻¹; ¹H-NMR (500 MHz, C₆D₆) δ 7.18-7.14 (m, 1H), 6.80 (tt, J=7.8, 1.7 Hz, 1H), 6.22 (s, 1H), 5.59-5.50 (m, 2H), 4.00 (s, 2H), 3.81 (p, J=6.1 Hz, 1H), 3.69 (t, J=6.1 Hz, 1H), 2.96 (d, J=21.2 Hz, 1H), 2.82 (d, J=21.2 Hz, 1H), 2.43-2.23 (m, 6H), 2.11-2.02 (m, 2H), 0.96 (t, J=7.5 Hz, 3H) ppm; ¹³C-NMR (125 MHz, C₆D₆) δ 196.83, 174.27, 137.83, 134.27, 132.35, 131.51, 125.33, 70.85, 59.86, 37.50, 36.51, 35.72, 35.32, 21.19, 14.53 ppm; HR-MS (ESI-TOF): calcd for C₁₅H₂₂O₃Na [M+Na]⁺: 273.1461. found: 273.1465.

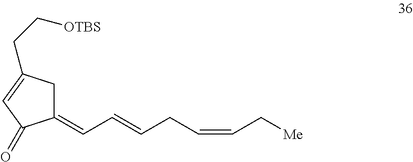 [SMILES: CC/C=C\C/C=C/C=C1\CC(CCO[Si](C)(C)C(C)(C)C)=CC1=O]

### Tetraene 36

To Al₂O₃ (23.5 mg; 240 μmol; 10 equiv.), activated by heating to 400° C. under vacuum for 5 min, was added a solution of mesylate 29 (13.8 mg; 24.0 μmol) in CH₂Cl₂ (1 mL) at 25° C. After vigorous stirring for 30 min, the resulting suspension was filtered through Celite®, washed with EtOAc, and the solution obtained was concentrated. Flash column chromatography (SiO₂; hexane:EtOAc, 10:1) yielded pure title compound (4.5 mg; 13.0 μmol; 54%) as a colorless oil.

36: R_{f}=0.69 (hexane:EtOAc, 3:1); IR (film): νₘₐₓ 2955, 2929, 2856, 1702, 1662, 1257, 1091 cm⁻¹; ¹H-NMR (500 MHz, C₆D₆) δ 6.81 (ddt, J=8.1, 7.3, 1.9 Hz, 1H), 6.26 (ddd, J=17.5, 10.6, 0.7 Hz, 1H), 6.08 (ddt, J=1.7, 1.2, 0.6 Hz, 1H), 5.56-5.39 (m, 2H), 5.21 (dt, J=17.5, 0.8 Hz, 1H), 5.02-4.92 (m, 1H), 3.80-3.68 (m, 1H), 3.02-2.76 (m, 2H), 2.33-2.15 (m, 4H), 2.04-1.91 (m, 2H), 0.95 (s, 9H), 0.89 (t, J=7.5 Hz, 3H), 0.05 (s, 3H), 0.05 (s, 3H) ppm; ¹³C-NMR (125 MHz, C₆D₆) δ 194.24, 163.95, 137.20, 133.96, 133.82, 133.11, 130.58, 125.00, 121.00, 71.92, 37.57, 35.84, 30.42, 26.04, 21.14, 18.25, 14.40, −4.41, −4.43 ppm; HR-MS (ESI-TOF): calcd for C₂₁H₃₄O₂SiNa [M+Na]⁺: 369.2220. found: 369.2216.

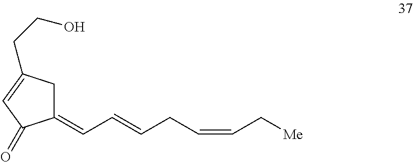 [SMILES: CC/C=C\C/C=C/C=C1\CC(CCO)=CC1=O]

### 15-Deoxy-Δ^{12,14}-PGJ₃ analog 37

To a stirred solution of tetraene 36 (5.0 mg; 14.4 μmol; 1.0 equiv.) in MeCN (0.15 mL) at 0° C. was dropwise added a solution of HF (50% aq.; 29 μL; ca. 0.72 mmol; ca. 50 equiv.) in MeCN (0.15 mL). After stirring for 1 h at this temperature, the reaction mixture was quenched with sat. aq. NaHCO₃ solution (3 mL), and extracted with EtOAc (3×3 mL). The combined organic extracts were washed with sat. brine (3 mL), dried (Na₂SO₄), filtered, and concentrated to a volume of ca. 0.1 mL (not to dryness!). Flash column chromatography (SiO₂; hexane:EtOAc, 1:1) yielded pure title compound (3.3 mg; 14.3 μmol; 99%) as a colorless oil.

37: R_{f}=0.74 (EtOAc); [α]_{D}²⁵=−10.0° (c=0.5 in C₆H₆); IR (film): νₘₐₓ 3419, 2962, 2930, 1693, 1650, 1621, 1564, 1418, 1349, 1271, 1200, 1049 cm⁻¹; ¹H-NMR (500 MHz, C₆D₆) δ 6.83 (tt, J=7.2, 1.8 Hz, 1H), 6.25 (ddd, J=17.5, 10.6, 0.7 Hz, 1H), 6.07 (td, J=1.7, 0.5 Hz, 1H), 5.57-5.42 (m, 1H), 5.36 (dtd, J=10.8, 7.3, 1.5 Hz, 1H), 5.18 (d, J=17.5 Hz, 1H), 4.97 (d, J=10.8 Hz, 1H), 3.53 (dtd, J=8.2, 6.3, 5.9, 2.9 Hz, 1H), 2.89-2.81 (m, 1H), 2.81-2.74 (m, 1H), 2.22-2.05 (m, 4H), 2.00-1.92 (m, 2H), 0.88 (t, J=7.5 Hz, 3H) ppm; ¹³C-NMR (125 MHz, C₆D₆) δ 194.76, 164.50, 137.43, 134.62, 133.63, 132.99, 130.72, 124.99, 121.34, 70.56, 37.28, 35.49, 30.40, 21.07, 14.43 ppm; HR-MS (ESI-TOF): calcd for C₁₅H₂₁O₂ [M+H]⁺: 233.1536. found: 233.1538.

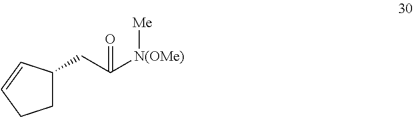

### (S)-2-(Cyclopent-2-en-1-yl)-N-methoxy-N-methylacetamide (30)

To a stirred suspension of N,O-dimethylhydroxylamine hydrochloride (195 mg; 2.00 mmol; 2.0 equiv.) in CH₂Cl₂ (3 mL) at 0° C. was dropwise added AlMe₃ (2 M in hexane; 1.0 mL; 2.00 mmol; 2.0 equiv.). After stirring for 45 min at this temperature the reaction mixture was allowed to warm to 25° C. and was stirred for an additional 45 min. The so obtained clear solution was cooled to 0° C., and a solution of (S)-methyl-2-(cyclopent-2-en-1-yl)acetate (12) (141 mg; 1.00 mmol; 1.0 equiv.) in CH₂Cl₂ (2 mL) was added dropwise. The reaction mixture was allowed to warm to 25° C. and stirred at this temperature for 4 h. It was then again cooled to 0° C. and carefully, dropwise quenched with aq. HCl (2 M; 5 mL). The phases were separated, the aq. layer was extracted with CH₂Cl₂ (2×10 mL), and the combined organic extracts were sequentially washed with H₂O (10 mL) and sat. brine (10 mL), dried (Na₂SO₄), filtered, and carefully concentrated (minimum pressure 100 mbar). Flash column chromatography (SiO₂; pentane:Et₂O, 2:1) yielded pure title compound (161 mg; 0.95 mmol; 95%) as a colorless oil.

30: R_{f}=0.37 (hexane:EtOAc, 3:1); [α]_{D}²⁵=+94.7° (c=1.0 in C₆H₆); IR (film): νₘₐₓ 3050, 2938, 2850, 1663, 1413, 1382, 1176, 1002 cm⁻¹; ¹H-NMR (500 MHz, CDCl₃) δ 5.75 (dq, J=5.7, 2.2 Hz, 1H), 5.70 (dq, J=5.7, 2.1 Hz, 1H), 3.66 (s, 3H), 3.17 (s, 3H), 3.16-3.09 (m, 1H), 2.53-2.37 (m, 2H), 2.39-2.25 (m, 2H), 2.14 (dtd, J=13.0, 8.5, 5.0 Hz, 1H), 1.45 (ddt, J=12.7, 9.0, 6.2 Hz, 1H) ppm; ¹³C-NMR (125 MHz, CDCl₃) δ 174.01, 134.55, 131.19, 61.30, 41.69, 38.04, 32.21, 31.97, 30.02 ppm; HR-MS (ESI-TOF): calcd for C₉H₁₆NO₂ [M+H]⁺: 170.1176. found: 170.1172.

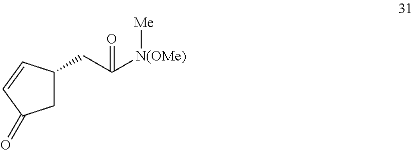

### (S)—N-Methoxy-N-methyl-2-(4-oxocyclopent-2-en-1-yl)acetamide (31)

To a stirred solution of (S)-2-(cyclopent-2-en-1-yl)-N-methoxy-N-methylacetamide (30) (110 mg; 0.65 mmol; 1.0 equiv.) in CH₂Cl₂ (3 mL) at 25° C. under an atmosphere of oxygen (balloon) were added sequentially K₂CO₃ (45 mg; 0.325 mmol; 0.5 equiv.) and dirhodium tetracaprolactamate (8.5 mg; 0.013 mmol; 0.02 equiv.). To the resulting light-blue suspension was then dropwise added t-butyl hydroperoxide (5.5 M in decane; 0.59 mL; 3.25 mmol; 5.0 equiv.). The resulting purple suspension was stirred for 1.5 h (oxygen evolution), and another portion of dirhodium tetracaprolactamate (8.5 mg; 0.013 mmol; 0.02 equiv.) and t-butyl hydroperoxide (5.5 M in decane; 0.59 mL; 3.25 mmol; 5.0 equiv.) were added. Stirring was continued for another 1.5 h (oxygen-evolution), the mixture was filtered through a plug of SiO₂, washed with CH₂Cl₂:Et₂O, 1:1, and then carefully concentrated (minimum pressure: 50 mbar). Flash column chromatography (SiO₂; C₆H₆:acetone, 10:1) yielded pure title compound (42 mg; 0.23 mmol; 35%) as a colorless oil.

31: R_{f}=0.24 (hexane:EtOAc, 1:2); [α]_{D}²⁵=+133.0° (c=1.0 in C₆H₆); IR (film): νₘₐₓ 3504, 2924, 2854, 1707, 1655, 1585, 1415, 1387, 1182, 997 cm′; ¹H-NMR (500 MHz, CDCl₃) δ 7.69 (dd, J=5.7, 2.5 Hz, 1H), 6.16 (dd, J=5.6, 2.0 Hz, 1H), 3.65 (s, 3H), 3.45-3.38 (m, 1H), 3.17 (s, 3H), 2.66 (dd, J=19.0, 6.5 Hz, 1H), 2.61 (dd, J=7.5, 4.9 Hz, 2H), 2.02 (dd, J=19.0, 2.3 Hz, 1H) ppm; ¹³C-NMR (125 MHz, CDCl₃) δ 209.41, 172.03, 167.71, 134.35, 61.38, 41.21, 37.38, 36.73, 32.21 ppm; HR-MS (ESI-TOF): calcd for C₉H₁₄NO₃ [M+H]⁺: 184.0968. found: 184.0960.

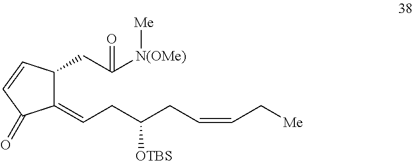

### Weinreb Dienone 38

To a stirred solution of diisopropylamine (33 μL; 0.229 mmol; 2.1 equiv.) in THF (1 mL) at 0° C. was dropwise added n-butyl lithium (2.5 M in hexanes; 88 μL; 0.218 mmol; 2.0 equiv.). After stirring for 20 min at this temperature, the clear solution was cooled to −78° C. and a solution of (S)—N-methoxy-N-methyl-2-(4-oxocyclopent-2-en-1-yl)acetamide (31) (20.0 mg; 109.2 μmol; 1.0 equiv.) in THF (1 mL) was added dropwise. After stirring the resulting yellow solution for an additional 20 min at this temperature, a solution of aldehyde 8 (33.6 mg; 131.0 μmol; 1.2 equiv.) in THF (1 mL) was added dropwise and stirring at this temperature was continued for an additional 30 min. The reaction mixture was then quenched with sat. aq. NR₄Cl solution (5 mL), diluted with EtOAc (10 mL), and allowed to warm to 25° C. The phases were separated, the aq. layer was extracted with EtOAc (2×5 mL), and the combined organic extracts were washed with sat. brine (5 mL), dried (Na₂SO₄), filtered, and concentrated. The crude aldol product 32 was filtered through a short column (SiO₂; hexane:EtOAc, 1:1) to obtain a mixture of diastereoisomers (ca. 3:1; 14.9 mg; 33.8 μmol; 31%) as a colorless oil which was taken to the next step without further purification.

To a stirred solution of aldol product 32 (14.9 mg; 33.8 μmol) in CH₂Cl₂ (0.5 mL) at 0° C. was added Et₃N (47 μL; 338.0 μmol; 10 equiv.), and then, slowly and dropwise, methanesulfonyl chloride (13 μL; 169.0 μmol; 5.0 equiv.). After stirring for 5 min at this temperature, the reaction mixture was quenched with sat. aq. NaHCO₃ solution (3 mL), diluted with CH₂Cl₂ (5 mL), and allowed to warm to 25° C. The phases were separated, the aq. layer was extracted with CH₂Cl₂ (2×5 mL), and the combined organic layers were washed with H₂O (3 mL), dried (Na₂SO₄), filtered, and concentrated. The crude mesylate 33 was filtered through a short column (SiO₂; hexane:EtOAc, 1:2) to obtain a mixture of diastereoisomers (ca. 3:1; 14.0 mg; 27.0 μmol; 80%) as a colorless oil which was taken to the next step without further purification.

To a vigorously stirred solution of mesylate 33 (14.0 mg; 27.0 μmol) in CH₂Cl₂ (0.5 mL) at 25° C. was added Al₂O₃ (19.3 mg; 189.0 μmol; 7.0 equiv.). After 2 h and 4 h time intervals two more portions of Al₂O₃ (2×19.3 mg; 2×189.0 μmol; 2×7.0 equiv.) were added and vigorous stirring was continued for a total of 8 h. The resulting suspension was then filtered through Celite, washed with EtOAc, and the solution obtained was concentrated. Flash column chromatography (SiO₂; hexane:EtOAc, 2:1) yielded pure title compound (55 mg; 0.115 mmol; 65%; 16% for three steps) as a colorless oil.

38: R_{f}=0.34 (hexane:EtOAc, 2:1); [α]_{D}²⁵=+125.0° (c=0.4 in C₆H₆); IR (film): νₘₐₓ 2957, 2929, 2856, 1705, 1659, 1582, 1463, 1255, 1086 cm⁻¹; ¹H-NMR (500 MHz, CDCl₃) δ 7.69 (ddd, J=6.0, 2.5, 1.0 Hz, 1H), 6.61 (tt, J=7.7, 1.2 Hz, 1H), 6.33 (dd, J=6.0, 1.8 Hz, 1H), 5.47 (dtt, J=10.4, 7.1, 1.6 Hz, 1H), 5.35 (dtt, J=10.7, 7.4, 1.6 Hz, 1H), 3.89 (td, J=9.6, 8.1, 4.3 Hz, 2H), 3.65 (s, 3H), 3.22 (s, 3H), 3.06 (dd, J=16.2, 3.6 Hz, 1H), 2.42-2.35 (m, 2H), 2.35-2.15 (m, 2H), 2.00 (p, J=7.5 Hz, 3H), 0.94 (t, J=7.5 Hz, 3H), 0.86 (s, 9H), 0.05 (s, 3H), 0.04 (s, 3H) ppm; ¹³C-NMR (125 MHz, CDCl₃) δ 196.00, 172.14, 162.05, 138.62, 134.93, 134.30, 133.30, 124.20, 71.65, 61.42, 39.43, 36.81, 35.69, 35.56, 32.31, 25.99, 20.91, 18.23, 14.33, −4.40, −4.41 ppm; HR-MS (ESI-TOF): calcd for C₂₃H₃₉NO₄SiNa [M+Na]⁺: 444.2541. found: 444.2519.

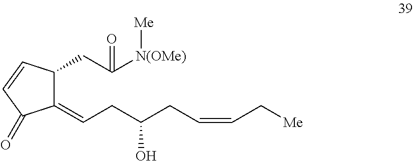

### Δ¹²-PGJ₃ Weinreb analog 39

To a stirred solution of Weinreb dienone 38 (3.5 mg; 8.3 μmol; 1.0 equiv.) in MeCN (0.1 mL) at 0° C. was dropwise added a solution of HF (50% aq.; 17 μL; ca. 415 μmol; ca. 50 equiv.) in MeCN (0.1 mL). After stirring for 1 h at this temperature, the reaction mixture was quenched with sat. aq. NaHCO₃ solution (1 mL), and extracted with EtOAc (3×3 mL). The combined organic extracts were washed with sat. brine (2 mL), dried (Na₂SO₄), filtered, and concentrated to a volume of ca. 0.1 mL (not to dryness!). Flash column chromatography (SiO₂; hexane:EtOAc, 1:3→0:1) yielded pure title compound (2.5 mg; 8.2 μmol; 99%) as a colorless oil.

39: R_{f}=0.23 (hexane:EtOAc, 1:2); [α]_{D}²⁵=+172.5° (c=0.2 in C₆H₆); IR (film): νₘₐₓ 3438, 2960, 2925, 2873, 2854, 1700, 1650, 1580, 1462, 1387, 1269, 1178, 1001 cm⁻¹; ¹H-NMR (500 MHz, CDCl₃) δ 7.73-7.67 (m, 1H), 6.69-6.62 (m, 1H), 6.34 (dd, J=6.0, 1.8 Hz, 1H), 5.67-5.55 (m, 1H), 5.36 (dddd, J=14.1, 8.3, 3.3, 1.6 Hz, 1H), 3.93 (d, J=10.1 Hz, 1H), 3.84 (q, J=6.6 Hz, 1H), 3.66 (s, 3H), 3.21 (s, 3H), 3.10 (dd, J=16.6, 4.0 Hz, 1H), 2.53-2.37 (m, 3H), 2.34-2.22 (m, 2H), 2.12-2.02 (m, 2H), 1.99 (s, 1H), 0.96 (t, J=7.5 Hz, 3H) ppm; ¹³C-NMR (125 MHz, CDCl₃) δ 196.11, 172.06, 162.11, 139.43, 136.00, 134.88, 132.21, 123.78, 70.54, 61.46, 39.44, 36.70, 35.50, 35.30, 32.31, 20.90, 14.37 ppm; HR-MS (ESI-TOF): calcd for C₁₇H₂₅NO₄Na [M+Na]⁺: 330.1676. found: 330.1688.

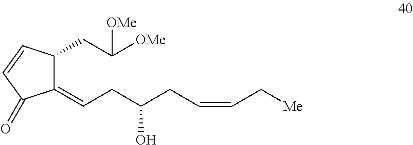 [SMILES: CC/C=C\C[C@H](O)C/C=C1/C(=O)C=C[C@@H]1CC(OC)OC]

### Δ¹²-PGJ₃ dimethylketal analog 40

To a stirred solution of t-butyldiemthylsilyl-protected Δ¹²-PGJ₃ dimethylketal analog 41 (15.0 mg; 36.7 μmol; 1.0 equiv.) in MeCN (0.65 mL) at −10° C. was dropwise added a solution of HF (50% aq.; 65 μL; ca. 1.84 mmol; ca. 50 equiv.) in MeCN (0.65 mL). After stirring for 1.5 h at this temperature, the reaction mixture was quenched with sat. aq. NaHCO₃ solution (3 mL), and extracted with EtOAc (3×5 mL). The combined organic extracts were washed with sat. brine (3 mL), dried (Na₂SO₄), filtered, and concentrated to a volume of ca. 0.1 mL (not to dryness!). Flash column chromatography (SiO₂; hexane:EtOAc, 1:2) yielded pure title compound (8.1 mg; 27.5 μmol; 75%) as a colorless oil.

40: R_{f}=0.35 (hexane:EtOAc, 1:2); [α]_{D}²⁵=+4.4° (c=0.8 in C₆H₆); IR (film): νₘₐₓ 3419, 2960, 2929, 2853, 1697, 1652, 1607, 1461, 1414, 1269, 1193, 1117, 1060 cm⁻¹; ¹H-NMR (500 MHz, CDCl₃) δ 7.35 (s, 1H), 6.59 (tt, J=7.7, 1.8 Hz, 1H), 6.21 (t, J=1.3 Hz, 1H), 5.58 (dddd, J=10.3, 8.7, 5.1, 1.5 Hz, 1H), 5.40-5.32 (m, 1H), 4.63 (t, J=5.6 Hz, 1H), 3.82 (tt, J=7.0, 5.4 Hz, 1H), 3.35 (s, 6H), 3.18 (ddd, J=3.7, 1.8, 0.9 Hz, 1H), 2.77 (dd, J=5.6, 1.1 Hz, 2H), 2.39 (ddd, J=7.7, 6.0, 3.1 Hz, 2H), 2.32-2.23 (m, 2H), 2.05 (pd, J=7.5, 1.6 Hz, 2H), 1.87 (s, 1H), 0.96 (t, J=7.5 Hz, 3H) ppm; ¹³C-NMR (125 MHz, CDCl₃) δ 196.04, 169.72, 137.49, 135.76, 133.21, 130.25, 123.93, 102.39, 70.63, 53.26, 53.23, 37.05, 36.71, 35.66, 35.10, 20.88, 14.35 ppm; HR-MS (ESI-TOF): calcd for C₁₇H₂₆O₄Na [M+Na]⁺: 317.1723. found: 317.1718.

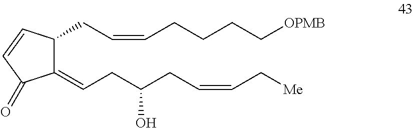

### Δ¹²-PGJ₃ para-methoxybenzylether analog 43

To a stirred solution of dienone 22 (18.0 mg; 32.6 μmol; 1.0 equiv.) in MeCN (0.3 mL) at −10° C. was dropwise added a solution of HF (50% aq.; 67 μL; ca. 1.63 mmol; ca. 50 equiv.) in MeCN (0.3 mL). After stirring for 2 h at this temperature, the reaction mixture was quenched with sat. aq. NaHCO₃ solution (5 mL), and extracted with EtOAc (3×5 mL). The combined organic extracts were washed with sat. brine (3 mL), dried (Na₂SO₄), filtered, and concentrated to a volume of ca. 0.1 mL (not to dryness!). Flash column chromatography (SiO₂; hexane:EtOAc, 2:1) yielded pure title compound (12.4 mg; 28.4 μmol; 87%) as a colorless oil.

40: R_{f}⁼0.51 (hexane:EtOAc, 1:1); [α]_{D}²⁵=+142.5° (c=1.0 in C₆H₆); IR (film): νₘₐₓ 3430, 3007, 2931, 2856, 1700, 1651, 1612, 1583, 1513, 1461, 1246, 1173, 1097, 1036 cm⁻¹; ¹H-NMR (500 MHz, CDCl₃) δ 7.50 (ddd, J=6.1, 2.6, 1.0 Hz, 1H), 7.25 (d, J=8.8 Hz, 2H), 6.87 (d, J=8.6 Hz, 2H), 6.63 (tt, J=7.0, 1.4 Hz, 1H), 6.33 (dd, J=6.0, 1.8 Hz, 1H), 5.63-5.54 (m, 1H), 5.51-5.44 (m, 1H), 5.41-5.27 (m, 2H), 4.42 (s, 2H), 3.80 (s, 3H), 3.48 (dq, J=6.6, 2.1 Hz, 1H), 3.42 (t, J=6.5 Hz, 2H), 2.60 (dddd, J=12.5, 6.4, 4.5, 2.1 Hz, 1H), 2.54-2.41 (m, 2H), 2.31-2.17 (m, 3H), 2.10-1.92 (m, 6H), 1.62-1.54 (m, 2H), 1.40 (dq, J=10.1, 7.5 Hz, 2H), 0.96 (t, J=7.5 Hz, 3H) ppm; ¹³C-NMR (125 MHz, CDCl₃) δ 196.53, 161.98, 159.22, 139.55, 135.85, 134.94, 132.72, 131.68, 130.73, 129.37, 125.05, 123.84, 113.86, 72.67, 70.57, 69.97, 55.40, 43.48, 36.58, 35.03, 30.43, 29.50, 27.30, 26.28, 20.89, 14.36 ppm; HRMS (ESI-TOF): calcd for C₂₈H₃₈O₄Na [M+Na]⁺: 461.2662. found: 461.2647.

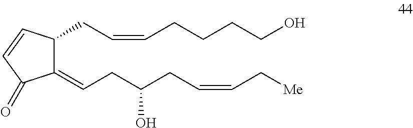 [SMILES: CC/C=C\C[C@H](O)C/C=C1/C(=O)C=C[C@@H]1C/C=C\CCCCO]

### Hydroxy dienone 44

To a vigorously stirred solution of Δ¹²-PGJ₃ para-methoxybenzylether analog 43 (9.0 mg; 20.5 μmol; 1.0 equiv.) in a mixture of CH₂Cl₂:H₂O (4:1; 0.5 mL) at 0° C. was added in one portion 2,3-dichloro-5,6-dicyano-1,4-benzoquinone (7.0 mg; 30.8 μmol; 1.5 equiv.). After stirring at this temperature for 60 min, the reaction mixture was diluted with Et₂O (5 mL), filtered through Celite, washed with Et₂O, and concentrated to a volume of ca. 0.1 mL (not to dryness!). Flash column chromatography (SiO₂; hexane:EtOAc, 1:1→0:1) yielded pure title compound (6.0 mg; 18.9 μmol; 92%) as a colorless oil.

23: R_{f}⁼0.21 (hexane:EtOAc, 1:1); [α]_{D}²⁵=+151.2° (c=0.6 in C₆H₆); IR (film): νₘₐₓ 3385, 3009, 2928, 2857, 1695, 1648, 1579, 1456, 1210, 1048 cm⁻¹; ¹H-NMR (500 MHz, CDCl₃) δ 7.52 (ddd, J=6.0, 2.6, 1.0 Hz, 1H), 6.63 (ddt, J=8.3, 7.0, 1.3 Hz, 1H), 6.34 (dd, J=6.0, 1.8 Hz, 1H), 5.60 (dtt, J=10.5, 7.3, 1.5 Hz, 1H), 5.53-5.44 (m, 1H), 5.40-5.29 (m, 2H), 3.84 (p, J=6.3 Hz, 1H), 3.63 (t, J=6.5 Hz, 2H), 3.54-3.47 (m, 1H), 2.63 (dddd, J=13.0, 6.4, 4.3, 2.2 Hz, 1H), 2.58-2.42 (m, 2H), 2.31-2.19 (m, 3H), 2.04 (dddd, J=17.6, 8.3, 7.3, 1.6 Hz, 4H), 1.60-1.51 (m, 2H), 1.46-1.37 (m, 2H), 0.96 (t, J=7.5 Hz, 3H) ppm; ¹³C-NMR (125 MHz, CDCl₃) δ 196.59, 161.97, 139.61, 135.93, 135.01, 132.67, 131.72, 125.15, 123.81, 70.65, 62.76, 43.53, 36.62, 35.01, 32.36, 30.45, 27.21, 25.80, 20.90, 14.37 ppm; HR-MS (ESI-TOF): calcd for C₂₀H₃₀O₃Na [M+Na]⁺: 341.2087. found: 341.2078.

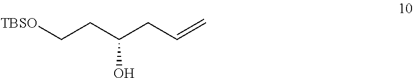

### Alcohol 10

This compound was prepared as described in the literature in Liniger, et al., 2011, which is incorporated herein by reference. A mixture of (S)-BINOL (114 mg, 0.398 mmol, 0.05 equiv.) and oven-dried (100° C.) powdered 4 Å molecular sieves (320 mg) in anhydrous toluene (16 mL) was treated with Ti(OiPr)₄ (60 μL, 0.20 mmol, 0.025 equiv.), and the reaction mixture was stirred for 2.5 h at 25° C. Aldehyde 9 (1.5 g, 8.0 mmol, 1.0 equiv.) was then added and the reaction mixture was stirred for 5 min. After cooling to −78° C., allyl tri-n-butyltin (3.7 mL, 12 mmol, 1.5 equiv.) was added, and the mixture was stirred for −78° C. for 10 min followed by 139 h at −20° C. Sat. aq. NaHCO₃ (10 mL) was then added and the resulting mixture was extracted with hexanes (2×10 mL). The combined organic extracts were washed with brine (20 mL), dried (MgSO₄) and concentrated. The residue was purified by flash column chromatography (SiO₂; hexanes:Et₂O, 5:1) to give 10 as a yellowish oil (820 mg, 3.58 mmol, 45%, 65% brsm, >95% ee as determined by analysis of the ¹H NMR of the corresponding (S)-Mosher ester). 10: Data were in agreement with those reported in Liniger, et al., 2011, which is incorporated herein by reference.

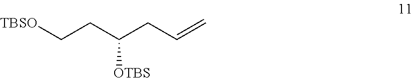 [SMILES: C=CC[C@@H](CCO[Si](C)(C)C(C)(C)C)O[Si](C)(C)C(C)(C)C]

### bis-TBS ether 11

To a stirred solution of alcohol 10 (500 mg, 2.18 mmol, 1 equiv.) in CH₂Cl₂ (6 mL) at 25° C. was added sequentially imidazole (443 mg, 6.78 mmol, 3.1 equiv.) and TBSCl (443 mg, 2.95 mmol, 1.4 equiv.). The resulting mixture was stirred for 90 min, and the reaction was then quenched with sat. aq. NH₄Cl (10 mL). The phases were separated and the aq. layer was extracted with CH₂Cl₂ (2×10 mL). The combined organic extracts were washed with H₂O (30 mL), dried (Na₂SO₄), filtered, and concentrated. Flash column chromatography (SiO₂; hexanes:EtOAc, 40:1) yielded the pure title compound (660 mg, 1.92 mmol, 88%) as a colorless oil. 11: R_{f}=0.8 (silica gel, hexanes:EtOAc, 19:1); [α]_{D}²⁵=+18.8 (c=1.0, CHCl₃); FT-IR (neat) νₘₐₓ=2954, 2929, 2857, 1472, 1254, 1091, 832, 772 cm⁻¹; ¹H NMR (600 MHz, CDCl₃) δ=5.82 (ddt, J=16.6, 10.6, 7.2 Hz, 1H), 5.06-5.01 (m, 2H), 3.87 (tt, J=6.6, 5.2 Hz, 1H), 3.69-3.64 (m, 2H), 2.29-2.17 (m, 2H), 1.69-1.60 (m, 2H), 0.889 (s, 9H), 0.887 (s, 9H), 0.05 (s, 6H), 0.04 (s, 6H) ppm; ¹³C NMR (151 MHz, CDCl₃) δ=135.33, 116.92, 69.04, 60.02, 42.32, 39.97, 26.11, 26.04, 18.44, 18.27, −4.23, −4.52, −5.14 ppm; HRMS (ESI) calcd for C₁₈H₄₀O₂Si₂H⁺ [M+H]⁺: 345.2640. found: 345.2638.

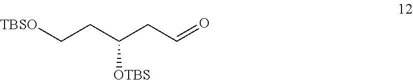 [SMILES: CC(C)(C)[Si](C)(C)OCC[C@H](CC=O)O[Si](C)(C)C(C)(C)C]

### Aldehyde 12

To a −78° C. stirred solution of olefin 11 (1.2 g, 3.5 mmol, 1 equiv.) in CH₂Cl₂ (350 mL) was added a spatula tip of NaHCO₃. A stream of ozone was bubbled through this mixture until the solution became blue in color. Nitrogen was bubbled through the mixture until the blue color dissipated. PPh₃ (1.85 g, 6.98 mmol, 2.0 equiv.) was added, and the mixture was allowed to warm to 25° C. and stirred for 3 h. The reaction mixture was concentrated, and the resulting crude product was purified by flash column chromatography (SiO₂; hexanes:Et₂O, 40:1→20:1→40:1) to yield the pure title compound (1.16 g, 3.37 mmol, 97%) as a colorless oil. 12: R_{f}=0.5 (silica gel, hexanes:EtOAc, 9:1); [α]_{D}²⁵=+7.8 (c=1.0, CHCl₃); FT-IR (neat) νₘₐₓ=2954, 2929, 2857, 1727, 1472, 1463, 1388, 1253, 1093, 1035, 1005, 832, 773 cm⁻¹; ¹H NMR (600 MHz, CDCl₃) δ=9.81 (dd, J=3.0, 2.1 Hz, 1H), 4.37 (p, J=5.9 Hz, 1H), 3.68 (t, J=6.1 Hz, 2H), 2.61 (ddd, J=15.7, 5.1, 2.1 Hz, 1H), 2.53 (ddd, J=15.7, 6.2, 3.0 Hz, 1H), 1.80 (dq, J=13.8, 6.1 Hz, 1H), 1.70 (dq, J=13.7, 6.1 Hz, 1H), 0.89 (s, 9H), 0.87 (s, 9H), 0.08 (s, 3H), 0.06 (s, 3H), 0.04 (s, 6H) ppm; ¹³C NMR (151 MHz, CDCl₃) δ=202.44, 65.72, 59.35, 51.18, 40.72, 26.05, 25.91, 18.37, 18.13, −4.42, −4.55, −5.21, −5.23 ppm; HRMS (ESI) calcd for C₁₇H₃₈O₃Si₂Na⁺ [M+Na]⁺: 369.2252. found: 369.2246.

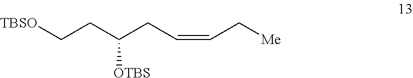 [SMILES: CC/C=C\C[C@@H](CCO[Si](C)(C)C(C)(C)C)O[Si](C)(C)C(C)(C)C]

### Olefin 13

To a stirred slurry of triphenyl-n-propylphosphonium bromide (1.45 g, 3.76 mmol, 2.0 equiv.) in THF (9 mL) was added a solution of NaHMDS (1 M in THF, 3.8 mL, 3.8 mmol, 2.0 equiv.) dropwise at 0° C. The resulting mixture was stirred at 0° C. for 30 min. The reaction mixture was then cooled to −78° C., and aldehyde 12 (654 mg, 1.89 mmol) was added dropwise as a solution in THF (1.0 mL). The resulting mixture was stirred at −78° C. for 30 min and then allowed to warm up to 25° C. over 2 h. The reaction was quenched with sat. aq. NH₄Cl solution (10 mL) and stirred for 20 min. The phases were then separated, and the aq. layer was extracted with Et₂O (2×10 mL). The combined organic extracts were washed with H₂O (30 mL), dried (Na₂SO₄), filtered, and concentrated. Flash column chromatography (SiO₂; hexanes:Et₂O, 40:1) yielded pure title compound (608 mg, 1.63 mmol, 87%) as a colorless oil. 13: R_{f}=0.83 (silica gel, hexanes:EtOAc, 7:1); [α]_{D}²⁵=+15.9 (c=1.17, CHCl₃); FT-IR (neat) νₘₐₓ=2956, 2929, 2886, 2858, 1472, 1463, 1388, 1361, 1255, 1092, 1035, 1005, 833, 773 cm⁻¹; ¹H NMR (600 MHz, CDCl₃) δ=5.47-5.41 (m, 1H), 5.39-5.33 (m, 1H), 3.84 (p, J=6.1 Hz, 1H), 3.71-3.63 (m, 2H), 2.21 (t, J=6.6 Hz, 2H), 2.04 (p, J=7.4 Hz, 2H), 1.72-1.57 (m, 2H), 0.96 (t, J=7.5 Hz, 3H), 0.89 (s, 18H), 0.06 (s, 3H), 0.05 (s, 3H), 0.04 (s, 6H) ppm; ¹³C NMR (151 MHz, CDCl₃) δ=133.40, 125.18, 69.42, 60.13, 40.07, 35.55, 26.10, 26.06, 20.89, 18.43, 18.26, 14.38, −4.20, −4.55, −5.12, −5.14 ppm; HRMS (ESI) calcd for C₂₀H₄₄O₂Si₂H⁺ [M+H]⁺: 373.2953. found: 373.2957.

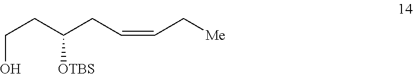 [SMILES: CC/C=C\C[C@@H](CCO)O[Si](C)(C)C(C)(C)C]

### Hydroxy Compound 14

To a stirred solution of bis-TBS ether 13 (438 mg, 1.18 mmol, 1.0 equiv.) in MeOH (12 mL) was added pyridinium tribromide (38 mg, 0.12 mmol, 0.1 equiv.) at −10° C. The reaction mixture was stirred at this temperature for 75 min, and the reaction was then quenched by addition of H₂O (20 mL) and EtOAc (20 mL) and allowed to warm to 25° C. The layers were separated, and the aq. phase was extracted with EtOAc (15 mL). The combined organics were dried (Na₂SO₄), filtered, and concentrated. Flash column chromatography (SiO₂; hexanes:EtOAc, 19:1→9:1→4:1) provided the title compound 14 (191 mg, 0.740 mmol, 63%) as a colorless oil. 14: R_{f}=0.4 (silica gel, hexanes:EtOAc, 4:1); [α]_{D}²⁵=+31.0 (c=1.0, CHCl₃); FT-IR (neat) νₘₐₓ=3356, 2957, 2930, 2857, 1472, 1462, 1254, 1059, 1022, 834, 774 cm⁻¹; ¹H NMR (600 MHz, CDCl₃) δ=5.48-5.44 (m, 1H), 5.33-5.28 (m, 1H), 3.94 (tdd, J=7.1, 5.4, 3.8 Hz, 1H), 3.83 (ddd, J=10.7, 8.4, 4.2 Hz, 1H), 3.72 (ddd, J=10.7, 6.3, 4.8 Hz, 1H), 2.32-2.25 (m, 2H), 2.07-2.02 (m, 2H), 1.81 (dddd, J=14.3, 8.4, 4.8, 3.8 Hz, 1H), 1.65 (dddd, J=14.3, 6.3, 5.4, 4.2 Hz, 1H), 0.96 (t, J=7.5 Hz, 3H), 0.90 (s, 9H), 0.104 (s, 3H), 0.096 (s, 3H) ppm; ¹³C NMR (151 MHz, CDCl₃) δ=133.96, 124.47, 72.06, 60.52, 37.71, 35.01, 25.98, 20.92, 18.13, 14.34, −4.20, −4.68 ppm; HRMS (ESI) calcd for C₁₄H₃₀O₂SiNa⁺ [M+Na]⁺: 281.1907. found: 281.1901.

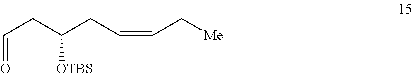 [SMILES: CC/C=C\C[C@@H](CC=O)O[Si](C)(C)C(C)(C)C]

### Aldehyde 15

To a stirred solution of primary alcohol 14 (36 mg, 0.13 mmol, 1.0 equiv.) in CH₂Cl₂ (2.5 mL) was added Dess-Martin periodinane (75 mg, 0.17 mmol, 1.3 equiv.) at 0° C. The resulting mixture was stirred for 30 min at the same temperature and then allowed to warm to 25° C. After stirring for 90 min, the reaction was quenched sequentially with sat. aq. NaHCO₃ solution (2.0 mL) followed by sat. aq. Na₂S₂O₃ solution (2.0 mL). After stirring for 20 min, the phases were separated and the aq. layer was extracted with CH₂Cl₂ (2×5 mL). The combined organic extracts were washed with brine (10 mL), dried (Na₂SO₄), filtered, and concentrated. Flash column chromatography (SiO₂; hexanes:EtOAc, 20:1) yielded pure title compound 15 (34 mg, 0.13 mmol, 99%) as a colorless oil. 15: Data were in agreement with those obtained above.

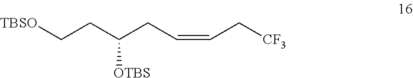 [SMILES: CC(C)(C)[Si](C)(C)OCC[C@H](C/C=C\CC(F)(F)F)O[Si](C)(C)C(C)(C)C]

### bis-TBS ether 16

To a stirred slurry of triphenyl(3,3,3-trifluoropropyl)phosphonium iodide (750 mg, 1.54 mmol, 1.4 equiv.) in THF (5.6 mL) was added a solution of LiHMDS (1 M in THF, 1.48 mL, 1.48 mmol, 1.3 equiv.) dropwise at 0° C. The resulting mixture was stirred at 0° C. for 30 min and was then cooled to −78° C. Aldehyde 12 (385 mg, 1.11 mmol) was added dropwise as a solution in THF (2.3 mL), and the resulting mixture was stirred at −78° C. for 1 h and then at 25° C. for 4 h. The reaction was quenched with sat. aq. NH₄Cl solution (5 mL) and stirred for 20 min. Then, the phases were separated and the aq. layer was extracted with Et₂O (2×5 mL). The combined organic extracts were washed with H₂O (20 mL), dried (Na₂SO₄), filtered, and concentrated. Flash column chromatography (SiO₂; hexanes:Et₂O, 40:1→20:1) yielded the title compound (458 mg, 1.07 mmol, 96%, mixture of inseparable isomers: Z:E ca. 7:1 by ¹H NMR analysis) as a colorless oil.

16: R_{f}=0.66 (silica gel, hexanes:Et₂O, 9:1); [α]_{D}²⁵=+19.1 (c=1.13, CHCl₃); FT-IR (neat) νₘₐₓ=2954, 2930, 2858, 1472, 1361, 1251, 1136, 1088, 1036, 833, 773 cm-1; ¹H NMR (500 MHz, CDCl₃, data for Z isomer) δ=5.82-5.75 (m, 1H), 5.53-5.45 (m, 1H), 3.93 (p, J=5.6 Hz, 1H), 3.66 (t, J=6.6 Hz, 2H), 2.91-2.78 (m, 2H), 2.30-2.19 (m, 2H), 1.66-1.60 (m, 2H), 0.89 (s, 9H), 0.88 (s, 9H), 0.06 (s, 3H), 0.05 (s, 3H), 0.04 (s, 6H) ppm; ¹³C NMR (126 MHz, CDCl₃, data for Z isomer) δ=132.91, 127.42 (q, J=276 Hz), 118.62 (q, J=3.4 Hz), 68.78, 59.81, 40.10, 35.62, 32.60 (q, J=32.5 Hz), 26.06, 25.99, 18.40, 18.20, 4.32, 4.57, 5.19, 5.21 ppm; HRMS (ESI) calcd for C₂₀H₄₁O₂F₃Si₂H⁺ [M+H]⁺: 427.2670. found: 427.2658.

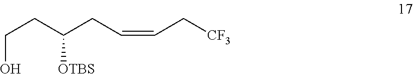 [SMILES: CC(C)(C)[Si](C)(C)O[C@@H](C/C=C\CC(F)(F)F)CCO]

### Hydroxy Compound 17

To a stirred solution of bis-TBS ether 16 (450 mg, 1.05 mmol) in MeOH (15 mL) was added pyridinium tribromide (17 mg, 0.053 mmol, 0.05 equiv) at 10° C. The resulting mixture was stirred for 5 h at 10° C., and the reaction was then quenched with H₂O (10 mL) and allowed to warm up to 25° C. The phases were separated and the aq. layer was extracted with EtOAc (2×10 mL). The combined organic extracts were washed with brine (20 mL), dried (Na₂SO₄), filtered, and concentrated. Flash column chromatography (SiO₂; hexanes:EtOAc, 20:1→9:1) yielded pure title compound (170 mg, 0.545 mmol, 51%, 62% brsm, mixture of inseparable isomers: Z:E ca. 7:1 by ¹H NMR analysis) as a colorless oil. 17: R_{f}⁼0.47 (silica gel, hexanes:EtOAc, 7:3); [α]_{D}²⁵=+17.9 (c=1.03, CHCl₃); FT-IR (neat) νₘₐₓ=2952, 2935, 2860, 1473, 1347, 1253, 1138, 1070, 836, 775 cm-1; ¹H NMR (500 MHz, CDCl₃, data for Z isomer) δ=5.78-5.68 (m, 1H), 5.53-5.46 (m, 1H), 4.03-3.94 (m, 1H), 3.84-3.77 (m, 1H), 3.75-3.69 (m, 1H), 2.92-2.77 (m, 2H), 2.30 (t, J=7.0 Hz, 2H), 2.11 (bs, 1H), 1.82-1.73 (m, 1H), 1.69-1.61 (m, 1H), 0.89 (s, 9H), 0.10 (s, 3H), 0.08 (s, 3H) ppm; ¹³C NMR (126 MHz, CDCl₃, data for Z isomer) δ=132.22, 126.20 (q, J=276.6 Hz), 119.09 (q, J=3.5 Hz), 70.81, 60.17, 38.10, 35.23, 32.60 (q, J=32.5 Hz, CF₃), 25.92, 18.09, −4.31, −4.68 ppm; HRMS (ESI) calcd for C₁₄H₂₇O₂F₃Si₂H⁺ [M+H]⁺: 313.1805. found: 313.1793.

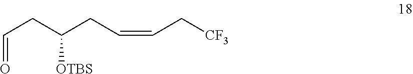 [SMILES: CC(C)(C)[Si](C)(C)O[C@H](CC=O)C/C=C\CC(F)(F)F]

### Aldehyde 18

To a stirred solution of primary alcohol 17 (170 mg, 0.545 mmol, 1.0 equiv.) in CH₂Cl₂ (6 mL) was added Dess-Martin periodinane (343 mg, 0.809 mmol, 1.5 equiv.) at 0° C. The resulting mixture was stirred for 30 min at 0° C. and then allowed to warm to 25° C. After stirring for 90 min, the reaction was quenched with a mixture of sat. aq. NaHCO₃ solution (3 mL) followed by sat. aq. Na₂S₂O₃ solution (3 mL). After stirring for 20 min, the phases were separated and the aq. layer was extracted with CH₂Cl₂ (2×10 mL). The combined organic extracts were washed with brine (10 mL), dried (Na₂SO₄), filtered, and concentrated. Flash column chromatography (SiO₂; hexanes:EtOAc, 20:1) yielded pure title compound (144 mg, 0.465 mmol, 86%, mixture of inseparable isomers: Z:E ca. 7:1 by ¹H NMR analysis) as a colorless oil. 18: R_{f}⁼0.66 (silica gel, hexanes:EtOAc, 8:2); [α]_{D}²⁵=+13.7 (c=1.6, CHCl₃); FT-IR (neat) νₘₐₓ=2956, 2931, 2859, 1726, 1473, 1348, 1252, 1135, 1102, 1085, 835, 776 cm⁻¹; ¹H NMR (500 MHz, CDCl₃, data for Z isomer) δ=9.78 (t, J=2.1 Hz, 1H), 5.78-5.70 (m, 1H), 5.56-5.49 (m, 1H), 4.27 (p, J=6.1 Hz, 1H), 2.88-2.74 (m, 2H), 2.55-2.50 (m, 2H), 2.34-2.27 (m, 2H), 0.86 (s, 9H), 0.07 (s, 3H), 0.05 (s, 3H) ppm; ¹³C NMR (126 MHz, CDCl₃, data for Z isomer) δ=201.62, 131.54, 126.14 (q, J=276.5 Hz), 119.86 (q, J=3.5 Hz), 67.47, 50.63, 35.69, 32.55 (q, J=32.5 Hz, CF₃), 25.81, 18.06, −4.40, −4.74 ppm; HRMS (ESI): this compound did not ionize.

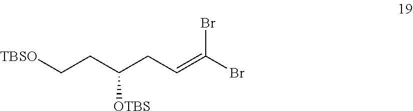 [SMILES: CC(C)(C)[Si](C)(C)OCC[C@H](CC=C(Br)Br)O[Si](C)(C)C(C)(C)C]

### Dibromide 19

To a stirred solution of PPh₃ (5.61 g, 21.4 mmol, 4.0 equiv.) in CH₂Cl₂ (43 mL) was added CBr₄ (3.54 g, 10.7 mmol, 2.0 equiv.) at 0° C. After stirring for 10 min, a solution of aldehyde 12 (1.85 g, 5.35 mmol) in CH₂Cl₂ (19 mL) was added dropwise. The reaction mixture was stirred at the same temperature for 30 min and then diluted with hexanes (40 mL), filtered through a pad of Celite®, and concentrated. Flash column chromatography (SiO₂; hexanes:EtOAc, 9:1) yielded the pure title compound (2.52 g, 5.04 mmol, 94%) as a colorless oil. 19: R_{f}=0.7 (silica gel, hexanes:EtOAc, 9:1); [α]_{D}²⁵=+13.5 (c=1.0, CHCl₃); FTIR (neat) νₘₐₓ=2954, 2929, 2857, 1471, 1462, 1387, 1361, 1254, 1094, 1035, 833, 773 cm⁻¹; ¹H NMR (500 MHz, CDCl₃) δ=6.48 (t, J=7.2 Hz, 1H), 3.99-3.94 (m, 1H), 3.66 (t, J=6.3 Hz, 2H), 2.29-2.26 (m, 2H), 1.71-1.58 (m, 2H), 0.89 (ap s, 18H), 0.07 (s, 3H), 0.06 (s, 3H), 0.04 (s, 6H) ppm; ¹³C NMR (126 MHz, CDCl₃) δ=135.81, 89.79, 67.70, 59.60, 40.97, 40.22, 26.08, 25.97, 18.40, 18.18, −4.40, −4.55, −5.15, −5.19 ppm; HRMS (ESI) calcd for C₁₈H₃₈O₂Si₂Br₂H⁺ [M+H]⁺: 501.0865. found: 501.0850.

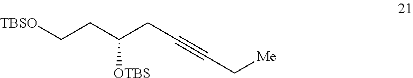 [SMILES: CCC#CC[C@@H](CCO[Si](C)(C)C(C)(C)C)O[Si](C)(C)C(C)(C)C]

### Alkyne 21

To a stirred solution of dibromide 19 (447 mg, 0.894 mmol) in THF (9 mL) at −78° C. was added n-BuLi (2.5 M in hexanes, 1.1 mL, 2.7 mmol, 3.0 equiv.) dropwise. The reaction mixture was warmed to 0° C. over 30 min and stirred for 30 additional min at that temperature. The mixture was recooled to −78° C., and EtI (0.72 mL, 8.9 mmol, 10 equiv.) was added. The mixture was allowed to warm to 25° C. and stirred for 3 h. The reaction was quenched by addition of sat. aq. NH₄Cl (10 mL) and then diluted with H₂O (5 mL) and EtOAc (15 mL). The organic layer was separated and washed with brine (10 mL), dried (Na₂SO₄), filtered, and concentrated. The crude ¹H NMR spectrum showed a mixture of hexyne derivative 20 and desired ethylalkyne 21. The mixture was resubjected to the reaction conditions and was stirred at 25° C. for 15 h to complete the alkylation. Following the same work-up as described above, flash column chromatography (SiO₂; hexanes:EtOAc, 20:1) provided the title compound (225 mg, 0.608 mmol, 68%) as a colorless oil. 21: R_{f}=0.6 (silica gel, hexanes:EtOAc, 19:1); [α]_{D}²⁵=+17.8 (c=1.0, CHCl₃); FT-IR (neat) νₘₐₓ=2954, 2929, 2857, 1472, 1463, 1252, 1091, 832, 772 cm⁻¹; ¹H NMR (500 MHz, CDCl₃) δ=3.93-3.87 (m, 1H), 3.72-3.67 (m, 2H), 2.31-2.27 (m, 2H), 2.18-2.12 (m, 2H), 1.86 (dtd, J=13.6, 7.4, 4.0 Hz, 1H), 1.69-1.62 (m, 1H), 1.11 (t, J=7.5 Hz, 3H), 0.89 (s, 9H), 0.88 (s, 9H), 0.08 (s, 3H), 0.06 (s, 3H), 0.049 (s, 3H), 0.045 (s, 3H) ppm; ¹³C NMR (126 MHz, CDCl₃) δ=83.45, 76.63, 68.67, 59.83, 39.97, 28.17, 26.09, 26.00, 18.42, 18.24, 14.34, 12.61, −4.35, −4.62, −5.11, −5.15 ppm; HRMS (ESI) calcd for C₂₀H₄₂O₂SiH⁺ [M+H]⁺: 371.2796. found: 371.2781.

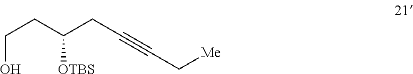 [SMILES: CCC#CC[C@@H](CCO)O[Si](C)(C)C(C)(C)C]

### Alcohol 21′

To a solution of bis-TBS ether 21 (225 mg, 0.608 mmol) in MeOH (6 mL) at −10° C. was added pyridinium tribromide (19 mg, 0.061 mmol, 0.10 equiv). The reaction mixture was stirred at −10° C. for 1 h, and the reaction was then quenched by addition of H₂O (10 mL). The mixture was extracted with EtOAc (5 mL), and the organic phase was dried (Na₂SO₄), filtered, and concentrated. Flash column chromatography (SiO₂; hexanes:EtOAc, 9:1→4:1) yielded the title compound (82 mg, 0.32 mmol, 53%) as a colorless oil. 21′: R_{f}=0.5 (silica gel, hexanes:EtOAc, 4:1); [α]_{D}²⁵=+26.7 (c=1.0, CHCl₃); FT-IR (neat) νₘₐₓ=3362, 2949, 2929, 2857, 1472, 1462, 1254, 1095, 1063, 1021, 834, 775 cm-1; ¹H NMR (500 MHz, CDCl₃) δ=4.01 (dddd, J=7.7, 6.6, 5.4, 3.9 Hz, 1H), 3.83 (ddd, J=10.8, 8.3, 4.3 Hz, 1H), 3.73 (ddd, J=10.8, 5.9, 4.9 Hz, 1H), 2.42-2.31 (m, 3H), 2.17-2.11 (m, 2H), 1.95 (dddd, J=14.2, 8.4, 4.9, 3.8 Hz, 1H), 1.78 (dddd, J=14.2, 6.7, 5.8, 4.3 Hz, 1H), 1.09 (t, J=7.6 Hz, 3H), 0.88 (s, 9H), 0.10 (ap s, 6H) ppm; ¹³C NMR (126 MHz, CDCl₃) δ=83.96, 75.96, 71.03, 60.18, 37.80, 27.41, 25.89, 18.09, 14.28, 12.55, −4.42, −4.75 ppm; HRMS (ESI) calcd for C₁₄H₂₈O₂SiH⁺ [M+H]⁺: 257.1931. found: 257.1925.

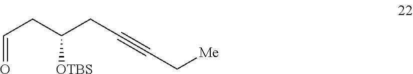 [SMILES: CCC#CC[C@@H](CC=O)O[Si](C)(C)C(C)(C)C]

### Aldehyde 22

To a solution of primary alcohol 21′ (80 mg, 0.31 mmol) in CH₂Cl₂ (4 mL) at 0° C. was added Dess-Martin periodinane (199 mg, 0.469 mmol, 1.5 equiv.). The reaction mixture was warmed to 25° C. and stirred for 90 min. The reaction was then quenched with sat. aq. Na₂S₂O₃ (4 mL) and sat. aq. NaHCO₃ (4 mL) and stirred for 10 min. The layers were separated, and the aqueous phase was extracted with CH₂Cl₂ (10 mL). The combined organics were dried (Na₂SO₄), filtered, and concentrated. Flash column chromatography (SiO₂; hexanes:EtOAc, 19:1→9:1) yielded the title aldehyde (73 mg, 0.29 mmol, 94%) as a colorless oil. 22: R_{f}=0.6 (silica gel, hexanes:EtOAc, 4:1); [α]_{D}²⁵=+22.7 (c=1.0, CHCl₃); FT-IR (neat) νₘₐₓ=2955, 2930, 2857, 1726, 1472, 1463, 1253, 1101, 1004, 835, 775 cm⁻¹; 1H NMR (500 MHz, CDCl3) δ=9.81 (dd, J=2.8, 1.9 Hz, 1H), 4.29 (dddd, J=8.0, 7.2, 5.0, 4.4 Hz, 1H), 2.72 (ddd, J=16.0, 4.4, 1.9 Hz, 1H), 2.61 (ddd, J=16.0, 7.2, 2.8 Hz, 1H), 2.41 (ddt, J=16.4, 4.9, 2.4 Hz, 1H), 2.32 (ddt, J=16.4, 8.0, 2.4 Hz, 1H), 2.17-2.11 (m, 2H), 1.10 (t, J=7.5 Hz, 3H), 0.85 (s, 9H), 0.09 (s, 3H), 0.06 (s, 3H) ppm; ¹³C NMR (126 MHz, CDCl₃) δ=201.88, 84.75, 75.32, 67.62, 50.57, 28.23, 25.82, 18.08, 14.19, 12.52, −4.37, −4.74 ppm; HRMS (ESI) calcd for C₁₄H₂₆O₂SiH⁺ [M+H]⁺: 255.1775. found: 255.1779.

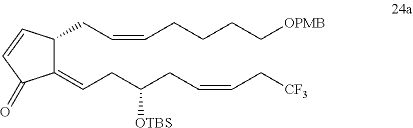

### Dienone 24a

To a stirred solution of diisopropylamine (123 μL; 0.872 mmol, 2.3 equiv.) in THF (3.8 mL) at 0° C. was dropwise added n-BuLi (2.5 M in hexanes, 320 μL; 0.800 mmol, 2.1 equiv.). After stirring for 20 min at this temperature, the clear solution was cooled to −78° C. and a solution of enone 23 (120 mg, 0.382 mmol, 1.0 equiv.) in THF (1.9 mL) was added dropwise. After stirring the resulting slightly yellow solution for an additional 20 min at this temperature, a solution of aldehyde 18 (141 mg, 0.454 mmol, 1.2 equiv.) in THF (1.9 mL) was added dropwise and stirring at this temperature was continued for an additional 30 min. The reaction was then quenched with sat. aq. NH₄Cl solution (5 mL), diluted with EtOAc (5 mL), and the mixture allowed to warm to 25° C. The phases were separated, the aq. layer was extracted with EtOAc (2×5 mL), and the combined organic extracts were washed with brine (10 mL), dried (Na₂SO₄), filtered, and concentrated. The crude mixture was filtered through a short column (SiO₂; hexanes:EtOAc, 17:3) to obtain a mixture of diastereomeric alcohols (210 mg) as a colorless oil which was taken to the next step without further purification. To a stirred solution of the so-obtained aldol product (210 mg, 0.336 mmol) in CH₂Cl₂ (4.3 mL) at 0° C. was added Et₃N (0.47 mL, 3.4 mmol, 10 equiv.), and then, methanesulfonyl chloride (130 μL, 1.7 mmol, 5.0 equiv.) dropwise. After stirring for 30 min at this temperature, the reaction was quenched with sat. aq. NaHCO₃ solution (5 mL), diluted with CH₂Cl₂ (5 mL), and allowed to warm to 25° C. The phases were separated, the aq. layer was extracted with CH₂Cl₂ (2×5 mL), and the combined organic layers were washed with H₂O (10 mL), dried (Na₂SO₄), filtered, and concentrated. The crude mesylate was filtered through a short column (SiO₂; hexanes:EtOAc, 3:2) to obtain the mixture of diastereomeric mesylates as a colorless oil which was taken to the next step without further purification. To a vigorously stirred solution of crude mesylate in CH₂Cl₂ (6.8 mL) at 25° C. was added Al₂O₃ (210 mg, 2.04 mmol, 7.0 equiv.). After 2 h and 4 h time intervals two more portions of Al₂O₃ (2×210 mg, 2×2.04 mmol, 2×7.0 equiv.) were added and vigorous stirring was continued for a total of 8 h. The resulting suspension was then filtered through Celite®, washed with EtOAc, and the solution obtained was concentrated. Flash column chromatography (SiO₂; hexanes:EtOAc, 9:1) yielded pure title compound (100 mg, 0.165 mmol, 43% for the three steps, mixture of inseparable isomers: Z:E ca. 7:1 by ¹H NMR analysis) as a colorless oil. 24a: R_{f}=0.50 (silica gel, hexanes:EtOAc, 2:1); [α]_{D}²⁵=+103.4 (c=0.99, CHCl₃); FT-IR (neat) νₘₐₓ=2930, 2856, 1704, 1656, 1613, 1584, 1513, 1463, 1249, 1132, 1095, 836, 776 cm⁻¹; ¹H NMR (500 MHz, CDCl₃) δ=7.49 (ddd, J=6.0, 2.7, 1.0 Hz, 1H), 7.25 (d, J=9.0 Hz, 2H), 6.87 (d, J=8.6 Hz, 2H), 6.59-6.53 (m, 1H), 6.32 (dd, J=6.0, 1.8 Hz, 1H), 5.82-5.73 (m, 1H), 5.54-5.44 (m, 2H), 5.36-5.28 (m, 1H), 4.42 (s, 2H), 3.93 (p, J=5.9 Hz, 1H), 3.80 (s, 3H), 3.46-3.41 (m, 1H), 3.42 (t, J=6.5 Hz, 2H), 2.90-2.71 (m, 1H), 2.63-2.53 (m, 1H), 2.48-2.36 (m, 2H), 2.30-2.22 (m, 2H), 2.21-2.12 (m, 1H), 2.03-1.95 (m, 2H), 1.62-1.54 (m, 2H), 1.41 (p, J=7.6 Hz, 2H), 1.30-1.20 (m, 1H), 0.87 (s, 9H), 0.05 (s, 6H) ppm; ¹³C NMR (126 MHz, CDCl₃) δ=196.35, 161.87, 159.22, 139.20, 134.93, 134.07, 132.70, 132.09, 131.68, 129.34, 126.21 (q, J=276.8 Hz), 125.13, 119.33 (q, J=3.5 Hz), 113.86, 72.68, 70.97, 70.01, 55.39, 43.48, 37.03, 35.38, 32.60 (q, J=32.6 Hz, CF₃), 30.66, 29.54, 27.30, 26.31, 25.91, 18.14, −4.42, −4.51 ppm; HRMS (ESI) calcd for C₃₄H₄₉O₄F₃SiNa⁺ [M+Na]⁺: 629.3244. found: 629.3240.

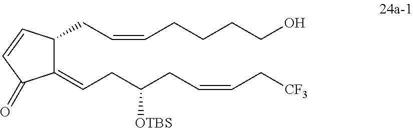 [SMILES: CC(C)(C)[Si](C)(C)O[C@@H](C/C=C\CC(F)(F)F)C/C=C1/C(=O)C=C[C@@H]1C/C=C\CCCCO]

### Hydroxy dienone 24a-1

To a vigorously stirred solution of dienone 24 (100 mg, 0.16 mmol, 1.0 equiv.) in a mixture of CH₂Cl₂:H₂O (16:1, 3.4 mL) at 0° C. was added in one portion 2,3-dichloro-5,6-dicyano-1,4-benzoquinone (56 mg, 0.24 mmol, 1.5 equiv.). After stirring at this temperature for 45 min, the reaction mixture was diluted with Et₂O (10 mL), filtered through Celite®, washed with Et₂O, and concentrated to a volume of ca. 1 mL (not to dryness!). Flash column chromatography (SiO₂; hexanes:EtOAc, 3:1→2:1) yielded pure title compound (72 mg, 0.15 mmol, 97%, mixture of inseparable isomers: Z:E ca. 7:1 by ¹H NMR analysis) as a colorless oil. 24a-1: R_{f}=0.13 (silica gel, hexanes:EtOAc, 2:1); [α]_{D}²⁵=+111.4 (c=1.02, CHCl₃); FT-IR (neat) νₘₐₓ=3444, 2930, 2858, 1702, 1654, 1580, 1463, 1347, 1253, 1200, 1135, 1083, 837, 776 cm-1; ¹H NMR (500 MHz, CDCl₃, data for Z isomer) δ=7.50 (ddd, J=5.5, 2.7, 1.0 Hz, 1H), 6.58-6.53 (m, 1H), 6.32 (dt, J=6.0, 1.8 Hz, 1H), 5.99 (t, J=11.3 Hz, 1H), 5.82-5.72 (m, 1H), 5.54-5.47 (m, 2H), 5.37-5.30 (m, 1H), 3.97-3.90 (m, 1H), 3.63 (t, J=6.5 Hz, 2H), 3.46-3.40 (m, 1H), 2.90-2.72 (m, 1H), 2.63-2.55 (m, 1H), 2.47-2.37 (m, 2H), 2.29-2.20 (m, 2H), 2.21-2.11 (m, 1H), 2.05-1.96 (m, 2H), 1.59-1.49 (m, 2H), 1.45-1.36 (m, 2H), 0.87 (s, 9H), 0.05 (s, 6H) ppm; ¹³C NMR (126 MHz, CDCl₃, data for Z isomer) δ=196.40, 161.88, 139.20, 134.92, 132.60, 132.07, 131.77, 126.29 (q, J=276.3 Hz), 125.30, 119.35 (q, J=3.4 Hz), 70.99, 62.82, 43.58, 43.50, 37.05, 35.38, 32.60 (q, J=32.6 Hz, CF₃), 32.42, 30.67, 27.21, 25.90, 18.14, −4.43, −4.53 ppm; HRMS (ESI) calcd for C₂₆H₄₁O₃F₃SiNa⁺ [M+Na]⁺: 509.2669. found: 509.2661.

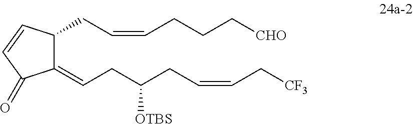 [SMILES: CC(C)(C)[Si](C)(C)O[C@@H](C/C=C\CC(F)(F)F)C/C=C1/C(=O)C=C[C@@H]1C/C=C\CCCC=O]

### Aldehyde dienone 24a-2

To a vigorously stirred solution of hydroxy dienone 24a-1 (55 mg, 0.11 mmol, 1.0 equiv.) in CH₂Cl₂ (1.2 mL) at 25° C. was added in one portion pyridinium chlorochromate (47 mg, 0.22 mmol, 2.0 equiv.). After stirring for 2 h, the reaction mixture was diluted with Et₂O (5 mL), filtered through Celite®, washed with Et₂O, and concentrated to a volume of ca. 1 mL (not to dryness!). Flash column chromatography (SiO₂; hexanes:EtOAc, 9:1) yielded pure title compound (50 mg, 0.10 mmol, 91%, mixture of inseparable isomers: Z:E ca. 7:1 by ¹H NMR analysis) as a colorless oil. 24a-2: R_{f}=0.52 (silica gel, hexanes:EtOAc, 3:1); [α]_{D}²⁵=+82.7 (c=0.88, CHCl₃); FT-IR (neat) νₘₐₓ=2952, 2929, 2857, 1719, 1703, 1655, 1581, 1463, 1348, 1251, 1132, 1083, 836, 775 cm⁻¹; ¹H NMR (500 MHz, CDCl₃, data for Z isomer) δ=9.74 (s, 1H), 7.48 (dt, J=6.0, 3.1 Hz, 1H), 6.58-6.51 (m, 1H), 6.35-6.29 (m, 1H), 5.99 (t, J=11.2 Hz, 1H), 5.82-5.72 (m, 1H), 5.54-5.40 (m, 2H), 5.40-5.30 (m, 1H), 3.97-3.87 (m, 1H), 3.48-3.40 (m, 1H), 2.90-2.72 (m, 1H), 2.63-2.53 (m, 1H), 2.45-2.36 (m, 4H), 2.29-2.20 (m, 2H), 2.21-2.11 (m, 1H), 2.06-1.96 (m, 2H), 1.67 (p, J=7.4 Hz, 2H), 0.86 (s, 9H), 0.04 (s, 6H) ppm; ¹³C NMR (126 MHz, CDCl₃, data for Z isomer) δ=202.23, 196.22, 161.58, 139.05, 135.06, 132.07, 131.83, 131.48, 126.18, 126.11 (q, J=278.3 Hz), 119.33 (q, J=3.5 Hz), 70.94, 43.34, 43.33, 36.99, 35.40, 32.60 (q, J=32.6 Hz, CF₃), 30.57, 26.70, 25.89, 21.91, 18.13, −4.44, −4.53 ppm; HRMS (ESI) calcd for C₂₆H₃₉O₃F₃SiNa⁺ [M+Na]⁺: 507.2513. found: 507.2505.

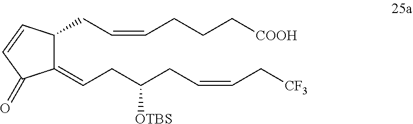 [SMILES: CC(C)(C)[Si](C)(C)O[C@@H](C/C=C\CC(F)(F)F)C/C=C1/C(=O)C=C[C@@H]1C/C=C\CCCC(=O)O]

### ω-Trifluoromethyl-Δ¹²-PGJ₃-14-t-butyldimethylsilyl-ether (25a)

To a vigorously stirred solution of aldehyde dienone 24a-2 (50 mg, 0.10 mmol, 1.0 equiv.) in t-BuOH (1.7 mL) at 25° C. were dropwise added sequentially 2-methyl-2-butene (116 μL, 1.03 mmol, 10 equiv.), a solution of NaH₂PO₄ (24 mg, 0.15 mmol, 1.5 equiv.) in H₂O (0.55 mL) and a solution of NaClO₂ (80%, 18 mg, 0.15 mmol, 1.5 equiv.) in H₂O (0.55 mL). After stirring for 30 min, the reaction mixture was diluted with a solution of NaH₂PO₄ (1.0 g) in H₂O (20 mL) and extracted with EtOAc (5×20 mL). The combined organic extracts were washed with brine (25 mL), dried (Na₂SO₄), filtered, and concentrated. Flash column chromatography (SiO₂; CH₂Cl₂:EtOH, 25:1→20:1) yielded pure title compound 25a (43 mg, 0.08 mmol, 86%, mixture of inseparable isomers: Z:E ca. 7:1 by ¹H NMR analysis) as a colorless oil. 25a: R_{f}⁼0.40 (silica gel, CH₂Cl₂:EtOH, 19:1); [α]_{D}²⁵=+110.0 (c=1.07, CHCl₃); FT-IR (neat) νₘₐₓ=3013, 2954, 2929, 2857, 1705, 1654, 1462, 1360, 1251, 1134, 1082, 836, 775 cm⁻¹; ¹H NMR (500 MHz, CDCl₃, data for Z isomers) δ=7.52-7.48 (m, 1H), 6.60-6.53 (m, 1H), 6.34 (dt, J=6.0, 1.9 Hz, 1H), 5.99 (t, J=11.4 Hz, 1H), 5.82-5.72 (m, 1H), 5.55-5.42 (m, 2H), 5.42-5.33 (m, 1H), 3.98-3.87 (m, 1H), 3.47-3.39 (m, 1H), 2.89-2.72 (m, 1H), 2.64-2.56 (m, 1H), 2.47-2.36 (m, 2H), 2.33 (t, J=2.3 Hz, 2H), 2.29-2.21 (m, 2H), 2.20-2.10 (m, 1H), 2.08-2.01 (m, 2H), 1.68 (p, J=7.0 Hz, 2H), 0.87 (s, 9H), 0.05 (s, 3H), 0.05 (s, 3H) ppm; ¹³C NMR (126 MHz, CDCl₃, data for Z isomer) δ=196.43, 179.13, 161.81, 139.11, 135.01, 132.05, 131.93, 131.47, 126.17, 126.11 (q, J=279.1 Hz), 119.38 (q, J=3.5 Hz), 71.04, 43.42, 37.04, 35.37, 33.43, 32.58 (q, J=32.6 Hz, CF₃), 30.60, 26.69, 25.90, 24.53, 18.15, −4.44, −4.52 ppm; HRMS (ESI) calcd for C₂₆H₃₉O₄F₃SiNa⁺ [M+Na]⁺: 523.2462. found: 523.2444.

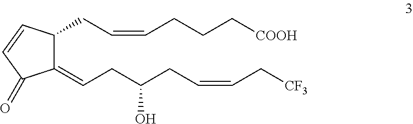 [SMILES: O=C(O)CCC/C=C\C[C@H]1C=CC(=O)/C1=C/C[C@@H](O)C/C=C\CC(F)(F)F]

### ω-Trifluoromethyl-Δ¹²-PGJ₃ (3)

To a stirred solution of ω-trifluoromethyl-Δ¹²-PGJ₃-14-t-butyldimethylsilyl-ether (25a) (40 mg, 79 μmol; 1.0 equiv.) in MeCN (0.8 mL) at 0° C. was dropwise added a solution of HF (50% aq., 150 μL, ca. 3.95 mmol, ca. 50 equiv.) in MeCN (0.8 mL). After stirring for 3 h at this temperature, the reaction was quenched with brine (5 mL) and extracted with EtOAc (5×5 mL). The combined organic extracts were dried (Na₂SO₄), filtered, and concentrated. Flash column chromatography (SiO₂; CH₂Cl₂:EtOH, 20:1→15:1) yielded the title compound 3 (21 mg, 54 μmol, 70%, inseparable mixture of isomers: Z:E ca. 7:1 by ¹H NMR analysis) as a colorless oil. 3: R_{f}⁼0.24 (silica gel, CH₂Cl₂:MeOH, 19:1); [α]_{D}²⁵=+89.8 (c=1.05, CHCl₃); FT-IR (neat) νₘₐₓ=3423, 2942, 1701, 1646, 1581, 1429, 1344, 1251, 1136, 1059, 916, 834 cm⁻¹; ¹H NMR (500 MHz, CDCl₃, data for Z isomer) δ=7.57 (ddd, J=6.0, 2.6, 1.1 Hz, 1H), 6.62-6.55 (m, 1H), 6.37-6.33 (m, 1H), 6.07 (t, J=11.4 Hz, 1H), 5.83-5.75 (m, 1H), 5.62-5.54 (m, 1H), 5.53-5.45 (m, 1H), 5.44-5.37 (m, 1H), 5.15 (dddd, J=23.8, 11.5, 1.8, 1.1 Hz, 1H), 3.97-3.87 (m, 1H), 3.48-3.43 (m, 1H), 2.93-2.82 (m, 1H), 2.73-2.64 (m, 1H), 2.57 (dq, J=12.6, 6.4 Hz, 1H), 2.52-2.43 (m, 1H), 2.35 (t, J=6.9 Hz, 2H), 2.32-2.29 (m, 2H), 2.16-2.02 (m, 3H), 1.68 (p, J=7.2 Hz, 2H) ppm; ¹³C NMR (126 MHz, CDCl₃, data for Z isomer) δ=196.65, 177.90, 162.17, 139.86, 134.97, 131.72, 131.65, 131.35, 126.16 (q, J=275.7 Hz), 126.07, 120.51 (q, J=3.5 Hz), 70.52, 43.73, 36.70, 34.87, 33.11, 32.57 (q, J=32.6 Hz), 30.48, 26.60, 24.52 ppm; HRMS (ESI) calcd for C₂₀H₂₅O₄F₃Na⁺ [M+Na]⁺: 409.1597. found: 409.1579.

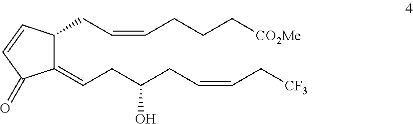 [SMILES: COC(=O)CCC/C=C\C[C@H]1C=CC(=O)/C1=C/C[C@@H](O)C/C=C\CC(F)(F)F]

### ω-Trifluoromethyl-Δ¹²-PGJ₃ methyl ester analog (4)

To a stirred solution of ω-trifluoromethyl-Δ¹²-PGJ₃ analog (3) (10.0 mg, 25.9 μmol, 1.0 equiv.) in C₆H₆:MeOH (3:2, 1.0 mL) at 25° C. was added dropwise a solution of trimethylsilyl diazomethane (2 M in Et₂O, 20 μL, 40 μmol, 1.5 equiv.) (yellow color persists). After stirring for 15 min, the reaction mixture was concentrated. Flash column chromatography (SiO₂; hexanes:EtOAc, 3:1→3:2) yielded the title compound (8.8 mg, 22.0 μmol, 85%, inseparable mixture of isomers: Z:E ca. 7:1 by ¹H NMR analysis) as a colorless oil. 4: R_{f}=0.32 (silica gel, hexanes:EtOAc, 2:1); [α]_{D}²⁵=+143.7 (c=0.80, C₆H₆); FT-IR (neat) νₘₐₓ=3445, 3011, 2930, 1735, 1720, 1700, 1651, 1580, 1437, 1349, 1252, 1135, 1065 cm⁻¹; ¹H NMR (500 MHz, CDCl₃, data for Z isomer) δ=7.52 (ddd, J=5.0, 2.6, 1.3 Hz, 1H), 6.63-6.58 (m, 1H), 6.35 (dt, J=6.1, 1.4 Hz, 1H), 6.12-6.05 (m, 1H), 5.85-5.78 (m, 1H), 5.59 (dtd, J=10.8, 7.4, 1.5 Hz, 1H), 5.50-5.43 (m, 1H), 5.35 (dddd, J=9.5, 8.0, 6.8, 3.3 Hz, 1H), 5.16 (dddd, J=23.8, 11.4, 2.0, 1.0 Hz, 1H), 3.89 (dd, J=9.3, 4.0 Hz, 1H), 3.66 (s, 3H), 3.52-3.47 (m, 1H), 2.93-2.83 (m, 1H), 2.64 (dt, J=15.1, 5.7 Hz, 1H), 2.54 (dt, J=14.4, 7.1 Hz, 1H), 2.46 (ddd, J=15.4, 8.5, 5.7 Hz, 1H), 2.30 (td, J=7.3, 1.1 Hz, 2H), 2.22 (ddd, J=15.2, 9.2, 5.0 Hz, 2H), 2.05 (q, J=7.5 Hz, 3H), 1.71-1.63 (m, 2H) ppm; ¹³C NMR (151 MHz, CDCl₃, data for Z isomer) δ=196.39, 174.22, 161.85, 139.79, 135.07, 133.75, 131.77, 131.19, 126.17 (q, J=276.3 Hz), 125.85, 120.48, 70.31, 51.72, 43.44, 36.87, 35.19, 33.46, 32.62 (q, J=28.7 Hz), 30.40, 26.83, 24.76 ppm; HRMS (ESI) calcd for C₂₁H₂₇F₃O₄Na⁺ [M+Na]⁺: 423.1754. found: 423.1738.

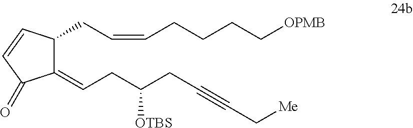

### Dienone 24b

To a stirred solution of diisopropylamine (74 μL, 0.53 mmol, 2.2 equiv. in THF (2.2 mL) at 0° C. was added dropwise n-BuLi (2.5 M in hexanes, 0.19 mL, 0.48 mmol, 2.0 equiv.). After stirring for 20 min at this temperature, the clear solution was cooled to −78° C., and a solution of enone 23 (75 mg, 0.24 mmol, 1.0 equiv.) in THF (2.2 mL) was added dropwise. After stirring the resulting slightly yellow solution for an additional 20 min at this temperature, a solution of aldehyde 22 (73 mg, 0.29 mmol, 1.2 equiv.) in THF (2.2 mL) was added dropwise, and stirring at this temperature was continued for an additional 30 min. The reaction was then quenched with sat. aq. NH₄Cl solution (8 mL), diluted with EtOAc (8 mL), and allowed to warm to 25° C. The phases were separated, the aq. layer was extracted with EtOAc (8 mL), and the combined organic extracts were washed with brine (8 mL), dried (Na₂SO₄), filtered, and concentrated. The crude aldol product was filtered through a short column (SiO₂; hexanes:EtOAc, 9:1→3:1) to obtain a mixture of diastereomeric alcohols (79 mg) as a colorless oil which was taken to the next step without further purification. To a stirred solution of the so-obtained aldol product (78 mg, 0.14 mmol) in CH₂Cl₂ (1.5 mL) at 0° C. was added Et₃N (0.20 mL, 1.4 mmol, 10 equiv.), and then, slowly and dropwise, methanesulfonyl chloride (53 μL, 0.69 mmol, 5.0 equiv.). After stirring for 25 min at this temperature, the reaction was quenched with sat. aq. NaHCO₃ solution (4 mL), diluted with CH₂Cl₂ (5 mL), and allowed to warm to 25° C. The phases were separated, and the organic layer was washed with H₂O (10 mL), dried (Na₂SO₄), filtered, and concentrated. The crude mesylate was filtered through a short column (SiO₂; hexanes:EtOAc, 3:1) to obtain the mixture of diastereomeric mesylates as a colorless oil which was taken to the next step without further purification. To a vigorously stirred solution of the so obtained mesylate in CH₂Cl₂ (3 mL) at 25° C. was added Al₂O₃ (100 mg, 0.98 mmol, 7.0 equiv.). After 2 h and 4 h time intervals two more portions of Al₂O₃ (2×100 mg, 2×0.98 mmol, 2×7.0 equiv.) were added, and vigorous stirring was continued for a total of 6 h. The resulting suspension was then filtered through Celite®, washed with EtOAc, and the solution obtained was concentrated. Flash column chromatography (SiO₂; hexanes:EtOAc, 19:1→9:1-4:1) yielded pure title compound (26 mg, 0.047 mmol, 20% for the three steps) as a colorless oil. 24b: R_{f}=0.65 (silica gel, hexanes:EtOAc, 7:3); [α]_{D}²⁵=+97.1 (c=0.86, CHCl₃); FT-IR (neat) νₘₐₓ=2929, 2855, 1704, 1656, 1613, 1512, 1246, 1095, 1036, 835, 776 cm⁻¹; ¹H NMR (500 MHz, CDCl₃) δ=7.49 (ddd, J=6.0, 2.6, 1.0 Hz, 1H), 7.25 (d, J=8.6 Hz, 2H), 6.87 (d, J=8.6 Hz, 2H), 6.64-6.61 (m, 1H), 6.31 (dd, J=6.0, 1.8 Hz, 1H), 5.51-5.45 (m, 1H), 5.37-5.31 (m, 1H), 4.42 (s, 2H), 3.93 (tt, J=6.6, 5.3 Hz, 1H), 3.80 (s, 3H), 3.50-3.47 (m, 1H), 3.42 (t, J=6.5 Hz, 2H), 2.67-2.59 (m, 2H), 2.50 (dt, J=14.6, 6.8 Hz, 1H), 2.33-2.29 (m, 2H), 2.21-2.11 (m, 3H), 1.99 (q, J=7.6 Hz, 2H), 1.61-1.56 (m, 2H), 1.43-1.37 (m, 2H), 1.09 (t, J=7.6 Hz, 3H), 0.87 (s, 9H), 0.08 (s, 3H), 0.05 (s, 3H) ppm; ¹³C NMR (126 MHz, CDCl₃) δ=196.42, 161.86, 159.21, 139.20, 134.92, 132.55, 132.22, 130.78, 129.33, 125.38, 113.85, 84.15, 75.97, 72.68, 70.92, 70.02, 55.39, 43.49, 36.63, 30.73, 29.53, 27.96, 27.28, 26.32, 25.91, 18.18, 14.30, 12.56, −4.47, −4.56 ppm; HRMS (ESI) calcd for C₃₄H₅₀O₄SiNa⁺ [M+Na]⁺: 573.3371. found: 573.3344.

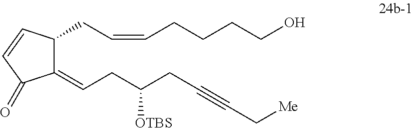 [SMILES: CCC#CC[C@@H](C/C=C1/C(=O)C=C[C@@H]1C/C=C\CCCCO)O[Si](C)(C)C(C)(C)C]

### Alcohol 24b-1

To a vigorously stirred solution of dienone 24b (25 mg, 0.045 mmol, 1.0 equiv.) in a mixture of CH₂Cl₂:H₂O (10:1, 1.1 mL) at 0° C. was added in one portion 2,3-dichloro-5,6-dicyano-1,4-benzoquinone (15 mg, 0.068 mmol, 1.5 equiv.). After stirring at this temperature for 45 min, an additional portion of 2,3-dichloro-5,6-dicyano-1,4-benzoquinone (5.0 mg, 0.023 mmol, 0.5 equiv.) was added. After stirring for an additional 60 min, the reaction mixture was diluted with Et₂O (3 mL), filtered through Celite®, washed with Et₂O, and concentrated to a volume of ca. 0.5 mL (not to dryness!). Flash column chromatography (SiO₂; hexanes:EtOAc, 9:1→3:1→3:2) yielded pure title compound (19 mg, 0.044 mmol, 98%) as a colorless oil. 24b-1: R_{f}=0.3 (silica gel, hexanes:EtOAc, 7:3); [α]_{D}²⁵=+121.2 (c=1.0, CHCl₃); FT-IR (neat) νₘₐₓ=3434, 2928, 2856, 1700, 1652, 1580, 1462, 1252, 1090, 967, 835, 775 cm⁻¹; ¹H NMR (500 MHz, CDCl₃) δ=7.51 (ddd, J=6.0, 2.6, 1.0 Hz, 1H), 6.62 (ddt, J=8.4, 7.1, 1.3 Hz, 1H), 6.32 (dd, J=6.0, 1.8 Hz, 1H), 5.51-5.46 (m, 1H), 5.38-5.33 (m, 1H), 3.94 (tt, J=6.7, 5.4 Hz, 1H), 3.63 (t, J=6.5 Hz, 2H), 3.52-3.48 (m, 1H), 2.69-2.60 (m, 2H), 2.50 (dt, J=14.6, 6.8 Hz, 1H), 2.33-2.29 (m, 2H), 2.22-2.11 (m, 3H), 2.02 (t, J=7.5 Hz, 2H), 1.65-1.53 (m, 3H), 1.44-1.38 (m, 2H), 1.09 (t, J=7.5 Hz, 3H), 0.87 (s, 9H), 0.08 (s, 3H), 0.05 (s, 3H) ppm; ¹³C NMR (126 MHz, CDCl₃) δ=196.49, 161.88, 139.21, 134.95, 132.45, 132.31, 125.51, 84.19, 75.96, 70.93, 62.86, 43.52, 36.68, 32.44, 30.75, 27.97, 27.21, 25.92, 25.84, 18.20, 14.30, 12.57, −4.51, −4.55 ppm; HRMS (ESI) calcd for C₂₆H₄₂O₃SiNa⁺ [M+Na]⁺: 453.2795. found: 453.2788.

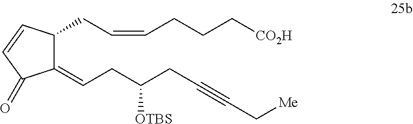 [SMILES: CCC#CC[C@@H](C/C=C1/C(=O)C=C[C@@H]1C/C=C\CCCC(=O)O)O[Si](C)(C)C(C)(C)C]

### Acid 25b

To a vigorously stirred solution of alcohol 24b-1 (19 mg, 0.044 mmol, 1.0 equiv.) in CH₂Cl₂ (1 mL) at 25° C. was added in one portion pyridinium chlorochromate (19 mg, 0.088 mmol, 2.0 equiv.). After stirring for 40 min, the reaction mixture was filtered through a short pad of SiO₂ (hexanes:EtOAc, 7:3) and concentrated to give intermediate aldehyde 24b-2 (18 mg, 0.042 mmol, 95%) which was used directly in the next step. To a vigorously stirred solution of aldehyde 24b-2 (18 mg, 0.042 mmol, 1.0 equiv.) in t-BuOH (0.8 mL) and H₂O (0.6 mL) at 25° C. were added sequentially 2-methyl-2-butene (47 μL, 0.44 mmol, 10 equiv.), NaH₂PO₄ (10 mg, 0.066 mmol, 1.5 equiv.) and NaClO₂ (6.0 mg, 0.066 mmol, 1.5 equiv.). After stirring for 20 min, the reaction mixture was diluted with a solution of NaH₂PO₄ (500 mg) in H₂O (10 mL) and extracted with EtOAc (2×7 mL). The combined organic extracts were dried (Na₂SO₄), filtered, and concentrated to a volume of ca. 1 mL (not to dryness!). Flash column chromatography (SiO₂; CH₂Cl₂:EtOH, 49:1→49:1) yielded pure title compound (15.6 mg, 0.0351 mmol, 80% for the two steps) as a colorless oil. 25b: R_{f}=0.5 (silica gel, CH₂Cl₂:EtOH, 19:1); [α]_{D}²⁵=+122.8 (c=1.0, CHCl₃); FT-IR (neat) νₘₐₓ=2929, 2856, 1705, 1654, 1462, 1433, 1361, 1251, 1091, 836, 808, 776 cm⁻¹; ¹H NMR (500 MHz, CDCl₃) δ=7.50 (ddd, J=6.0, 2.6, 1.0 Hz, 1H), 6.63 (ddt, J=8.3, 7.0, 1.2 Hz, 1H), 6.34 (dd, J=6.0, 1.8 Hz, 1H), 5.49-5.37 (m, 2H), 3.94 (p, J=6.0 Hz, 1H), 3.52-3.48 (m, 1H), 2.69-2.60 (m, 2H), 2.51 (dt, J=14.7, 6.7 Hz, 1H), 2.36-2.29 (m, 4H), 2.21-2.11 (m, 3H), 2.06 (q, J=7.5 Hz, 2H), 1.71-1.65 (m, 2H), 1.09 (t, J=7.5 Hz, 3H), 0.87 (s, 9H), 0.08 (s, 3H), 0.06 (s, 3H) ppm; ¹³C NMR (126 MHz, CDCl₃) δ=196.48, 178.71, 161.77, 139.12, 135.04, 132.41, 131.35, 126.42, 84.23, 75.94, 71.00, 43.46, 36.67, 33.38, 30.69, 27.93, 26.70, 25.92, 24.58, 18.22, 14.29, 12.57, −4.48, −4.54 ppm; HRMS (ESI) calcd for C₂₆H₄₀O₄SiNa⁺ [M+Na]⁺: 467.2588. found: 467.2571.

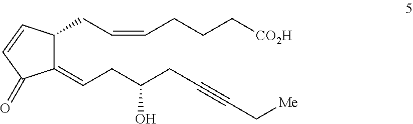 [SMILES: CCC#CC[C@H](O)C/C=C1/C(=O)C=C[C@@H]1C/C=C\CCCC(=O)O]

### 17,18-Didehydro-Δ¹²-PGJ₃ (5)

To a stirred solution of TBS ether 25b (15 mg, 0.034 mmol, 1.0 equiv.) in MeCN (0.8 mL) at 0° C. was added dropwise a solution of HF (50% aq., 60 μL, ca. 1.7 mmol, ca. 50 equiv.) in MeCN (0.2 mL). After stirring for 40 min, additional HF (50% aq., 60 μL, ca. 1.7 mmol, ca. 50 equiv.) in MeCN (0.2 mL) was added. After a further 90 min, a final portion of HF (50% aq., 40 μL, ca. 1.1 mmol, ca. 33 equiv.) in MeCN (0.2 mL) was added. After stirring for 30 min, the reaction was quenched with brine (3 mL) and extracted with EtOAc (5 mL). The organic extract was dried (Na₂SO₄), filtered, and concentrated to a volume of ca. 0.5 mL (not to dryness!). Flash column chromatography (SiO₂; CH₂Cl₂:EtOH, 99:1→97:3→95:5→93:7) yielded pure title compound (7.6 mg, 0.023 mmol, 68%) as a colorless oil. 5: R_{f}=0.4 (silica gel, CH₂Cl₂:EtOH, 19:1); [α]D²⁵=+88.0 (c=0.62, CHCl₃); FT-IR (neat)=3407, 2974, 2935, 1698, 1646, 1433, 1406, 1237, 1182, 1061, 1047 cm⁻¹; ¹H NMR (600 MHz, CDCl₃) δ=7.55 (dd, J=6.0, 2.5 Hz, 1H), 6.57 (t, J=7.7 Hz, 1H), 6.35 (d, J=6.0 Hz, 1H), 5.51-5.47 (m, 1H), 5.44-5.40 (m, 1H), 3.95 (p, J=6.3 Hz, 1H), 3.52-3.48 (m, 1H), 2.70 (dt, J=12.7, 5.9 Hz, 1H), 2.65-2.54 (m, 2H), 2.52-2.44 (m, 2H), 2.40-2.33 (m, 3H), 2.20-2.17 (m, 2H), 2.11 (q, J=7.5 Hz, 2H), 1.72-1.67 (m, 2H), 1.13 (t, J=7.5 Hz, 3H) ppm; ¹³C NMR (151 MHz, CDCl₃) δ=196.42, 177.01, 161.86, 139.93, 135.06, 131.67, 130.93, 126.18, 85.64, 74.58, 69.67, 43.72, 35.85, 33.05, 30.61, 27.22, 26.62, 24.62, 14.30, 12.57 ppm; HRMS (ESI) calcd for C₂₀H₂₆O₄Na⁺ [M+Na]⁺: 353.1723. found: 353.1729.

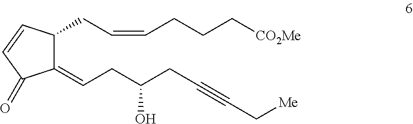 [SMILES: CCC#CC[C@H](O)C/C=C1/C(=O)C=C[C@@H]1C/C=C\CCCC(=O)OC]

### 17,18-Didehydro-Δ¹²-PGJ₃ methyl ester (6)

To a stirred solution of 17,18-didehydro-Δ¹²-PGJ₃ (5) (2.0 mg, 6.1-1.0 equiv.) in C₆H₆:MeOH (3:2, 0.25 mL) at 25° C. was added dropwise a solution of trimethylsilyl diazomethane (2 M in Et₂O, 6 μL, 12 μmol, 2.0 equiv.) (yellow color persists). After stirring for 15 min, the reaction mixture was concentrated. Flash column chromatography (SiO₂; hexanes:EtOAc, 3:1→3:2) yielded the pure title compound (1.8 mg, 5.2 μmol, 90%) as a colorless oil. 6: R_{f}=0.26 (silica gel, hexanes:EtOAc, 2:1); [α]_{D}²⁵=+129.2 (c=0.18, C₆H₆); FT-IR (neat) νₘₐₓ=3444, 2922, 2852, 1735, 1699, 1651, 1580, 1436, 1367, 1209, 1176, 1068 cm⁻¹; ¹H NMR (600 MHz, CDCl₃) δ=7.51 (dd, J=6.0, 2.6 Hz, 1H), 6.61 (t, J=7.8 Hz, 1H), 6.35 (dd, J=6.0, 1.8 Hz, 1H), 5.51-5.44 (m, 1H), 5.36 (dtt, J=10.9, 8.2, 1.6 Hz, 1H), 3.91 (dt, J=6.5, 5.3 Hz, 1H), 3.67 (s, 3H), 3.58-3.52 (m, 1H), 2.64 (dtd, J=14.5, 5.2, 4.7, 2.4 Hz, 1H), 2.57 (dd, J=7.8, 6.4 Hz, 2H), 2.47 (ddt, J=16.5, 4.8, 2.4 Hz, 1H), 2.35 (ddt, J=16.5, 6.2, 2.4 Hz, 1H), 2.31 (t, J=7.3 Hz, 2H), 2.28-2.21 (m, 2H), 2.19 (qt, J=7.5, 2.4 Hz, 2H), 2.05 (q, J=7.0 Hz, 1H), 1.67 (p, J=7.4 Hz, 2H), 1.13 (t, J=7.5 Hz, 3H) ppm; ¹³C NMR (151 MHz, CDCl₃) δ=196.39, 174.13, 161.78, 139.71, 135.09, 131.72, 131.16, 125.95, 85.50, 74.74, 69.44, 51.73, 43.41, 36.08, 33.50, 30.46, 27.51, 26.85, 24.81, 14.34, 12.58 ppm; HRMS (ESI) calcd for C₂₁H₂₈O₄Na⁺ [M+Na]⁺: 367.1880. found: 367.1883.

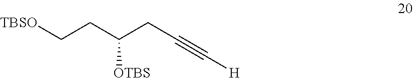 [SMILES: C#CC[C@@H](CCO[Si](C)(C)C(C)(C)C)O[Si](C)(C)C(C)(C)C]

### Terminal alkyne 20

To a solution of dibromide 19 (1.2 g, 2.4 mmol, 1.0 equiv.) in THF (24 mL) at −78° C. was added n-BuLi (2.5 M in hexanes, 2.9 mL, 7.2 mmol, 3.0 equiv.) dropwise. The resulting solution was allowed to warm to 0° C., and the reaction was then quenched with sat. aq. NH₄Cl solution (15 mL). The mixture was extracted with EtOAc (2×20 mL), and the combined organics were washed with brine (20 mL), dried (Na₂SO₄), filtered, and concentrated to yield analytically pure alkyne 20 (828 mg, 2.42 mmol, quant.).

20: R_{f}=0.5 (silica gel, hexanes:EtOAc, 19:1); [α]_{D}²⁵=+23.7 (c=1.0, CHCl₃); FT-IR (neat) νₘₐₓ=2954, 2929, 2857, 1472, 1463, 1253, 1092, 833, 772 cm⁻¹; ¹H NMR (600 MHz, CDCl₃) δ=3.97 (dtd, J=7.4, 6.0, 4.2 Hz, 1H), 3.72-3.67 (m, 2H), 2.35 (dd, J=6.0, 2.7 Hz, 2H), 1.97 (t, J=2.7 Hz, 1H), 1.85 (dtd, J=13.6, 7.1, 4.3 Hz, 1H), 1.71 (ddt, J=13.6, 7.4, 5.8 Hz, 1H), 0.89 (ap s, 18H), 0.09 (s, 3H), 0.07 (s, 3H), 0.048 (s, 3H), 0.045 (s, 3H) ppm; ¹³C NMR (151 MHz, CDCl₃) δ=81.75, 70.09, 68.03, 59.63, 39.81, 27.80, 26.09, 25.99, 18.41, 18.23, −4.36, −4.63, −5.13, −5.16 ppm; HRMS (ESI) calcd for C₁₈H₃₈O₂Si₂H⁺ [M+H]⁺: 343.2483. found: 343.2490.

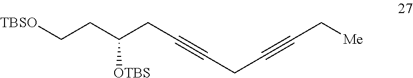 [SMILES: CCC#CCC#CC[C@@H](CCO[Si](C)(C)C(C)(C)C)O[Si](C)(C)C(C)(C)C]

### bis-Alkyne 27

To a solution of terminal alkyne 20 (828 mg, 2.42 mmol, 1.0 equiv.) in N,N-dimethylformamide (3 mL) was added sequentially 1-bromo-2-pentyne (0.30 mL, 2.9 mmol, 1.2 equiv.), K₂CO₃ (434 mg, 3.12 mmol, 1.3 equiv.), CuI (598 mg, 3.14 mmol, 1.3 equiv.), and NaI (468 mg, 3.14 mmol, 1.3 equiv.). The resulting heterogeneous mixture was sealed tightly with a cap and vigorously stirred in the dark for 15 h. The reaction mixture was then diluted with H₂O (3 mL) and Et₂O (3 mL), filtered through Celite® (Et₂O), and concentrated. The crude residue was resuspended in Et₂O (10 mL) and washed sequentially with sat. aq. NH₄Cl solution (10 mL) and brine (10 mL). The organic layer was dried (Na₂SO₄), filtered, and concentrated. Flash column chromatography (SiO₂; hexanes:EtOAc, 49:1→19:1) yielded the pure title compound (774 mg, 1.90 mmol, 79%) as a colorless oil. 27: R_{f}=0.7 (silica gel, hexanes:Et₂O, 19:1); [α]_{D}²⁵=+14.0 (c=0.64, CHCl₃); FT-IR (neat) νₘₐₓ=2954, 2929, 2857, 1472, 1361, 1255, 1094, 1035, 835, 806, 775 cm⁻¹; ¹H NMR (600 MHz, CDCl₃) δ=3.95-3.91 (m, 1H), 3.72-3.65 (m, 2H), 3.11 (p, J=2.4 Hz, 2H), 2.35-2.27 (m, 2H), 2.16 (qt, J=7.5, 2.4 Hz, 2H), 1.83 (dtd, J=14.0, 7.2, 4.2 Hz, 1H), 1.67 (ddt, J=14.0, 7.4, 6.0 Hz, 1H), 1.12 (t, J=7.5 Hz, 3H), 0.892 (s, 9H), 0.887 (s, 9H), 0.08 (s, 3H), 0.06 (s, 3H), 0.05 (s, 3H), 0.04 (s, 3H) ppm; ¹³C NMR (151 MHz, CDCl₃) δ=82.00, 77.72, 76.29, 73.74, 68.45, 59.78, 40.06, 28.15, 26.09, 26.00, 18.42, 18.24, 14.03, 12.54, 9.90, −4.33, −4.63, −5.10, −5.14 ppm; HRMS (ESI) calcd for C₂₃H₄₄O₂Si₂H⁺ [M+H]⁺: 409.2953. found: 409.2950.

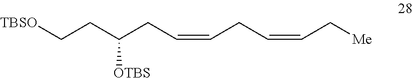 [SMILES: CC/C=C\C/C=C\C[C@@H](CCO[Si](C)(C)C(C)(C)C)O[Si](C)(C)C(C)(C)C]

### bis-Alkene 28

To a suspension of Ni(OAc)₂.4H₂O (151 mg, 0.607 mmol, 0.32 equiv.) in EtOH (20 mL) under a hydrogen atmosphere was added NaBH₄ (55 mg, 1.5 mmol, 0.77 equiv.) as a solution in EtOH (1.75 mL), and the flask headspace was evacuated and refilled with H₂ three times. After the black suspension was stirred for 10 min at 25° C., a solution of 1,2-diaminoethane (0.45 mL, 6.7 mmol, 3.6 equiv.) in EtOH (6 mL) was added, and the flask headspace was evacuated and refilled with H₂ three times. After stirring for 10 min, a solution of bis-alkyne 27 (773 mg, 1.89 mmol, 1.0 equiv.) in EtOH (20 mL) was added, and the headspace was evacuated and refilled with H₂ three more times. The reaction flask was then shielded from light with aluminum foil and stirred at 25° C. for 18 h. The reaction mixture typically turned from a black suspension to a homogeneous orange solution during this time. The H₂ atmosphere was removed, and the solution was concentrated. The crude material was redissolved in Et₂O (50 mL) and washed sequentially with sat. aq. NH₄Cl (30 mL) and brine (30 mL). The organic phase was dried (Na₂SO₄), filtered, and concentrated. Flash column chromatography (SiO₂; hexanes:Et₂O, 98:2→97:3) provided the title bis-alkene 28 (608 mg, 1.48 mmol, 78% yield) as a colorless oil. On a few occasions, small quantities of mono-reduction product 27′ could be isolated and characterized. 28: R_{f}=0.8 (silica gel, hexanes:Et₂O, 19:1); [α]_{D}²⁵=+19.5 (c=0.76, CHCl₃); FT-IR (neat) νₘₐₓ=2955, 2929, 2857, 1472, 1463, 1253, 1091, 1036, 832, 772, 713 cm⁻¹; ¹H NMR (600 MHz, CDCl₃) δ=5.45-5.37 (m, 3H), 5.33-5.28 (m, 1H), 3.86 (p, J=6.1 Hz, 1H), 3.69-3.65 (m, 2H), 2.82-2.74 (m, 2H), 2.27-2.22 (m, 2H), 2.07 (p, J=7.4 Hz, 2H), 1.69-1.60 (m, 2H), 0.87 (t, J=7.5 Hz, 3H), 0.89 (ap s, 18H), 0.06 (s, 3H), 0.05 (s, 3H), 0.04 (s, 6H) ppm; ¹³C NMR (151 MHz, CDCl₃) δ=132.08, 129.89, 127.29, 126.06, 69.27, 60.09, 40.09, 35.64, 26.10, 26.05, 25.90, 20.73, 18.43, 18.26, 14.43, 4.21, 4.55, 5.13, 5.14 ppm; HRMS (ESI) calcd for C₂₃H₄₈O₂Si₂H⁺ [M+H]⁺: 413.3266. found: 413.3251.

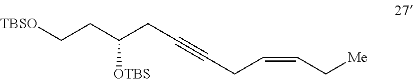 [SMILES: CC/C=C\CC#CC[C@@H](CCO[Si](C)(C)C(C)(C)C)O[Si](C)(C)C(C)(C)C]

27′: R_{f}=0.75 (silica gel, hexanes:Et₂O, 19:1); [α]_{D}²⁵=+16.9 (c=0.59, CHCl₃); FT-IR (neat) νₘₐₓ=2955, 2929, 2857, 1472, 1463, 1253, 1093, 834, 774 cm¹; ¹H NMR (600 MHz, CDCl₃) δ=5.45-5.41 (m, 1H), 5.39-5.35 (m, 1H), 3.92-3.88 (m, 1H), 3.72-3.66 (m, 2H), 2.91-2.88 (m, 2H), 2.34-2.26 (m, 2H), 2.05 (p, J=7.4 Hz, 2H), 1.85 (dtd, J=14.0, 7.3, 4.1 Hz, 1H), 1.66 (ddt, J=14.0, 7.6, 1.0 Hz, 1H), 0.97 (t, J=7.5 Hz, 3H), 0.89 (s, 9H), 0.88 (s, 9H), 0.07 (s, 3H), 0.06 (s, 3H), 0.05 (s, 3H), 0.04 (s, 3H) ppm; ¹³C NMR (151 MHz, CDCl₃) δ=133.05, 124.34, 80.30, 77.12, 68.63, 59.83, 40.00, 28.22, 26.10, 26.00, 20.58, 18.43, 18.24, 17.26, 14.16, 4.32, 4.64, 5.10, 5.13 ppm; HRMS (ESI) calcd for C₂₃H₄₆O₂Si₂H⁺ [M+H]⁺: 411.3109. found: 411.3112.

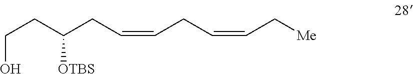 [SMILES: CC/C=C\C/C=C\C[C@@H](CCO)O[Si](C)(C)C(C)(C)C]

### Alcohol 28′

To a solution of bis-TBS-bis-alkene 28 (294 mg, 0.714 mmol, 1.0 equiv.) in MeOH (7 mL) at −10° C. was added pyridinium tribromide (23 mg, 0.072 mmol, 0.1 equiv.). The reaction mixture was stirred between −10→−5° C. for 70 min. The reaction was quenched with H₂O (10 mL), extracted with EtOAc (20 mL) and washed with brine (10 mL). The organic phase was dried (Na₂SO₄), filtered, and concentrated. Flash column chromatography (SiO₂; hexanes:EtOAc, 19:1→9:1→4:1) yielded primary alcohol 28′ (128 mg, 0.430 mmol, 60%) as a colorless oil. 28′: R_{f}=0.4 (silica gel, hexanes:EtOAc, 4:1); [α]_{D}²⁵=+31.5 (c=1.0, CHCl₃); FT-IR (neat) νₘₐₓ=3350, 3011, 2956, 2929, 2857, 1472, 1463, 1361, 1254, 1091, 1067, 834, 774 cm¹; ¹H NMR (600 MHz, CDCl₃) δ=5.47-5.34 (m, 3H), 5.31-5.27 (m, 1H), 3.98-3.94 (m, 1H), 3.83 (ddd, J=10.7, 8.3, 4.3 Hz, 1H), 3.72 (dt, J=10.7, 5.4 Hz, 1H), 2.83-2.74 (m, 2H), 2.36-2.28 (m, 2H), 2.23 (bs, 1H), 2.07 (p, J=7.5 Hz, 2H), 1.81 (ddt, J=14.2, 8.6, 4.3 Hz, 1H), 1.65 (dtd, J=14.2, 6.4, 4.3 Hz, 1H), 0.97 (t, J=7.5 Hz, 3H), 0.90 (s, 9H), 0.104 (s, 3H), 0.097 (s, 3H) ppm; ¹³C NMR (151 MHz, CDCl₃) δ=132.28, 130.46, 126.98, 125.36, 71.87, 60.49, 37.79, 35.11, 25.97, 25.90, 20.74, 18.13, 14.40, −4.21, −4.67 ppm; HRMS (ESI) calcd for C₁₇H₃₄O₂SiH⁺ [M+H]⁺: 299.2401. found: 299.2410.

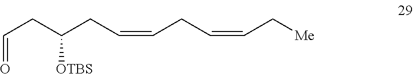 [SMILES: CC/C=C\C/C=C\C[C@@H](CC=O)O[Si](C)(C)C(C)(C)C]

### Aldehyde 29

To a solution of primary alcohol 28′ (127 mg, 0.426 mmol, 1.0 equiv.) in CH₂Cl₂ (4.5 mL) at 0° C. was added Dess-Martin periodinane (271 mg, 0.639 mmol, 1.5 equiv.) in one portion. The resulting mixture was allowed to warm to 25° C. and stirred for 90 min. The reaction was quenched sequentially by addition of sat. aq. NaHCO₃ (4 mL) and sat. aq. Na₂S₂O₃ (4 mL) and stirred for 10 min. The layers were separated, and the aq. phase was extracted with CH₂Cl₂ (10 mL). The combined organics were dried (Na₂SO₄), filtered, and concentrated. Flash column chromatography (SiO₂; hexanes:EtOAc, 98:2→95:5→93:7) yielded the title aldehyde 29 (101 mg, 0.341 mmol, 80%) as a colorless oil. 29: R_{f}=0.5 (silica gel, hexanes:EtOAc, 9:1); [α]_{D}²⁵=+23.7 (c=1.0, CHCl₃); FT-IR (neat) νₘₐₓ=3012, 2957, 2930, 2857, 1727, 1472, 1463, 1255, 1101, 836, 776 cm⁻¹; ¹H NMR (600 MHz, CDCl₃) δ=9.80 (bs, 1H), 5.51-5.47 (m, 1H), 5.42-5.36 (m, 2H), 5.30-5.26 (m, 1H), 4.25 (p, J=5.8 Hz, 1H), 2.81-2.73 (m, 2H), 2.54-2.49 (m, 2H), 2.38-2.27 (m, 2H), 2.06 (p, J=7.5 Hz, 2H), 0.97 (t, J=7.5 Hz, 3H), 0.87 (s, 9H), 0.09 (s, 3H), 0.06 (s, 3H) ppm; ¹³C NMR (151 MHz, CDCl₃) δ=202.27, 132.39, 131.27, 126.78, 124.66, 68.23, 50.63, 35.80, 25.89, 25.87, 20.75, 18.13, 14.39, −4.22, −4.67 ppm; HRMS (ESI) calcd for C₁₇H₃₂O₂SiH⁺ [M+H]⁺: 297.2244. found: 297.2236.

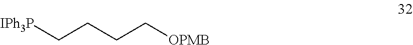

### {5-[(4-Methoxybenzyl)oxy]-n-butyl}triphenyl-phosphonium iodide 32

To a stirred solution of 1-{[(5-iodobutyl)oxy]methyl}-4-methoxybenzene 4 (2.1 g, 6.6 mmol, 1.0 equiv.) in benzene (50 mL) at 25° C. was added triphenylphosphine (8.6 g, 33 mmol, 5.0 equiv.). The reaction mixture was heated at reflux (90° C.) for 18 h, allowed to cool to 25° C., and the benzene layer was decanted from the solidified crude product. Flash column chromatography (SiO₂; CH₂Cl₂:MeOH, 98:2→95:5) yielded the title compound (3.06 g, 5.25 mmol, 81%) as a colorless amorphous solid along with some traces of IPh₃PPMB. 32: R_{f}=0.40 (silica gel, CH₂Cl₂:MeOH, 19:1); FT-IR (neat) νₘₐₓ=3055, 3010, 2932, 2861, 2793, 2187, 1611, 1586, 1511, 1437, 1302, 1246, 1178, 1111, 1028, 915, 718 cm⁻¹; ¹H NMR (600 MHz, CDCl₃) δ=7.82-7.74 (m, 10H), 7.69-7.62 (m, 5H), 7.17 (d, J=8.6 Hz, 2H), 6.81 (d, J=8.6 Hz, 2H), 4.39 (s, 2H), 3.78 (s, 3H), 3.74-3.67 (m, 2H), 3.58 (t, J=5.7 Hz, 2H), 1.99 (p, J=6.4 Hz, 2H), 1.82-1.72 (m, 2H) ppm; ¹³C NMR (151 MHz, CDCl₃) δ=159.24, 135.13 (d, J=2.9 Hz), 133.86 (d, J=10.0 Hz), 130.60 (d, J=12.5 Hz), 129.51, 118.34 (d, J=86.1 Hz), 113.86, 72.56, 68.57, 55.46, 29.63 (d, J=16.4 Hz), 22.49 (d, J=50.0 Hz), 19.74 (d, J=4.1 Hz) ppm; ³¹P NMR (162 MHz, CDCl₃) δ=24.81 ppm; HRMS (ESI) calcd for C₃₀H₃₂O₂P⁺ [M-I]⁺: 455.2134. found: 455.2138.

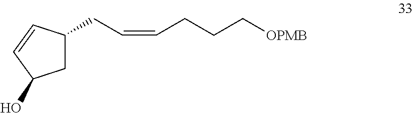

### Diene 33

To a stirred solution of t-butyldimethylsilyl-alcohol 30 (660 mg, 2.44 mmol, 1.0 equiv.) in CH₂Cl₂ (10 mL) at −78° C. was dropwise added diisobutylaluminum hydride (1 M in CH₂Cl₂, 2.6 mL, 2.6 mmol, 1.1 equiv.). After stirring for 45 min, the clear solution was quenched with H₂O (0.9 mL, 50 mmol, 20 equiv.), diluted with Et₂O (5.6 mL) and allowed to warm to 25° C. Under vigorous stirring, NaF (1.0 g, 24 mmol, 10 equiv.) was added and the resulting mixture was stirred for an additional 30 min before it was filtered through Celite®, washed with Et₂O, and concentrated. Flash column chromatography (SiO₂; pentane:Et₂O, 10:1) yielded the corresponding aldehyde (550 mg, 2.29 mmol), which was taken to the next step without further purification. To a stirred solution of {5-[(4-methoxybenzyl)oxy]-n-butyl}-triphenyl-phosphonium iodide 32 (2.0 g, 3.4 mmol, 1.5 equiv.) in THF (23 mL) at 0° C. was added dropwise sodium bis(trimethylsilyl)amide (1 M in THF, 4.6 mL, 4.6 mmol, 2.0 equiv.). After stirring for 30 min at this temperature, the resulting bright orange solution was cooled to −78° C., and the aldehyde obtained above (550 mg, 2.29 mmol, 1.0 equiv.) in THF (1.5 mL) was added dropwise. After stirring at this temperature for 1 h, the reaction mixture was allowed to warm to 25° C. over 2 h and stirred for an additional 12 h. The reaction was then quenched with sat. aq. NH₄Cl solution (20 mL) and extracted with hexane (4×20 mL). The combined organic extracts were dried (Na₂SO₄), filtered, and concentrated. Flash column chromatography (SiO₂; pentane:Et₂O, 25:1→8:1) yielded the title compound along with a small impurity as a colorless oil which was taken to the next step without further purification. To a stirred solution of the obtained diene (700 mg, 1.68 mmol, 1.0 equiv.) in THF (20 mL) at 0° C. was added dropwise tetra-n-butylammonium fluoride (1 M in THF, 2.0 mL, 2.0 mmol, 1.2 equiv.). After warming the reaction mixture to 25° C., stirring was continued for 3 h. The brown-colored reaction was then quenched with sat. aq. NH₄Cl solution (20 mL) and diluted with EtOAc (20 mL). The phases were separated, the aq. layer was extracted with EtOAc (3×20 mL), and the combined organic extracts were washed with brine (20 mL), dried (MgSO₄), filtered, and concentrated. Flash column chromatography (SiO₂; hexanes:EtOAc, 5:2) yielded the pure title compound (500 mg, 1.65 mmol, 68% for the three steps, d.r. >20:1 by ¹H NMR analysis) as a colorless oil. 33: R_{f}=0.32 (silica gel, hexanes:EtOAc, 2:1); [α]_{D}²⁵=+17.2 (c=1.0, CHCl₃); FT-IR (neat) νₘₐₓ=3391, 3004, 2931, 2853, 1612, 1586, 1512, 1301, 1245, 1172, 1095, 1033, 819, 758 cm⁻¹; ¹H NMR (600 MHz, CDCl₃) δ=7.26 (d, J=8.3 Hz, 2H), 6.89 (d, J=8.3 Hz, 2H), 5.88-5.83 (m, 1H), 5.81-5.78 (m, 1H), 5.49-5.43 (m, 1H), 5.43-5.36 (m, 1H), 4.77 (t, J=5.9 Hz, 1H), 4.43 (s, 2H), 3.80 (s, 3H), 3.44 (t, J=6.5 Hz, 2H), 2.63 (p, J=7.1 Hz, 1H), 2.45 (dt, J=13.7, 7.7 Hz, 1H), 2.25-2.18 (m, 1H), 2.16-2.09 (m, 3H), 1.80 (bs, 1H), 1.66 (p, J=7.0 Hz, 2H), 1.26 (dt, J=13.7, 4.9 Hz, 1H) ppm; ¹³C NMR (151 MHz, CDCl₃) δ=159.19, 138.22, 133.59, 130.76, 130.75, 129.34, 128.07, 113.85, 77.29, 72.62, 69.52, 55.36, 44.52, 39.81, 33.66, 29.74, 24.08 ppm; HRMS (ESI) calcd for C₁₉H₂₆O₃Na⁺ [M+Na]⁺: 325.1774. found: 325.1781.

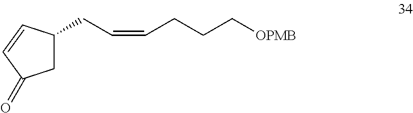

### Enone 34

To a vigorously stirred solution of allylic alcohol 33 (120 mg, 0.397 mmol, 1.0 equiv.) in CH₂Cl₂ (3.8 mL) at 25° C. was added in one portion pyridinium chlorochromate (164 mg, 0.76 mmol, 1.9 equiv.). After stirring for 2 h, the reaction mixture was diluted with Et₂O (10 mL), filtered through Celite®, washed with Et₂O, and concentrated. Flash column chromatography (SiO₂; hexanes:EtOAc, 3:1) yielded the pure title compound (110 mg, 0.367 mmol, 92%) as a colorless oil. 34: R_{f}=0.59 (silica gel, hexanes:EtOAc, 3:2); [α]_{D}²⁵=+86.1 (c=1.1, CHCl₃); FT-IR (neat) νₘₐₓ=3006, 2934, 2855, 1707, 1612, 1586, 1512, 1302, 1246, 1179, 1097, 1034, 820, 783 cm⁻¹; ¹H NMR (500 MHz, CDCl₃) δ=7.59 (dd, J=5.7, 2.5 Hz, 1H), 7.24 (d, J=8.6 Hz, 2H), 6.86 (d, J=8.6 Hz, 2H), 6.14 (dd, J=5.7, 2.0 Hz, 1H), 5.53-5.46 (m, 1H), 5.39-5.32 (m, 1H), 4.41 (s, 2H), 3.79 (s, 3H), 3.42 (t, J=6.4 Hz, 2H), 3.01-2.94 (m, 2H), 2.49 (dd, J=18.8, 6.4 Hz, 1H), 2.32-2.25 (m, 1H), 2.23-2.13 (m, 1H), 2.10 (q, J=7.3 Hz, 2H), 1.99 (dd, J=18.8, 2.2 Hz, 1H), 1.65 (dt, J=13.5, 6.6 Hz, 2H) ppm; ¹³C NMR (126 MHz, CDCl₃) δ=209.83, 168.00, 159.20, 134.16, 132.03, 130.67, 129.28, 126.17, 113.83, 72.65, 69.34, 55.34, 41.45, 40.56, 31.92, 29.62, 24.08 ppm; HRMS (ESI) calcd for C₁₉H₂₄O₃Na⁺ [M+Na]⁺: 323.1618. found: 323.1614.

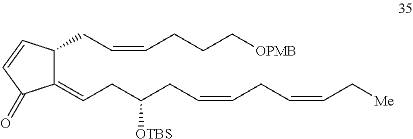

### Dienone 35

To a stirred solution of diisopropylamine (86 μL, 0.62 mmol, 2.2 equiv. in THF (2.5 mL) at 0° C. was added dropwise n-BuLi (2.5 M in hexanes, 0.22 mL, 0.56 mmol, 2.0 equiv.). After stirring for 20 min at this temperature, the clear solution was cooled to −78° C., and a solution of enone 34 (82 mg, 0.27 mmol, 1.0 equiv.) in THF (2.5 mL) was added dropwise. After stirring the resulting slightly yellow solution for an additional 20 min at this temperature, a solution of aldehyde 29 (100 mg, 0.338 mmol, 1.3 equiv.) in THF (2.5 mL) was added dropwise, and stirring at this temperature was continued for an additional 30 min. The reaction was then quenched with sat. aq. NH₄Cl solution (7 mL), diluted with EtOAc (7 mL), and allowed to warm to 25° C. The phases were separated, the aq. layer was extracted with EtOAc (8 mL), and the combined organic extracts were washed with brine (8 mL), dried (Na₂SO₄), filtered, and concentrated. The crude aldol product (34a) was filtered through a short column (SiO₂; hexanes:EtOAc, 19:1→7:3) to obtain a mixture of diastereomeric alcohols (83 mg, 0.14 mmol, 52%) as a colorless oil which was taken to the next step without further purification. To a stirred solution of aldol product 34a (82 mg, 0.14 mmol, 1.0 equiv.) in CH₂Cl₂ (2 mL) at 0° C. was added Et₃N (0.19 mL, 1.4 mmol, 10 equiv.), and then, slowly and dropwise, methanesulfonyl chloride (53 μL; 0.69 mmol, 5.0 equiv.). After stirring for 60 min at this temperature, the reaction was quenched with sat. aq. NaHCO₃ solution (4 mL), diluted with CH₂Cl₂ (5 mL), and allowed to warm to 25° C. The phases were separated, and the organic layer was washed with H₂O (10 mL), dried (Na₂SO₄), filtered, and concentrated. The crude mixture (34b) was filtered through a short column (SiO₂; hexanes:EtOAc, 3:1) to obtain a mixture of diastereomeric mesylates as a colorless oil which was taken to the next step without further purification. To a vigorously stirred solution of mesylate 34b (mixture of diastereomers) in CH₂Cl₂ (3 mL) at 25° C. was added Al₂O₃ (100 mg, 0.98 mmol, 7.0 equiv.). After 2 h and 4 h time intervals two more portions of Al₂O₃ (2×100 mg, 2×0.98 mmol, 2×7.0 equiv.) were added and vigorous stirring was continued for a total of 6 h. The resulting suspension was then filtered through Celite®, washed with EtOAc, and the solution so obtained was concentrated. Flash column chromatography (SiO₂; hexanes:EtOAc, 19:1→9:1→6:1) yielded pure title compound (47 mg, 0.081 mmol, 30% for the three steps) as a colorless oil.

35: R_{f}=0.6 (silica gel, hexanes:EtOAc, 4:1); [α]_{D}²⁵=+108.8 (c=1.0, CHCl₃); FT-IR (neat) νₘₐₓ=3009, 2954, 2931, 2856, 1704, 1656, 1613, 1513, 1462, 1248, 1095, 1038, 835, 775 cm⁻¹; ¹H NMR (600 MHz, CDCl₃) δ=7.47 (ddd, J=5.9, 2.4, 0.8 Hz, 1H), 7.24 (d, J=8.5 Hz, 2H), 6.87 (d, J=8.5 Hz, 2H), 6.60 (dd, J=8.5, 6.9 Hz, 1H), 6.31 (dd, J=6.0, 1.8 Hz, 1H), 5.51-5.32 (m, 5H), 5.28-5.24 (m, 1H), 4.41 (s, 2H), 3.90 (p, J=6.0 Hz, 1H), 3.80 (s, 3H), 3.46-3.41 (m, 3H), 2.78-2.69 (m, 2H), 2.63-2.59 (m, 1H), 2.45-2.39 (m, 2H), 2.30-2.15 (m, 3H), 2.10-2.01 (m, 4H), 1.64 (p, J=7.0 Hz, 2H), 0.96 (t, J=7.5 Hz, 3H), 0.88 (s, 9H), 0.06 (s, 3H), 0.05 (s, 3H) ppm; ¹³C NMR (151 MHz, CDCl₃) δ=196.39, 161.72, 159.28, 138.95, 134.99, 132.51, 132.25, 132.15, 130.74, 130.66, 129.34, 127.00, 125.64, 125.34, 113.91, 72.74, 71.60, 69.53, 55.42, 43.54, 36.97, 35.39, 30.65, 29.74, 25.99, 25.90, 24.18, 20.74, 18.22, 14.40, −4.39, −4.41 ppm; HRMS (ESI) calcd for C₃₆H₅₄O₄SiNa⁺ [M+Na]⁺: 601.3684. found: 601.3661.

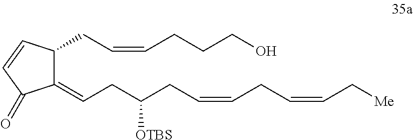 [SMILES: CC/C=C\C/C=C\C[C@@H](C/C=C1/C(=O)C=C[C@@H]1C/C=C\CCCO)O[Si](C)(C)C(C)(C)C]

### Primary alcohol 35a

To a vigorously stirred solution of dienone 35 (39 mg, 0.067 mmol, 1.0 equiv.) in a mixture of CH₂Cl₂:H₂O (10:1, 1.98 mL) at 0° C. was added in one portion 2,3-dichloro-5,6-dicyano-1,4-benzoquinone (23 mg, 0.10 mmol, 1.5 equiv.). After stirring at this temperature for 75 min, an additional portion of 2,3-dichloro-5,6-dicyano-1,4-benzoquinone (5 mg, 0.02 mmol, 0.2 equiv.) was added. After stirring for an additional 20 min, the reaction mixture was diluted with Et₂O (3 mL), filtered through Celite®, washed with Et₂O, and concentrated to a volume of ca. 0.5 mL (not to dryness!). Flash column chromatography (SiO₂; hexanes:EtOAc, 9:1→3:1→3:2) yielded pure title compound (20 mg, 0.044 mmol, 66%) as a colorless oil. 35a: R_{f}=0.3 (silica gel, hexanes:EtOAc, 7:3); [α]_{D}²⁵=+138.3 (c=1.0, CHCl₃); FT-IR (neat) νₘₐₓ=3444, 3010, 2955, 2929, 2856, 1701, 1652, 1462, 1254, 1071, 835, 775 cm⁻¹; ¹H NMR (600 MHz, CDCl₃) δ=7.50 (ddd, J=6.0, 2.5, 0.8 Hz, 1H), 6.62-6.59 (m, 1H), 6.33 (dd, J=6.0, 1.8 Hz, 1H), 5.53-5.35 (m, 5H), 5.29-5.24 (m, 1H), 3.91 (p, J=6.0 Hz, 1H), 3.64 (t, J=6.4 Hz, 2H), 3.48-3.44 (m, 1H), 2.79-2.69 (m, 2H), 2.67-2.63 (m, 1H), 2.47-2.42 (m, 2H), 2.31-2.18 (m, 3H), 2.12-2.08 (m, 2H), 2.04 (p, J=7.3 Hz, 2H), 1.61 (p, J=7.1 Hz, 2H), 0.96 (t, J=7.5 Hz, 3H), 0.88 (s, 9H), 0.07 (s, 3H), 0.06 (s, 3H) ppm; ¹³C NMR (151 MHz, CDCl₃) δ=196.37, 161.61, 138.94, 135.07, 132.58, 132.28, 131.99, 130.69, 126.99, 125.87, 125.32, 71.63, 62.47, 43.54, 36.97, 35.42, 32.58, 30.65, 25.99, 25.90, 23.84, 20.74, 18.23, 14.40, −4.38, −4.40 ppm; HRMS (ESI) calcd for C₂₈H₄₆O₃SiNa⁺ [M+Na]⁺: 481.3108. found: 481.3103.

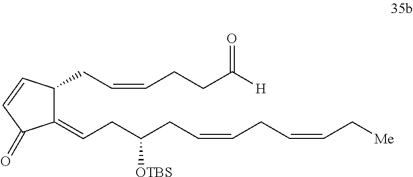 [SMILES: CC/C=C\C/C=C\C[C@@H](C/C=C1/C(=O)C=C[C@@H]1C/C=C\CCC=O)O[Si](C)(C)C(C)(C)C]

### Aldehyde 35b

To a vigorously stirred solution of alcohol 35a (19 mg, 0.041 mmol, 1.0 equiv.) in CH₂Cl₂ (1 mL) at 25° C. was added in one portion pyridinium chlorochromate (18 mg, 0.082 mmol, 2.0 equiv.). After stirring for 75 min, the reaction mixture was filtered through a short pad of Celite® (Et₂O), and concentrated. Flash column chromatography (SiO₂; hexanes:EtOAc, 19:1→9:1-4:1) provided the title aldehyde 35b (15 mg, 0.033 mmol, 80%) as a colorless oil. 35b: R_{f}=0.5 (silica gel, hexanes:EtOAc, 7:3); [α]_{D}²⁵=+136.3 (c=1.0, CHCl₃); FT-IR (neat)=3011, 2957, 2930, 2856, 1726, 1704, 1656, 1255, 1089, 836, 808, 776 cm⁻¹; ¹H NMR (600 MHz, CDCl₃) δ=9.76 (bs, 1H), 7.48 (dd, J=6.0, 2.6 Hz, 1H), 6.61 (t, J=7.7 Hz, 1H), 6.34 (dd, J=6.0, 1.2 Hz, 1H), 5.49-5.36 (m, 5H), 5.29-5.23 (m, 1H), 3.91 (p, J=6.0 Hz, 1H), 3.48-3.46 (m, 1H), 2.79-2.69 (m, 2H), 2.67-2.63 (m, 1H), 2.50-2.46 (m, 2H), 2.45-2.41 (m, 2H), 2.36-2.21 (m, 5H), 2.04 (p, J=7.4 Hz, 2H), 0.96 (t, J=7.5 Hz, 3H), 0.88 (s, 9H), 0.06 (s, 3H), 0.05 (s, 3H) ppm; ¹³C NMR (151 MHz, CDCl₃) δ=201.54, 196.24, 161.34, 138.79, 135.20, 132.70, 132.27, 130.69, 130.26, 126.98, 126.75, 125.29, 71.58, 43.68, 43.32, 36.97, 35.42, 30.60, 25.99, 25.90, 20.74, 20.22, 18.22, 14.40, −4.39, −4.41 ppm; HRMS (ESI) calcd for C₂₈H₄₄O₃SiNa⁺ [M+Na]⁺: 479.2952. found: 479.2935.

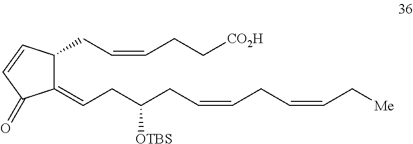 [SMILES: CC/C=C\C/C=C\C[C@@H](C/C=C1/C(=O)C=C[C@@H]1C/C=C\CCC(=O)O)O[Si](C)(C)C(C)(C)C]

### Acid 36

To a vigorously stirred solution of aldehyde 35b (15 mg, 0.033 mmol, 1.0 equiv.) in t-BuOH (0.8 mL) and H₂O (0.6 mL) at 25° C. were added sequentially 2-methyl-2-butene (35 μL, 0.33 mmol, 10 equiv.), NaH₂PO₄ (7.6 mg, 0.049 mmol, 1.5 equiv.) and NaClO₂ (4.4 mg, 0.049 mmol, 1.5 equiv.). After stirring for 30 min, the reaction mixture was diluted with a solution of NaH₂PO₄ (500 mg) in H₂O (10 mL) and extracted with EtOAc (2×7 mL). The combined organic extracts were dried (Na₂SO₄), filtered, and concentrated. Flash column chromatography (SiO₂; CH₂Cl₂:EtOH, 49:1→19:1) yielded pure title compound (15 mg, 0.032 mmol, 97%) as a colorless oil. 36: R_{f}=0.45 (silica gel, CH₂Cl₂:EtOH, 19:1); [α]_{D}²⁵=+93.4 (c=1.0, CHCl₃); FT-IR (neat)=3011, 2956, 2929, 2856, 1707, 1654, 1462, 1253, 1087, 966, 835, 808, 775 cm⁻¹; ¹H NMR (600 MHz, CDCl₃) δ=7.48 (dd, J=6.0, 2.6, 1.0 Hz, 1H), 6.61 (t, J=7.7 Hz, 1H), 6.34 (dd, J=6.0, 1.8 Hz, 1H), 5.49-5.35 (m, 5H), 5.29-5.24 (m, 1H), 3.91 (p, J=6.0 Hz, 1H), 3.49-3.46 (m, 1H), 2.78-2.70 (m, 2H), 2.67-2.63 (m, 1H), 2.45-2.43 (m, 2H), 2.40-2.38 (m, 2H), 2.35-2.20 (m, 5H), 2.04 (p, J=7.3 Hz, 2H), 0.96 (t, J=7.5 Hz, 3H), 0.88 (s, 9H), 0.06 (s, 3H), 0.05 (s, 3H) ppm; ¹³C NMR (151 MHz, CDCl₃) δ=196.33, 176.70, 161.44, 138.81, 135.16, 132.74, 132.27, 130.69, 130.12, 127.00, 126.96, 125.32, 71.63, 43.36, 36.96, 35.40, 33.54, 30.59, 25.99, 25.90, 22.75, 20.74, 18.23, 14.40, −4.39, −4.41 ppm; HRMS (ESI) calcd for C₂₈H₄₄O₄SiNa⁺ [M+Na]⁺: 495.2901. found: 495.2882.

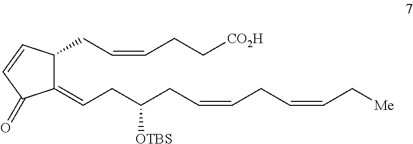 [SMILES: CC/C=C\C/C=C\C[C@@H](C/C=C1/C(=O)C=C[C@@H]1C/C=C\CCC(=O)O)O[Si](C)(C)C(C)(C)C]

### Δ¹¹-NPJ₄ (7)

To a stirred solution of TBS ether 36 (15 mg, 0.032 mmol, 1.0 equiv.) in MeCN (0.6 mL) at 0° C. was added dropwise a solution of HF (50% aq., 60 μL, ca. 1.7 mmol, ca. 50 equiv.) in MeCN (0.1 mL). After stirring for 30 min, additional HF (50% aq., 60 μL, ca. 1.7 mmol, ca. 50 equiv.) in MeCN (0.1 mL) was added. After stirring for an additional 45 min, the reaction was quenched with brine (3 mL) and extracted with EtOAc (3 mL). The organic extract was dried (Na₂SO₄), filtered, and concentrated. Flash column chromatography (SiO₂; CH₂Cl₂:EtOH, 99:1→98:2→97:3→96:4) yielded the pure title compound (8.9 mg, 0.026 mmol, 81%) as a colorless oil. 7: R_{f}⁼0.6 (silica gel, CH₂Cl₂:EtOH, 9:1); [α]_{D}²⁵=+65.1 (c=0.59, CHCl₃); UV(EtOH) λₘₐₓ (log ε)=245 (3.89) nm; FT-IR (neat) νₘₐₓ=3408, 3011, 2963, 2932, 1705, 1646, 1430, 1240, 1210, 1068, 1035, 839, 719 cm⁻¹; ¹H NMR (600 MHz, CDCl₃) δ=7.51 (dd, J=5.8, 2.5 Hz, 1H), 6.64 (t, J=7.6 Hz, 1H), 6.34 (dd, J=5.9, 1.3 Hz, 1H), 5.60-5.54 (m, 1H), 5.50-5.34 (m, 4H), 5.32-5.26 (m, 1H), 3.88 (p, J=6.2 Hz, 1H), 3.55-3.52 (m, 1H), 2.82-2.78 (m, 2H), 2.70-2.64 (m, 1H), 2.56-2.45 (m, 2H), 2.40-2.30 (m, 7H), 2.06 (p, J=7.3 Hz, 2H), 0.97 (t, J=7.5 Hz, 3H) ppm; ¹³C NMR (151 MHz, CDCl₃) δ=196.54, 176.76, 161.79, 139.56, 135.15, 132.53, 132.37, 131.95, 130.32, 126.71, 126.66, 124.73, 70.83, 43.32, 36.56, 35.00, 33.68, 30.16, 25.88, 23.03, 20.76, 14.40 ppm; HRMS (ESI) calcd for C₂₂H₃₀O₄H⁺ [M+H]⁺: 359.2217. found: 359.2215.

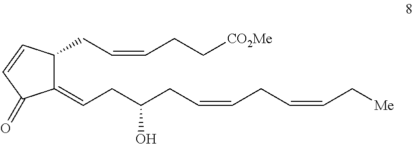 [SMILES: CC/C=C\C/C=C\C[C@H](O)C/C=C1/C(=O)C=C[C@@H]1C/C=C\CCC(=O)OC]

### Δ¹¹-NPJ₄ methyl ester (8)

To a stirred solution of Δ¹¹-NPJ₄ (7) (23.0 mg, 67.0 μmol, 1.0 equiv.) in C₆H₆:MeOH (3:2, 2.2 mL) at 25° C. was added dropwise a solution of trimethylsilyl diazomethane (2 M in Et₂O, 50 μL, 100 μmol, 1.5 equiv.) (yellow color persists). After stirring for 30 min, the reaction mixture was concentrated. Flash column chromatography (SiO₂; hexanes:EtOAc, 3:1→2:1) yielded the pure title compound (18.0 mg, 48.3 μmol, 72%) as a colorless oil. 8: R_{f}=0.57 (silica gel, hexanes:EtOAc, 1:1); [α]_{D}²⁵=+147.7 (c=0.39, CHCl₃); UV(EtOH) λₘₐₓ (log ε)=242 (4.15) nm; FT-IR (neat) νₘₐₓ=3402, 3016, 2961, 2924, 2853, 1737, 1701, 1650, 1579, 1437, 1370, 1263, 1163, 1063 cm⁻¹; ¹H NMR (600 MHz, CDCl₃) δ=7.50 (dd, J=6.0, 2.6 Hz, 1H), 6.54 (t, J=6.9 Hz, 1H), 6.34 (dd, J=6.0, 1.8 Hz, 1H), 5.61-5.53 (m, 1H), 5.48-5.33 (m, 4H), 5.31-5.24 (m, 1H), 3.86 (p, J=6.2 Hz, 1H), 3.66 (s, 3H), 3.54-3.50 (m, 1H), 2.80 (t, J=7.3 Hz, 2H), 2.71-2.63 (m, 1H), 2.56-2.43 (m, 2H), 2.37-2.24 (m, 7H), 2.09-2.01 (m, 2H), 1.97 (bs, 1H), 0.96 (t, J=7.5 Hz, 3H) ppm; ¹³C NMR (151 MHz, CDCl₃) δ=196.39, 173.61, 161.63, 139.50, 135.13, 132.48, 132.25, 131.84, 130.60, 126.72, 126.45, 124.82, 70.65, 51.78, 43.36, 36.72, 35.15, 33.92, 30.32, 25.87, 23.03, 20.74, 14.37 ppm; HRMS (ESI) calcd for C₂₃H₃₂O₄Na⁺ [M+Na]⁺: 395.2193. found: 395.2183.

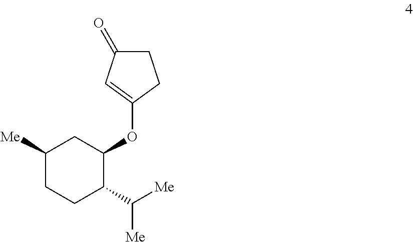 [SMILES: CC(C)[C@@H]1CC[C@@H](C)C[C@H]1OC1=CC(=O)CC1]

### Menthyl enol ether 4

To a stirred solution of 1,3-cyclopentanedione (10.0 g, 100.0 mmol, 1.0 equiv) and L-(−)-menthol (18.9 g, 120.0 mmol, 1.2 equiv) in benzene (250 mL) was added p-toluenesulfonic acid (1.9 g, 10 mmol, 0.1 equiv). The resulting mixture was heated to 80° C. for 12 h using a Dean-Stark trap. The reaction mixture was allowed to cool to 25° C., diluted with Et₂O (300 mL), and the combined organic layers were washed sequentially with saturated aqueous NaHCO₃ solution (50 mL) and brine (30 mL). The organic phase was dried (Na₂SO₄), filtered, and concentrated under reduced pressure. Purification by flash column chromatography (SiO₂; hexane:EtOAc, 19:1→4:1) gave pure title compound (4, 21.0 g, 81.0 mmol, 81% yield) as colorless crystals. 4: R_{f}⁼0.50 (hexane:EtOAc, 3:1); mp=59-63° C.; [α]_{D}²⁵=−152.8° (c=1.00, CHCl₃); IR (film): νₘₐₓ=2954, 2926, 2869, 1705, 1679, 1585, 1332, 1248, 1189, 962 cm⁻¹; ¹H NMR (600 MHz, CDCl₃) δ 5.28 (s, 1H), 3.93 (td, J=10.8, 7.4 Hz, 1H), 2.61-2.56 (m, 2H), 2.41 (t, J=5.2 Hz, 2H), 2.10 (bd, J=12.0 Hz, 1H), 1.99-1.96 (m, 1H), 1.84-1.77 (m, 1H), 1.70 (bd, J=14.0 Hz, 2H), 1.55-1.38 (m, 2H), 1.10-1.00 (m, 2H), 0.92 (d, J=6.8 Hz, 3H), 0.90 (d, J=6.8 Hz, 3H), 0.75 ppm (d, J=6.8 Hz, 3H); ¹³C NMR (150 MHz, CDCl₃) δ 206.23, 189.62, 104.35, 82.51, 47.50, 39.52, 34.20, 33.79, 31.44, 29.03, 26.38, 23.64, 22.05, 20.65, 16.72 ppm; HRMS (ESI-TOF) calcd for C₁₅H₂₄O₂Na⁺ [M+Na]⁺: 259.1673. found: 259.1664.

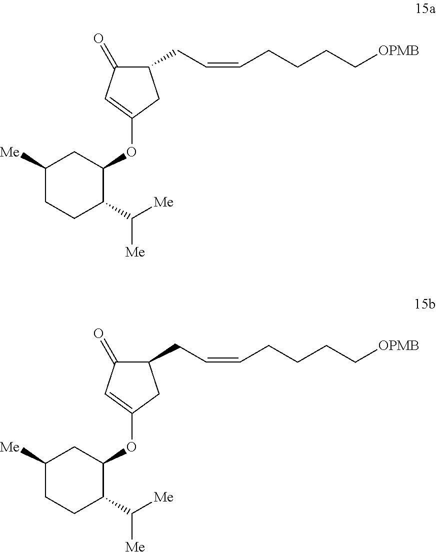

### PMB ether 15a and 15b

To a stirred solution of diisopropylamine (5.4 mL, 38.4 mmol, 1.2 equiv) in THF (60 mL) at 0° C. was dropwise added n-butyl lithium (2.5 M in hexane, 14.7 mL, 36.8 mmol, 1.15 equiv). The resulting solution was stirred at 0° C. for 1 h and then cooled at −78° C. A solution of ketone 4 (8.1 g, 32.0 mmol, 1.00 equiv) in THF (60 mL) was added dropwise over a period of 30 min. The resulting yellow solution was stirred at −78° C. for 45 min and allylic bromide 5 (13.0 g, 41.6 mmol, 1.3 equiv) in THF (10 mL) was added dropwise over a period of 30 min After another 5 min 1,3-dimethyl-2-imidazolidinone (4.38 mL, 38.4 mmol, 1.2 equiv) was added dropwise over a period of 15 min After stirring at the same temperature for an additional 2 h, the reaction mixture was slowly warmed to −40° C. and stirred for an additional 8 h. The reaction mixture was warmed to 25° C. and partitioned between saturated aqueous NH₄Cl solution (200 mL) and EtOAc (200 mL). The organic layer was separated and washed sequentially with H₂O (100 mL) and saturated aqueous NaCl solution (100 mL), and the combined aqueous layers were back-extracted with EtOAc (2×200 mL). The combined organic layers were dried (Na₂SO₄), filtered, and concentrated under reduced pressure. Purification by flash column chromatography (SiO₂, hexane:EtOAc, 19:1→6:1) gave pure title compound (15a, 4.5 g, 9.6 mmol, 30% yield) as well as 15a and 15b as a mixture of diastereoisomers (2.9 g, 15a:15b, 1:4; 2.1 g, 15a:15b, 3:2; 6.9 g, 15a:15b, 6:1) contaminated with bis-alkylation product (app. 5-10%, inseparable) as colorless oils. 15a: R_{f}⁼0.60 (hexane:EtOAc, 3:1); [α]_{D}²⁵=−97.4° (c=2.00, CHCl₃); IR (film): νₘₐₓ=3006, 2929, 2866, 1686, 1583, 1512, 1456, 1244, 1172, 728 cm⁻¹; ¹H NMR (600 MHz, CDCl₃) δ 7.25 (d, J=8.5 Hz, 2H), 6.87 (d, J=8.5 Hz, 2H), 5.45 (dt, J=10.7, 7.8 Hz, 1H), 5.31-5.27 (m, 1H), 5.25 (s, 1H), 4.42 (s, 2H), 3.97 (td, J=10.7, 4.2 Hz, 2H), 3.80 (s, 3H), 3.43 (t, J=6.5 Hz, 2H), 2.65 (dd, J=7.6, 6.9 Hz, 1H), 2.55-2.49 (m, 2H), 2.26 (dd, J=17.6, 2.3 Hz, 1H), 2.21-2.16 (m, 1H), 2.11-2.04 (m, 3H), 1.97 (pd, J=7.0, 2.7 Hz, 1H), 1.71-1.69 (m, 2H), 1.61-1.58 (m, 2H), 1.49 (ddt, J=13.3, 10.3, 3.2 Hz, 1H), 1.45-1.40 (m, 3H), 1.09-1.01 (m, 2H), 0.93 (d, J=6.6 Hz, 3H), 0.90 (d, J=7.1 Hz, 3H), 0.76 ppm (d, J=6.9 Hz, 3H); ¹³C NMR (150 MHz, CDCl₃) δ=207.76, 188.58, 159.22, 132.19, 130.87, 129.33, 125.98, 113.87, 103.74, 82.51, 72.65, 70.08, 55.40, 47.51, 44.69, 39.58, 34.78, 34.24, 31.47, 29.52, 28.95, 27.25, 26.55, 26.39, 23.82, 22.08, 20.62, 16.87 ppm; HR-MS (ESI-TOF): calcd for C₃₀H₄₄O₄Na⁺ [M+Na]⁺: 491.3137. found: 491.3118. 15b: R_{f}⁼0.65 (hexane:EtOAc, 3:1); [c]_{D}²⁵=−40.7° (c=1.00, CHCl₃); IR (film): νₘₐₓ=3005, 2932, 2866, 1691, 1587, 1513, 1458, 1332, 1246, 1098, 820 cm⁻¹; ¹H NMR (600 MHz, CDCl₃) δ 7.26 (d, J=8.5 Hz, 2H), 6.87 (d, J=8.5 Hz, 2H), 5.45 (dt, J=10.7, 7.8 Hz, 1H); 5.32-5.28 (m, 1H), 5.25 (s, 1H), 4.42 (s, 2H), 3.97 (td, J=10.7, 4.2 Hz, 2H), 3.80 (s, 3H), 3.43 (t, J=6.5 Hz, 2H), 2.69 (dd, J=18.1, 7.3 Hz, 1H), 2.55-2.49 (m, 2H), 2.23 (dd, J=18.1, 2.5 Hz, 1H), 2.20-2.15 (m, 1H), 2.11 (dt, J=12.4, 3.6 Hz, 1H), 2.06 (q, J=6.7 Hz, 2H), 2.00-1.95 (m, 1H), 1.72-1.69 (m, 2H), 1.63-1.58 (m, 2H), 1.49 (ddt, J=13.3, 10.3, 3.2 Hz, 1H), 1.45-1.39 (m, 3H), 1.09-1.01 (m, 2H), 0.93 (d, J=6.6 Hz, 3H), 0.90 (d, J=7.1 Hz, 3H), 0.75 ppm (d, J=6.9 Hz, 3H); ¹³C NMR (150 MHz, CDCl₃) δ 207.74, 188.52, 159.23, 132.13, 130.87, 129.34, 126.15, 113.88, 103.62, 82.44, 72.64, 70.07, 55.39, 47.51, 44.74, 39.56, 34.81, 34.22, 31.47, 29.48, 28.83, 27.24, 26.41, 26.36, 23.68, 22.07, 20.66, 16.77 ppm; HR-MS (ESI-TOF): calcd for C₃₀H₄₅O₄⁺ [M+H]⁺: 469.3319. found: 469.3313.

### Epimerization of 15b to 15a

To a stirred solution of PMB-ethers 15a and 15b (15a:15b, 1:4, 2.90 g, 6.20 mmol, 1.0 equiv) in THF (6 mL) at 0° C. was added t-BuOH (0.30 mL, 3.70 mmol, 0.6 equiv) and t-BuOK (347 mg, 3.1 mmol, 0.5 equiv). The resulting brown mixture was stirred for 2 h and then diluted with saturated aqueous NH₄Cl solution (6 mL) and stirred for 10 min. The reaction mixture was partitioned between saturated aqueous NH₄Cl solution (10 mL) and EtOAc (10 mL). The organic layer was separated and washed with H₂O (10 mL) and brine (10 mL), and the combined aqueous layers were extracted with EtOAc (2×10 mL). The combined organic layers were dried (Na₂SO₄), filtered, and concentrated under reduced pressure to give a mixture of 15a and 15b (15a:15b, 1:1, 2.6 g, 5.6 mmol, 91% yield). Purification by flash column chromatography (SiO₂, hexane:EtOAc, 19:1→6:1) gave pure 15a as a colorless oil.

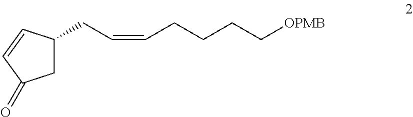

### Enone 2

To a stirred solution of PMB ether 15a (4.60 g, 9.80 mmol, 1.0 equiv) in Et₂O (50 mL) at −10° C. was dropwise added diisobutylaluminum hydride (1.0 M in CH₂Cl₂, 14.7 mL, 14.7 mmol, 1.5 equiv). After stirring for 30 min, the clear solution was cooled to −78° C. and quenched with MeOH (2 mL), diluted with Et₂O (50 mL) and allowed to warm to 25° C. Under vigorous stirring, saturated Na—K tartrate-solution (50 ml) was added and the resulting mixture was stirred for an additional 3 h. The resultant biphasic mixture was extracted with Et₂O (3×100 mL), and the combined organic layers were washed sequentially with H₂O (30 mL) and brine (30 mL). The organic phase was dried (Na₂SO₄), filtered, and concentrated under reduced pressure. Purification by flash column chromatography (SiO₂, hexane:EtOAc, 19:1→4:1) gave pure title compound (2, 2.30 g, 7.44 mmol, 76% yield) as a colorless oil. 2: R_{f}=0.50 (hexane:EtOAc, 3:1); [α]_{D}²⁵=91.7° (c=1.00, C₆H₆); IR (film): νₘₐₓ=3006, 2932, 2855, 1710, 1662, 1612, 1586, 1512, 1246, 1097, 1034 cm⁻¹; ¹H NMR (600 MHz, CDCl₃) δ 7.61 (dd, J=5.6, 2.5 Hz, 1H), 7.25 (d, J=8.9 Hz, 2H), 6.87 (d, J=8.6 Hz, 2H), 6.16 (dd, J=5.6, 2.0 Hz, 1H), 5.53-5.46 (m, 1H), 5.38-5.31 (m, 1H), 4.42 (s, 2H), 3.80 (s, 3H), 3.43 (t, J=6.5 Hz, 2H), 2.99 (dddt, J=11.6, 6.8, 4.5, 2.2 Hz, 1H), 2.50 (dd, J=18.9, 6.4 Hz, 1H), 2.32-2.24 (m, 1H), 2.23-2.15 (m, 1H), 2.07-1.97 (m, 3H), 1.65-1.56 (m, 2H), 1.46-1.39 ppm (m, 2H); ¹³C NMR (150 MHz, CDCl₃) δ 210.00, 168.13, 159.21, 134.21, 132.56, 130.77, 129.35, 125.84, 113.85, 72.68, 69.98, 55.39, 41.52, 40.61, 32.03, 29.49, 27.25, 26.30 ppm; HR-MS (ESI-TOF): calcd for C₂₀H₂₆O₃Na⁺ [M+Na]⁺: 337.1779. found: 337.1759.

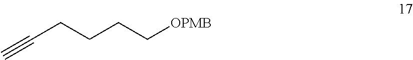

### PMB ether 17

To a stirred slurry of NaH (60% dispersion in mineral oil, 13.2 g, 306 mmol, 1.2 equiv) in THF (200 mL) was added a solution of 5-hexyn-1-ol 7 (25.0 g, 255 mmol, 1.0 equiv) at 0° C. and the mixture was stirred at 0° C. for 2 h. 4-Methoxybenzyl chloride (44.4 mL, 306 mmol, 1.2 equiv.) and tetra-n-butylammonium iodide (5.0 g, 30.6 mmol, 0.1 equiv) were then added and the mixture was stirred at 25° C. for 36 h. The reaction mixture was quenched with saturated aqueous NH₄Cl solution (100 mL) at 0° C., allowed to warm to 25° C. and extracted with EtOAc (2×200 mL). The combined organic layers were washed sequentially with H₂O (100 mL) and brine (100 mL), dried (Na₂SO₄), filtered and concentrated under reduced pressure. Purification by flash column chromatography (SiO₂, hexane:EtOAc, 12:1) gave pure title compound (17 (Hayashi, et al., 1997), 46.1 g, 214 mmol, 84% yield) as a colorless oil. 17: R_{f}=0.40 (hexane:EtOAc, 10:1); IR (film): νₘₐₓ=3291, 2954, 2938, 2861, 1612, 1513, 1246, 1033, 821 cm⁻¹; ¹H NMR (600 MHz, CDCl₃) δ 7.26 (d, J=8.6 Hz, 2H), 6.88 (d, J=8.6 Hz, 2H), 4.43 (s, 2H), 3.80 (s, 3H), 3.47 (t, J=6.5 Hz, 2H), 2.21 (tt, J=7.0, 2.6 Hz, 2H), 1.94 (t, J=2.6 Hz, 1H), 1.74-1.70 (m, 2H), 1.64-1.60 ppm (m, 2H); ¹³C NMR (150 MHz, CDCl₃) δ 159.25, 130.78, 129.33, 113.88, 84.49, 72.65, 69.54, 68.48, 55.39, 28.88, 25.36, 18.32 ppm; HRMS (ESI-TOF) calcd for C₁₄H₁₈O₂Na⁺ [M+Na]⁺: 241.1202. found: 241.1198.

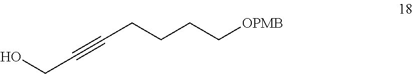

### Propargyl alcohol 18

To a stirred solution of PMB ether 17 (18.0 g, 82.0 mmol, 1.0 equiv.) in THF (160 mL) at −78° C. was added dropwise n-butyl lithium (2.5 M solution in hexane, 40.0 mL, 100.0 mmol, 1.2 equiv) and the mixture was stirred at −78° C. for 30 min and then warmed to −45° C. and stirred there for 30 min. The reaction mixture was again cooled to −78° C., paraformaldehyde (3.0 g, 100.0 mmol, 1.2 equiv) was added and the resulting orange solution was warmed to 25° C. and stirred for 2 h. Saturated aqueous NH₄Cl-solution (100 mL) was added to the mixture at 0° C., the phases were separated and the aqueous layer was extracted with EtOAc (2×200 mL). The combined organic layers were washed with H₂O (100 mL), brine (100 mL), dried (Na₂SO₄), filtered and concentrated under reduced pressure. Purification by flash column chromatography (SiO₂, hexane-EtOAc, 4:1) gave pure title compound (18 (Iimura, et al., 2006), 18.1 g, 89.0 mmol, 89% yield) as a colorless oil. 18: R_{f}⁼0.40 (hexane:EtOAc, 3:1); IR (film): νₘₐₓ=3409, 2936, 2862, 1612, 1586, 1513, 1456, 1246, 1174, 1092, 1031, 820 cm⁻¹; ¹H NMR (600 MHz, CDCl₃) δ 7.26 (d, J=8.6 Hz, 2H), 6.88 (d, J=8.6 Hz, 2H), 4.43 (s, 2H), 4.42 (s, 2H), 3.80 (s, 3H), 3.45 (t, J=6.5 Hz, 2H), 2.23 (tt, J=7.0, 2.6 Hz, 1H), 1.88 (bs, 1H), 1.94 (t, J=2.6 Hz, 1H), 1.72-1.68 (m, 2H), 1.61-1.56 ppm (m, 2H); ¹³C NMR (150 MHz, CDCl₃) δ 159.24, 130.68, 129.35, 113.88, 86.21, 78.76, 72.66, 69.57, 55.38, 51.40, 28.94, 25.41, 18.63 ppm; HRMS (ESI-TOF) calcd for C₁₅H₂₀O₃Na⁺ [M+Na]⁺: 271.1309. found: 271.1300.

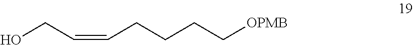

### Allylic alcohol 19

To a stirred suspension of Ni(OAc)₂.4H₂O (2.56 g, 10.3 mmol, 0.16 equiv) in EtOH (400 mL) under an atmosphere of H₂ was added NaBH₄ (882 mg, 24.5 mmol, 0.38 equiv) as a solution in EtOH (30 mL), and the flask headspace was evacuated and refilled with H₂ three times. The black suspension was stirred for 20 min at 25° C. and then a solution of 1,2-ethylenediamine (8.64 mL, 116.1 mmol, 1.8 equiv) in EtOH (30 mL) was added, and the flask headspace was again evacuated and refilled with H₂ three times. After stirring for 20 min, a solution of propargyl alcohol 18 (16.0 g, 64.5 mmol, 1.0 equiv) in EtOH (100 mL) was added, and the headspace was evacuated and refilled with H₂ three more times. The reaction flask was then shielded from light with aluminum foil and stirred at 25° C. for 6 h. The H₂ atmosphere was removed, the solution was filtered and the filtrate was concentrated under reduced pressure. The crude material was re-dissolved in EtOAc (500 mL) and washed sequentially with saturated aqueous NH₄Cl solution (100 mL), H₂O (2×100 mL) and brine (100 mL). The organic phase was dried (Na₂SO₄), filtered and concentrated under reduced pressure. Purification by flash column chromatography (SiO₂, hexane:EtOAc, 3:2) gave pure title compound (19 (Lindsay, et al., 2002), 14.8 g, 59.3 mmol, 92% yield) as a colorless oil. 19: R_{f}=0.35 (hexane:EtOAc, 3:1); IR (film): νₘₐₓ=3394, 3011, 2933, 2858, 1612, 1586, 1513, 1463, 1247, 1097, 1035, 820 cm⁻¹; ¹H NMR (600 MHz, CDCl₃) δ 7.26 (d, J=8.6 Hz, 2H), 6.88 (d, J=8.6 Hz, 2H), 5.76-5.70 (m, 1H), 5.59 (dt, J=10.6, 7.5 Hz, 1H), 4.43 (s, 2H), 3.98 (d, J=8.3 Hz, 2H), 3.81 (s, 3H), 3.45 (t, J=6.4 Hz, 2H), 2.15 (qd, J=7.5, 1.6 Hz, 2H), 1.66-1.60 (m, 2H), 1.52-1.47 ppm (m, 2H); ¹³C NMR (150 MHz, CDCl₃) δ 159.25, 132.93, 130.69, 129.34, 128.79, 113.89, 72.69, 69.99, 58.63, 55.40, 29.32, 27.28, 26.38 ppm; HRMS (ESI-TOF) calcd for C₁₅H₂₂O₃Na⁺ [M+Na]⁺: 273.1466. found: 273.1452.

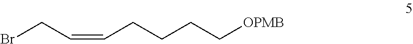

### Bromide 5

To a stirred solution of allylic alcohol 19 (8.0 g, 32.0 mmol, 1.0 equiv) in MeCN (90 mL) were added PPh₃ (13.6 g, 52.0 mmol, 1.6 equiv) and CBr₄ (17.3 g, 52.0 mmol, 1.6 equiv) at −10° C. After stirring at 0° C. for 30 min, the solvent was removed under reduced pressure keeping water bath temperature below 25° C. Purification by flash column chromatography (SiO₂, hexane:EtOAc, 19:1) gave pure title compound (5, 8.8 g, 28.2 mmol, 88% yield) as a colorless oil. 5: R_{f}=0.60 (hexane:EtOAc, 10:1); IR (film): νₘₐₓ=2935, 2857, 1612, 1512, 1462, 1247, 1099, 1035, 820 cm⁻¹; ¹H NMR (600 MHz, CDCl₃) δ 7.26 (d, J=8.6 Hz, 2H), 6.88 (d, J=8.6 Hz, 2H), 5.75-5.70 (m, 1H), 5.59 (dt, J=10.6, 7.5 Hz, 1H), 4.43 (s, 2H), 3.98 (d, J=8.3 Hz, 2H), 3.80 (s, 3H), 3.45 (t, J=6.4 Hz, 2H), 2.15 (qd, J=7.5, 1.6 Hz, 2H), 1.66-1.60 (m, 2H), 1.52-1.47 ppm (m, 2H); ¹³C NMR (150 MHz, CDCl₃) δ 159.23, 135.74, 130.77, 129.34, 125.59, 113.87, 72.67, 69.83, 55.38, 29.39, 27.39, 26.77, 25.89 ppm.

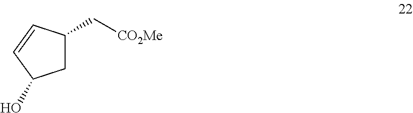 [SMILES: COC(=O)C[C@H]1C=C[C@@H](O)C1]

### Hydroxyester 22

To a stirred solution of triphenylmethyl hydroperoxide (4.45 g, 16.1 mmol, 1.33 equiv) in THF (60 mL) at −78° C. was added n-butyl lithium (2.5 M in hexane, 6.10 mL, 15.1 mmol, 1.25 equiv) dropwise. After stirring at −78° C. for 1.5 h, (−)-norbomenone 21 (1.31 g, 12.1 mmol, 1.0 equiv) was added dropwise and the resulting solution was stirred at −78° C. for 6 h. Then a solution of sodium methoxide (1.31 g, 24.2 mmol, 2.0 equiv) in MeOH (48 mL) was added dropwise and the resulting mixture was warmed to 0° C. After stirring at that temperature for 4 h, the reaction mixture was quenched with saturated aqueous NH₄Cl solution (60 mL) and warmed to 25° C. The aqueous layer was extracted with EtOAc (3×50 mL). The combined organic layers were washed with brine (50 mL), dried over MgSO₄ and concentrated under reduced pressure. Purification by flash column chromatography (SiO₂, hexane:EtOAc, 2:1) gave pure title compound (22, 1.55 g, 9.9 mmol, 82% yield) as colorless oil. 22: R_{f}=0.37 (hexane:EtOAc, 1:1); [α]_{D}²⁵=+17.0° (c=0.85 in CHCl₃); IR (film): νₘₐₓ=3387, 2954, 1733, 1437, 1262, 1196, 1158, 1072, 1021, 763 cm⁻¹; ¹H NMR (600 MHz, CDCl₃) δ 5.85 (br s, 2H), 4.80 (ddd, J=7.6, 4.2, 1.6 Hz, 1H), 3.67 (s, 3H), 3.03-2.93 (m, 1H), 2.57 (ddd, J=13.9, 8.3, 7.6 Hz, 1H), 2.50 (dd, J=15.8, 6.5 Hz, 1H), 2.44 (dd, J=15.8, 7.3 Hz, 1H), 2.05 (br s, 1H), 1.39 ppm (ddd, J=13.9, 5.0, 4.1 Hz, 1H); ¹³C NMR (150 MHz, CDCl₃) δ 173.23, 136.87, 134.49, 77.18, 51.69, 40.68, 40.21, 39.87 ppm; HR-MS (ESI-TOF) calcd for C₈H₁₂O₃Na⁺ [M+Na]⁺: 179.0679. found: 179.0684.

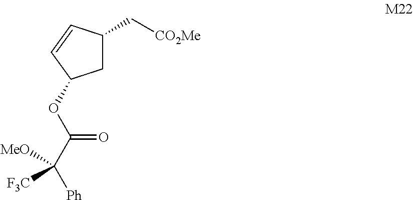 [SMILES: COC(=O)C[C@H]1C=C[C@@H](OC(=O)[C@@](OC)(c2ccccc2)C(F)(F)F)C1]

### Mosher ester M22

To a stirred solution of hydroxyester 22 (6.1 mg, 0.039 mmol, 1.0 equiv) in CH₂Cl₂ (0.4 mL) at 25° C. were added 4-dimethylaminopyridine (14.3 mg, 0.117 mmol, 3.0 equiv) and (R)-(−)-Mosher acid chloride (15 μL, 0.078 mmol, 2.0 equiv). After stirring for 8 h, the reaction mixture was directly subjected to flash column chromatography (SiO₂, hexane:EtOAc, 7:1) to give pure Mosher ester (M22, 13.6 mg, 0.0368 mmol, 94% yield) as a colorless oil. M22: R_{f}=0.74 (hexane:EtOAc, 2:1); [α]_{D}²⁵=−97.2° (c=1.00 in CHCl₃); IR (film): νₘₐₓ=1736, 1252, 1163, 1121, 1015, 991, 713, 698 cm⁻¹; ¹H NMR (600 MHz, CDCl₃) δ 7.54-7.48 (m, 2H), 7.42-7.36 (m, 3H), 6.13 (dd, J=5.8, 2.4 Hz, 1H), 5.93 (dt, J=5.7, 2.0 Hz, 1H), 5.87-5.83 (m, 1H), 3.66 (s, 3H), 3.55 (s, 3H), 3.11-3.03 (m, 1H), 2.59 (dt, J=14.6, 7.8 Hz, 1H), 2.38 (dd, J=15.8, 6.9 Hz, 1H), 2.28 (dd, J=15.8, 8.1 Hz, 1H), 1.55 ppm (dt, J=14.6, 3.6 Hz, 1H); ¹³C NMR (150 MHz, CDCl₃) δ 172.66, 166.31, 141.64, 132.49, 129.68, 128.91, 128.52, 127.42, 123.45 (q, J=288.4 Hz), 84.62 (q, J=27.5 Hz), 81.89, 55.53, 51.74, 40.57, 40.05, 36.06 ppm; HR-MS (ESI-TOF) calcd for C₁₈H₁₉F₃O₅Na⁺ [M+Na]⁺: 395.1077. found: 395.1067.

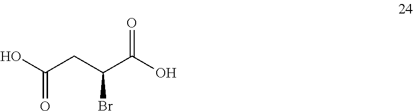

### Bromosuccinic acid 24

(Method modified from Frick, et al., 1992) To a stirred solution of L-aspartic acid (81.0 g, 616 mmol, 1.0 equiv) and KBr (315.5 g, 2.65 mol, 4.3 equiv) in aqueous H₂SO₄ (2.5 M, 1.70 L) at −5° C. was added a solution of NaNO₂ (72.2 g, 1.05 mol, 1.7 equiv) in H₂O (120 mL) dropwise over 1.5 h. The temperature was maintained between −5 and 0° C. over the addition period. After the addition was complete the reaction mixture was stirred for 2 h at −5° C. The reaction mixture was then extracted with EtOAc (5×300 mL), the combined organic layers were washed with half-saturated brine (300 mL), dried (Na₂SO₄) and concentrated under reduced pressure to afford crude title compound (24, 102.0 g, 530 mmol, 86% yield) as a colorless solid. The crude material was used in the next step without further purification.

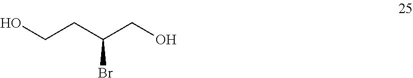

### Bromo diol 25

(Method modified from Frick, et al., 1992) To a stirred solution of bromosuccinic acid 24 (101.0 g, 520 mmol, 1.0 equiv) in THF (600 mL) at −10° C. was added dropwise BH₃.Me₂S (2.0 M in THF, 780 mL, 1.56 mol, 3.0 equiv). The reaction mixture was warmed to 25° C. and stirred for 12 h. The reaction mixture was then quenched by the slow addition of MeOH (150 mL) at 0° C. under vigorous stirring. The resulting clear solution was concentrated under reduced pressure and the residue was redissolved in MeOH (150 mL) and concentrated under reduced pressure. This process was repeated two more times and the crude diol was filtered through a short column (SiO₂, acetone:CH₂Cl₂, 1:1) to afford crude title compound (25, 81.0 g, 489 mmol, 94% yield) as a light yellow oil. The crude material was used in the next step without further purification.

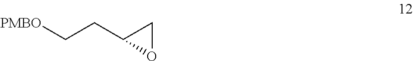

### Epoxide 12

To a stirred slurry of NaH (60% in mineral oil, 14.2 g, 354 mmol, 3.0 equiv) in THF (200 mL) was added a solution of bromo diol 25 (20.0 g, 118 mmol, 1.0 equiv) in THF (30 mL) dropwise at 0° C. After stirring at 0° C. for 1 h, PMBBr (17.0 mL, 118 mmol, 2.0 equiv) was added dropwise followed by TBAI (3.80 g, 11.8 mmol, 0.1 equiv). The reaction mixture was allowed to warm to 25° C. and stirred for 12 h. The reaction mixture was then cooled to 0° C. and quenched by the slow addition of saturated aqueous NH₄Cl solution (50 mL) and diluted with EtOAc (400 mL). The layers were separated and the aqueous layer was extracted with EtOAc (3×100 mL). The combined organic layers were washed sequentially with H₂O (2×100 mL) and brine (100 mL), dried (Na₂SO₄), filtered and concentrated under reduced pressure. Purification by flash column chromatography (hexane:EtOAc, 4:1) gave pure title compound (12 (Simpson, et al., 1997), 17.4 g, 255 mmol, 72% yield) as a colorless oil. 12: R_{f}=0.50 (hexane:EtOAc, 3:1); [α]_{D}²⁵=+12.3° (c=1.00, CHCl₃); IR (film): νₘₐₓ=2998, 2921, 2857, 1612, 1512, 1246, 1173, 1090, 1033, 821 cm⁻¹; ¹H NMR (600 MHz, CDCl₃) δ 7.26 (d, J=8.6 Hz, 2H), 6.91 (d, J=8.6 Hz, 2H), 4.46 (s, 2H), 3.80 (s, 3H), 3.62-3.56 (m, 2H), 3.07-3.04 (m, 1H), 2.77 (t, J=4.3 Hz, 1H), 2.52 (dd, J=4.3, 2.7 Hz, 1H), 1.92-1.87 (m, 1H), 1.79-1.74 ppm (m, 1H); ¹³C NMR (150 MHz, CDCl₃) δ 159.32, 130.42, 129.36, 113.82, 72.87, 66.84, 55.38, 50.21, 47.21, 33.06 ppm; HRMS (ESI-TOF) calcd for C₁₂H₁₆O₃Na⁺ [M+Na]⁺: 231.0996. found: 231.0982.

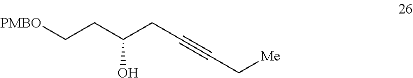

### Alcohol 26

To a stirred solution of 1-butyne (5.2 mL, 96.0 mmol, 2.0 equiv) in THF (230 mL) at −78° C. was added n-butyl lithium (2.5 M in hexane, 28.8 mL, 72.0 mmol, 1.5 equiv) dropwise and the reaction mixture was stirred for 20 min at −78° C. and then for 1 h at −40° C. A solution of epoxide 12 (10.0 g, 48.0 mmol, 1.0 equiv) in THF (25 mL) was added all at once followed by dropwise addition of BF₃.Et₂O (8.3 mL, 57.6 mmol, 1.4 equiv) at −78° C. After stirring at that temperature for an additional 2 h, the reaction mixture was partitioned between saturated aqueous NH₄Cl solution (100 mL) and EtOAc (200 mL). The organic layer was separated and washed sequentially with H₂O (100 mL) and brine (100 mL), and the combined aqueous layers were re-extracted with EtOAc (2×100 mL). The combined organic layers were dried (Na₂SO₄), filtered, and concentrated under reduced pressure. Purification by flash column chromatography (hexane:EtOAc, 4:1) gave pure title compound (26, 10.6 g, 40.8 mmol, 85% yield) as a colorless oil. 26: R_{f}=0.37 (hexane:EtOAc, 3:1). [α]_{D}²⁵=+2.0° (c=2.00, CHCl₃); IR (film): νₘₐₓ=3435, 2936, 2862, 1612, 1586, 1512, 1461, 1301, 1245, 1173, 1080, 819 cm⁻¹; ¹H NMR (600 MHz, CDCl₃) δ 7.24 (d, J=8.8 Hz, 2H), 6.87 (d, J=8.7 Hz, 2H), 4.44 (s, 2H), 3.89 (dd, J=9.1, 3.0 Hz, 1H), 3.79 (d, J=1.4 Hz, 3H), 3.69 (tdd, J=6.1, 4.8, 2.4 Hz, 1H), 3.65-3.58 (m, 1H), 3.02 (s, 1H), 2.38-2.29 (m, 2H), 2.16 (qt, J=7.5, 2.4 Hz, 2H), 1.87 (dddd, J=14.2, 6.1, 4.6, 3.2 Hz, 1H), 1.85-1.75 (m, 1H), 1.11 ppm (t, J=7.5 Hz, 3H); ¹³C NMR (150 MHz, CDCl₃) δ 159.37, 130.17, 129.42, 113.95, 84.24, 75.69, 73.06, 69.93, 68.47, 55.38, 35.50, 27.60, 14.33, 12.54 ppm; HRMS (ESI-TOF) calcd for C₁₆H₂₂O₃Na⁺ [M+Na]⁺: 285.1466. found: 285.1450.

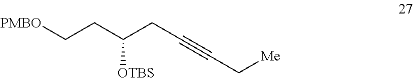

### Silyl ether 27

To a stirred solution of alcohol 26 (9.00 g, 34.0 mmol, 1.0 equiv) in CH₂Cl₂ (60 mL) at 0° C. was added imidazole (6.90 g, 102.0 mmol, 3.0 equiv) and TBSCl (7.72 g, 51.5 mmol, 1.5 equiv). The reaction mixture was warmed to 25° C. and stirred for 4 h. The resulting mixture was then diluted with H₂O (100 mL) and stirred vigorously for 30 min. The layers were separated and the aqueous layer was extracted with CH₂Cl₂ (3×50 mL). The organic layers were combined and washed sequentially with H₂O (2×100 mL) and brine (100 mL), dried (Na₂SO₄), filtered and concentrated under reduced pressure. Purification by flash column chromatography (hexane:EtOAc, 19:1) gave pure title compound (27, 12.10 g, 32.0 mmol, 94% yield) as a colorless oil. 27: R_{f}=0.40 (hexane:EtOAc, 10:1); [α]_{D}²⁵=+18.0° (c=1.00, CHCl₃); IR (film): νₘₐₓ=2954, 2929, 2857, 1472, 1361, 1255, 1094, 1035, 835, 806, 775 cm⁻¹; ¹H NMR (600 MHz, CDCl₃) δ 7.26 (d, J=8.6 Hz, 2H), 6.87 (d, J=8.6 Hz, 2H), 4.44 (d, J=11.5 Hz, 1H), 4.40 (d, J=11.5 Hz, 1H), 3.94-3.90 (m, 1H), 3.80 (s, 3H), 3.55-3.52 (m, 2H), 2.33-2.25 (m, 2H), 2.14 (qt, J=7.5, 2.4 Hz, 2H), 1.99-1.93 (m, 1H), 1.78-1.72 (m, 1H), 1.10 (t, J=7.5 Hz, 3H), 0.88 (s, 9H), 0.07 (s, 3H), 0.05 ppm (s, 3H); ¹³C NMR (150 MHz, CDCl₃) δ 159.23, 130.82, 129.38, 113.87, 83.54, 76.45, 72.67, 68.79, 66.76, 55.38, 36.80, 28.21, 25.96, 18.19, 14.30, 12.59, −4.33, −4.68 ppm; HRMS (ESI-TOF) calcd for C₂₂H₃₇O₃Si⁺ [M+H]⁺: 377.2513. found: 377.2509.

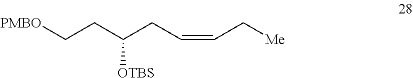

### (Z)-alkene 28

To a stirred suspension of Ni(OAc)₂.4H₂O (835 mg, 3.36 mmol, 0.16 equiv) in EtOH (200 mL) under a hydrogen atmosphere was added NaBH₄ (303 mg, 7.98 mmol, 0.38 equiv) as a solution in EtOH (20 mL). The flask headspace was evacuated and refilled with H₂ three times. After the black suspension was stirred for 10 min at 25° C., a solution of 1,2-ethylenediamine (2.52 mL, 37.8 mmol, 1.8 equiv) in EtOH (20 mL) was added, and the flask headspace was evacuated and refilled with H₂ three times. After stirring for 20 min, a solution of silyl ether 27 (8.00 g, 21.0 mmol, 1.0 equiv) in EtOH (20 mL) was added, and the headspace was evacuated again and refilled with H₂ three more times. The reaction flask was then shielded from light with aluminum foil and stirred at 25° C. for 6 h. The H₂ atmosphere was removed, and the solution was concentrated under reduced pressure. The crude material was redissolved in Et₂O (200 mL) and washed sequentially with saturated aqueous NH₄Cl-solution (30 mL) and brine (30 mL). The aqueous layers were re-extracted with Et₂O (2×100 mL) and the combined organic layers were washed with brine (50 mL), dried (Na₂SO₄), filtered and concentrated under reduced pressure. Purification by flash column chromatography (SiO₂, hexane-EtOAc, 19:1) gave pure title compound (28, 7.50 g, 19.5 mmol, 93% yield) as a colorless oil. 28: R_{f}⁼0.45 (hexane:EtOAc, 10:1); [α]_{D}²⁵=+17.0° (c=1.00, CHCl₃); IR (film): νₘₐₓ=2954, 2929, 2857, 1472, 1361, 1255, 1094, 1035, 835, 806, 775 cm⁻¹; ¹H NMR (600 MHz, CDCl₃) δ 7.26 (d, J=8.6 Hz, 2H), 6.87 (d, J=8.6 Hz, 2H), 5.46-5.42 (m, 1H), 5.38-5.34 (m, 1H), 4.44 (d, J=11.5 Hz, 1H), 4.39 (d, J=11.5 Hz, 1H), 3.88-3.84 (m, 1H), 3.80 (s, 3H), 3.53-3.50 (m, 2H), 2.25-2.17 (m, 2H), 2.05-2.00 (m, 2H), 1.81-1.75 (m, 1H), 1.71-1.65 (m, 1H), 0.95 (t, J=7.5 Hz, 3H), 0.89 (s, 9H), 0.06 (s, 3H), 0.05 ppm (s, 3H); ¹³C NMR (150 MHz, CDCl₃) δ 159.23, 133.48, 130.83, 129.39, 124.93, 113.86, 72.73, 69.47, 67.01, 55.37, 36.87, 35.61, 26.00, 20.83, 18.20, 14.33, −4.21, −4.63 ppm; HRMS (ESI-TOF) calcd for C₂₂H₃₉O₂Si⁺ [M+H]⁺: 379.2669. found: 379.2645.

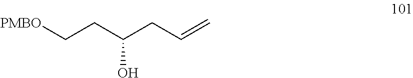

### Secondary alcohol 101

To a stirred slurry of CuI (0.31 g, 1.63 mmol, 0.17 equiv) in THF (70 mL) at −78° C. was added vinyl-MgBr (1.0 M in THF, 16.0 mL, 16.0 mmol, 1.7 equiv) with stirring. After stirring for 15 min at this temperature, a solution of epoxide 12 (2.00 g, 9.6 mmol, 1.0 equiv) in THF (5 mL) was added in one portion, and stirring was continued for 1 h at this temperature. The reaction mixture was gradually warmed to −30° C. and then to 0° C. over 2 h, before being quenched by the addition of saturated aqueous NH₄Cl solution (100 mL) and allowed to warm to 25° C. The phases were separated and the aqueous layer was extracted with Et₂O (2×20 mL). The combined organic extracts were dried (Na₂SO₄), filtered, and concentrated under reduced pressure. Purification by flash column chromatography (SiO₂, hexane:EtOAc, 1:1) gave pure title compound (101 (Smith, et al., 2001), 2.00 g, 8.7 mmol, 93% yield) as a colorless oil. 101: R_{f}=0.46 (hexane:EtOAc, 7:3); [α]_{D}²⁵=−3.9 (c=0.76 in CHCl₃); IR (film): νₘₐₓ=3431, 2936, 2862, 1612, 1512, 1245, 1083, 1033 cm⁻¹; ¹H NMR (600 MHz, CDCl₃) δ 7.29-7.26 (m, 2H), 6.94-6.85 (m, 2H), 5.85 (ddt, J=17.3, 10.3, 7.1 Hz, 1H), 5.18-5.06 (m, 2H), 4.48 (s, 2H), 3.92-3.85 (m, 1H), 3.83 (s, 3H), 3.71 (dt, J=9.5, 5.3 Hz, 1H), 3.64 (ddd, J=9.3, 7.2, 5.3 Hz, 1H), 2.26 (td, J=6.7, 6.0, 1.3 Hz, 2H), 1.77 ppm (ddd, J=9.3, 7.5, 5.1 Hz, 2H); ¹³C NMR (150 MHz, CDCl₃) δ 159.27, 130.06, 129.31, 117.52, 113.85, 72.97, 70.48, 68.68, 55.29, 41.93, 35.85 ppm; HR-MS (ESI-TOF): calcd for C₁₄H₂₀O₃Na⁺ [M+Na]⁺: 259.1305. found: 259.1297.

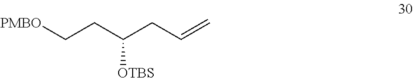

### Alkene 30

To a stirred solution of alcohol 101 (1.75 g, 7.42 mmol, 1.0 equiv) in CH₂Cl₂ (15 mL) at 0° C. was added imidazole (1.31 g, 19.3 mmol, 2.6 equiv) followed by TBSCl (1.45 g, 9.64 mmol, 1.3 equiv). After stirring for 10 min at this temperature the reaction mixture was warmed to 25° C. and stirred for 15 h, the resulting mixture was quenched with H₂O (20 mL). The phases were separated and the aqueous layer was extracted with CH₂Cl₂ (3×10 mL), and the combined organic layers were dried (Na₂SO₄), filtered, and concentrated under reduced pressure. Purification by flash column chromatography (SiO₂, hexane:EtOAc, 8:2) gave pure title compound (30 (Greene, et al., 1980; Corey, et al., 1988), 2.13 g, 6.0 mmol, 80% yield) as a light yellow oil. 30: R_{f}=0.94 (hexane:EtOAc, 8:2); [α]_{D}²⁵=+17.7 (c=1.00 in CHCl₃); IR (film): νₘₐₓ=2952, 2928, 2856, 1613, 1513, 1246, 1088, 1034, 833 cm⁻¹; ¹H NMR (600 MHz, CDCl₃) δ 7.21 (d, J=7.8 Hz, 2H), 6.89-6.77 (m, 2H), 5.85-5.69 (m, 1H), 5.03-4.93 (m, 2H), 4.45-4.28 (m, 2H), 3.90-3.80 (m, 1H), 3.76 (s, 3H), 3.47 (t, J=6.8 Hz, 2H), 2.25-2.11 (m, 2H), 1.73 (dtd, J=14.2, 7.2, 4.3 Hz, 1H), 1.65 (ddt, J=13.8, 7.6, 6.0 Hz, 1H), 0.84 (s, 9H), 0.01 ppm (d, J=6.6 Hz, 6H); ¹³C NMR (150 MHz, CDCl₃) δ 159.11, 134.97, 130.69, 129.27, 116.94, 113.75, 72.61, 68.98, 66.77, 55.29, 42.30, 36.73, 25.88, 18.10, −4.33, −4.70 ppm; HR-MS (ESI-TOF): calcd for C₂₀H₃₄O₃SiNa⁺ [M+Na]⁺: 373.2169. found: 373.2165.

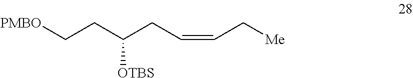

### (Z)-Alkene 28

To a stirred solution of alkene 30 (1.00 g, 2.85 mmol, 1.0 equiv) in THF:H₂O (2:1, 16 mL) at 25° C. was added a solution of OsO₄ (2.5 wt % in t-BuOH, 1.44 mL, 1.42 mmol, 0.05 equiv). After stirring for 15 min at this temperature, NaIO₄ (1.83 g, 8.55 mmol, 3.0 equiv) was added and the reaction mixture was stirred for 15 h and then quenched with aqueous 10% Na₂S₂O₃-solution. The phases were separated and the aqueous layer was extracted with EtOAc (3×15 mL). The combined organic layers were dried (Na₂SO₄), filtered, and concentrated under reduced pressure. The crude aldehyde was used immediately in the next step without further purification. To a stirred solution of BrPPh₃(CH₂)₂CH₃ (2.96 g, 7.69 mmol, 2.7 equiv) in THF at 0° C. was added NaHMDS (1.0 M in THF, 7.69 mL, 7.69 mmol, 2.7 equiv) and the resulting mixture was allowed to warm to 25° C. After stirring for 1 h at this temperature, the orange solution was cooled to −78° C. and a solution of crude aldehyde (1.00 g, 2.85 mmol, 1.0 equiv) was added. After stirring for 2 h at this temperature, the reaction mixture was quenched with saturated aqueous NH₄Cl solution (100 mL). The phases were separated and the aqueous layer was extracted with Et₂O (3×50 mL), and the combined organic layers were dried (Na₂SO₄), filtered, and concentrated under reduced pressure. Purification by flash column chromatography (SiO₂, hexane:EtOAc, 8:2) gave pure title compound (28, 0.80 g, 2.11 mmol, 74% yield). Data were in agreement with those obtained for the alternative route (vide supra).

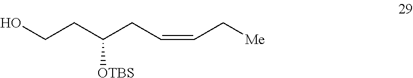 [SMILES: CC/C=C\C[C@@H](CCO)O[Si](C)(C)C(C)(C)C]

### Alcohol 29

To a vigorously stirred solution of (Z)-alkene 28 (7.40 g, 19.4 mmol, 1.0 equiv) in CH₂Cl₂:pH=7 buffer (20:1, 63 mL) was added DDQ (6.56 g, 29.0 mmol, 1.5 equiv) at 0° C. The reaction mixture was stirred for 2 h before being quenched by the addition of saturated aqueous NaHCO₃ solution (100 mL) and the mixture was stirred vigorously for 30 min. The layers were separated and the aqueous layer was extracted with Et₂O (3×200 mL). The combined organic layers were dried (Na₂SO₄) and concentrated under reduced pressure. Purification by flash column chromatography (SiO₂, hexane:EtOAc, 9:1) gave pure title compound (29, 4.58 g, 17.5 mmol, 90% yield) as a light yellow oil. 29: R_{f}=0.40 (hexane:EtOAc, 5:1); [α]_{D}²⁵=+34.2° (c=1.00, CHCl₃); IR (film): νₘₐₓ=3355, 3011, 2958, 2857, 1472, 1361, 1255, 1080, 1060, 774 cm⁻¹; ¹H NMR (600 MHz, CDCl₃) δ 5.47-5.43 (m, 1H), 5.32-5.27 (m, 1H), 3.96-3.92 (m, 1H), 3.84-3.80 (m, 1H), 3.73-3.69 (m, 1H), 2.41 (t, J=7.5 Hz, 1H), 2.32-2.24 (m, 2H), 2.06-2.02 (m, 2H), 1.83-1.79 (m, 1H), 1.67-1.62 (m, 1H), 0.96 (t, J=7.5 Hz, 3H), 0.89 (s, 9H), 0.10 (s, 3H), 0.09 ppm (s, 3H); ¹³C NMR (150 MHz, CDCl₃) δ 133.92, 124.46, 71.98, 60.45, 37.71, 34.99, 25.94, 20.88, 18.09, 14.30, −4.24, −4.72 ppm; HRMS (ESI-TOF) calcd for C₁₄H₃₁O₂Si⁺ [M+H]⁺: 259.2094. found: 259.2077.

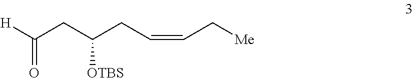 [SMILES: CC/C=C\C[C@@H](CC=O)O[Si](C)(C)C(C)(C)C]

### Aldehyde 3

To a stirred solution of alcohol 29 (2.10 g, 2.64 mmol, 1.0 equiv) in CH₂Cl₂ (15 mL) was added DMP (1.68 g, 3.96 mmol, 1.5 equiv) at 0° C. and the reaction mixture was warmed to 25° C. and stirred for 2 h. The reaction mixture was quenched by the addition of saturated aqueous Na₂S₂O₃-solution (15 mL) and saturated aqueous NaHCO₃-solution (15 mL) and the reaction mixture was stirred vigorously for 20 min. The layers were separated and the aqueous layer was extracted with Et₂O (3×50 mL). The organic layers were combined, dried (Na₂SO₄) and concentrated under reduced pressure. Purification by flash column chromatography (SiO₂, hexane:EtOAc, 20:1) gave pure title compound (3, 2.05 g, 2.61 mmol, 99% yield) as a colorless oil. 3: R_{f}=0.70 (hexane:EtOAc, 10:1); [α]_{D}²⁵=+24.5° (c=1.00, CHCl₃); IR (film): νₘₐₓ=3011, 2931, 2858, 1727, 1463, 1254, 1098, 1005, 776 cm¹; ¹H NMR (600 MHz, CDCl₃) δ 9.80 (t, J=2.5 Hz, 1H), 5.52-5.47 (m, 1H), 5.35-5.30 (m, 1H), 4.24-4.20 (m, 1H), 2.51 (dd, J=5.9, 2.4 Hz, 2H), 2.34-2.24 (m, 2H), 2.02 (p, J=7.6 Hz, 2H), 0.96 (t, J=7.6 Hz, 3H), 0.87 (s, 9H), 0.08 (s, 3H), 0.06 ppm (s, 3H); ¹³C NMR (150 MHz, CDCl₃) δ 202.34, 134.74, 123.74, 68.34, 50.56, 35.71, 25.87, 20.86, 18.10, 14.24, −4.25, −4.71 ppm; HRMS (ESI-TOF) calcd for C₁₄H₂₉O₂Si⁺ [M+H]⁺: 257.1938. found: 257.1929.

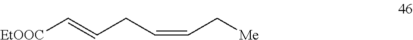 [SMILES: CC/C=C\C/C=C/C(=O)OCC]

### Ethyl ester 46

(For example, see Honda, et al., 1997) To a stirred solution of (Z)-3-hexene-1-ol (5.00 g, 50.0 mmol, 1.0 equiv) and PhI(OAc)₂ (21.0 g, 65.0 mmol, 1.3 equiv) in pentane:CH₂Cl₂ (9:2, 100 mL) was added TEMPO (1.56 g, 10.0 mmol, 0.2 equiv) at 25° C. The reaction mixture was stirred for 45 min and ethyl 2-(triphenylphosphoranylidene) acetate (26.1 g, 75.0 mmol, 1.5 equiv) was added in portions. After 12 h of stirring at 25° C., the solvent was removed under reduced pressure. Purification by flash column chromatography (SiO₂, pentane:Et₂O, 1:0→19:1) gave pure title compound (46, 5.46 g, 32.5 mmol, 65% yield) as a colorless oil. 46: R_{f}=0.60 (hexane:EtOAc, 10:1); IR (film): νₘₐₓ=3014, 2935, 2966, 2875, 1720, 1652, 1367, 1325, 1269, 1164, 1047 cm¹; ¹H NMR (600 MHz, CDCl₃) δ 6.95 (dt, J=15.6, 6.3 Hz, 1H), 5.83 (dt, J=15.6, 1.8 Hz, 1H), 5.56-5.52 (m, 1H), 5.37-5.32 (m, 1H), 4.18 (q, J=7.1 Hz, 2H), 2.93 (t, J=13.7, 6.8 Hz, 2H), 2.04 (pd, J=7.6, 1.5 Hz, 2H), 1.28 (t, J=7.2 Hz, 3H), 0.97 ppm (t, J=7.5 Hz, 3H); ¹³C NMR (150 MHz, CDCl₃) δ 166.85, 147.38, 134.63, 123.61, 121.52, 60.30, 29.97, 20.65, 14.40, 14.23 ppm; HRMS (ESI-TOF) calcd for C₁₀H₁₇O₂⁺ [M+H]⁺: 169.1229. found: 169.1227.

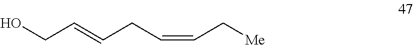

### Allylic alcohol 47

(See Frick, et al., 1992) To a stirred solution of ethyl ester 46 (5.00 g, 29.7 mmol, 1.0 equiv) in Et₂O (60 mL) at −78° C. was dropwise added diisobutylaluminum hydride (1.0 M in CH₂Cl₂, 68.5 mL, 68.5 mmol, 2.3 equiv). After stirring for 4 h at this temperature, the clear solution was cooled to −78° C. and quenched with MeOH (10 mL), diluted with Et₂O (5.6 mL) and allowed to warm to 25° C. Under vigorous stirring, saturated aqueous Na—K tartrate-solution (50 ml) was added and the resulting mixture was stirred for an additional 3 h. The resulting biphasic mixture was extracted with Et₂O (3×100 mL), and the combined organic layers were washed sequentially with H₂O (30 mL) and brine (30 mL). The organic phase was dried (Na₂SO₄), filtered, and concentrated under reduced pressure. Purification by flash column chromatography (SiO₂; hexane:EtOAc, 19:1→4:1) gave pure title compound (47, 3.25 g, 25.8 mmol, 87% yield) as a colorless oil. 47: R_{f}=0.40 (pentane:Et₂O, 4:1); IR (film): νₘₐₓ=3309, 3010, 2963, 2873, 1435, 1371, 1089, 1002, 967 cm⁻¹; ¹H NMR (600 MHz, CDCl₃) δ 5.71-5.64 (m, 2H), 5.48-5.43 (m, 1H), 5.37-5.32 (m, 1H), 4.10-4.09 (m, 2H), 2.80-2.78 (m, 2H), 2.07-2.02 (m, 2H), 0.97 ppm (t, J=7.4 Hz, 3H); ¹³C NMR (150 MHz, CDCl₃) δ 133.01, 131.58, 129.30, 126.07, 63.86, 30.02, 20.59, 14.36 ppm.

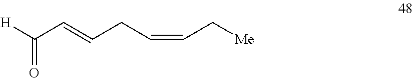

### Aldehyde 48

(See Frick, et al., 1992) To a vigorously stirred solution of allylic alcohol 47 (2.00 g, 16.0 mmol, 1.0 equiv) in CH₂Cl₂ (30 mL) at 25° C. was added in one portion pyridinium chlorochromate (6.80 g, 32 mmol, 2.0 equiv). After stirring for 4 h, the mixture was directly, and without any further work-up, loaded onto a column. Purification by flash column chromatography (SiO₂, pentane:Et₂O, 100:1→19:1) gave pure title compound (48, 1.46 g, 11.5 mmol, 72% yield) as a colorless oil. 24: R_{f}=0.40 (hexane:EtOAc, 10:1); IR (film): νₘₐₓ=3014, 2965, 2873, 5, 2813, 2733, 1686, 1633, 1462, 1129, 981, 731 cm⁻¹; ¹H NMR (600 MHz, CDCl₃) δ 9.52 (dd, J=7.9, 1.0 Hz, 1H), 6.83 (dt, J=15.6, 6.1 Hz, 1H), 6.14 (ddt, J=15.6, 7.9, 1.8 Hz, 1H), 5.61-5.57 (m, 1H), 5.39-5.35 (m, 1H), 3.07 (t, J=6.6 Hz, 2H), 2.08-2.03 (m, 2H), 0.98 ppm (t, J=7.5 Hz, 3H); ¹³C NMR (150 MHz, CDCl₃) δ 194.05, 156.79, 135.30, 132.99, 122.80, 30.56, 20.71, 14.16 ppm; HR-MS (ESI-TOF): calcd for C₈H₁₃O⁺ [M+H]⁺: 125.0967. found: 125.0966.

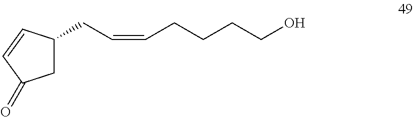 [SMILES: O=C1C=C[C@H](C/C=C\CCCCO)C1]

### Hydroxy enone 49

To a vigorously stirred solution of enone 2 (500 mg, 1.59 mmol, 1.0 equiv) in a mixture of CH₂Cl₂:H₂O (20:1, 8 mL) at 0° C. was added in one portion DDQ (540 mg, 2.40 mmol, 1.5 equiv). After stirring at this temperature for 45 min, the reaction mixture was diluted with EtOAc (30 mL), filtered through Celite®, washed with EtOAc (50 mL), and concentrated under reduced pressure. Purification by flash column chromatography (SiO₂, hexane:EtOAc, 10:1→2:1) gave pure title compound (49, 291 mg, 1.49 mmol, 94% yield) as a colorless oil. 49: R_{f}=0.38 (hexane:EtOAc, 2:1); [α]_{D}²⁵=+80.1° (c=0.47 in CHCl₃); IR (film): νₘₐₓ=3421, 3008, 2931, 2860, 1706, 1670, 1585, 1184, 1056, 785 cm⁻¹; ¹H NMR (600 MHz, CDCl₃) δ 7.62 (dd, J=5.8, 2.7 Hz, 1H), 6.17 (dd, J=5.9, 2.0 Hz, 1H), 5.53-5.49 (m, 1H), 5.39-5.34 (m, 1H), 3.64 (t, J=6.5 Hz, 3H), 3.03-2.98 (m, 1H), 2.51 (dd, J=19.7, 6.5 Hz, 1H), 2.33-2.28 (m, 1H), 2.24-2.19 (m, 1H), 2.08-2.00 (m, 3H), 1.60-1.55 (m, 2H), 1.46-1.41 ppm (m, 2H); ¹³C NMR (150 MHz, CDCl₃) δ 210.00, 168.06, 134.25, 132.46, 126.00, 62.88, 41.51, 40.62, 32.43, 32.09, 27.19, 25.82 ppm; HR-MS (ESI-TOF): calcd for C₁₂H₁₉O₂⁺ [M+H]⁺: 195.1386. found: 195.1377.

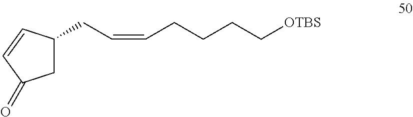 [SMILES: CC(C)(C)[Si](C)(C)OCCCC/C=C\C[C@H]1C=CC(=O)C1]

### Silyl ether 50

To a stirred solution of hydroxy enone 49 (220 mg, 1.10 mmol, 1.0 equiv) in CH₂Cl₂ (3 mL) at 25° C. were added sequentially imidazole (440 mg, 3.30 mmol, 3.0 equiv) and TBSCl (280 mg, 1.65 mmol, 1.5 equiv). The reaction mixture was stirred for 4 h at the same temperature, quenched with saturated aqueous NH₄Cl-solution (3 mL), the phases were separated and the aqueous layer was extracted with CH₂Cl₂ (2×50 mL). The combined organic extracts were washed with H₂O (10 mL), dried (MgSO₄), filtered, and concentrated under reduced pressure. Purification by flash column chromatography (SiO₂, hexane:EtOAc, 8:1) gave pure title compound (50, 300 mg, 0.98 mmol, 89% yield) as a colorless oil. 50: R_{f}=0.40 (hexane:EtOAc, 4:1); [α]_{D}²⁵=+93.3° (c=1.00 in C₆H₆); IR (film): νₘₐₓ=3008, 2929, 2857, 1716, 1587, 1472, 1408, 1254, 1180, 1099, 1006, 835, 776 cm⁻¹; ¹H NMR (600 MHz, CDCl₃) δ 7.62 (dd, J=5.8, 2.7 Hz, 1H), 6.16 (dd, J=5.9, 2.0 Hz, 1H), 5.53-5.48 (m, 1H), 5.37-5.33 (m, 1H), 3.59 (td, J=6.5, 1.3 Hz, 2H), 3.01-2.98 (m, 1H), 2.51 (dd, J=18.8, 6.5 Hz, 1H), 2.32-2.27 (m, 1H), 2.24-2.19 (m, 1H), 2.05-1.99 (m, 3H), 1.53-1.48 (m, 2H), 1.42-1.36 (m, 2H), 0.88 (s, 9H), 0.03 ppm (s, 6H); ¹³C NMR (150 MHz, CDCl₃) δ 209.97, 168.08, 134.23, 132.77, 125.74, 63.11, 41.55, 40.64, 32.58, 32.08, 27.26, 26.09, 25.77, 18.47, −5.15 ppm; HR-MS (ESI-TOF): calcd for C₁₈H₃₃O₂Si⁺ [M+H]⁺: 309.2251. found: 309.2234.

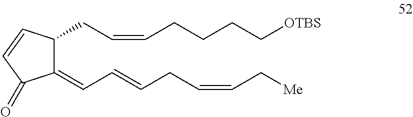 [SMILES: CC/C=C\C/C=C/C=C1/C(=O)C=C[C@@H]1C/C=C\CCCCO[Si](C)(C)C(C)(C)C]

### Trienone 52

To a stirred solution of diisopropylamine (230 μL, 1.64 mmol, 2.05 equiv) in THF (12 mL) at 0° C. was dropwise added n-butyl lithium (2.5 M in hexane, 640 μL, 1.60 mmol, 2.0 equiv). After stirring for 20 min at this temperature, the clear solution was cooled to −78° C. and a solution of enone 50 (240 mg, 0.80 mmol, 1.0 equiv) in THF (8 mL) was added dropwise. After stirring for an additional 20 min at this temperature, a solution of aldehyde 48 (147 mg, 1.20 mmol, 1.5 equiv) in THF (8 mL) was added dropwise and stirring at this temperature was continued for an additional 30 min. The reaction mixture was then quenched with saturated aqueous NH₄Cl solution (75 mL), diluted with EtOAc (75 mL), and allowed to warm to 25° C. The phases were separated, the aqueous layer was extracted with EtOAc (2×75 mL), and the combined organic extracts were washed with brine (50 mL), dried (Na₂SO₄), filtered, and concentrated under reduced pressure. The crude aldol product was filtered through a short column (SiO₂, hexane:EtOAc, 3:1) to obtain a mixture of diastereoisomers (274 mg, 0.63 mmol, 79% yield) as a colorless oil, which was taken to the next step without further purification. To a stirred solution of the aldol product (274 mg, 0.63 mmol, 1.0 equiv) in CH₂Cl₂ (6 mL) at −10° C. was added DMAP (770 mg, 6.3 mmol, 10 equiv), and then, slowly and dropwise, methanesulfonyl chloride (100 μL, 1.26 mmol, 2.0 equiv). After stirring for 30 min at this temperature, the reaction mixture was brought to 25° C. and stirred for 6 h. The reaction mixture was quenched with saturated aqueous NaHCO₃ solution (10 mL), diluted with CH₂Cl₂ (50 mL), the phases were separated, the aqueous layer was extracted with CH₂Cl₂ (2×50 mL), and the combined organic layers were washed with H₂O (20 mL), dried (Na₂SO₄), filtered, and concentrated under reduced pressure. Purification by flash column chromatography (SiO₂, hexane:EtOAc, 7:1) gave pure title compound (52, 102 mg; 0.25 mmol, 31% yield for 2 steps) as a colorless oil. 52: R_{f}=0.70 (hexane:EtOAc, 4:1); [α]_{D}²⁵=+58.1° (c=0.90 in C₆H₆); IR (film): νₘₐₓ=2941, 2865, 1697, 1633, 1462, 1207, 1104, 1067, 882 cm⁻¹; ¹H NMR (600 MHz, CDCl₃) δ 7.48 (dd, J=6.0, 2.4 Hz, 1H), 6.96 (d, J=11.8 Hz, 1H), 6.37-6.33 (m, 2H), 6.21 (dt, J=13.1, 6.1 Hz, 1H), 5.56-5.47 (m, 2H), 5.40-5.31 (m, 2H), 3.60-3.55 (m, 3H), 2.97 (t, J=6.9 Hz, 2H), 2.63-2.58 (m, 1H), 2.27 (dt, J=15.3, 8.7 Hz, 1H), 2.09-2.04 (m, 2H), 2.02-1.90 (m, 2H), 1.52-1.55 (m, 2H), 1.40-1.34 (m, 2H), 0.98 (t, J=7.8 Hz, 3H), 0.89 (s, 9H), 0.04 (s, 3H), 0.04 ppm (s, 3H); ¹³C NMR (150 MHz, CDCl₃) δ 197.48, 160.95, 144.24, 135.68, 135.36, 134.10, 132.90, 131.42, 125.84, 125.07, 124.60, 63.15, 43.77, 32.65, 31.14, 30.99, 27.28, 26.08, 25.99, 20.70, 18.44, 14.32, −5.22 ppm; HR-MS (ESI-TOF): calcd for C₂₆H₄₃O₂Si⁺ [M+H]⁺: 415.3027. found: 415.3021.

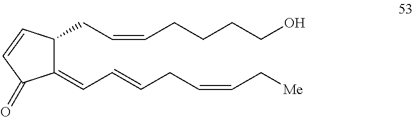 [SMILES: CC/C=C\C/C=C/C=C1/C(=O)C=C[C@@H]1C/C=C\CCCCO]

### Hydroxy dienone 53

To a stirred solution of trienone 52 (95 mg, 0.23 mmol, 1.0 equiv) in MeCN (3.5 mL) at 0° C. was dropwise added a solution of HF (50% aqueous, 460 μL, ca. 11.5 mmol, ca. 50 equiv) in MeCN (3.5 mL). After stirring for 10 min at this temperature, the reaction mixture was quenched with brine (30 mL) and extracted with EtOAc (5×50 mL). The combined organic extracts were dried (Na₂SO₄), filtered, and concentrated to a volume of ca. 1 mL (not to dryness!). Purification by flash column chromatography (SiO₂, hexane:EtOAc, 1:3) gave pure title compound (53, 61 mg, 0.20 mmol, 87% yield) as a colorless oil. 53: R_{f}=0.30 (hexane:EtOAc, 2:1); [α]_{D}²⁵=+134.2° (c=0.40 in C₆H₆); IR (film): νₘₐₓ=3423, 3009, 2930, 2859, 1686, 1626, 1578, 1456, 1298, 1206, 1067, 976, 818, 725, 519 cm⁻¹; ¹H NMR (600 MHz, CDCl₃) δ 7.48 (dd, J=6.0, 2.4 Hz, 1H), 6.96 (d, J=11.8 Hz, 1H), 6.37-6.33 (m, 2H), 6.22 (dt, J=13.5, 6.7 Hz, 1H), 5.55-5.46 (m, 2H), 5.40-5.32 (m, 2H), 3.63 (t, J=6.5 Hz, 3H), 3.57 (m, 1H), 2.98 (t, J=6.7 Hz, 2H), 2.60 (dt, J=12.4, 5.9 Hz, 1H), 2.29 (dt, J=15.1, 9.0 Hz, 1H), 2.09-2.04 (m, 4H), 1.57-1.53 (m, 2H), 1.43-1.38 (m, 2H), 0.98 ppm (t, J=7.8 Hz, 3H); ¹³C NMR (150 MHz, CDCl₃) δ 197.49, 160.88, 144.32, 135.68, 135.42, 134.12, 132.62, 131.46, 125.81, 125.27, 124.60, 62.93, 43.70, 32.47, 31.15, 30.94, 27.19, 25.84, 20.71, 14.32 ppm; HR-MS (ESI-TOF): calcd for C₂₀H₂₈O₂Na⁺ [M+Na]⁺: 323.1986. found: 323.1976.

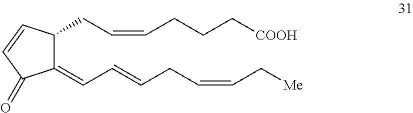 [SMILES: CC/C=C\C/C=C/C=C1/C(=O)C=C[C@@H]1C/C=C\CCCC(=O)O]

### Δ^{12,14}-PGJ₃ (31)

To a vigorously stirred solution of hydroxy trienone 53 (20.0 mg, 0.06 mmol, 1.0 equiv) in CH₂Cl₂ (2 mL) at 25° C. was added in one portion pyridinium chlorochromate (30.0 mg, 0.13 mmol, 2.0 equiv). After stirring for 2 h, the reaction mixture was directly, and without any further work-up, loaded onto a column. Flash column chromatography (SiO₂, hexane:EtOAc, 7:1→5:1) gave the intermediate aldehyde which was immediately used in the next reaction. To a vigorously stirred solution of the aldehyde in t-BuOH (1.5 mL) at 25° C. were sequentially added 2-methyl-2-butene (46 μL, 0.66 mmol, 10 equiv), a solution of NaH₂PO₄ (0.30 M in H₂O, 0.33 mL, 0.10 mmol, 1.5 equiv) and a solution of NaClO₂ (80% purity, 11.2 mg, 0.10 mmol, 1.5 equiv) in H₂O (0.5 mL). After stirring for 30 min, the reaction mixture was diluted with a solution of NaH₂PO₄ (0.30 M, 4 mL) and extracted with EtOAc (3×10 mL). The combined organic extracts were washed with brine (10 mL), dried (Na₂SO₄), filtered, and concentrated to a volume of ca. 1 mL (not to dryness!). Purification by flash column chromatography (SiO₂, hexane:EtOAc, 1:9) gave pure title compound (31, 13.5 mg, 0.04 mmol, 67% yield for the 2 steps) as a colorless oil. 31: R_{f}=0.60 (EtOAc); [α]_{D}²⁵=+134.2° (c=0.40 in C₆H₆); IR (film): νₘₐₓ=3015, 2962, 2932, 1706, 1624, 1406, 1211, 997, 526 cm⁻¹; ¹H NMR (600 MHz, CDCl₃) δ 7.47 (dd, J=6.0, 2.4 Hz, 1H), 6.96 (d, J=11.8 Hz, 1H), 6.37-6.32 (m, 2H), 6.22 (dt, J=13.5, 6.7 Hz, 1H), 5.56-5.51 (m, 1H), 5.48-5.43 (m, 1H), 5.40-5.35 (m, 2H), 3.60-3.57 (m, 1H), 2.97 (t, J=6.7 Hz, 2H), 2.59 (dt, J=14.8, 5.8 Hz, 1H), 2.34-2.27 (m, 3H), 2.09-2.03 (m, 4H), 1.68 (p, J=7.5 Hz, 2H), 0.98 ppm (t, J=7.6 Hz, 3H); ¹³C NMR (150 MHz, CDCl₃) δ 197.52, 178.35, 160.83, 144.49, 135.53, 135.49, 134.13, 131.60, 131.48, 126.16, 125.76, 124.58, 43.58, 33.26, 31.14, 30.84, 26.68, 24.55, 20.70, 14.31 ppm; HR-MS (ESI-TOF): calcd for C₂₀H₂₆O₃Na⁺ [M+Na]⁺: 337.1774. found: 337.1769.

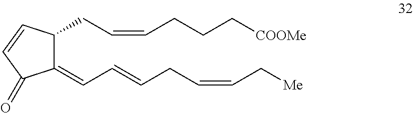 [SMILES: CC/C=C\C/C=C/C=C1/C(=O)C=C[C@@H]1C/C=C\CCCC(=O)OC]

### Δ^{12,14}-PGJ₃ methyl ester (32)

To a stirred solution of Δ^{12,14}-PGJ₃ (31) (4.0 mg, 13.0-1.0 equiv) in C₆H₆:MeOH (3:2, 0.5 mL) at 25° C. was dropwise added a solution of trimethylsilyl diazomethane (2.0 M in Et₂O, 10 μL, 19 μmol, 1.5 equiv) (yellow color persists). After stirring for 30 min, the reaction mixture was concentrated under reduced pressure. Purification by flash column chromatography (SiO₂, hexane:EtOAc, 2:1→3:2) gave pure title compound (32, 3.6 mg, 11.7 μmol, 90% yield) as a colorless oil. 32: R_{f}=0.53 (hexane:EtOAc, 1:1); [α]_{D}²⁵=+189.2° (c=0.70 in C₆H₆); IR (film): νₘₐₓ=3010, 2932, 1736, 1693, 1631, 1579, 1436, 1365, 1205, 983, 837, 728 cm⁻1; ¹H NMR (600 MHz, CDCl₃) δ 7.47 (dd, J=6.0, 2.4 Hz, 1H), 6.96 (d, J=11.8 Hz, 1H), 6.37-6.32 (m, 2H), 6.21 (dt, J=13.5, 6.7 Hz, 1H), 5.56-5.51 (m, 1H), 5.48-5.43 (m, 1H), 5.40-5.35 (m, 2H), 3.66 (s, 3H), 3.59-3.56 (m, 1H), 2.97 (t, J=6.7 Hz, 2H), 2.59 (dt, J=14.8, 5.8 Hz, 1H), 2.30-2.25 (m, 3H), 2.09-2.01 (m, 4H), 1.67 (p, J=7.5 Hz, 2H), 0.98 ppm (t, J=7.5 Hz, 3H); ¹³C NMR (150 MHz, CDCl₃) δ 197.42, 174.01, 160.78, 144.40, 135.57, 135.47, 134.10, 131.63, 131.49, 126.03, 125.78, 124.59, 51.63, 43.62, 33.52, 31.14, 30.87, 26.79, 24.82, 20.70, 14.31 ppm; HR-MS (ESI-TOF): calcd for C₂₁H₂₈O₃Na⁺ [M+Na]⁺: 351.1931. found: 351.1923.

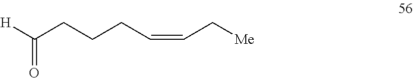

### Aldehyde 56

To a vigorously stirred solution of allylic alcohol 55 (2.00 g, 15.6 mmol, 1.0 equiv) in CH₂Cl₂ (40 mL) at 25° C. was added in one portion pyridinium chlorochromate (5.00 g, 23.0 mmol, 2.0 equiv). After stirring for 4 h, the mixture was directly, and without any further work-up, loaded onto a column. Purification by flash column chromatography (SiO₂, pentane:Et₂O, 100:0→19:1) gave pure title compound (56 (Waelchli and Eugster, 1973), 1.21 g, 9.7 mmol, 62% yield) as a colorless oil. 56: R_{f}=0.30 (hexane:EtOAc, 9:1); IR (film): νₘₐₓ=3006, 2962, 2934, 2874, 1708, 1457, 1303, 1238, 1068, 712 cm⁻¹; ¹H NMR (500 MHz, CDCl₃) δ 9.76 (t, J=1.8 Hz, 1H), 5.44-5.38 (m, 1H), 5.31-5.25 (m, 1H), 2.42 (td, J=7.3, 1.2 Hz, 2H), 2.11-1.99 (m, 4H), 1.69 (p, J=7.4 Hz, 2H), 0.94 ppm (t, J=7.5 Hz, 3H); ¹³C NMR (150 MHz, CDCl₃) δ 202.59, 133.02, 127.70, 43.33, 26.41, 22.13, 20.58, 14.31 ppm; HR-MS (ESI-TOF): calcd for C₈H₁₅O⁺ [M+H]⁺: 127.1124. found: 127.1114.

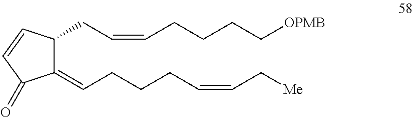

### Dienone 58

To a stirred solution of diisopropylamine (74 μL, 0.52 mmol, 2.1 equiv) in THF (3 mL) at 0° C. was dropwise added n-butyl lithium (2.5 M in hexane, 0.20 mL; 0.50 mmol; 2.0 equiv). After stirring for 20 min at this temperature, the clear solution was cooled to −78° C. and a solution of enone 2 (80 mg, 0.25 mmol, 1.0 equiv) in THF (2 mL) was added dropwise. After stirring the resulting slightly yellow solution for an additional 20 min at this temperature, a solution of aldehyde 56 (50 mg; 0.38 mmol; 1.5 equiv.) in THF (1 mL) was added dropwise and stirring at this temperature was continued for an additional 30 min. The reaction mixture was then quenched with saturated aqueous NH₄Cl solution (5 mL), diluted with EtOAc (30 mL), and allowed to warm to 25° C. The phases were separated, the aqueous layer was extracted with EtOAc (2×10 mL), and the combined organic extracts were washed with brine (50 mL), dried (Na₂SO₄), filtered, and concentrated under reduced pressure. The crude aldol product was filtered through a short column (SiO₂, hexane:EtOAc, 3:1) to obtain a mixture of diastereoisomers (98 mg, 0.22 mmol, 88% yield) as a colorless oil which was taken to the next step without further purification. To a stirred solution of the aldol product (98 mg, 0.22 mmol) in CH₂Cl₂ (4 mL) at 25° C. was added DMAP (269 mg, 2.20 mmol, 10 equiv), and then, slowly and dropwise, methanesulfonyl chloride (34 μL, 0.44 mmol, 2.0 equiv). After stirring for 12 h at this temperature, the reaction mixture was quenched with saturated aqueous NaHCO₃-solution (5 mL) and diluted with CH₂Cl₂ (20 mL). The phases were separated, the aqueous layer was extracted with CH₂Cl₂ (2×10 mL), and the combined organic layers were washed with H₂O (20 mL), dried (Na₂SO₄), filtered, and concentrated under reduced pressure. Purification by flash column chromatography (SiO₂, hexane:EtOAc, 7:1) gave pure title compound (58, 66 mg, 0.15 mmol, 61% yield for the 2 steps) as a colorless oil. 58: R_{f}=0.60 (hexane:EtOAc, 3:1); [α]_{D}²⁵=+136.3° (c=1.00 in C₆H₆); IR (film): νₘₐₓ=3005, 2933, 2858, 1702, 1654, 1613, 1583, 1513, 1458, 1301, 1247, 1172, 1099, 1036, 820 cm⁻¹; ¹H NMR (600 MHz, CDCl₃) δ 7.47 (dd, J=6.0, 2.4 Hz, 1H), 7.25 (d, J=8.6 Hz, 2H), 6.87 (d, J=8.6 Hz, 2H), 6.57 (t, J=7.5 Hz, 1H), 6.32 (dd, J=6.0, 1.7 Hz, 1H), 5.50-5.45 (m, 1H), 5.43-5.38 (m, 1H), 5.34-5.28 (m, 2H), 4.42 (s, 2H), 3.80 (s, 3H), 3.49-3.46 (m, 1H), 3.43 (t, J=6.6 Hz, 2H), 2.59-2.54 (m, 1H), 2.33-2.17 (m, 3H), 2.09 (q, J=7.3 Hz, 2H), 2.04-1.98 (m, 4H), 1.61-1.53 (m, 4H), 1.44-1.39 (m, 2H), 0.95 ppm (t, J=7.6 Hz, 3H); ¹³C NMR (150 MHz, CDCl₃) δ 196.55, 161.73, 159.26, 137.83, 135.92, 135.04, 132.74, 132.62, 130.82, 129.34, 128.21, 125.19, 113.89, 72.70, 70.03, 55.41, 43.52, 30.49, 29.54, 28.90, 28.79, 27.29, 26.89, 26.31, 20.70, 14.45 ppm; HR-MS (ESI-TOF): calcd for C₂₈H₃₈O₃Na⁺ [M+Na]⁺: 445.2725. found: 445.2728.

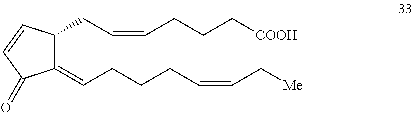 [SMILES: CC/C=C\CCC/C=C1/C(=O)C=C[C@@H]1C/C=C\CCCC(=O)O]

### 15-deoxy-Δ¹²-PGJ₃ (33)

To a stirred solution of dienone 58 (20 mg, 47 μmol, 1.0 equiv) in MeCN:H₂O (9:1, 0.4 mL) at 25° C. was dropwise added 4-(acetyl amino)-2,2,6,6-tetramethyl-1-oxo-piperidinium tetrafluoroborate (85 mg, 282 μmol, 6.0 equiv). After stirring for 30 min, the reaction mixture was diluted with a solution of H₂O (2 mL) and extracted with EtOAc (3×10 mL). The combined organic extracts were washed with brine (5 mL), dried (Na₂SO₄), filtered, and concentrated to a volume of ca. 1 mL (not to dryness!). Purification by flash column chromatography (SiO₂, hexane:EtOAc, 1:9) gave pure title compound (33, 6.9 mg, 21 μmol, 45% yield) as a colorless oil. 33: R_{f}=0.40 (hexane:EtOAc, 1:9); [α]_{D}²⁵=+171.5° (c=1.00 in C₆H₆); IR (film): νₘₐₓ=3007, 2961, 2932, 1704, 1652, 1579, 1479, 1237, 679 cm⁻¹; ¹H NMR (600 MHz, CDCl₃) δ 7.48 (dd, J=6.0, 2.4 Hz, 1H), 6.58 (t, J=8.0 Hz, 1H), 6.34 (dd, J=6.0, 1.8 Hz, 1H), 5.48-5.43 (m, 1H), 5.42-5.34 (m, 1H), 5.32-5.27 (m, 2H), 3.51-3.48 (m, 1H), 2.57 (dt, J=14.6, 5.5 Hz, 1H), 2.33 (t, J=7.6 Hz, 2H), 2.31-2.19 (m, 3H), 2.10-1.99 (m, 6H), 1.68 (p, J=7.6 Hz, 2H), 1.56 (p, J=7.6 Hz, 2H), 0.94 ppm (t, J=7.6 Hz, 3H); ¹³C NMR (150 MHz, CDCl₃) δ 196.94, 179.19, 161.68, 137.69, 136.20, 135.10, 132.74, 131.36, 128.17, 126.19, 43.37, 33.40, 30.38, 28.89, 28.74, 26.84, 26.68, 24.50, 20.68, 14.42 ppm; HR-MS (ESI-TOF): calcd for C₂₀H₂₈O₃Na⁺ [M+Na]⁺: 339.1931. found: 339.1937.

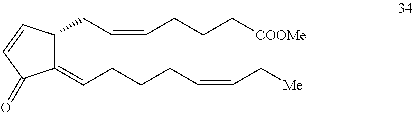 [SMILES: CC/C=C\CCC/C=C1/C(=O)C=C[C@@H]1C/C=C\CCCC(=O)OC]

### 15-deoxy-Δ¹²-PGJ₃ methyl ester (34)

To a stirred solution of 15-deoxy Δ¹²-PGJ₃ (33) (3.3 mg, 10 μmol, 1.0 equiv) in C₆H₆:MeOH (3:2, 0.5 mL) at 25° C. was dropwise added a solution of trimethylsilyl diazomethane (2.0 M in Et₂O, 10 μL, 20 μmol, 1.5 equiv) (yellow color persists). After stirring for 30 min, the reaction mixture was concentrated under reduced pressure. Purification by flash column chromatography (SiO₂, hexane:EtOAc, 6:1→3:2) gave pure title compound (34, 3.0 mg, 9 μmol, 90% yield) as a colorless oil. 34: R_{f}=0.53 (hexane:EtOAc, 1:1); [α]_{D}²⁵=+162.5° (c=0.40 in C₆H₆); IR (film): νₘₐₓ=3006, 1932, 1737, 1702, 1654, 1581, 1436, 1365, 1215, 1173, 517 cm⁻¹; ¹H NMR (600 MHz, CDCl₃) δ 7.46 (dd, J=6.0, 2.4 Hz, 1H), 6.56 (t, J=8.0 Hz, 1H), 6.31 (dd, J=6.0, 1.8 Hz, 1H), 5.46-5.42 (m, 1H), 5.41-5.27 (m, 3H), 3.64 (s, 3H), 3.50-3.47 (m, 1H), 2.56 (dt, J=15.1, 5.8 Hz, 1H), 2.31-2.18 (m, 5H), 2.08 (q, J=7.4 Hz, 2H), 2.04-1.98 (m, 4H), 1.68 (p, J=7.6 Hz, 2H), 1.57 (p, J=7.6 Hz, 2H), 0.93 ppm (t, J=7.6 Hz, 3H); ¹³C NMR (150 MHz, CDCl₃) δ 196.73, 173.94, 161.51, 137.69, 135.93, 135.09, 132.70, 131.48, 128.16, 126.02, 51.59, 43.36, 34.46, 30.37, 28.85, 28.74, 26.83, 26.76, 24.75, 20.66, 14.40 ppm; HR-MS (ESI-TOF): calcd for C₂₁H₃₀O₃Na⁺ [M+Na]⁺: 353.2087. found: 353.2094.

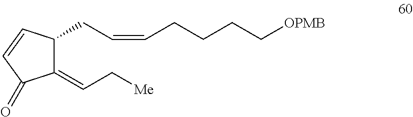

### Dienone 60

To a stirred solution of diisopropylamine (60 μL, 0.42 mmol, 2.2 equiv) in THF (2 mL) at 0° C. was dropwise added n-butyl lithium (2.5 M in hexane, 150 μL, 0.38 mmol, 2.0 equiv). After stirring for 20 min at this temperature, the clear solution was cooled to −78° C. and a solution of enone 2 (60 mg, 0.19 mmol, 1.0 equiv) in THF (1 mL) was added dropwise. After stirring the resulting slightly yellow solution for an additional 20 min at this temperature, a solution of propionaldehyde (21 μL, 0.28 mmol, 1.5 equiv) in THF (1 mL) was added dropwise and stirring at this temperature was continued for an additional 30 min. The reaction mixture was then quenched with saturated aqueous NH₄Cl solution (5 mL), diluted with EtOAc (25 mL), and allowed to warm to 25° C. The phases were separated, the aqueous layer was extracted with EtOAc (2×15 mL), and the combined organic extracts were washed with brine (50 mL), dried (Na₂SO₄), filtered, and concentrated under reduced pressure. The crude aldol product was filtered through a short column (SiO₂, hexane:EtOAc, 3:1) to obtain a mixture of diastereoisomers (55 mg, 0.15 mmol, 79% yield) as a colorless oil which was taken to the next step without further purification. To a stirred solution of the aldol product (55 mg, 0.15 mmol) in CH₂Cl₂ (2 mL) at 25° C. was added DMAP (183 mg, 1.50 mmol, 10 equiv), and then, slowly and dropwise, methanesulfonyl chloride (73 μL, 3.00 mmol, 2.0 equiv). After stirring for 12 h at this temperature, the reaction mixture was quenched with saturated aqueous NaHCO₃-solution (5 mL) and diluted with CH₂Cl₂ (25 mL). The phases were separated, the aqueous layer was extracted with CH₂Cl₂ (2×10 mL), and the combined organic layers were washed with H₂O (20 mL), dried (Na₂SO₄), filtered, and concentrated under reduced pressure. Purification by flash column chromatography (SiO₂, hexane:EtOAc, 7:1) gave pure title compound (60, 42 mg, 0.12 mmol, 63% yield for the 2 steps) as a colorless oil. 60: R_{f}=0.68 (hexane:EtOAc, 3:1); [α]_{D}²⁵=+141.2° (c=1.00 in C₆H₆); IR (film): νₘₐₓ=2934, 2857, 1702, 1655, 1613, 1513, 1460, 1301, 1247, 1099, 1034, 811 cm⁻¹; ¹H NMR (600 MHz, CDCl₃) δ 7.47 (ddd, J=6.2, 2.7 Hz, 1H), 7.25 (d, J=8.6 Hz, 2H), 6.87 (d, J=8.6 Hz, 2H), 6.54 (t, J=7.9 Hz, 1H), 6.32 (dd, J=6.0, 1.6 Hz, 1H), 5.50-5.45 (m, 1H), 5.34-5.30 (m, 1H), 4.42 (s, 2H), 3.80 (s, 3H), 3.50-3.47 (m, 1H), 3.43 (t, J=6.5 Hz, 2H), 2.56 (dt, J=14.2, 5.0 Hz, 1H), 2.33-2.25 (m, 2H), 2.21 (dt, J=14.8, 8.3 Hz, 1H), 2.00 (q, J=7.0 Hz, 2H), 1.59 (p, J=7.6 Hz, 2H), 1.41 (p, J=7.6 Hz, 2H), 1.11 ppm (t, J=7.5 Hz, 3H); ¹³C NMR (150 MHz, CDCl₃) δ 197.00, 161.70, 159.25, 137.47, 137.09, 135.03, 132.59, 130.81, 129.33, 125.20, 113.88, 72.69, 70.01, 55.40, 43.45, 30.50, 29.52, 27.27, 26.29, 22.75, 13.27 ppm; HR-MS (ESI-TOF): calcd for C₂₃H₃₀O₃Na⁺ [M+Na]⁺: 377.2087. found: 377.2098.

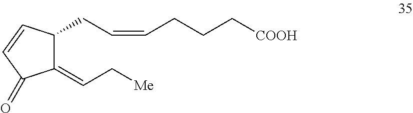 [SMILES: CC/C=C1/C(=O)C=C[C@@H]1C/C=C\CCCC(=O)O]

### Δ¹²-PGJ₃ analog (35)

To a stirred solution of dienone 60 (10 mg, 30 μmol, 1.0 equiv) in MeCN:H₂O (9:1, 0.4 mL) at 25° C. was dropwise added 4-(acetyl amino)-2,2,6,6-tetramethyl-1-oxo-piperidinium tetrafluoroborate (54 mg, 15 μmol, 6.0 equiv). After stirring for 30 min, the reaction mixture was diluted with H₂O (2 mL) and extracted with EtOAc (3×10 mL). The combined organic extracts were washed with brine (5 mL), dried (Na₂SO₄), filtered, and concentrated to a volume of ca. 1 mL (not to dryness!). Purification by flash column chromatography (SiO₂, hexane:EtOAc, 1:9) gave pure title compound (35, 4.2 mg, 17 μmol, 57% yield) as a colorless oil. 35: R_{f}=0.40 (hexane:EtOAc, 1:10); [α]_{D}²⁵=+142.5° (c=1.00 in C₆H₆); IR (film): νₘₐₓ=3010, 2966, 2934, 2874, 1728, 1700, 1641, 1578, 1215, 1150, 810 cm⁻¹; ¹H NMR (600 MHz, CDCl₃) δ 7.47 (ddd, J=6.2, 2.7 Hz, 1H), 6.55 (t, J=7.9 Hz, 1H), 6.33 (dd, J=6.0, 1.6 Hz, 1H), 5.47-5.43 (m, 1H), 5.38-5.34 (m, 1H), 3.52-3.49 (m, 1H), 2.57 (dt, J=14.2, 5.3 Hz, 1H), 2.33 (t, J=7.6 Hz, 2H), 2.32-2.26 (m, 2H), 2.22 (dt, J=15.2, 8.3 Hz, 1H), 2.05 (q, J=7.0 Hz, 2H), 1.68 (p, J=7.6 Hz, 2H), 1.10 ppm (t, J=7.5 Hz, 3H); ¹³C NMR (150 MHz, CDCl₃) δ 196.94, 174.00, 161.54, 137.56, 136.99, 135.14, 131.50, 126.08, 43.34, 33.50, 30.42, 26.78, 24.78, 22.76, 13.27 ppm; HR-MS (ESI-TOF): calcd for C₁₅H₂₀O₃Na⁺ [M+Na]⁺: 271.1305. found: 271.1304.

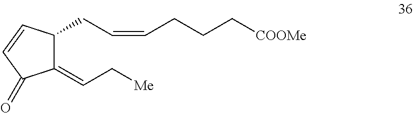 [SMILES: CC/C=C1/C(=O)C=C[C@@H]1C/C=C\CCCC(=O)OC]

### Δ¹²-PGJ₃ analog 36

To a stirred solution of compound 35 (10.0 mg; 40 μmol, 1.0 equiv) in C₆H₆:MeOH (3:2, 0.5 mL) at 25° C. was dropwise added a solution of trimethylsilyl diazomethane (2.0 M in Et₂O, 30 μL, 60 μmol, 1.5 equiv) (yellow color persists). After stirring for 30 min, the reaction mixture was concentrated under reduced pressure. Purification by flash column chromatography (SiO₂, hexane:EtOAc, 2:1→3:2) gave pure title compound (36, 9.0 mg, 36 μmol, 90% yield) as a colorless oil. 36: R_{f}=0.53 (hexane:EtOAc, 1:1); [α]_{D}²⁵=+168.3° (c=0.30 in C₆H₆); IR (film): νₘₐₓ=3009, 2950, 2874, 1736, 1702, 1654, 1580, 1436, 1212, 1151, 809 cm⁻¹; ¹H NMR (600 MHz, CDCl₃) δ 7.48 (ddd, J=6.2, 2.7 Hz, 1H), 6.55 (t, J=7.9 Hz, 1H), 6.33 (dd, J=6.0, 1.6 Hz, 1H), 5.47-5.43 (m, 1H), 5.38-5.34 (m, 1H), 3.66 (s, 3H), 3.52-3.49 (m, 1H), 2.57 (dt, J=14.2, 5.3 Hz, 1H), 2.33-2.26 (m, 4H), 2.22 (dt, J=15.2, 8.3 Hz, 1H), 2.03 (q, J=7.5 Hz, 2H), 1.67 (p, J=7.6 Hz, 2H), 1.11 ppm (t, J=7.5 Hz, 3H); ¹³C NMR (150 MHz, CDCl₃) δ 196.93, 173.99, 161.53, 137.55, 136.98, 135.13, 131.50, 126.07, 51.64, 43.35, 33.50, 30.43, 26.79, 24.78, 22.77, 13.27 ppm; HR-MS (ESI-TOF): calcd for C₁₆H₂₂O₃Na⁺ [M+Na]⁺: 285.1461. found: 285.1472.

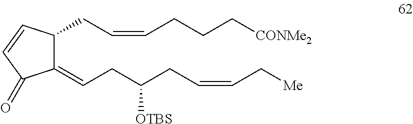

### Dienone N,N-dimethylamide 62

To a stirred solution of carboxylic acid 61 (12 mg, 0.030 mmol, 1.0 equiv) in CH₂Cl₂ (1 mL) at 0° C. was added EDCI (80% aq., 8.0 mg, 0.045 mmol, 1.5 equiv) and 1-hydroxy benzotriazole (8.0 mg, 0.045 mmol, 1.5 equiv). After stirring for 20 min at this temperature, dimethyl amine (2.0 M in THF, 16 μL, 0.032 mmol, 1.2 equiv) was added. After stirring the resulting solution for an additional 6 h at 25° C., the reaction mixture was then quenched with saturated aqueous NH₄Cl solution (75 mL) and diluted with CH₂Cl₂ (15 mL). The phases were separated, the aqueous layer was extracted with CH₂Cl₂ (2×15 mL). The combined organic extracts were washed with brine (50 mL), dried (Na₂SO₄), filtered, and concentrated under reduced pressure. Purification by flash column chromatography (SiO₂, hexane:EtOAc, 1:4) gave pure title compound (62, 8.8 mg, 21 μmol, 71% yield) as a colorless oil. 62: R_{f}=0.60 (EtOAc); [α]_{D}²⁵=+123.3° (c=0.60 in C₆H₆); IR (film): νₘₐₓ=3009, 2955, 2929, 2856, 1703, 1653, 1462, 1397, 1256, 1083, 836, 776 cm⁻¹; ¹H NMR (600 MHz, CDCl₃) δ 7.49 (dd, J=5.8, 2.7 Hz, 1H), 6.60 (t, J=7.6 Hz, 1H), 6.31 (dd, J=6.0, 1.8 Hz, 1H), 5.55-5.45 (m, 2H), 5.38-5.33 (m, 2H), 3.88 (p, J=6.2 Hz, 1H), 3.47-3.44 (m, 1H), 2.98 (s, 3H), 2.93 (s, 3H), 2.62 (dt, J=14.4, 5.7 Hz, 1H), 2.47-2.38 (m, 2H), 2.28 (t, J=7.5 Hz, 2H), 2.25-2.15 (m, 3H), 2.10-1.97 (m, 4H), 1.68 (p, J=7.5 Hz, 2H), 0.93 (t, J=7.5 Hz, 3H), 0.87 (s, 9H), 0.06 (s, 3H), 0.05 ppm (s, 3H); ¹³C NMR (150 MHz, CDCl₃) δ 196.41, 172.78, 161.70, 138.87, 135.00, 134.13, 132.61, 132.05, 125.85, 124.41, 71.68, 43.48, 37.33, 36.91, 35.50, 35.30, 32.78, 30.65, 27.12, 25.97, 24.99, 20.88, 18.20, 14.31, −4.43 ppm; HR-MS (ESI-TOF): calcd for C₂₈H₄₇NO₃SiNa⁺ [M+Na]⁺: 496.3217. found: 496.3201.

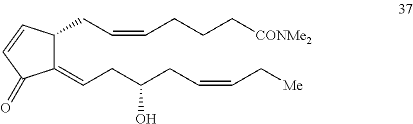

### Δ¹²-PGJ₃ analog 37

To a stirred solution of dienone N,N-dimethylamide 62 (6.0 mg, 12.6-1.0 equiv) in MeCN (1 mL) at 0° C. was dropwise added a solution of HF (50% aqueous; 25 μL, ca. 0.63 mmol, ca. 50 equiv) in MeCN (0.5 mL). After stirring for 45 min at this temperature, the reaction mixture was quenched with brine (3 mL) and extracted with EtOAc (5×5 mL). The combined organic extracts were dried (Na₂SO₄), filtered, and concentrated to a volume of ca. 1 mL (not to dryness!). Purification by flash column chromatography (SiO₂, hexane:EtOAc, 10:1) gave pure title compound (37, 3.8 mg, 10.6 μmol, 84% yield) as a colorless oil. 37: R_{f}=0.30 (EtOAc); [α]_{D}²⁵=+116.0° (c=0.30 in CHCl₃); IR (film): νₘₐₓ=3419, 3409, 3008, 2959, 2927, 2872, 1697, 1626, 1579, 1400, 1260, 1048, 801 cm⁻¹; ¹H NMR (600 MHz, CDCl₃) δ 7.53 (dd, J=5.8, 2.7 Hz, 1H), 6.62 (t, J=7.6 Hz, 1H), 6.34 (dd, J=6.0, 1.8 Hz, 1H), 5.59-5.54 (m, 1H), 5.52-5.47 (m, 1H), 5.43-5.33 (m, 2H), 3.86-3.81 (m, 1H), 3.52-3.49 (m, 1H), 2.99 (s, 3H), 2.93 (s, 3H), 2.71-2.67 (m, 2H), 2.58 (dt, J=14.6, 7.1 Hz, 1H), 2.48 (ddd, J=14.6, 8.2, 6.5 Hz, 1H), 2.31-2.28 (m, 4H), 2.21 (dt, J=15.8, 8.8 Hz, 1H), 2.11-2.03 (m, 4H), 1.71-1.64 (m, 2H), 0.96 ppm (t, J=7.5 Hz, 3H); ¹³C NMR (150 MHz, CDCl₃) δ 196.50, 172.91, 161.75, 139.47, 135.35, 135.07, 132.22, 131.97, 125.68, 124.19, 70.71, 43.72, 37.40, 36.95, 35.64, 34.86, 32.61, 30.43, 27.20, 24.95, 20.89, 14.34 ppm; HR-MS (ESI-TOF): calcd for C₂₂H₃₃NO₃Na⁺ [M+Na]⁺: 382.2353. found: 382.2365.

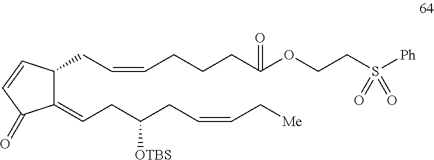 [SMILES: CC/C=C\C[C@@H](C/C=C1/C(=O)C=C[C@@H]1C/C=C\CCCC(=O)OCCS(=O)(=O)c1ccccc1)O[Si](C)(C)C(C)(C)C]

### Dienone sulfonate ester 64

To a stirred solution of carboxylic acid 61 (12 mg, 0.027 mmol, 1.0 equiv) in CH₂Cl₂ (1 mL) at 0° C. was added EDCI (8 mg, 40 μmol, 1.5 equiv) and DMAP (0.2 mg, 1.3 μmol, 0.05 equiv). After stirring for 20 min at this temperature, alcohol 63 (8 mg, 40 μmol, 1.5 equiv) was added. After stirring the resulting solution for an additional 8 h at 0° C., the reaction mixture was quenched with saturated aqueous NH₄Cl solution (5 mL) and diluted with CH₂Cl₂ (5 mL). The phases were separated, the aqueous layer was extracted with CH₂Cl₂ (2×5 mL), and the combined organic extracts were washed with brine (50 mL), dried (Na₂SO₄), filtered, and concentrated under reduced pressure. Purification by flash column chromatography (SiO₂, hexane:EtOAc, 3:2) gave pure title compound (64, 8.1 mg, 0.013 mmol, 48% yield) as a colorless oil. 64: R_{f}=0.60 (EtOAc, hexane:EtOAc, 2:3); [α]_{D}²⁵=+114.8° (c=0.45 in C₆H₆); IR (film): νₘₐₓ=3008, 2956, 2929, 2856, 1740, 1702, 1655, 1462, 1447, 1323, 1251, 1086, 836 cm⁻¹; ¹H NMR (600 MHz, CDCl₃) δ 7.93-7.91 (m, 2H), 7.67 (tt, J=7.4, 1.1 Hz, 1H), 7.58 (t, J=7.5 Hz, 2H), 7.47 (dd, J=6.1, 2.7 Hz, 1H), 6.60 (t, J=7.6 Hz, 1H), 6.32 (dd, J=6.0, 1.8 Hz, 1H), 5.49-5.44 (m, 1H), 5.43-5.34 (m, 3H), 4.40 (t, J=6.2 Hz, 2H), 3.88 (p, J=6.2 Hz, 1H), 3.45 (t, J=6.2 Hz, 3H), 2.59 (dt, J=14.4, 5.4 Hz, 1H), 2.46-2.37 (m, 2H), 2.28-2.15 (m, 3H), 2.07 (t, J=7.6 Hz, 2H), 2.03-1.94 (m, 4H), 1.54 (p, J=7.5 Hz, 2H), 0.94 (t, J=7.5 Hz, 3H), 0.88 (s, 9H), 0.06 (s, 3H), 0.05 ppm (s, 3H); ¹³C NMR (150 MHz, CDCl₃) δ 196.33, 172.80, 161.50, 139.59, 138.79, 135.10, 134.16, 134.07, 132.70, 131.24, 129.49, 128.26, 126.33, 124.39, 71.67, 57.60, 55.17, 43.38, 36.91, 35.30, 33.23, 30.63, 26.69, 25.97, 24.47, 20.89, 18.20, 14.31, −4.43 ppm; HR-MS (ESI-TOF): calcd for C₃₄H₅₀O₆SSiNa⁺ [M+Na]⁺: 637.2994. found: 637.2982.

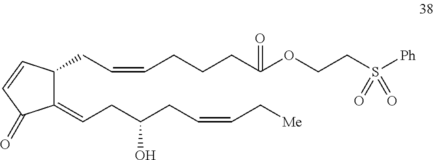 [SMILES: CC/C=C\C[C@H](O)C/C=C1/C(=O)C=C[C@@H]1C/C=C\CCCC(=O)OCCS(=O)(=O)c1ccccc1]

### Δ¹²-PGJ₃ analog 38

To a stirred solution of dienone sulfonate ester 64 (4.0 mg, 6.5-1.0 equiv) in MeCN (1 mL) at 0° C. was dropwise added a solution of HF (50% aqueous, 13.1 μL, ca. 0.3 mmol, ca. 50 equiv) in MeCN (0.5 mL). After stirring for 45 min at this temperature, the reaction mixture was quenched with brine (3 mL) and extracted with EtOAc (5×5 mL). The combined organic extracts were dried (Na₂SO₄), filtered, and concentrated to a volume of ca. 1 mL (not to dryness!). Purification by flash column chromatography (SiO₂, hexane:EtOAc, 2:3) gave pure title compound (38, 2.3 mg, 4.9 μmol, 76% yield) as a colorless oil. 38: R_{f}=0.20 (hexane:EtOAc, 2:3); [α]_{D}²⁵=+99.3° (c=0.20 in CHCl₃); IR (film): νₘₐₓ=3425, 2925, 1738, 1697, 1651, 1447, 1321, 1142, 1085, 726 cm⁻¹; ¹H NMR (600 MHz, CDCl₃) δ 7.93-7.91 (m, 2H), 7.67 (tt, J=7.4, 1.1 Hz, 1H), 7.58 (t, J=7.5 Hz, 2H), 7.49 (dd, J=6.1, 2.7 Hz, 1H), 6.63 (t, J=7.6 Hz, 1H), 6.34 (dd, J=6.0, 1.8 Hz, 1H), 5.62-5.57 (m, 1H), 5.43-5.34 (m, 3H), 4.41 (t, J=6.2 Hz, 2H), 3.88-3.82 (m, 1H), 3.52-3.50 (m, 1H), 3.45 (t, J=6.3 Hz, 2H), 2.61 (dt, J=14.4, 5.4 Hz, 1H), 2.54-2.43 (m, 2H), 2.28 (t, J=6.8 Hz, 2H), 2.23 (dt, J=15.6, 7.2 Hz, 1H), 2.10-2.03 (m, 4H), 1.97 (q, J=7.7 Hz, 2H), 1.90 (d, J=4.4 Hz, 1H), 1.55 (p, J=6.8 Hz, 2H), 0.96 ppm (t, J=7.5 Hz, 3H); ¹³C NMR (150 MHz, CDCl₃) δ 196.43, 172.93, 161.70, 139.56, 139.48, 135.91, 135.11, 134.10, 131.80, 131.44, 129.51, 128.26, 126.08, 123.82, 70.66, 57.61, 55.17, 43.35, 36.66, 35.05, 33.19, 30.39, 26.69, 24.43, 20.89, 14.34 ppm; HR-MS (ESI-TOF): calcd for C₂₈H₃₆O₆SNa⁺ [M+Na]⁺: 523.2125. found: 521.2122.

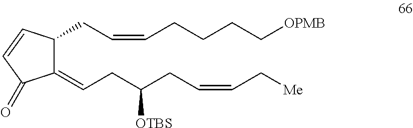

### Dienone 66

To a stirred solution of diisopropyl amine (270 μL, 1.93 mmol, 2.2 equiv) in THF (8 mL) at 0° C. was added n-butyl lithium (1.1 mL, 1.6 M in hexane, 1.76 mmol, 2.0 equiv) dropwise. After stirring at 0° C. for 20 min the resulting solution was cooled to −78° C. and a solution of enone 2 (275 mg, 0.875 mmol, 1.0 equiv) in THF (6 mL) was added dropwise. After stirring at −78° C. for 30 min a solution of aldehyde ent-3 (270 mg, 1.06 mmol, 1.2 equiv) in THF (6 mL) was added dropwise. After stirring at the same temperature for 30 min the reaction mixture was quenched with saturated aqueous NH₄Cl solution (30 mL) and warmed to 25° C. The aqueous layer was extracted with EtOAc (3×30 mL). The combined organic layers were washed with brine (30 mL), dried (Na₂SO₄) and concentrated under reduced pressure. The residue was filtered through a short column (SiO₂, hexane:EtOAc, 3:1) to obtain a mixture of diastereomers (ca. 6:1, judged by ¹H HMR, 430 mg, 0.750 mmol, 86%) as light yellow oil which was used in the next step without further purification. The crude aldol product 65 (418 mg, 0.732 mmol, 1.0 equiv) was dissolved in CH₂Cl₂ (9 mL) and cooled to 0° C. Triethylamine (1.02 mL, 7.32 mmol, 10 equiv) was added to the above solution followed by dropwise addition of methanesulfonyl chloride (283 μL, 3.66 mmol, 5.0 equiv). After stirring at the same temperature for 5 min, the reaction mixture was quenched with saturated aqueous NaHCO₃-solution (40 mL), diluted with CH₂Cl₂ (40 mL) and warmed to 25° C. The aqueous layer was extracted with CH₂Cl₂ (2×50 mL). The combined organic layers were washed with water (20 mL), dried over Na₂SO₄ and concentrated under reduced pressure. The residue was filtered through a short column (SiO₂, hexane:EtOAc, 2:1) to obtained the crude mesylate (438 mg, 0.674 mmol, 92%) as yellow oil which was used in the next step without further purification. The above crude mesylate (438 mg, 0.674 mmol, 1.0 equiv) was dissolved in CH₂Cl₂ (15 mL) and neutral alumina (481 mg, 4.72 mmol, 7.0 equiv) was added at 25° C. The reaction mixture was stirred at 25° C. for 8 h during which time neutral alumina (5×481 mg, 5×4.72 mmol, 5×7.0 equiv) was added every 1.5 h. Upon completion of the reaction as indicated by thin-layer chromatography (TLC), the reaction mixture was filtered through a pad of Celite®, rinsed with EtOAc and concentrated under reduced pressure. Purification by flash column chromatography (SiO₂, hexane:EtOAc, 10:1) gave pure title compound (66, 264 mg, 0.477 mmol, 65% overall yield) as yellow oil. 66: R_{f}=0.58 (hexane:EtOAc, 3:1); [α]_{D}²⁵=+87.2° (c=1.0 in C₆H₆); IR (film): νₘₐₓ=2930, 2856, 1704, 1656, 1513, 1247, 1095, 835, 775 cm⁻¹; ¹H NMR (600 MHz, CDCl₃) δ 7.46 (ddd, J=6.1, 2.7, 1.0 Hz, 1H), 7.25 (d, J=8.5 Hz, 2H), 6.87 (d, J=8.5 Hz, 2H), 6.65-6.59 (m, 1H), 6.31 (dd, J=6.0, 1.8 Hz, 1H), 5.52-5.43 (m, 2H), 5.38-5.27 (m, 2H), 4.42 (s, 2H), 3.85 (tt, J=6.8, 4.8 Hz, 1H), 3.80 (s, 3H), 3.52-3.45 (m, 1H), 3.42 (t, J=6.5 Hz, 2H), 2.54 (dddd, J=14.5, 6.7, 4.2, 1.6 Hz, 1H), 2.46-2.34 (m, 2H), 2.31-2.15 (m, 4H), 2.07-1.95 (m, 4H), 1.63-1.54 (m, 2H), 1.41 (tt, J=10.0, 6.3 Hz, 2H), 0.94 (t, J=7.5 Hz, 3H), 0.85 (s, 9H), 0.04 (s, 3H), 0.00 ppm (s, 3H); ¹³C NMR (150 MHz, CDCl₃) δ 196.37, 161.67, 159.24, 139.25, 135.00, 134.16, 133.04, 132.60, 130.81, 129.31, 125.18, 124.49, 113.88, 72.68, 71.76, 70.02, 55.39, 43.51, 36.85, 35.76, 30.57, 29.54, 27.29, 26.31, 25.92, 20.88, 18.13, 14.32, −4.39, −4.53 ppm; HR-MS (ESI-TOF) calcd for C₃₄H₅₂O₄SiNa⁺ [M+Na]⁺: 575.3527. found: 575.3507.

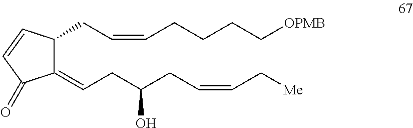

### Hydroxydienone 67

To a stirred solution of dienone 66 (132 mg, 0.239 mmol, 1.0 equiv) in THF (4.4 mL) at 0° C. was added 3HF.Et₃N (900 μL, 5.52 mmol, 23 equiv) dropwise. The resulting mixture was warmed to 25° C. and stirred for three days. Upon completion of the reaction as indicated by thin-layer chromatography (TLC), the mixture was cooled to 0° C., quenched with excess solid NaHCO₃, warmed to 25° C. and diluted with saturated aqueous NaHCO₃ solution (10 mL). The aqueous layer was extracted with EtOAc (3×10 mL). The combined organic layers were dried over Na₂SO₄ and concentrated under reduced pressure. Purification by flash column chromatography (SiO₂, hexane:EtOAc, 1:1) gave pure title compound (67, 92.7 mg, 0.210 mmol, 88% yield) as light yellow oil. 67: R_{f}=0.39 (hexane:EtOAc, 1:1); [α]_{D}²⁵=+133.8° (c=1.0 in C₆H₆); IR (film): νₘₐₓ=3430, 2933, 2859, 1699, 1651, 1513, 1247, 1097, 1036, 819 cm⁻¹; ¹H NMR (600 MHz, CDCl₃) δ 7.49 (ddd, J=6.1, 2.6, 1.0 Hz, 1H), 7.25 (d, J=8.6 Hz, 2H), 6.87 (d, J=8.6 Hz, 2H), 6.64 (dd, J=9.1, 6.6 Hz, 1H), 6.33 (dd, J=5.9, 1.8 Hz, 1H), 5.64-5.56 (m, 1H), 5.51-5.44 (m, 1H), 5.40-5.34 (m, 1H), 5.34-5.28 (m, 1H), 4.42 (s, 2H), 3.84-3.78 (m, 1H), 3.80 (s, 3H), 3.56-3.50 (m, 1H), 3.42 (t, J=6.5 Hz, 2H), 2.62-2.54 (m, 1H), 2.48 (ddd, J=15.1, 6.4, 4.8 Hz, 1H), 2.45-2.40 (m, 1H), 2.35-2.17 (m, 3H), 2.10-2.03 (m, 2H), 2.03-1.96 (m, 2H), 1.85 (br s, 1H), 1.63-1.55 (m, 2H), 1.41 (tt, J=7.5, 7.5 Hz, 2H), 0.97 ppm (t, J=7.5 Hz, 3H); ¹³C NMR (150 MHz, CDCl₃) δ 196.47, 161.95, 159.26, 139.76, 135.78, 134.96, 132.73, 131.93, 130.79, 129.35, 125.04, 123.92, 113.90, 72.70, 70.77, 70.00, 55.42, 43.50, 36.58, 35.26, 30.46, 29.54, 27.32, 26.32, 20.90, 14.35 ppm; HR-MS (ESI-TOF) calcd for C₂₈H₃₈O₄Na⁺ [M+Na]⁺: 461.2662. found: 461.2657.

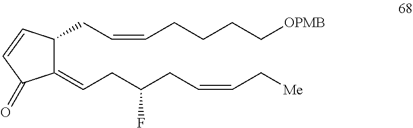

### Fluorodienone 68

To an oven-dried flask was added potassium fluoride (34.0 mg, 0.593 mmol, 9.0 equiv) and the flask, along with the potassium fluoride, was flame-dried under vacuum. After cooling to 25° C., PhenoFluor (169 mg, 0.395 mmol, 6.0 equiv) was added, followed by a solution of hydroxydienone 67 (28.9 mg, 0.0659 mmol, 1.0 equiv) in anhydrous toluene (1.3 mL). N-ethyldiisopropylamine (102 μL, 0.593 mmol, 9.0 equiv) was added and the reaction mixture was heated to 80° C. and stirred for 2.5 h. After cooling to 25° C., the reaction mixture was concentrated under reduced pressure. Purification by flash column chromatography (SiO₂, hexane:EtOAc:dichloromethane, 5:1:1) gave a mixture of the desired product and elimination by-product. The mixture was further purified by preparative thin-layer chromatography (SiO₂, hexane:acetone, 6:1) to give the pure title compound (68, 10.0 mg, 0.0227 mmol, 35% yield) as yellow oil. 68: R_{f}=0.27 (hexane:EtOAc, 4:1); [α]_{D}²⁵=+137.9° (c=0.80 in C₆H₆); IR (film): νₘₐₓ=2933, 2857, 1703, 1657, 1513, 1246, 1098, 1035, 820 cm⁻¹; ¹H NMR (600 MHz, CDCl₃) δ 7.50 (ddd, J=6.1, 2.6, 1.0 Hz, 1H), 7.25 (d, J=8.6 Hz, 2H), 6.87 (d, J=8.6 Hz, 2H), 6.59 (dd, J=8.2, 6.8 Hz, 1H), 6.33 (dd, J=6.0, 1.8 Hz, 1H), 5.60-5.53 (m, 1H), 5.52-5.45 (m, 1H), 5.40-5.34 (m, 1H), 5.34-5.29 (m, 1H), 4.65 (dtt, J=47.9, 6.7, 5.3 Hz, 1H), 4.42 (s, 2H), 3.80 (s, 3H), 3.51-3.45 (m, 1H), 3.43 (t, J=6.5 Hz, 2H), 2.70-2.51 (m, 3H), 2.51-2.36 (m, 2H), 2.25-2.17 (m, 1H), 2.08-1.96 (m, 4H), 1.63-1.55 (m, 2H), 1.46-1.38 (m, 2H), 0.96 ppm (t, J=7.6 Hz, 3H); ¹³C NMR (150 MHz, CDCl₃) δ 196.31, 161.92, 159.27, 139.94, 135.47, 134.95, 132.86, 130.83, 129.59 (d, J=6.7 Hz), 129.35, 124.95, 122.21 (d, J=6.3 Hz), 113.90, 92.15 (d, J=174.3 Hz), 72.71, 70.02, 55.42, 43.42, 34.50 (d, J=22.6 Hz), 32.75 (d, J=21.4 Hz), 30.44, 29.54, 27.31, 26.31, 20.88, 14.21 ppm; HR-MS (ESI-TOF) calcd for C₂₈H₃₇FO₃Na⁺ [M+Na]⁺: 463.2619. found: 463.2618.

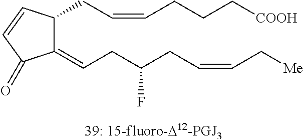 [SMILES: CC/C=C\C[C@H](F)C/C=C1/C(=O)C=C[C@@H]1C/C=C\CCCC(=O)O]

### 15-Fluoro-Δ¹²-PGJ₃ (39)

To a stirred solution of fluorodienone 68 (10.0 mg, 0.0227 mmol, 1.0 equiv) in CH₃CN (90 μL) and H₂O (10 μL) at 25° C. was added 4-acetylamino-2,2,6,6-tetramethyl-1-oxo-piperidinium tetrafluoroborate (15.5 mg, 0.0517 mmol, 6.0 equiv). After stirring at 25° C. for 35 min, the reaction mixture was diluted with water (2 mL). The aqueous layer was extracted with ether (3×5 mL). The combined organic layers were washed with brine (10 mL), dried over Na₂SO₄ and concentrated under reduced pressure. Purification by preparative thin layer chromatography (SiO₂, EtOAc) gave pure title compound (39, 3.4 mg, 0.010 mmol, 45% yield) as yellow oil. 39: R_{f}=0.27 (EtOAc); [α]_{D}²⁵=+106.5° (c=0.2 in C₆H₆); IR (film): νₘₐₓ=2926, 1705, 1657, 1213, 1033 cm⁻¹; ¹H NMR (600 MHz, CDCl₃) δ 7.51 (ddd, J=6.0, 2.6, 1.0 Hz, 1H), 6.60 (t, J=7.6 Hz, 1H), 6.35 (dd, J=6.0, 1.8 Hz, 1H), 5.62-5.52 (m, 1H), 5.52-5.44 (m, 1H), 5.44-5.32 (m, 2H), 4.67 (dtt, J=47.9, 6.9, 5.3 Hz, 1H), 3.56-3.45 (m, 1H), 2.73-2.51 (m, 3H), 2.51-2.38 (m, 2H), 2.35 (t, J=7.3 Hz, 2H), 2.28-2.20 (m, 1H), 2.10-2.01 (m, 4H), 1.70 (tt, J=7.4 Hz, 2H), 0.97 ppm (t, J=7.5 Hz, 3H); ¹³C NMR (150 MHz, CDCl₃) δ 196.31, 177.09, 161.75, 139.86, 135.51, 135.09, 131.60, 129.76 (d, J=6.4 Hz), 126.03, 122.20 (d, J=6.2 Hz), 92.21 (d, J=174.3 Hz), 43.31, 34.51 (d, J=22.5 Hz), 32.98, 32.76 (d, J=21.4 Hz), 30.39, 26.70, 24.53, 20.89, 14.21 ppm; HR-MS (ESI-TOF) calcd for C₂₀H₂₇FO₃Na⁺ [M+Na]⁺: 357.1836. found: 357.1827.

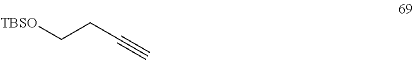

### Alkyne 69

To a stirred solution of 3-butyne-1-ol (2.00 g, 28.5 mmol, 1.0 equiv) in CH₂Cl₂ (60 mL) at 25° C. was added imidazole (5.00 g, 74.2 mmol, 2.6 equiv) followed by TBSCl (5.50 g, 37.1 mmol, 1.3 equiv) in portions. After stirring for 20 min at this temperature the reaction mixture was quenched by the addition of H₂O (10 mL). The phases were separated, the aqueous layer was extracted with CH₂Cl₂ (3×20 mL), and the combined organic layers were dried (Na₂SO₄), filtered, and concentrated under reduced pressure. Purification by flash column chromatography (SiO₂, hexane:EtOAc, 9:1) gave pure title compound (69, 4.42 g, 23.9 mmol, 84% yield). Data were in agreement with those previously reported (Oehlschlager, et al., 1983).

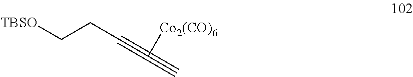

### Cobalt alkyne complex 102

To a stirred solution of alkyne 69 (1.38 g, 7.5 mmol, 1.0 equiv) in CH₂Cl₂ (35 mL) at 0° C. was added Co₂(CO)₈ (2.50 g, 7.5 mmol, 1.0 equiv). The reaction mixture was slowly warmed to 25° C. and stirred at that temperature for 2 h. The resulting deep red solution was filtered through a pad of Celite®, the filter cake was washed with Et₂O (100 mL) and the filtrate was concentrated under reduced pressure. Purification by flash column chromatography (SiO₂, pentane:Et₂O, 9:1) gave pure title compound (102, 3.50 g, 7.5 mmol, quantitative yield). 102: R_{f}=0.95 (pentane:Et₂O, 9:1); IR (film): νₘₐₓ=2957, 2930, 2860, 2092, 2047, 1997, 1256, 1098, 834 cm⁻¹; ¹H NMR (600 MHz, CDCl₃) δ 5.94 (s, 1H), 3.75 (t, J=7.0 Hz, 2H), 2.97 (d, J=7.0 Hz, 2H), 0.83 (s, 9H), 0.00 ppm (s, 6H); ¹³C NMR (150 MHz, CDCl₃) δ 199.93, 92.39, 73.65, 63.59, 37.16, 25.91, 18.33, −5.38 ppm.

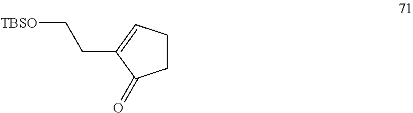 [SMILES: CC(C)(C)[Si](C)(C)OCCC1=CCCC1=O]

### Enone 71

To a stirred solution of cobalt alkyne complex 102 (1.10 g, 3.10 mmol, 1.0 equiv) at 25° C. in vinyl benzoate (20.0 mL, 202 mmol, 65 equiv) was added a solution of NMO.H₂O (2.50 g, 18.9 mmol, 6.1 equiv) in CH₂Cl₂ (35 mL) via addition funnel over 1 h. After stirring for 15 h at this temperature the crude reaction mixture was filtered through a short column (SiO₂, Et₂O) and the filtrate was concentrated under reduced pressure. Purification by flash column chromatography (SiO₂, hexane:Et₂O, 9.5:0.5→7:3) to give pave pure title compound (71, 0.46 g, 1.91 mmol, 62% yield). 71: R_{f}=0.17 (hexane:Et₂O, 9:1); IR (film): νₘₐₓ=2954, 2928, 2856, 1703, 1251, 1098 cm⁻¹; ¹H NMR (600 MHz, CDCl₃) δ 7.44-7.37 (m, 1H), 3.69 (t, J=6.4 Hz, 2H), 2.55 (dq, J=4.5, 2.2 Hz, 2H), 2.37 (ddd, J=12.3, 5.8, 3.8 Hz, 4H), 0.85 (s, 9H), 0.00 ppm (s, 6H); ¹³C NMR (150 MHz, CDCl₃) δ 209.85, 159.42, 143.28, 61.04, 34.36, 28.34, 26.65, 25.88, 18.25, −5.34 ppm; HR-MS (ESI-TOF): calcd for C₁₃H₂₅O₂Si⁺ [M+H]⁺: 241.1618. found: 241.1607.

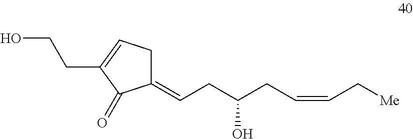 [SMILES: CC/C=C\C[C@H](O)C/C=C1\CC=C(CCO)C1=O]

### Analog 40

To a stirred solution of diisopropylamine (0.13 mL, 0.98 mmol, 1.28 equiv) in THF (8 mL) at 0° C. was dropwise added n-butyl lithium (2.5 M in hexane, 0.33 mL, 0.83 mmol, 1.08 equiv). After stirring for 20 min at this temperature, the clear solution was cooled to −78° C. and a solution of enone 71 (0.18 g, 0.77 mmol, 1.0 equiv) in THF (2 mL) was added dropwise. After stirring the resulting slightly yellow solution for an additional 20 min at this temperature, a solution of aldehyde 3 (0.19 g, 0.77 mmol, 1.0 equiv) in THF (2 mL) was added dropwise and stirring at this temperature was continued for an additional 30 min. The reaction mixture was then quenched with saturated aqueous NH₄Cl solution (20 mL), diluted with Et₂O (20 mL), and allowed to warm to 25° C. The phases were separated, the aqueous layer was extracted with Et₂O (2×25 mL), and the combined organic extracts were washed with brine (50 mL), dried (Na₂SO₄), filtered, and concentrated under reduced pressure. The crude aldol product was taken to the next step without further purification. To a stirred solution of aldol product (0.38 g, 0.77 mmol, 1.0 equiv) in CH₂Cl₂ (10 mL) at 0° C. was added Et₃N (1.0 mL, 7.7 mmol, 10 equiv), and then, slowly and dropwise, methanesulfonyl chloride (0.29 mL, 3.85 mmol, 5.0 equiv). After stirring for 2 h at 25° C., the reaction mixture was quenched with H₂O (20 mL) and diluted with CH₂Cl₂ (20 mL). The phases were separated, the aqueous layer was extracted with CH₂Cl₂ (2×20 mL), and the combined organic layers were washed with H₂O (20 mL), dried (Na₂SO₄), filtered, and concentrated. The crude mesylate was taken to the next step without further purification. To a vigorously stirred solution of mesylate (0.44 g, 0.77 mmol, 1.0 equiv) in CH₂Cl₂ (25 mL) at 25° C. was added Al₂O₃ (0.78 g, 7.7 mmol, 10 equiv). After 16 h the resulting suspension was then filtered through Celite®, washed with EtOAc, and the filtrate was concentrated under reduced pressure. Flash column chromatography (SiO₂, hexane:EtOAc, 8.5:1.5) gave pure TBS protected analog (0.147 g, 0.31 mmol 40% yield) as a light yellow oil. To a stirred solution of TBS protected analog (66 mg, 0.13 mmol, 1.0 equiv) in MeCN (1.5 mL) at 0° C. was added dropwise HF (50% aqueous, 0.14 mL, 4.1 mmol, 30 equiv). After stirring for 30 min at this temperature the reaction mixture was quenched with brine (5 mL) and diluted with EtOAc (10 mL). The phases were separated and the aqueous layer was extracted with EtOAc (3×5 mL), and the combined organic extracts were washed with saturated aqueous NaHCO₃ solution (20 mL), dried (Na₂SO₄), filtered, and concentrated under reduced pressure. Purification by flash column chromatography (SiO₂, hexane:EtOAc, 8:2) gave pure title compound (40, 13 mg, 0.05 mmol, 15% yield) as a colorless oil. 40: R_{f}=0.70 (hexane:EtOAc, 9:1); [α]_{D}²⁵=−3.53 (c=0.34 in CHCl₃); IR (film): νₘₐₓ=2947, 2921, 1727, 1546, 1343, 1272, 1169 cm⁻¹; ¹H NMR (600 MHz, CDCl₃) δ 7.34 (s, 1H), 6.73 (t, J=7.8 Hz, 1H), 5.66-5.54 (m, 1H), 5.44-5.32 (m, 1H), 3.93-3.81 (m, 1H), 3.78 (t, J=5.8 Hz, 2H), 3.23-3.13 (m, 2H), 2.59 (t, J=6.0 Hz, 2H), 2.44 (q, J=7.5, 6.7 Hz, 2H), 2.29 (p, J=7.9 Hz, 2H), 2.14-2.01 (m, 2H), 0.99 ppm (t, J=7.5 Hz, 3H); ¹³C NMR (150 MHz, CDCl₃) δ 196.84, 152.83, 145.81, 136.74, 135.68, 132.36, 123.74, 70.49, 61.21, 36.90, 35.05, 30.71, 30.05, 20.76, 14.20 ppm; HR-MS (ESI-TOF): calcd for C₁₅H₂₂O₃Na⁺ [M+Na]⁺: 273.1461. found: 273.1452.

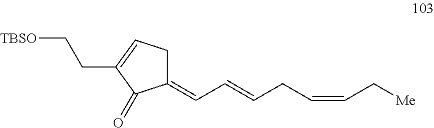 [SMILES: CC/C=C\C/C=C/C=C1\CC=C(CCO[Si](C)(C)C(C)(C)C)C1=O]

### TBS Protected Analog 103

To a stirred solution of diisopropylamine (0.28 mL, 2.06 mmol, 1.28 equiv) in THF (16 mL) at 0° C. was dropwise added n-butyl lithium (2.5 M in hexane, 0.69 mL, 1.74 mmol, 1.08 equiv). After stirring for 20 min at this temperature, the clear solution was cooled to −78° C. and a solution of enone 71 (0.38 g, 1.61 mmol, 1.0 equiv) in THF (4 mL) was added dropwise. After stirring the resulting slightly yellow solution for an additional 20 min at this temperature, a solution of aldehyde 48 (0.20 g, 1.61 mmol, 1.0 equiv) in THF (2 mL) was added dropwise and stirring at this temperature was continued for an additional 30 min. The reaction mixture was then quenched with saturated aqueous NH₄Cl-solution (20 mL), diluted with Et₂O (20 mL), and allowed to warm to 25° C. The phases were separated, the aqueous layer was extracted with Et₂O (2×25 mL), and the combined organic extracts were washed with brine (50 mL), dried (Na₂SO₄), filtered, and concentrated under reduced pressure. The crude aldol product was taken to the next step without further purification. To a stirred solution of aldol product (0.58 g, 1.61 mmol, 1.0 equiv) in CH₂Cl₂ (10 mL) at 0° C. was added Et₃N (0.50 mL, 3.54 mmol, 2.2 equiv.), and then, slowly and dropwise, methanesulfonyl chloride (0.13 mL, 1.77 mmol, 1.1 equiv). After stirring for 2 h at 25° C., the reaction mixture was quenched with H₂O (20 mL) and diluted with CH₂Cl₂ (20 mL). The phases were separated, the aqueous layer was extracted with CH₂Cl₂ (2×20 mL), and the combined organic layers were washed with H₂O (20 mL), dried (Na₂SO₄), filtered, and concentrated under reduced pressure. The crude mesylate was taken to the next step without further purification. To a vigorously stirred solution of mesylate (0.71 g, 1.61 mmol, 1.0 equiv) in CH₂Cl₂ (25 mL) at 25° C. was added Al₂O₃ (1.64 g, 16.0 mmol, 10 equiv). After 16 h the resulting suspension was filtered through Celite®, washed with EtOAc, and the filtrate was concentrated under reduced pressure. Purification by flash column chromatography (SiO₂, hexane:EtOAc, 8.5:1.5) gave pure title compound (103, 126 mg, 0.31 mmol, 23% yield for the 3 steps) as a light yellow oil. 103: R_{f}=0.78 (hexane:EtOAc, 8:2); IR (film): νₘₐ=3012, 2955, 2927, 2855, 1689, 1638, 1627, 1253, 1093, 831 cm⁻¹; ¹H NMR (600 MHz, CDCl₃) δ 7.25 (m, 1H), 6.95 (d, J=10.8 Hz, 1H), 6.26-6.12 (m, 2H), 5.56-5.44 (m, 1H), 5.42-5.27 (m, 1H), 3.79-3.68 (m, 2H), 3.18-3.11 (m, 2H), 2.94 (t, J=6.7 Hz, 2H), 2.48 (t, J=6.4 Hz, 2H), 2.10-1.97 (m, 2H), 1.03-0.92 (m, 3H), 0.86-0.85 (m, 9H), 0.00 ppm (s, 6H); ¹³C NMR (150 MHz, CDCl₃) δ 196.62, 150.74, 145.43, 143.21, 133.75, 132.73, 131.00, 126.36, 124.62, 61.03, 30.98, 30.56, 29.06, 25.88, 20.55, 18.23, 14.16, −5.35 ppm; HR-MS (ESI-TOF): calcd for C₂₁H₃₄O₂SiNa⁺ [M+Na]⁺: 369.2220. found: 369.2223.

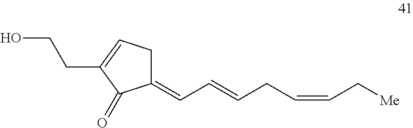 [SMILES: CC/C=C\C/C=C/C=C1\CC=C(CCO)C1=O]

### Analog 41

To a stirred solution of TBS protected analog 95 (15 mg, 0.04 mmol, 1.0 equiv) in MeCN (0.5 mL) at 0° C. was added dropwise HF (50% aqueous, 0.04 mL, 1.3 mmol, 30 equiv). After stirring for 30 min at this temperature the reaction mixture was then quenched with brine (5 mL) and diluted with EtOAc (10 mL). The phases were separated and the aqueous layer was extracted with EtOAc (3×5 mL), and the combined organic extracts were washed with saturated aqueous NaHCO₃ solution (20 mL), dried (Na₂SO₄), filtered, and concentrated under reduced pressure. Purification by flash column chromatography (SiO₂, hexane:EtOAc, 8:2) gave pure title compound (41, 7 mg, 0.03 mmol, 70% yield) as a colorless oil. 41: R_{f}=0.60 (EtOAc); IR (film): νₘₐₓ=3405, 2962, 2930, 2874, 1776, 1679, 1624, 1046 cm⁻¹; ¹H-NMR (600 MHz, CDCl₃) δ ¹H NMR (600 MHz, CDCl₃) δ 7.23 (s, 1H), 6.97 (d, J=8.5 Hz, 1H), 6.23-6.13 (m, 2H), 5.48 (q, J=7.8 Hz, 1H), 5.38-5.27 (m, 1H), 3.72 (t, J=5.8 Hz, 2H), 3.20-3.10 (m, 2H), 2.92 (t, J=5.7 Hz, 2H), 2.54 (t, J=5.9 Hz, 2H), 2.02 (p, J=7.4 Hz, 2H), 0.93 ppm (t, J=7.5 Hz, 3H); ¹³C NMR (150 MHz, CDCl₃) δ 197.84, 151.74, 146.36, 144.25, 133.94, 132.52, 132.16, 126.19, 124.46, 61.36, 31.05, 30.76, 30.29, 20.58, 14.18 ppm; HR-MS (ESI-TOF): calcd for C₁₅H₂₀O₂Na⁺ [M+Na]⁺: 255.1356. found: 255.1365.

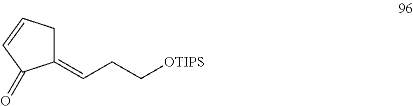

### TIPS Protected Analog 96

To a stirred solution of diisopropylamine (2.49 mL, 17.7 mmol, 1.28 equiv) in THF (350 mL) at 0° C. was dropwise added n-butyl lithium (2.5 M in hexane, 6.0 mL, 15.0 mmol, 1.1 equiv). After stirring for 20 min at this temperature, the clear solution was cooled to −78° C. and a solution of 2-cyclopenten-1-one (72, 1.16 mL, 13.8 mmol, 1.0 equiv) in THF (45 mL) was added dropwise. After stirring the resulting slightly yellow solution for an additional 20 min at this temperature, a solution of aldehyde 73 (Kim, et al., 2012) (4.00 g, 17.3 mmol, 1.25 equiv) in THF (5 mL) was added dropwise and stirring at this temperature was continued for an additional 30 min. The reaction mixture was then quenched with saturated aqueous NH₄Cl solution (200 mL), diluted with Et₂O (100 mL), and allowed to warm to 25° C. The phases were separated, the aqueous layer was extracted with Et₂O (2×100 mL), and the combined organic extracts were washed with brine (150 mL), dried (Na₂SO₄), filtered, and concentrated under reduced pressure. The crude aldol product was taken to the next step without further purification. To a stirred solution of aldol product (3.00 g, 9.59 mmol, 1.0 equiv) in CH₂Cl₂ (70 mL) at 0° C. was added Et₃N (2.0 mL, 14.4 mmol, 1.5 equiv), and then, slowly and dropwise, methanesulfonyl chloride (0.89 mL, 11.5 mmol, 1.2 equiv). After stirring for 2 h at 25° C., the reaction mixture was quenched with H₂O (75 mL) and diluted with CH₂Cl₂ (75 mL). The phases were separated, the aqueous layer was extracted with CH₂Cl₂ (2×50 mL), and the combined organic layers were washed with H₂O (20 mL), dried (Na₂SO₄), filtered, and concentrated under reduced pressure. The crude mesylate was taken to the next step without further purification. To a vigorously stirred solution of mesylate (3.74 g, 9.59 mmol, 1.0 equiv) in CH₂Cl₂ (120 mL) at 25° C. was added Al₂O₃ (9.70 g, 95.9 mmol, 10 equiv). After 16 h the resulting suspension was then filtered through Celite®, washed with EtOAc, and the filtrate was concentrated under reduced pressure. Purification by flash column chromatography (SiO₂, hexane:EtOAc, 8:2) gave pure title compound (96, 0.95 g, 3.22 mmol, 27% yield for the 3 steps) as a light yellow oil. 96: R_{f}=0.60 (hexane:EtOAc, 8:2); IR (film): νₘₐₓ=2942, 2892, 2865, 1703, 1654, 1463, 1100, 881 cm⁻¹; ¹H NMR (600 MHz, CDCl₃) δ 7.60 (dtd, J=6.1, 2.6, 1.0 Hz, 1H), 6.66 (ddt, J=7.7, 6.2, 1.4 Hz, 1H), 6.40 (dt, J=6.0, 2.2 Hz, 1H), 3.85 (t, J=6.6 Hz, 2H), 3.25 (q, J=2.2 Hz, 2H), 2.49 (q, J=6.9 Hz, 2H), 1.13-1.08 (m, 3H), 1.06 ppm (d, J=6.2 Hz, 18H); ¹³C NMR (150 MHz, CDCl₃) δ 196.25, 156.92, 136.22, 135.63, 132.35, 61.94, 33.67, 32.25, 17.96, 11.94 ppm; HR-MS (ESI-TOF): calcd for C₁₇H₃₀O₂SiNa⁺ [M+Na]⁺: 295.2088. found: 295.2078.

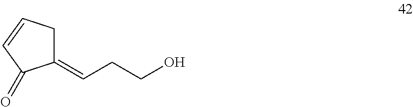

### Analog 42

To a stirred solution of TIPS protected analog 96 (0.10 g, 0.34 mmol, 1.0 equiv) in MeCN (10 mL) at 0° C. was added dropwise HF (50% aqueous, 0.03 mL, 1.0 mmol, 3.0 equiv). After stirring for 30 min at this temperature the reaction mixture was quenched with brine (5 mL) and diluted with EtOAc (10 mL). The phases were separated and the aqueous layer was extracted with EtOAc (3×5 mL), and the combined organic extracts were washed with saturated aqueous NaHCO₃ solution (20 mL), dried (Na₂SO₄), filtered, and concentrated under reduced pressure. Purification by flash column chromatography (SiO₂, hexane:EtOAc, 8:2) gave pure title compound (42, 30 mg, 0.03 mmol, 65% yield) as a colorless oil. 42: R_{f}=0.40 (hexane:EtOAc, 2:8); IR (film): νₘₐₓ=3393, 2922, 1693, 1647, 1578, 1217, 1045 cm⁻¹; ¹H NMR (600 MHz, CDCl₃) δ 7.66-7.54 (m, 1H), 6.62 (ddd, J=9.1, 6.9, 1.4 Hz, 1H), 6.45-6.32 (m, 1H), 3.80 (t, J=6.4 Hz, 2H), 3.24 (q, J=2.2 Hz, 2H), 2.49 ppm (q, J=6.7 Hz, 2H); ¹³C NMR (150 MHz, CDCl₃) δ 196.28, 157.31, 136.34, 136.13, 131.72, 61.25, 33.15, 32.24 ppm; HR-MS (ESI-TOF): calcd for C₈H₁₁O₂ [M+H]⁺: 139.0754. found: 139.0757.

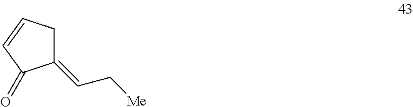

### Analog 43

To a stirred solution of diisopropylamine (4.92 mL, 35.1 mmol, 1.28 equiv.) in THF (350 mL) at 0° C. was dropwise added n-butyl lithium (2.5 M in hexane, 12.1 mL, 30.1 mmol; 1.10 equiv). After stirring for 20 min at this temperature, the clear solution was cooled to −78° C. and a solution of 2-cyclopenten-1-one (72, 2.35 mL, 27.7 mmol, 1.0 equiv) in THF (90 mL) was added dropwise. After stirring the resulting slightly yellow solution for an additional 20 min at this temperature, a solution of propionaldehyde (1.90 g, 27.7 mmol, 1.0 equiv) in THF (5 mL) was added dropwise and stirring at this temperature was continued for an additional 30 min. The reaction mixture was then quenched with saturated aqueous NH₄Cl solution (200 mL), diluted with Et₂O (100 mL), and allowed to warm to 25° C. The phases were separated, the aqueous layer was extracted with Et₂O (2×100 mL), and the combined organic extracts were washed with brine (150 mL), dried (Na₂SO₄), filtered, and concentrated under reduced pressure. The crude aldol product was taken to the next step without further purification (3.85 g, 21.4 mmol, 78% yield). To a stirred solution of aldol product (1.00 g, 7.13 mmol, 1.0 equiv) in CH₂Cl₂ (30 mL) at 0° C. was added Et₃N (2.0 mL, 14.4 mmol, 2.0 equiv), and then, slowly and dropwise, methanesulfonyl chloride (0.77 mL, 9.9 mmol, 1.4 equiv). After stirring for 2 h at 25° C., the reaction mixture was quenched with H₂O (75 mL) and diluted with CH₂Cl₂ (75 mL). The phases were separated, the aqueous layer was extracted with CH₂Cl₂ (2×50 mL), and the combined organic layers were washed with H₂O (20 mL), dried (Na₂SO₄), filtered, and concentrated under reduced pressure. The crude mesylate was taken to the next step without further purification. To a vigorously stirred solution of mesylate (1.55 g, 7.13 mmol, 1.0 equiv) in CH₂Cl₂ (140 mL) at 25° C. was added Al₂O₃ (7.27 g, 71.3 mmol, 10 equiv). After 16 h the resulting suspension was filtered through Celite®, washed with EtOAc, and the filtrate was concentrated under reduced pressure. Purification by flash column chromatography (SiO₂, hexane:EtOAc, 8:2) gave pure title compound (43 (Block, et al., 1985.), 1.00 g, 8.1 mmol, 67% for the 3 steps) as a light yellow oil. 43: R_{f}=0.53 (pentane:Et₂O, 6:4); IR (film): νₘₐₓ=3552, 2967, 2934, 1698, 1654, 1580, 1211, 935, 783 cm⁻¹; ¹H NMR (600 MHz, CDCl₃) δ 7.59 (dtd, J=6.2, 2.6, 1.2 Hz, 1H), 6.68-6.57 (m, 1H), 6.40 (dt, J=6.0, 2.2 Hz, 1H), 3.22 (t, J=2.2 Hz, 2H), 2.34-2.18 (m, 2H), 1.12 ppm (t, J=7.6 Hz, 3H); ¹³C NMR (150 MHz, CDCl₃) δ 196.69, 156.79, 137.28, 136.27, 133.44, 31.96, 23.05, 12.96 ppm; HR-MS (ESI-TOF): calcd for C₈H₁₁O⁺ [M+H]⁺: 123.0804. found 123.0799.

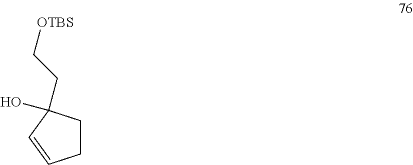 [SMILES: CC(C)(C)[Si](C)(C)OCCC1(O)C=CCC1]

### TBS ether 76

To a stirred solution of ethyl 2-(1-hydroxycyclopent-2-en-1-yl)acetate (1.70 g, 10.0 mmol, 1.0 equiv) in THF (20 mL) at 0° C. was dropwise added lithium borohydride (2.0 M in THF, 10 mL, 20.0 mmol, 2.0 equiv). After stirring for 12 h at 25° C., the clear solution was cooled to 0° C., quenched with saturated aqueous NH₄Cl-solution (10 mL) and allowed to warm to 25° C. The resulting mixture was extracted with CH₂Cl₂ (3×30 mL), and the combined organic layers were washed sequentially with H₂O (30 mL) and brine (30 mL). The organic phase was dried (Na₂SO₄), filtered, and concentrated under reduced pressure to give crude diol which was used directly in the next step without further purification. To a stirred solution of crude diol in CH₂Cl₂ (15 mL) at 0° C. was added Et₃N (4.2 mL, 30.0 mmol, 3.0 equiv), DMAP (122 mg, 1.0 mmol, 0.1 equiv) and TBSCl (2.30 g, 15.0 mmol, 1.5 equiv). After stirring for 3 h at this temperature, the reaction mixture was quenched with saturated aqueous NH₄Cl solution (15 mL), diluted with CH₂Cl₂ (50 mL), and allowed to warm to 25° C. The phases were separated, the aqueous layer was extracted with CH₂Cl₂ (2×50 mL), and the combined organic layers were washed with H₂O (20 mL), dried (Na₂SO₄), filtered, and concentrated under reduced pressure. Purification by flash column chromatography (SiO₂, hexane:EtOAc, 10:1) gave pure title compound (76, 1.50 g, 6.2 mmol, 62% yield for the 2 steps) as a colorless oil. 76: R_{f}=0.40 (hexane:EtOAc, 5:1); IR (film): νₘₐₓ=3421, 3055, 2954, 2857, 1541, 1472, 1360, 1254, 1084, 1033, 1006, 835, 775 cm⁻¹; ¹H NMR (600 MHz, CDCl₃) δ 5.84 (dt, J=5.7, 2.3 Hz, 1H), 5.78 (dt, J=5.6, 2.1 Hz, 1 H), 3.95 (bs, 1H), 3.90-3.88 (m, 2H), 2.50-2.45 (m, 1H), 2.27-2.21 (m, 1H), 1.96 (ddd, J=13.3, 8.4, 4.2 Hz, 1H), 1.93-1.80 (m, 3H), 0.89 (s, 9H), 0.08 (s, 3H), 0.08 ppm (s, 3H); ¹³C NMR (150 MHz, CDCl₃) δ 136.21, 132.93, 85.78, 61.37, 41.31, 38.11, 30.85, 25.93, 18.15, −5.50 ppm; HR-MS (ESI-TOF): calcd for C₁₃H₂₆O₂SiNa⁺ [M+Na]⁺: 265.1599. found: 265.1589.

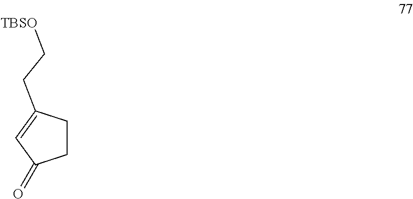 [SMILES: CC(C)(C)[Si](C)(C)OCCC1=CC(=O)CC1]

### Enone 77

To a stirred solution of TBS ether 76 (560 mg, 2.15 mmol, 1.0 equiv) in CH₂Cl₂ (12 mL) at 25° C. was added NaIO₄—SiO₂ (6.0 g, 4.3 mmol, 2.0 equiv) and TEMPO (30 mg, 0.21 mmol, 0.1 equiv). After stirring for 4 h, the reaction mixture was filtered through Celite®, washed with CH₂Cl₂ (50 mL) and concentrated under reduced pressure. Purification by flash column chromatography (SiO₂, hexane:EtOAc, 3:1) gave pure title compound (77¹, 470 mg, 1.81 mmol, 84% yield) as a colorless oil. 77: R_{f}=0.50 (hexane:EtOAc, 3:2); IR (film): νₘₐₓ=2954, 2928, 2857, 1709, 1674, 1617, 1472, 1255, 1094, 776 cm⁻¹; ¹H NMR (600 MHz, CDCl₃) δ 6.00 (s, 1H), 3.84 (t, J=6.2 Hz, 2H), 2.63-2.61 (m, 4H), 2.40-2.39 (m, 2H), 0.87 (s, 9H), 0.04 ppm (s, 6H); ¹³C NMR (150 MHz, CDCl₃) δ 210.37, 180.45, 130.75, 60.84, 36.82, 35.35, 32.12, 25.94, 18.32, −5.29, −5.30 ppm; HRMS (ESI-TOF): calcd for C₁₃H₂₅O₂Si⁺ [M+H]⁺: 241.1618. found: 241.1622.

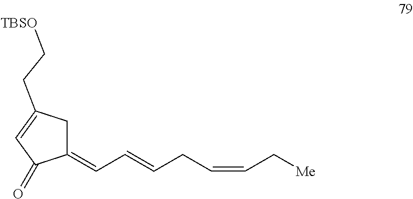 [SMILES: CC/C=C\C/C=C/C=C1\CC(CCO[Si](C)(C)C(C)(C)C)=CC1=O]

### TBS-Protected trienone 79

To a stirred solution of diisopropylamine (129 μL, 0.92 mmol, 2.2 equiv) in THF (12 mL) at 0° C. was dropwise added n-butyl lithium (2.5 M in hexane, 340 μL, 0.84 mmol, 2.0 equiv). After stirring for 20 min at this temperature, the clear solution was cooled to −78° C. and a solution of enone 77 (100 mg, 0.42 mmol, 1.0 equiv) in THF (2 mL) was added dropwise. After stirring the resulting slightly yellow solution for an additional 20 min at this temperature, a solution of aldehyde 48 (78 mg, 0.62 mmol, 1.5 equiv) in THF (8 mL) was added dropwise and stirring at this temperature was continued for an additional 30 min. The reaction mixture was then quenched with saturated aqueous NH₄Cl solution (75 mL), diluted with EtOAc (75 mL), and allowed to warm to 25° C. The phases were separated, the aqueous layer was extracted with EtOAc (2×75 mL), and the combined organic extracts were washed with brine (50 mL), dried (Na₂SO₄), filtered, and concentrated under reduced pressure. The crude aldol product was filtered through a short column (SiO₂, hexane:EtOAc, 3:1) to obtain a mixture of diastereoisomers as a colorless oil which was taken to the next step without further purification. To a stirred solution of the aldol product in CH₂Cl₂ (5 mL) at −10° C. was added DMAP (513 mg, 4.20 mmol, 10 equiv), and then, slowly and dropwise, methanesulfonyl chloride (65 μL, 0.84 mmol, 2.0 equiv). After stirring for 30 min at this temperature, the reaction mixture was slowly warmed to 25° C. and stirred at this temperature for 6 h. The reaction mixture was quenched with saturated aqueous NaHCO₃ solution (5 mL) and diluted with CH₂Cl₂ (50 mL). The phases were separated, the aqueous layer was extracted with CH₂Cl₂ (2×20 mL), and the combined organic layers were washed with H₂O (20 mL), dried (Na₂SO₄), filtered, and concentrated under reduced pressure. Purification by flash column chromatography (SiO₂, hexane:EtOAc, 7:1) gave pure title compound (79¹, 49 mg, 0.14 mmol, 34% yield for the 2 steps) as a colorless oil. 79: R_{f}=0.69 (hexane:EtOAc, 3:1); IR (film): νₘₐₓ=2955, 2929, 2856, 1702, 1662, 1257, 1091 cm⁻¹; ¹H NMR (500 MHz, C₆D₆) δ 6.81 (ddt, J=8.1, 7.3, 1.9 Hz, 1H), 6.26 (ddd, J=17.5, 10.6, 0.7 Hz, 1H), 6.08 (ddt, J=1.7, 1.2, 0.6 Hz, 1H), 5.56-5.39 (m, 2H), 5.21 (dt, J=17.5, 0.8 Hz, 1H), 5.02-4.92 (m, 1H), 3.80-3.68 (m, 1H), 3.02-2.76 (m, 2H), 2.33-2.15 (m, 4H), 2.04-1.91 (m, 2H), 0.95 (s, 9H), 0.89 (t, J=7.5 Hz, 3H), 0.05 (s, 3H), 0.05 ppm (s, 3H); ¹³C NMR (125 MHz, C₆D₆) δ 194.24, 163.95, 137.20, 133.96, 133.82, 133.11, 130.58, 125.00, 121.00, 71.92, 37.57, 35.84, 30.42, 26.04, 21.14, 18.25, 14.40, −4.41, −4.43 ppm; HR-MS (ESI-TOF): calcd for C₂₁H₃₄O₂SiNa⁺ [M+Na]⁺: 369.2220. found: 369.2216.

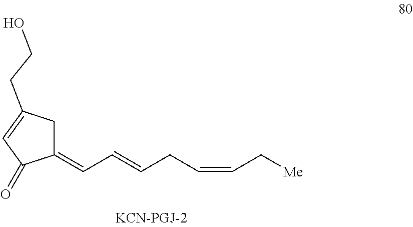 [SMILES: CC/C=C\C/C=C/C=C1\CC(CCO)=CC1=O]

### Δ¹²-PGJ₃ analog 80

To a stirred solution of TBS-protected trienone 79 (20.0 mg, 0.06 mmol, 1.0 equiv) in MeCN (2 mL) at 0° C. was dropwise added a solution of HF (50% aqueous; 100 μL, ca. 2.8 mmol, ca. 50 equiv) in MeCN (1 mL). After stirring for 15 min at this temperature, the reaction mixture was quenched with brine (5 mL) and extracted with EtOAc (5×5 mL). The combined organic extracts were dried (Na₂SO₄), filtered, and concentrated to a volume of ca. 1 mL (not to dryness!). Purification by flash column chromatography (SiO₂, hexane:EtOAc, 1:4) gave pure title compound (80, 11.5 mg, 0.05 mmol, 87% yield) as a colorless oil. 80: R_{f}=0.74 (EtOAc); IR (film): νₘₐₓ=3419, 2962, 2930, 1693, 1650, 1621, 1564, 1418, 1349, 1271, 1200, 1049 cm⁻¹; ¹H NMR (600 MHz, CDCl₃) δ 6.90 (d, J=10.0 Hz, 1H), 6.22-6.14 (m, 3H), 5.53-5.49 (m, 1H), 5.37-5.33 (m, 1H), 3.92 (t, J=6.3 Hz, 2H), 3.24 (s, 3H), 2.94 (t, J=6.1 Hz, 2H), 2.72 (t, J=6.5 Hz, 2H), 2.05 (p, J=7.2 Hz, 2H), 0.97 ppm (t, J=7.6 Hz, 3H); ¹³C NMR (125 MHz, CDCl₃) δ 197.19, 171.39, 143.49, 133.95, 133.21, 132.83, 130.48, 126.32, 124.70, 60.31, 36.23, 35.39, 31.08, 20.67, 14.29 ppm; HR-MS (ESI-TOF): calcd for C₁₅H₂₁O₂⁺ [M+H]⁺: 233.1536. found: 233.1538.

### Example 3

### Discussion

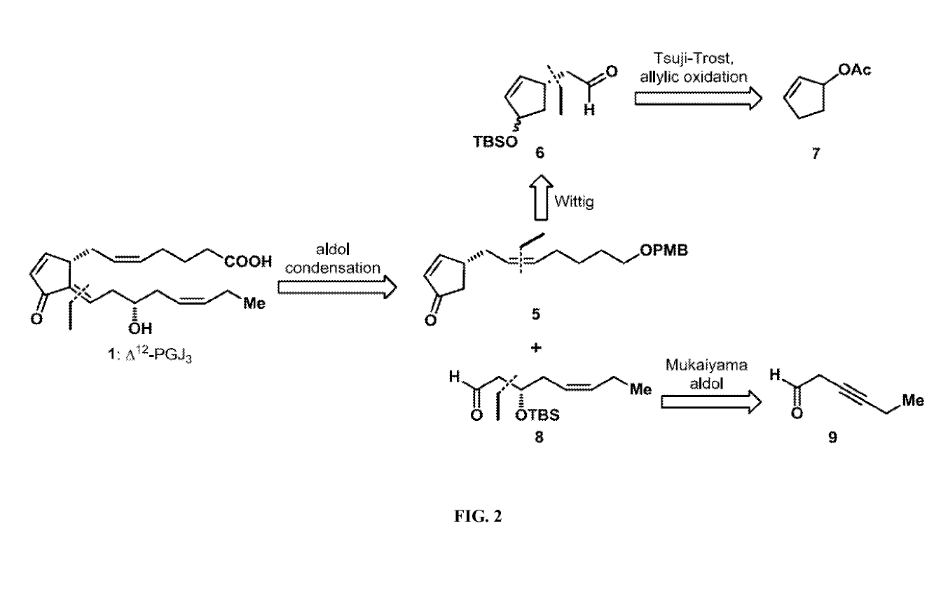

The synthetic strategy for the synthesis of Δ¹²-PGJ₃ (1) was based on the retrosynthetic analysis shown in FIG. 2. Thus, 1 was disconnected to enone 5 and aldehyde 8 through a formal aldol-condensation. Cyclopentenone 5 was then traced back to the corresponding aldehyde 6 through a (Z)-selective Wittig reaction and the latter to allylic acetate 7 through the intermediacy of malonate adduct 11 (see Scheme 1), potentially accessible through an enantioselective Tsuji-Trost reaction and an allylic oxidation. The other coupling partner, aldehyde 8, was traced back to 3-hexyn-1-ol (16, see Scheme 2) through the intermediacy of rather labile hex-3-ynal (9) employing an enantioselective Mukaiyama aldol reaction as the key transformation.

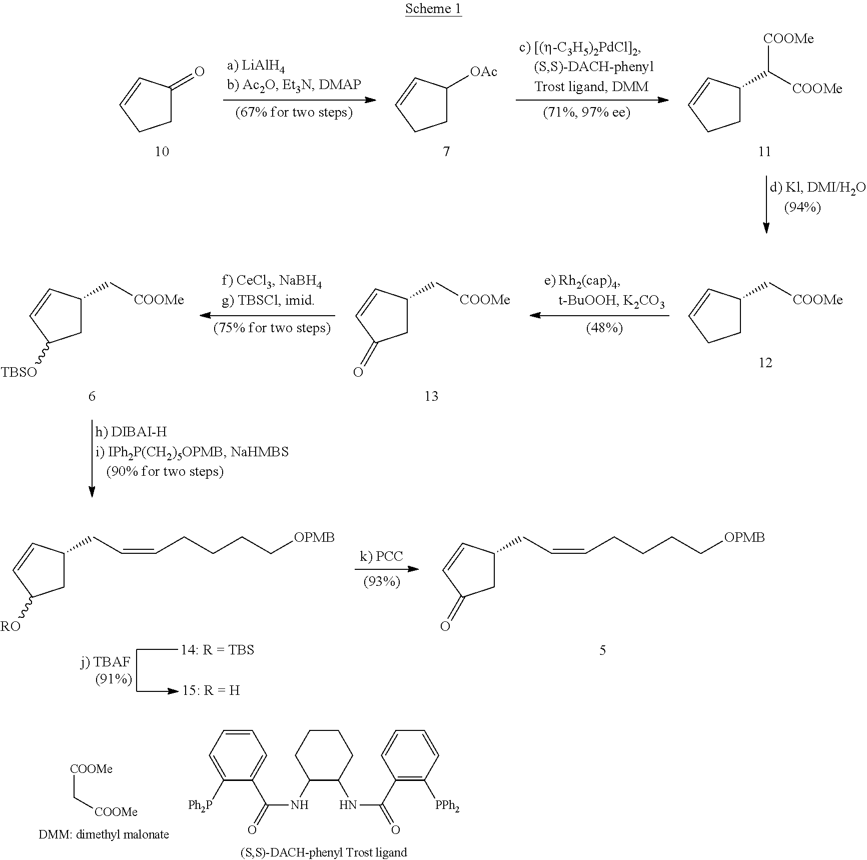

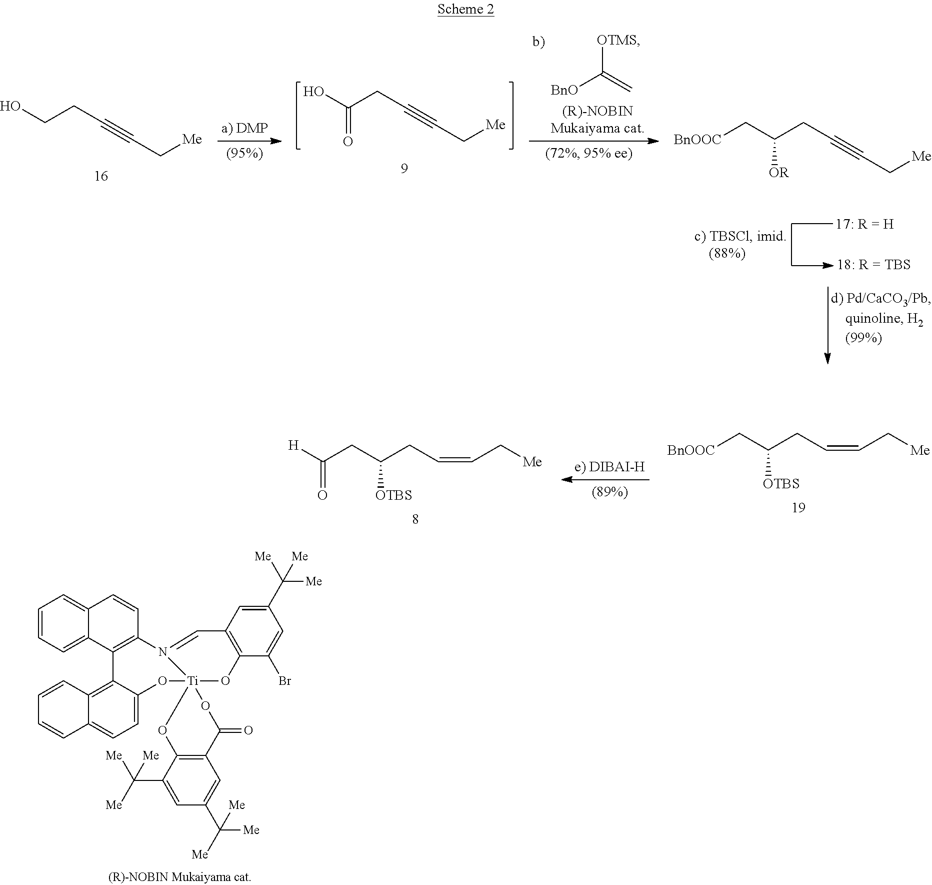

The preparation of enone 5 for the synthesis Δ¹²-PGJ₃ (1) and its methyl ester 2 from 2-cyclopentenone (10, commercially available) is shown in Scheme 1. Thus, reduction of 10 with LiAlH₄ followed by acetylation (Ac₂O, DMAP, Et₃N) led to allylic acetate 7 (67% yield for two steps). Enantioselective Tsuji-Trost reaction (Trost and Bunt, 1996; Miyazaki, et al., 2007) of the latter ((S,S)-DACH-phenyl Trost ligand, allylpalladium(II) chloride dimer, dimethyl malonate, Cs₂CO₃), gave enantioenriched malonate adduct 11 (71% yield, 97% ee, assigned by comparison with the reported optical rotation for the other enantiomer (Miyazaki, et al., 2007)). Decarboxylation of one of the methyl esters in 11 (KI, DMI:H₂O, 10:1, 130° C., 94% yield) set the stage for the envisioned allylic oxidation. Under optimized conditions (Catino, et al., 2004) (Rh₂(cap)₄, t-BuOOH, K₂CO₃), the desired oxidation product was obtained in 48% yield. In preparation of the pending Wittig reaction, Luche-reduction (Luche, 1978) of enone 13 (NaBH₄, CeCl₃) and protection of the so-obtained allylic alcohol as a t-butyldimethylsilyl ether gave intermediate 6 (as a mixture of epimers, with the major one possessing the syn-configuration as expected on steric grounds; inconsequential) in 75% yield for two steps. Selective partial reduction of the methyl ester in 6 to the aldehyde (DIBAl-H) and exposure to the ylide generated from {5-[(4-methoxybenzyl)oxy]pentyl}triphenyl-phosphonium iodide and NaHMDS at low temperature gave alkene 14 with high (Z)-selectivity (≧10:1, judged by ¹H-NMR). Removal of the TBS-protecting group (TBAF, 91% yield) and oxidation (PCC, 93%) gave enone 5 as a single isomer after chromatography.

The preparation of aldehyde 8 for the aldol addition to enone 5 is shown in Scheme 2. Thus, Dess-Martin oxidation (Dess and Martin, 1983) of 3-hexyn-1-ol (16) gave hex-3-ynal (9, 95% yield) (Wavrin and Viala, 2002). The latter was immediately used in an enantioselective Mukaiyama aldol reaction employing (R)-NOBIN catalyst (Carreira, et al., 1994) (see Scheme 3) and the trimethylsilylacetal of benzyl acetate (Kiyooka, et al., 2010) to give, after work-up with TBAF, enantioenriched β-hydroxyester 17 in 72% yield and ≧95% ee judged by ¹⁹F-NMR of the corresponding Mosher esters. The absolute configuration of 17 was unequivocally established to be (3S) by a full Mosher analysis (Hoye, et al., 2007). The β-hydroxy group in 17 was then protected to afford silyl ether 18 (88% yield), the alkyne moiety in 18 was partially hydrogenated using Lindlar's catalyst and quinoline to give the desired (Z)-olefin as the sole product (99% yield), and finally, the aldehyde group was unveiled by reduction of the benzyl ester moiety in 19 with DIBAl-H (89% yield).

With both fragments 5 and 8 in hand, their coupling and completion of the synthesis of Δ¹²-PGJ₃ (1, Scheme 3) were carried out. Thus, treatment of enone 5 with excess lithium diisopropylamide (LDA) followed by addition of aldehyde 8 at low temperature smoothly yielded the aldol product 20 as a mixture of epimers at C12 (ca. 3:1 with the major one possessing the anti-configuration as expected on steric grounds; inconsequential; 79% yield). Mesylation of 20 (MsCl, Et₃N) followed by treatment with Al₂O₃ yielded the dienone 22 with an (E)-configuration at the newly formed double bond as the sole product (62% yield for two steps). Neither the corresponding (Z)-enone nor the his-elimination product, hypothetically arising from the loss of the C14 alcohol moiety, could be detected under these reaction conditions. All that remained was the installation of the carboxylic acid moiety at C1 and the removal of the t-butyldimethylsilyl ether. A three-step sequence was employed to achieve this goal, namely the removal of the para-methoxybenzyl group (DDQ, 87% yield), oxidation of the so-obtained primary alcohol in 23 with PCC to the aldehyde 24 (91% yield), and further oxidation of the latter to the carboxylic acid 25 using Pinnick's protocol (Bal, et al., 1981) (NaClO₂, 95% yield). Finally, upon exposure of 25 to aq. HF, Δ¹²-PGJ₃ (1) was obtained in 92% yield. The corresponding methyl ester 2 was prepared by treatment of hydroxy acid 1 with trimethylsilyldiazomethane in 93% yield.

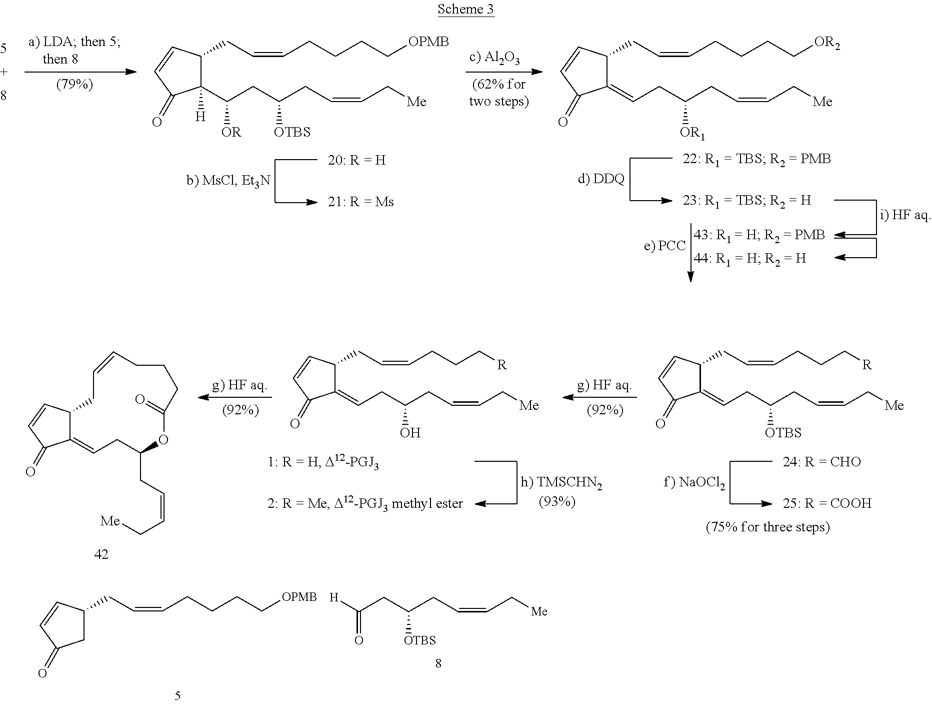

### Description of Synthesis of Additional Compounds.

An alternative process for the synthesis of the key building block aldehyde 15 (Scheme 1) and b) new Δ¹²-PGJ₃ analogs, including compounds 3-8 (shown below) are disclosed herein.

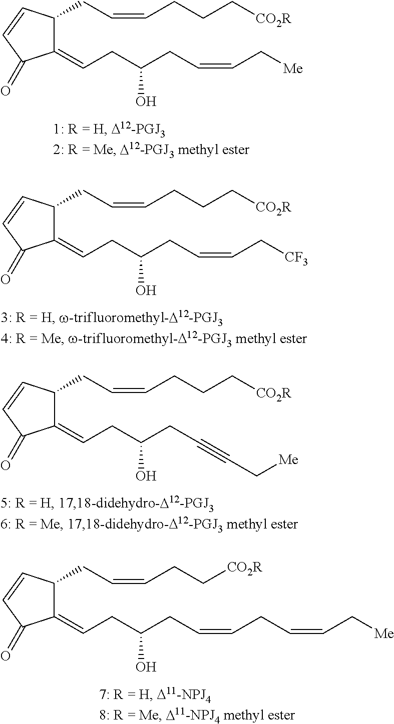

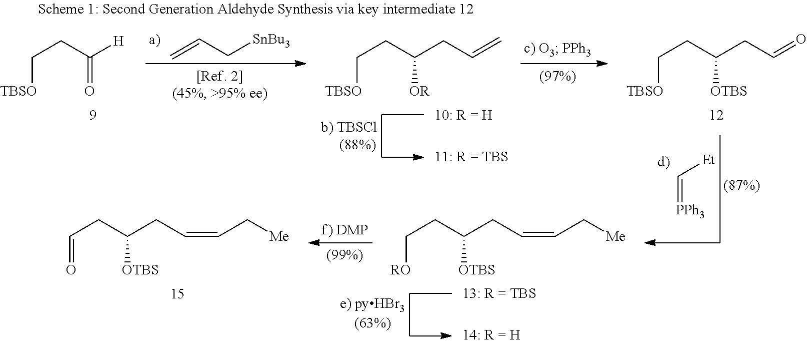

Scheme 1 summarizes the second generation synthesis of aldehyde 15, used as a key building block for the original synthesis of Δ¹²-PGJ₃ and its analogs. Thus, treatment of aldehyde 9 with allyl tri-n-butylstannane in the presence of catalytic amounts of Ti(OiPr)₄ and (S)-BINOL, according to the known allylation procedure (Liniger, et al., 2011, which is incorporated herein by reference), provided alcohol 10 (45%, unoptimized) and ≧95% ee. Silylation of 10 (TBSCl, imidazole) gave TBS ether 11 (88% yield). The latter compound was subjected to ozonolytic cleavage (O₃) followed by reduction (PPh₃) to afford aldehyde 12 in 97% yield. Aldehyde 12 serves as a divergent point from which a variety of lower chain Δ¹²-PGJ₃ derivatives and analogs can be synthesized. Wittig olefination of 12 with the appropriate ylide, generated from the corresponding phosphonium salt, led to olefin 13 in 87% yield, possessing predominantly the required Z-configuration. Selective desilylation of the primary TBS ether from 13 then led to alcohol 14 (py.HBr₃, 63% yield, unoptimized; a number of other acid- or fluoride-based reagents can be used to effect this transformation) which was oxidized with DMP to the desired aldehyde 15 in 99% yield. A number of other oxidizing agents can be used to carry out this transformation. The aldehyde 12 was used to prepare all six analogs, ω-trifluoromethyl-Δ¹²-PGJ₃ (3), 17,18-didehydro-Δ¹²-PGJ₃ (5), Δ¹¹-NPJ₄ (7), and their methyl esters (4, 6, and 8). Scheme 2 summarizes the synthesis of the trifluoromethyl aldehyde 18 required to construct ω-trifluoromethyl-Δ¹²-PGJ₃ (3) and its methyl ester 4.

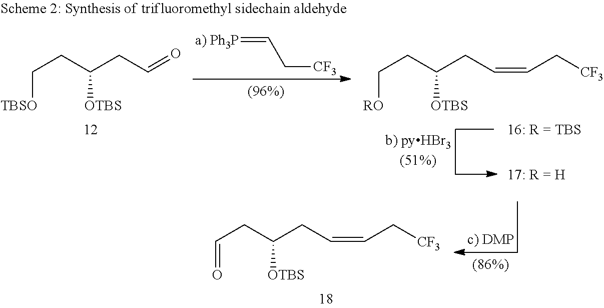

Wittig olefination of aldehyde of acetylenic aldehyde 22 required for the synthesis of analog 5 and 6. Thus, Corey-Fuchs reaction of aldehyde 12 (CBr₄, PPh₃) afforded dibromo olefin 19 in 94% yield, which was reacted sequentially with nBuLi and Ed to afford ethyl acetylene derivative 21 in 68% overall yield via the lithio derivative of acetylene 20. Selective desilylation of 21 (py.HBr₃, 53% yield) furnished the corresponding primary alcohol, whose oxidation with DMP gave the desired alcohol 22 in 94% yield.

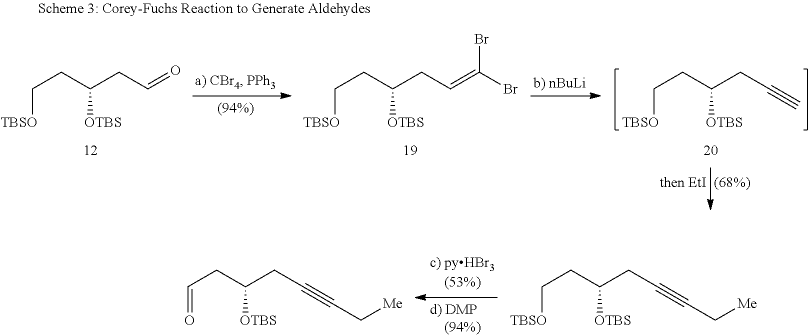

The synthesis of Δ¹²-PGJ₃ analogs 3 and 5, and their methyl esters 4 and 6 proceeded along the previously developed routes as shown in Scheme 4.

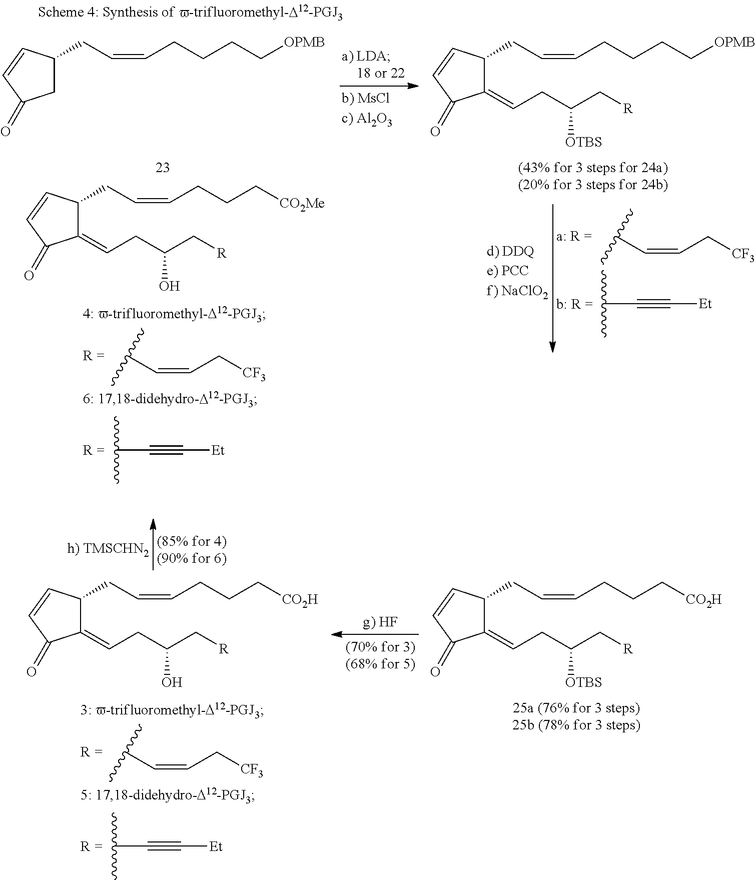

Thus, aldol reaction between enone 23 and aldehyde 18 or 22 in the presence of LDA furnished a mixture of diastereomeric alcohol coupling products which were converted to the bis-enones 24a (43% overall yield, unoptimized) and 24b (20% overall yield, unoptimized) through their corresponding mesylates (MsCl, Et₃N) and Al₂O₃-induced elimination (Al₂O₃). Compounds 24a and 24b were separately converted to the carboxylic acid derivatives 25a and 25b, respectively, through the standard 3-step sequence (DDQ removal of the PMB group, PCC oxidation and NaClO₂ oxidation, 76% overall for 25a; 78% overall for 25b) proceeding through the corresponding alcohols and aldehydes. Finally, 25a and 25b were desilylated in the presence of aq. HF to afford ω-trifluoromethyl-Δ¹²-PGJ₃ (3, 70% yield, Z:E ca. 7:1 Δ^{16,17} mixture of isomers) and 17,18-didehydro-Δ¹²-PGJ₃ (5, 68% yield). Methylation of 3 and 5, respectively, (TMSCHN 2, 85% yield for 4, 90% yield for 6) formed ω-trifluoromethyl-Δ¹²-PGJ₃ methyl ester (4) and 17,18-didehydro-Δ¹²-PGJ₃ methyl ester (6).

The synthesis of Δ¹¹-NPJ₄ (7, Δ¹-neuroprostane J₄, a possible naturally occurring metabolite of docosahexaenoic acid) and its methyl ester Δ¹¹-NPJ₄ methyl ester (8) is shown in Schemes 5-7. Scheme 5 summarizes the synthesis of the required C11 aldehyde 29 starting with dibromide 19 (see Scheme 3). Thus, treatment of 19 with nBuLi led to terminal acetylene 20 (quantitative yield) which was alkylated with propargylic bromide 26 in the presence of K₂CO₃, CuI, and NaI to afford bis-acetylene 27 (79% yield). The latter was selectively reduced to bisolefin 28 with P-2 Ni [Ni(OAc)₂, NaBH₄, 1,2-diaminoethane, H₂] in 78% yield (Oger, et al., 2010; Brown and Ahuja, 1973) Selective desilylation (py.HBr₃, 60% yield) followed by oxidation with DMP (80% yield) gave aldehyde 29.

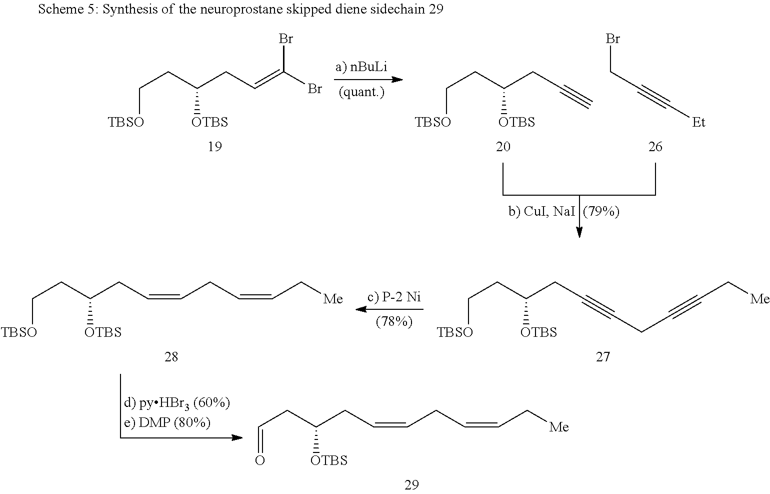

The required PMB ether derivative of the C11 enone 34 was synthesized as shown in Scheme 6. Thus, DIBAL-H reduction of the methyl ester 30 furnished aldehyde 31, which was subjected to Wittig olefination with the ylide derived from phosphonium iodide 32 (NaHMDS) to afford, after desilylation (TBAF) bis-olefin 33 (68% overall for the three steps from 30). PCC oxidation of 33 then afforded desired enone 34 (92% yield).

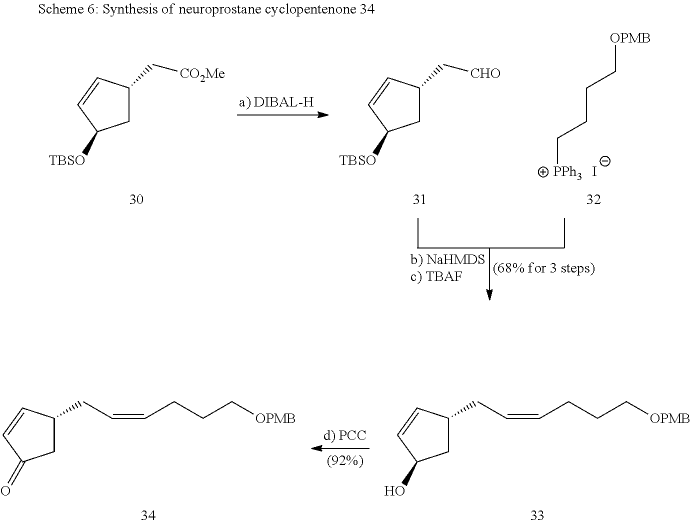

Scheme 7 shows the completion of the synthesis of Δ¹-NPJ₄ (7) and its methyl ester Δ¹¹-NPJ₄ methyl ester (8). Thus, aldol coupling of 34 and 29 in the presence of LDA, followed by mesylation (MsCl, Et₃N) and elimination (Al₂O₃) led to enone 35 (30% overall yield, unoptimized). Removal of the PMB group (DDQ, 66% yield) followed by sequential oxidation of the resulting alcohol with PCC to the aldehyde stage (80% yield) and NaClO₂ furnished carboxylic acid silyl ether 36 (97% yield). Fluoride-induced desilylation of the latter compound (aq. HF) gave Δ¹¹-NPJ₄ (7) in 81% yield. Methylation of 7 (TMSCHN₂, 72% yield, unoptimized) formed Δ¹¹-NPJ₄ methyl ester (8).

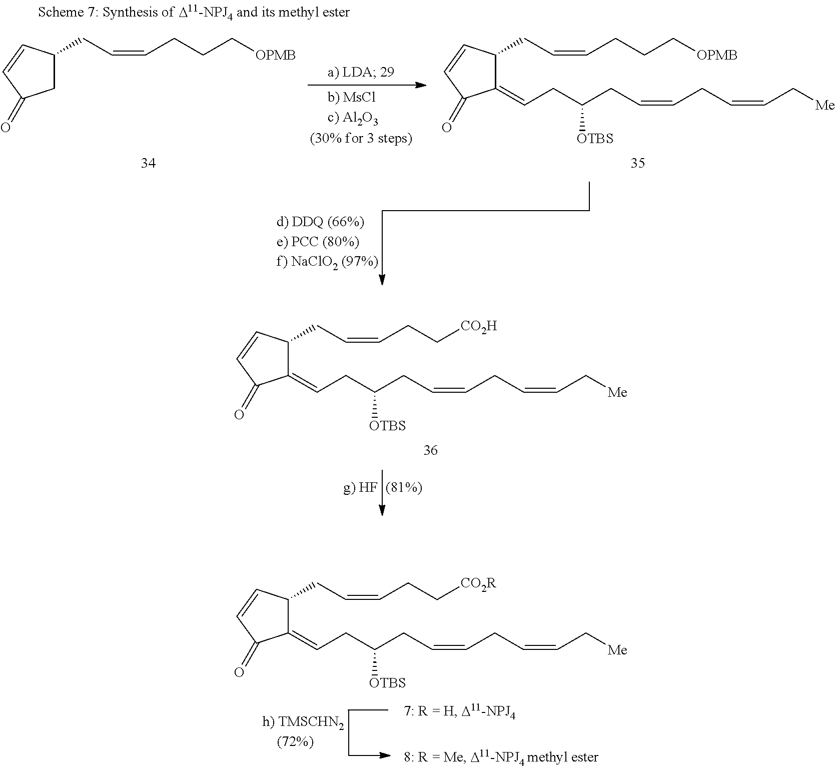

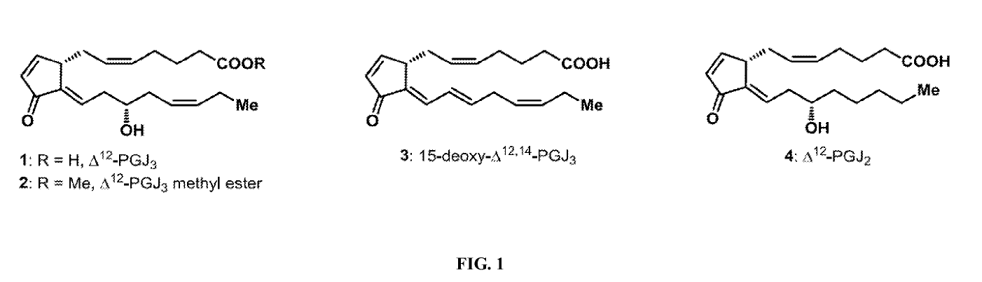

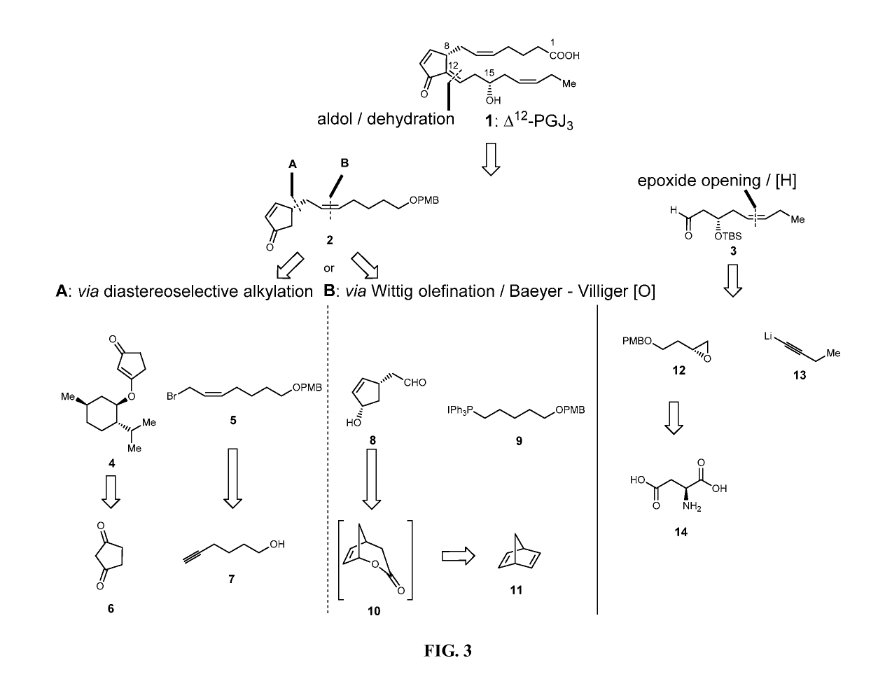

Additionally, alternative synthetic routes to the key C1-12 fragment (enone 2, FIG. 1) and C13-20 fragment (aldehyde 3, FIG. 1) of Δ¹²-PGJ₃ (1) employing cost-effective starting materials and reagents. FIG. 3 shows, in retrosynthetic format, these alternative synthetic strategies. Disconnection at the Δ¹²-olefinic bond through an aldol/dehydration sequence and functional group transforms at C1 lead to enone 2 and aldehyde 3. Enone 2 and aldehyde 3 were then traced back to 1-(−)-menthyl enol ether 4 and allylic bromide 5 (see route A, FIG. 3), and epoxide 12 and lithio acetylide 13 (see FIG. 3), respectively. Cyclopentenone derivative 4 and epoxide 12 are readily available from 1-(−)-menthol and 1-aspartic acid, respectively. Alternatively, enone 2 can also be traced back, retro synthetically, to norbornadiene (11) through the intermediacy of lactone 10 via Baeyer-Villiger oxidation and Wittig olefination of hydroxy aldehyde 8 and phosphonium salt 9 (see route B, FIG. 3).

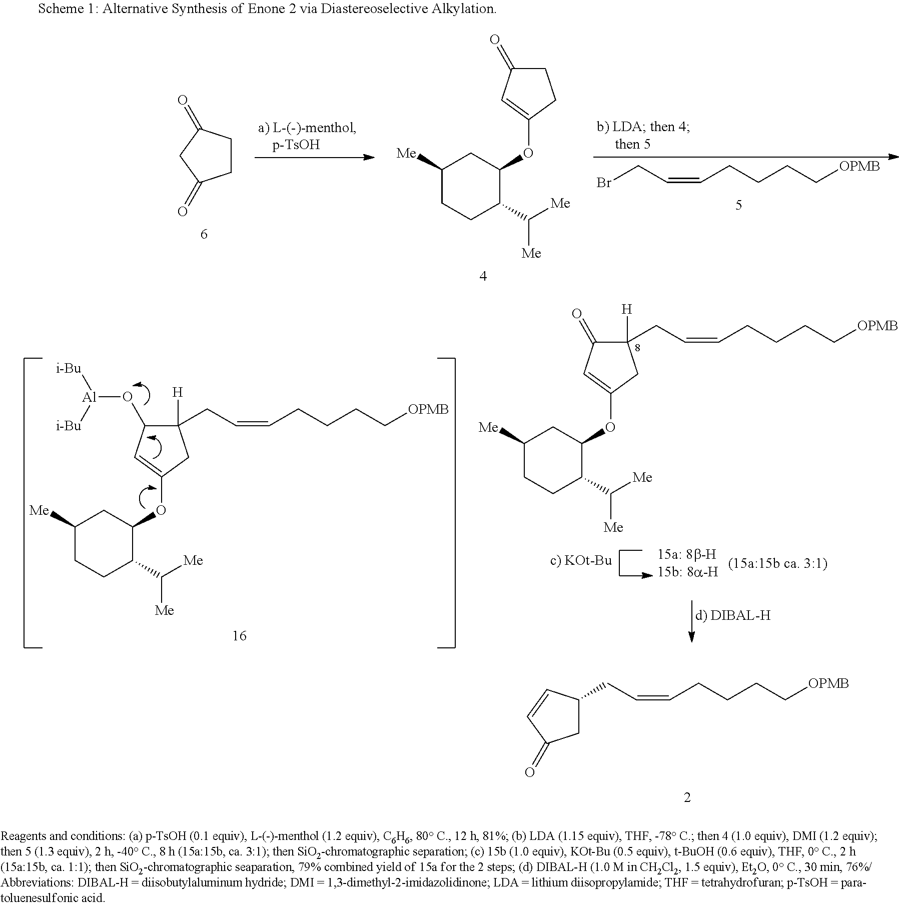

Scheme 1 shows the preparation and conversion of menthyl enol ether 4 to enone 2. Thus, treatment of 1,3-cyclopentanedione (6) with 1-(−)-menthol under acidic conditions (p-TsOH) gave enolether 4 in 81% yield (Iimura, et al., 2006). Diastereoselective alkylation (Stork and Danheiser, 1973) of the enolate generated from 4 (LDA, DMI, THF, −78° C.) with allylic bromide 5 furnished a chromatographically separable mixture of diastereomers 15a (8-βH) and 15b (8-αH) in 91% yield and ca. 3:1 d.r. (judged by ¹H NMR spectroscopic analysis). Exposure of the undesired diastereomer 15b to KOt-Bu in t-BuOH:THF led to a 1:1 mixture of 15a and 15b from which additional 15a was isolated chromatographically for a combined yield of 79% of the desired diastereoisomer 15a. DIBAL-H reduction of the latter followed by acidic workup gave enone 2 (via transient intermediate 16, 76% yield) the enantiomeric identity of which was proven by comparison [α_{D}²⁵] with an authentic sample prepared according to other synthetic route.

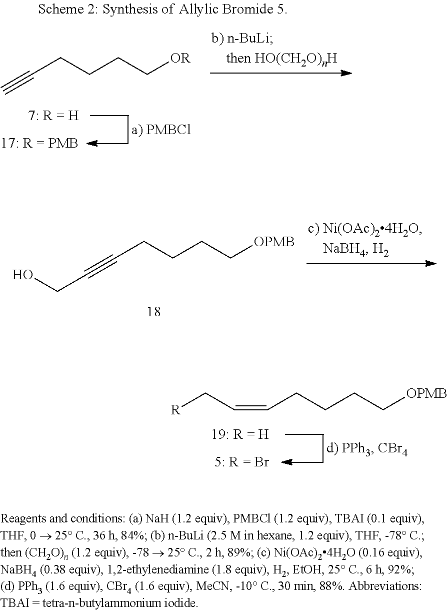

Scheme 2 summarizes the synthesis of allylic bromide 5 starting from commercially available 5-hexyne-1-ol (7). Thus, treatment of 7 with p-methoxybenzyl chloride and NaH in the presence of catalytic amounts of TBAI provided the PMB ether 17 in 84% yield. Formation of the lithio derivative of 17 with n-BuLi, followed by addition of paraformaldehyde afforded propargyl alcohol 18 in 89% yield. Partial reduction of the alkyne moiety within 18 using nickel boride (generated in situ from Ni(OAc)₂ and NaBH₄) (Brown and Ahuja, 1973a; Brown and Ahuja, 1973b) in the presence of 1,2-ethylenediamine and H₂ furnished (Z)-olefin 19 in 92% yield (Z:E≧45:1, judged by ¹H NMR spectroscopic analysis) which was converted to allylic bromide 5 through the action of CBr₄ and PPh₃ in 88% yield.

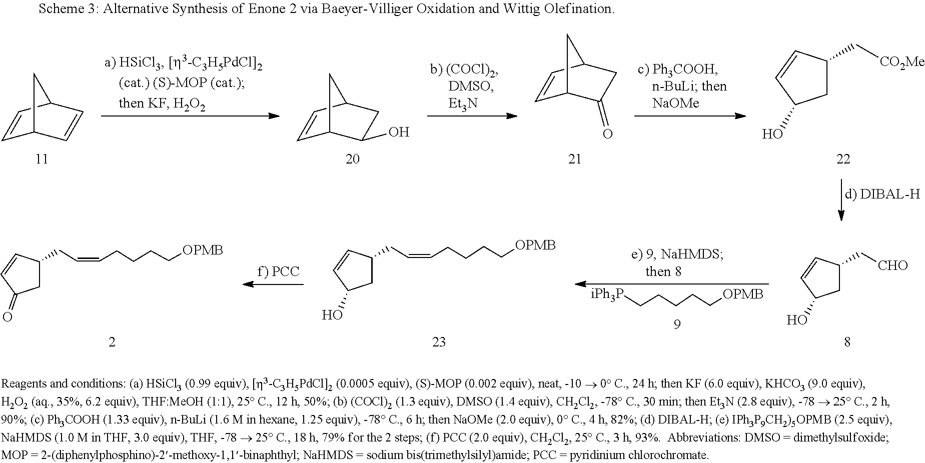

Another synthetic route to key intermediate enone 2 is shown in Scheme 3. Starting from norbornadiene (11), enantiopure norbornenone (21) was obtained by a two-step sequence consisting of enantioselective hydrosilylation/oxidation and Swern-oxidation of the resulting alcohol 20 (45% yield for the two steps) (Uozumi, et al., 1992; Ghosh, et al., 2009). Baeyer-Villiger oxidation (Greene, et al., 1980; Corey, et al., 1988) of the latter and opening of the so-obtained lactone with NaOMe furnished hydroxyester 22. The enantiomeric purity of the growing molecule was determined at this point as 94.5:5.5 e.r. using ¹H NMR spectroscopic analysis of the corresponding Mosher esters of 22. Hydroxy ester 22 was converted to enone 2 through our previously developed synthetic route involving partial reduction of the methylester in 22 to hydroxy aldehyde 8, Wittig reaction with the ylide generated from phosphonium salt 9 to give (Z)-alkene 23, and PCC-oxidation of the latter, in 73% yield for the three steps.

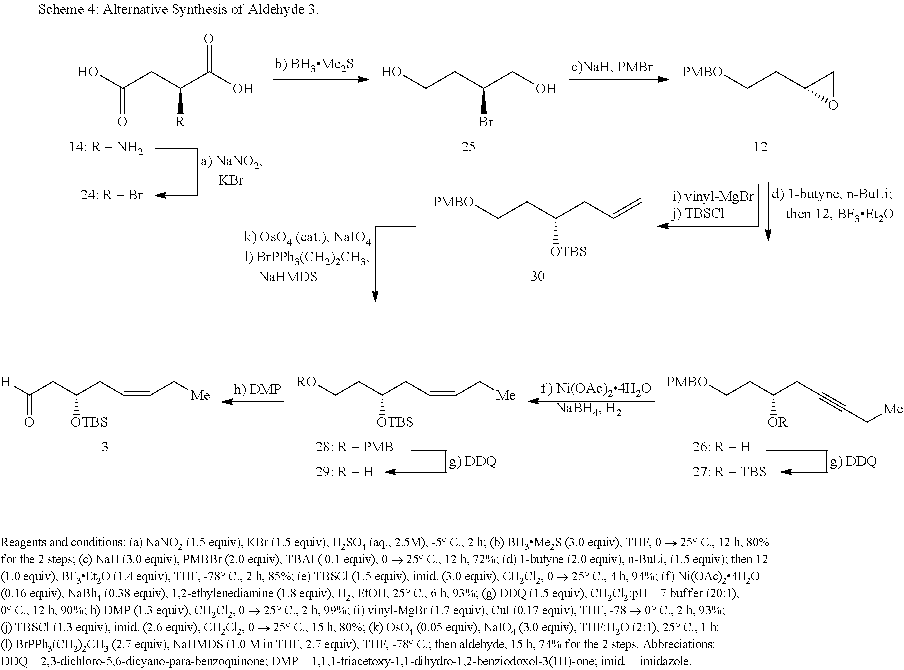

The preparation of aldehyde 3 is summarized in Scheme 4. Thus, diazotization of 1-aspartic acid (14) followed by bromination using NaNO₂ and KBr gave bromosuccinic acid 24, which upon treatment with BH₃.Me₂S afforded bromodiol 25 (80% for the two steps) (Frick, et al., 1992). Epoxide formation followed by PMB protection of the intermediate epoxyalcohol (NaH, PMBBr, TBAI, 0→25° C.) furnished 12 in one pot in 72% overall yield. Opening of the latter with the lithioalkyne derivative generated from 1-butyne and n-BuLi in the presence of BF₃.Et₂O gave alcohol 26 in 85% yield. Protection of 26 as a TBS ether (27, TBSCl, imid., 94% yield) followed by partial reduction (Ni(OAc)₂, NaBH₄, 1,2-ethylenediamine, H₂) provided (Z)-alkene 28 in 93% yield with excellent Z/E selectivity (ca. 30:1, judged by ¹H NMR spectroscopic analysis). Deprotection of the PMB ether in 29 (DDQ) and oxidation of the so-obtained alcohol (Dess-Martin periodinane) furnished aldehyde 3 in 90% yield. Alternatively, (Z)-alkene 28 was also obtained from epoxide 12 (see Scheme 4). Thus, the latter was opened with vinylmagnesium bromide (93% yield) and the so-obtained homoallylic alcohol was protected as a TBS ether (30, TBSCl, imidazole, 80% yield). Bis-hydroxylation/periodate cleavage of 30 furnished the intermediate aldehyde which was directly reacted with the ylide generated from BrPPh₃(CH₂)₂CH₃ and NaHMDS to give (Z)-alkene 28 in 74% yield for the two steps.

After optimization of the synthesis of enone 2 and aldehyde 3, focus was turned to the synthesis of novel Δ¹²-PGJ₃ analogs 31-41 as well as the simple analogs 42 and 43.

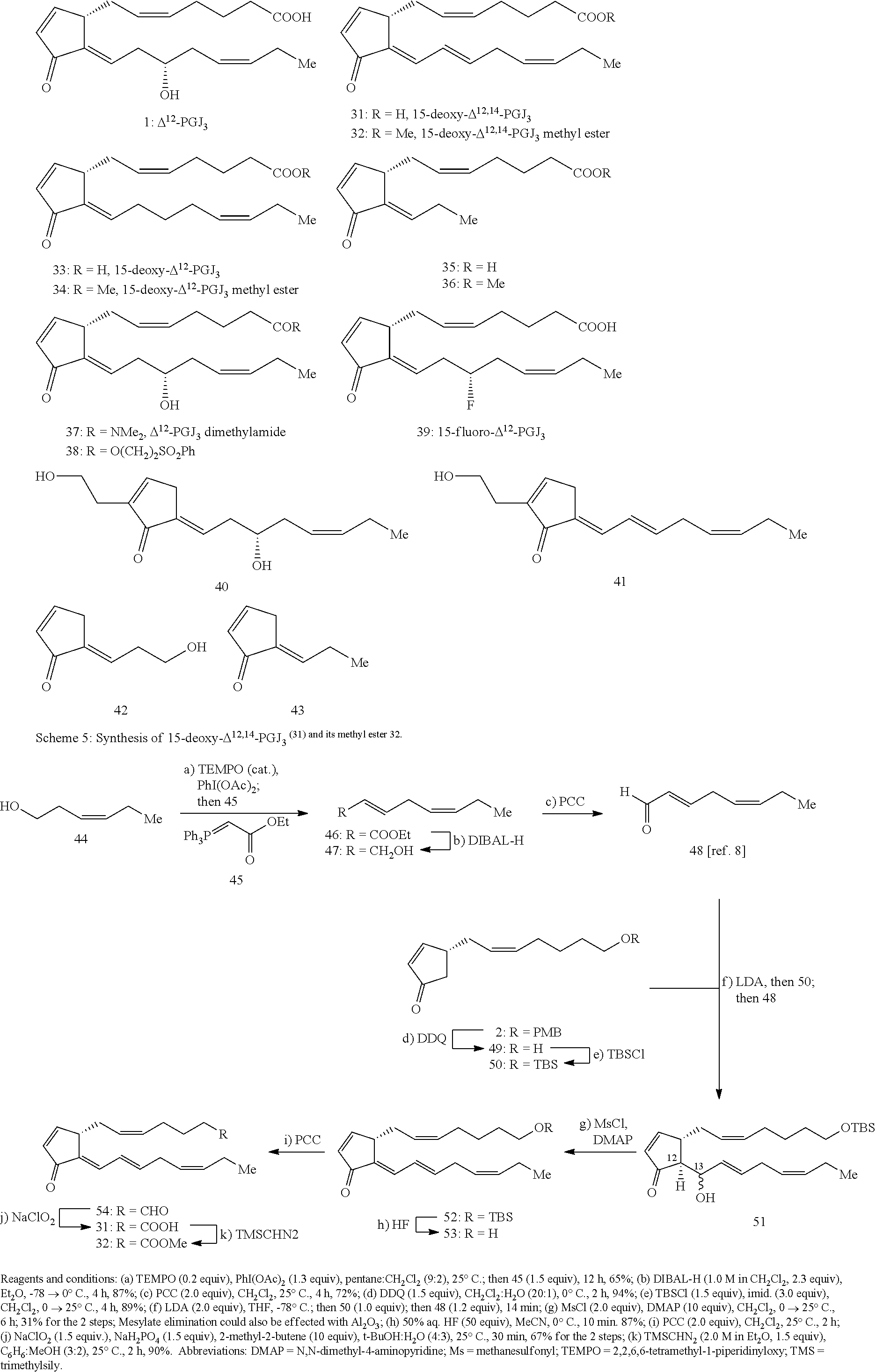

The synthesis of 15-deoxy-Δ^{12,14}-PGJ₃ (31) and its methyl ester 32 commenced with the preparation of double unsaturated aldehyde 48 (Scheme 5) according to a known procedure (Honda, et al., 1997). Thus, one-pot oxidation and Wittig olefination of (Z)-hex-3-en-1-ol (44) furnished ester 46 which in turn was reduced (DIBAL-H) and then reoxidized to aldehyde 48 (41% yield for the four steps) as shown in Scheme 5. Aldol coupling of the latter with the enolate generated from enone 50 [synthesized in two steps from enone 2 by removal of the PMB ether with DDQ and re-protection of the intermediate alcohol 49 as a TBS ether (TBSCl, 84% yield for the two steps)] then furnished aldol product 51 as a mixture of epimers at C13 (inconsequential). Treatment of 51 (mixture of epimers) with MsCl and DMAP furnished extended enone 52 in 31% yield for the two steps. Removal of the TBS ether (50% aq. HF) and oxidation of the so-obtained alcohol 53 to the carboxylic acid through the corresponding aldehyde 54 (PCC; then NaClO₂) gave 15-deoxy-Δ_{12,14}-PGJ₃ (31) in 58% yield for the three steps. Methyl ester 32 was obtained by treatment of 31 with TMSCHN₂ (90% yield).

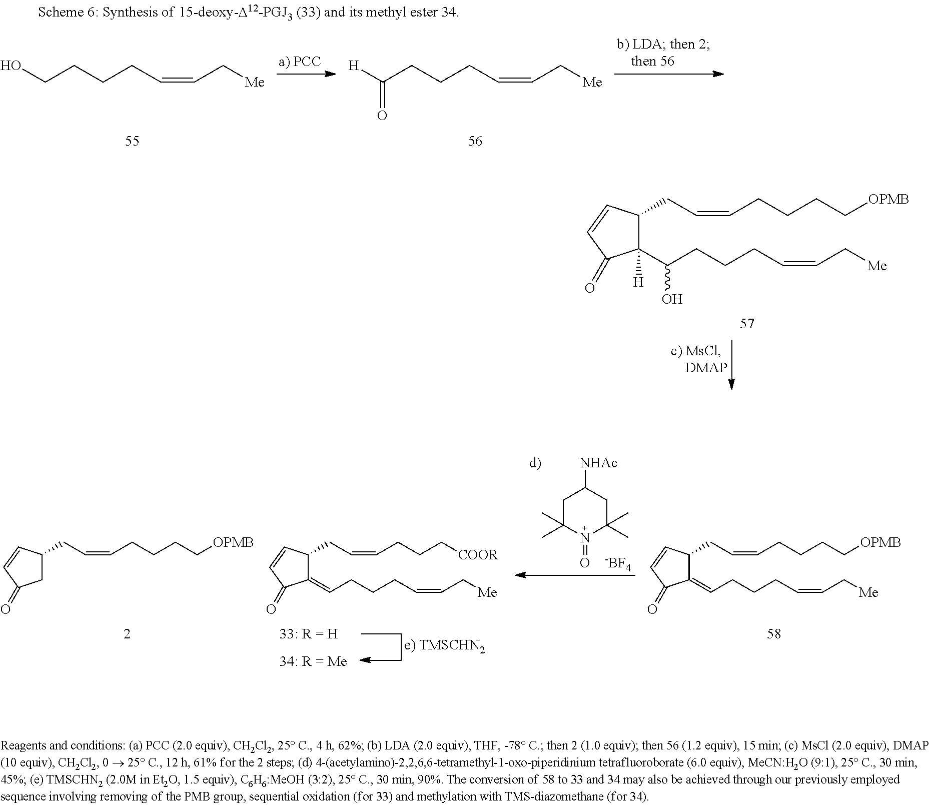

The synthesis of 15-deoxy-Δ¹²-PGJ₃ (33) and its methyl ester 34 is summarized in Scheme 6. Thus, PCC-oxidation of (Z)-oct-5-en-1-ol (55) produced aldehyde 56. Coupling of the latter with the enolate of enone 2 gave aldol product 57 as a mixture of epimers at C13 (inconsequential). Mesylate formation (MsCl, DMAP) and elimination in the same pot proceeded smoothly to give 58 in 61% overall yield and as a single isomer. Deprotection of the PMB ether and oxidation of the so-obtained primary alcohol to the corresponding carboxylic acid was achieved by treatment with 4-(acetylamino)-2,2,6,6-tetramethyl-1-oxo-piperidinium tetrafluoroborate (Pradham, et al., 2009) leading to 15-deoxy-Δ¹²-PGJ₃ (33) in 45% yield. Methylester 34 was obtained by exposure of the latter with TMSCHN₂ in 90% yield.

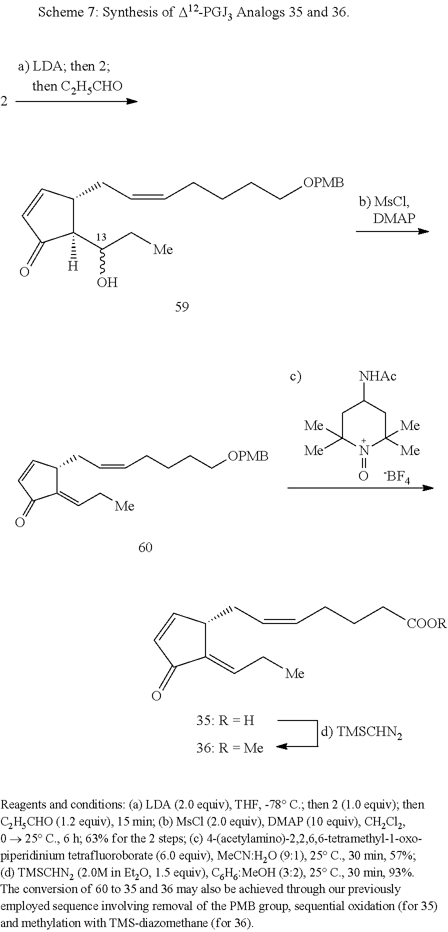

The synthesis of truncated Δ¹²-PGJ₃ analogs 35 and 36 containing only the C1-15 framework of the natural product is shown in Scheme 7. Thus, reaction of the enolate of enone 2 with propionaldehyde gave aldol product 59 as a mixture of epimers at C13 (inconsequential) Elimination and oxidation using previously established conditions [i.e. MsCl, DMAP, 63% yield for the two steps; then 4-(acetylamino)-2,2,6,6-tetramethyl-1-oxo-piperidinium tetrafluoroborate, 57% yield] gave analog 35. Its methylester 36 was obtained in 93% yield by treatment with TMSCHN₂.

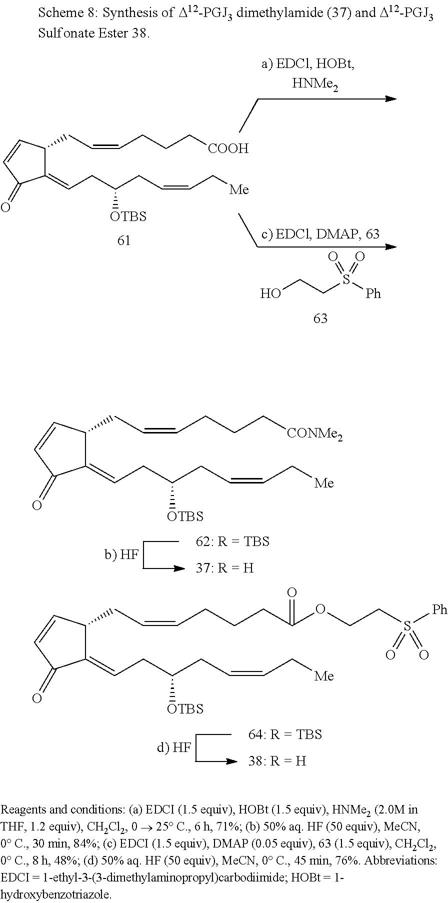

Δ₁₂-PGJ₃ dimethylamide (37) and sulfonate ester 38 were synthesized as shown in Scheme 8. Thus, coupling of TBS protected Δ¹²-PGJ₃ 61 with dimethylamine (EDCI, HOBt) led to Δ¹²-PGJ₃ dimethyl amide silyl ether (62) in 71% yield. Desilylation of the latter (50% aq. HF) led to 37 in 84% yield. Similarly, coupling of 61 with alcohol 63 (EDCI, DMAP) gave intermediate 64 (48% yield), whose deprotection (50% aq. HF) led to sulfonate ester 38 in 76% yield.

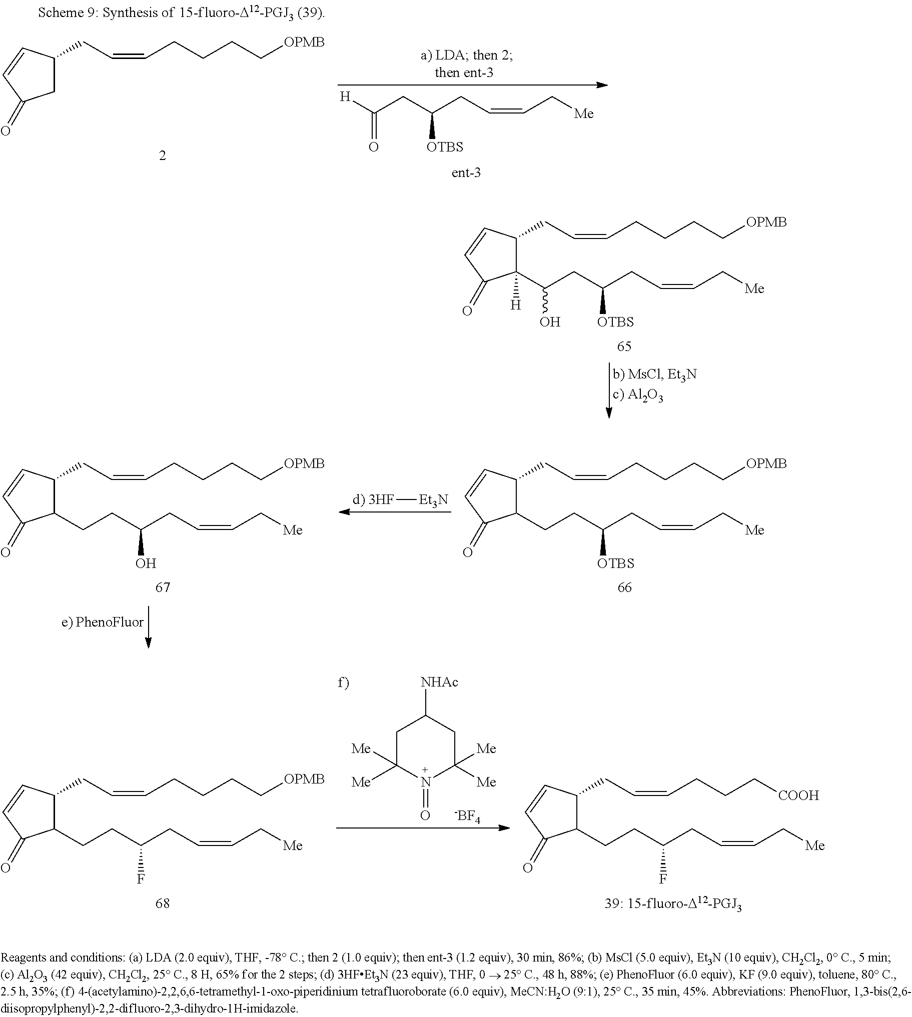

The synthesis of a 15-fluoro-Δ¹²-PGJ₃ (39), a fluorinated analog of Δ¹²-PGJ₃, commenced with the aldol reaction of the enolate of enone 2 and the enantiomer of aldehyde 3 (anticipating inversion of configuration at C15 in the fluorination step) as shown in Scheme 9. Enantiomeric aldehyde ent-3 was synthesized by the same sequence as shown for 3 in Scheme 4 using d-aspartic acid instead of 1-aspartic acid as a starting material. Aldol reaction of enone 2 with ent-3 afforded aldol product 65 as a mixture of C13-epimers (inconsequential) in 86% yield. Treatment of the latter mixture with MsCl and Et₃N followed by Al₂O₃-induced elimination of the so-formed mesylate yielded 65 stereoselectively and in 65% overall yield for the two steps. Desilylation of the latter (3HF.Et₃N, 88% yield) gave hydroxy compound 67. Nucleophilic substitution of the hydroxyl group in 67 using PhenoFluor (Tang, et al., 2012) furnished fluorinated precursor 68 presumed to possess the correct 15(S) configuration in 35% yield. One-pot PMB-deprotection/oxidation [4-(acetylamino)-2,2,6,6-tetramethyl-1-oxo-piperidinium tetrafluoroborate] then gave desired 15-fluoro-Δ¹²-PGJ₃ (39) in 45% yield.

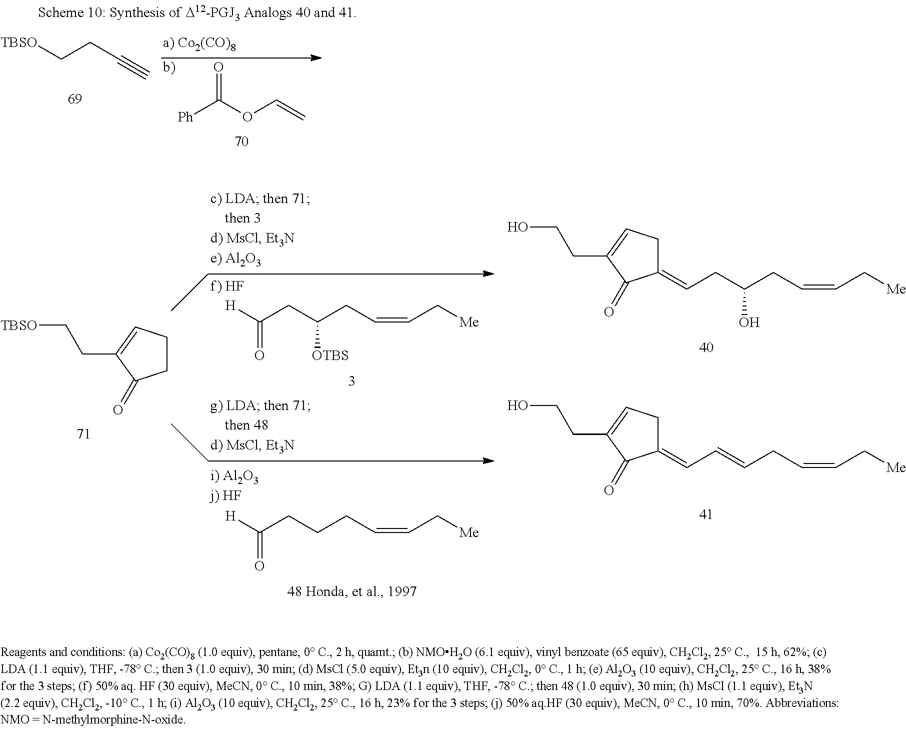

Analogs 40 and 41 missing the C1-7 chain of Δ¹²-PGJ₃, but with an additional hydroxyethyl group at C10 were synthesized as shown in Scheme 10. Thus, Pauson-Khand reaction of TBS protected 3-butyne-1-ol (69) with vinyl benzoate (70) afforded cyclopentenone 71 in 62% yield. Aldol reactions with the enolate generated from cyclopentenone 71 and aldehyde 3, or aldehyde 48, (Honda, et al., 1997) respectively, gave rise to the respective aldol products (structures not shown) Elimination and deprotection using previously developed conditions (i.e. MsCl, Et₃N; then Al₂O₃, 38% yield for the three steps; then 50% aq. HF, 38% yield) then furnished analogs 40 and 41 (16% yield for the four steps), respectively.

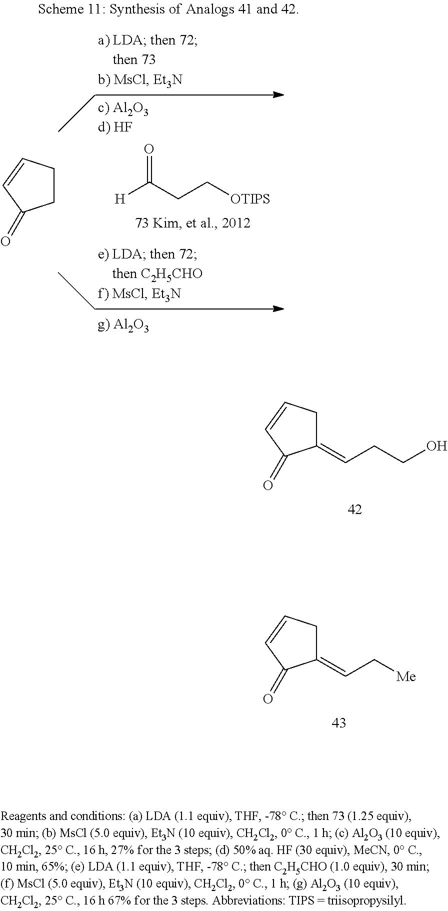

Analogs 42 and 43 were synthesized in a similar manner (see Scheme 11) starting from 2-cyclopenten-1-one (72) and using aldehyde 73 (Kim, et al., 2012) or propionaldehyde, respectively, as coupling partners in the aldol reaction. Thus, analog 42 was synthesized in four steps and 18% overall yield, and analog 43 was synthesized in three steps and 61% overall yield.

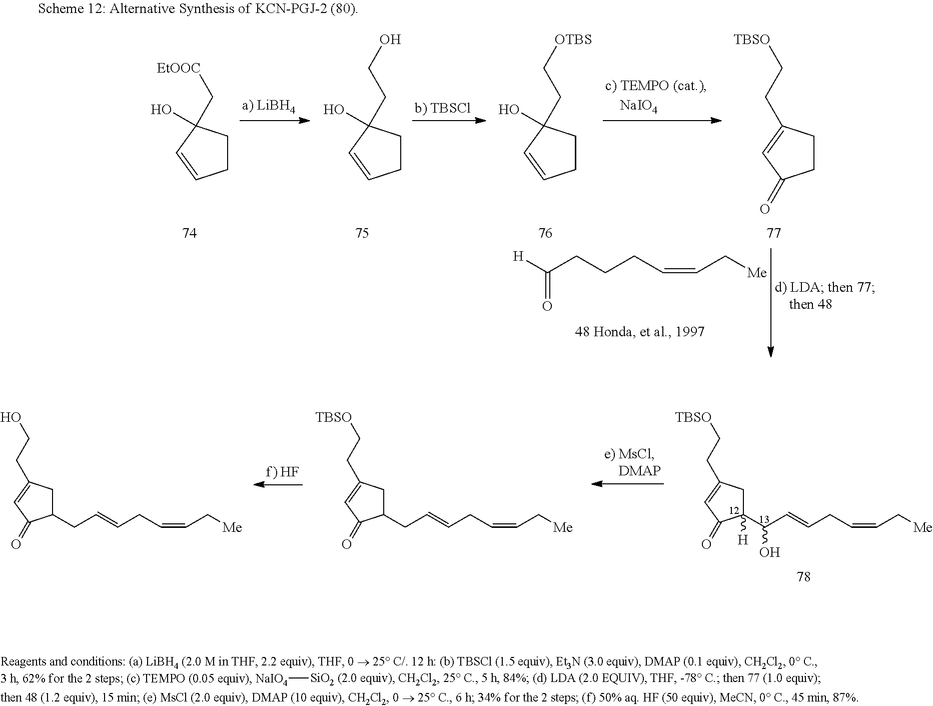

An alternative synthetic route to the analog KCN-PGJ-2 (compound 80, Scheme 12) was developed. Thus, starting with known hydroxy ester 74, (Liu, et al., 1991) reduction with LiBH₄ gave diol 75 which was silylated with TBSCl (Et₃N, cat. DMAP) to afford mono-TBS ether 76 in 62% overall yield for the two steps. Exposure of 76 to NaIO₄ in the presence of catalytic amounts of TEMPO led to enone 77 (84% yield) in which the olefinic bond was transpositioned into the trisubstituted site. This material (77) was identical to that obtained by Rh-catalyzed CH functionalization. Aldol reaction of 77 with unsaturated aldehyde 48 (Honda, et al., 1997) gave aldol product 78 (mixture of C12 and C13 epimers, inconsequential) whose exposure to MsCl and DMAP gave tetraene 79 as a single isomer in 34% yield for the two steps. Finally, desilylation of 79 with 50% aq. HF furnished compound 80 (previously designated as KCN-PGJ-2) in 87% yield.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### V. REFERENCES

The following references, to the extent that they provide exemplary procedural or other details supplementary to those set forth herein, are specifically incorporated herein by reference:
U.S. Pat. No. 5,440,013
U.S. Pat. No. 5,446,128
U.S. Pat. No. 5,475,085
U.S. Pat. No. 5,597,457
U.S. Pat. No. 5,618,914
U.S. Pat. No. 5,670,155
U.S. Pat. No. 5,672,681
U.S. Pat. No. 5,674,976
U.S. Pat. No. 5,710,245
U.S. Pat. No. 5,790,421
U.S. Pat. No. 5,840,833
U.S. Pat. No. 5,859,184
U.S. Pat. No. 5,889,155
U.S. Pat. No. 5,929,237
U.S. Pat. No. 6,093,573
U.S. Pat. No. 6,261,569
U.S. Patent Appln. 2005/0015232
U.S. Patent Appln. 2013/0005737
Aaronson and Horvath, Science, 296(5573):1653-5, 2002.
Abe and Kufe, Cancer Res., 49(11):2834-2839, 1989.
Acharya and Kobayashi, Tetrahedron Lett., 45:1199-1202, 2004
Acharya and Kobayashi, Tetrahedron, 62:3329-3343, 2006.
Agata et al., Cancer Res., 68:6136-44, 2008.
Ahmad et al., Cancer Res., 68:2920-2926, 2008.
Ahmad et al., J. Biol. Chem., 281:35764-9, 2006.
Ahmad et al., Nat. Cell Biol., 9:1419-1427, 2007.
Alvarez et al., Cancer Res., 65(12):5054-62, 2005.
Alvarez et al., Cancer Res., 66(6):3162-8, 2006.
Anderson, N. G., Practical Process Research & Development—A Guide For Organic Chemists, 2^{nd} ed., Academic Press, New York, 2012.
Bal, et al., Tetrahedron, 37:2091-2096, 1981.
Baldus et al., Clin. Cancer Res., 10(8):2790-2796, 2004.
Bickley, et al., Synlett, 1170-1174, 2003.
Bodanszky et al., J. Antibiot, 29(5):549-53, 1976.
Bowman et al., Oncogene, 19(21):2474-88, 2000.
Bromberg et al., Cell, 98(3):295-303, 1999.
Brown and Ahuja, J. Chem. Soc., Chem Comm., 2(15):553-554, 1973.
Brummond, et al., Org. Lett., 6:149-152, 2004.
Buerger et al., J. Biol. Chem., 278(39):37610-21, 2003.
By Jacquet, et al., Tetrahedron, 66:222-223, 2010.
Carreira, et at, J. Am. Chem. Soc., 116:8837-8838, 1994.
Catino, et at, J. Am. Chem. Soc., 126:13622-1623, 2004.
Chen & Greene, Mol. Cell. Biol. 5:392-401, 2004.
Cohen et at, J. Med. Chem., 33:883-894, 1990.
Dess and Martin, J. Org. Chem., 48:4155-4156, 1983.
Duraisamy et al., Gene, 373:28-34, 2006.
Fischer, Med. Res. Rev., 27(6):755-796, 2007.
Gaemers et al., J. Biol. Chem., 276:6191-6199, 2001.
Gendler et al., J. Biol. Chem., 263:12820-12823, 1988.
Germain and Frank, Clin. Cancer Res., 13(19):5665-9, 2007.
Gerondakis et al., Oncogene 25(51):6781-99, 2006.
Ghosh et al., Annu. Rev. Cell. Dev. Biol., 16:225-60, 1998.
Gilmore, available from NF-kB.org, 2008.
Grillot et al., J. Immunol., 158:4750-7, 1997.
Gronenborn et al., Anal. Chem., 62(1):2-15, 1990.
Hayden and Ghosh, Cell, 132:344-62, 2008.
Hegde, et al., Blood, 118:6909-6919, 2011.
Hodel et al., Mol. Cell, 10(2):347-58, 2002.
Hoffman et al., Oncogene, 25:6706-16, 2006.
Hoye, et al., Nat. Protoc., 2:2451-2458, 2007.
Huang et al., Cancer Biol Ther., 2:702-706, 2003.
Huang et al., Cancer Res., 65:10413-10422, 2005.
Huxford et al., Cell 95(6):759-70, 1998.
Jackson, Seminars in Oncology, 24:L164-172, 1997.
Jacobs et al., Cell, 95:749-58, 1998.
Jiang, et al., Nature, 391:82-86, 1998.
Johnson et al., In: Biotechnology And Pharmacy, Pezzuto et al. (Eds.), Chapman and Hall, NY, 1993.
Jones et al., J. Med. Chem., 39:904-917, 1996.
Karin & Lin, Nat. Immunol., 3:221-7, 2002.
Kau et al., Nat. Rev. Cancer, 4(2):106-17, 2004.
Kawano et al., Cancer Res., 67:11576-84, 2007.
Kerstein, et al., Nature, 405:421-425, 2000.
Kinlough et al., J. Biol. Chem., 279(51):53071-53077, 2004.
Kiyooka, et al., Tetrahedron, 66:1806-1816, 2010.
Kufe et al., Hybridoma, 3:223-232, 1984.
Lagow and Carson, J. Cell. Biochem., 86:759-72, 2002.
Lee et al., Cancer Cell, 15(4):283-293, 2009.
Leng et al., J. Biol. Chem., 282:19321-19330, 2007.
Levitan et al., J. Biol. Chem., 280:33374-33386, 2005.
Li et al., Cancer Biol. Ther., 2:187-193, 2003b.
Li et al., J. Biol. Chem., 276:35239-35242, 2001.
Li et al., J. Biol. Chem., 276:6061-6064, 2001.
Li et al., Mol. Cancer Res., 1:765-775, 2003c.
Li et al., Mol. Cell Biol., 18:7216-7224, 1998.
Li et al., Oncogene, 22:6107-6110, 2003a.
Ligtenberg et al., J. Biol. Chem., 267, 6171-6177, 1992.
Liniger, et al., ACS Med. Chem. Lett., 2:22-27, 2011.
Luche, J Am. Chem. Soc., 100:2226-2227, 1978.
Macao, Nat. Struct. Mol. Biol., 13, 71-76, 2006.
March's Advanced Organic Chemistry: Reactions, Mechanisms, and Structure, 2007.
McPherson, J Biol. Chem., 251:6300-6306, 1976.
Merlo et al., Cancer Res., 49, 6966-6971, 1989.
Merrifield, J. Am. Chem. Soc., 85:2149-2154, 1963.
Micheau & Tschopp, Cell, 114:181-90, 2003.
Miyazaki, et al., Org. Lett., 9:4737-4740, 2007.
Muthuswamy, Nat. Cell Biol., 3(9):785-92, 2001.
Narumiya and Fukushima, J. Pharmacol. Exp. Ther., 239:500-505, 1986.
Narumiya, et al., J. Pharmacol. Exp. Ther., 239:560-511, 1986.
Narumiya, et al., J. Pharmacol. Exp. Ther., 242:306-311, 1987.
Natoli et al., Nat. Immunol., 6:439-45, 2005.
Navia et al., Curr. Opin. Struct. Biol., 2:202-210, 1992.
Oger, et al., Chem. Euro. J., 16:13976-13980, 2010.
Ohtani-Fujita, et al., Biochem. Biophys. Res. Commun., 251:648-652, 1998.
Pasparakis et al., Cell Death Differ. 13:861-72, 2006.
PCT Appln. PCT/US00/03745
PCT Appln. PCT/US00/14667
PCT Appln. PCT/US99/11913
PCT Appln. PCT/US99/18441
Peptide Synthesis, 1985
Percipalle et al., J. Mol. Biol., (4):722-32, 1997.
Perey et al., Cancer Res., 52(22):6365-6370, 1992.
Protective Groups in Organic Chemistry, 1973
Protein NMR Spectroscopy, Principles and Practice, J. Cavanagh et al., Academic Press, San Diego, 1996.
Raina et al., EMBO J., 25:3774-3783, 2006.
Raina et al., J. Biol. Chem., 279:20607-20612, 2004.
Ramasamy et al., Mol. Cell, 27:992-1004, 2007.
Remington's Pharmaceutical Sciences, 15^{th} Ed., 1035-1038 and 1570-1580, 1990.
Remington's Pharmaceutical Sciences, 15^{th} Ed., 3:624-652, 1990.
Ren et al., Cancer Cell, 5:163-175, 2004.
Ren et al., J. Biol. Chem., 277:17616-17622, 2002.
Ricote, et al., Nature, 391:79-82, 1998.
Rocchi and Auwerx, J Ann. Med., 10:342-351, 1999.
Rossi, et al., Nature, 403:103-108, 2000.
Ryan and Wente, Curr. Opin. Cell Biol., 12(3):361-71, 2000.
Sasaki and Fukushima, Anti-Cancer Drug, 5:131-138, 1994.
Schneider-Brachert et al., Immunity, 21:415-28, 2004.
Schroeder et al., J. Biol. Chem., 276(16):13057-13064 2001.
Schroeder et al., Oncogene, 23:5739-5747, 2004.
Shuai, Oncogene, 19(21):2638-44, 2000.
Siddiquee et al., Proc. Natl. Acad. Sci. USA, 104(18):7391-6, 2007.
Siddiqui et al., Proc. Natl. Acad. Sci. USA, 85:2320-2323, 1988.
Smith, et al., Org. Lett., 7:3099-3102, 2005.
Solid Phase Peptide Synthelia, 1984.
Song et al., Proc. Natl. Acad. Sci. USA, 102(13):4700-5, 2005.
Soule et al., Cancer Res., 50(18):6075-6086, 1990.
Suh and Gumbiner, Exp. Cell Res., 290(2):447-56, 2003.
Suzuki, et al., Biosci., Biotechnol., Biochem., 62:2073-2075, 1998.
Trost and Bunt, Angew. Chem. Int. Ed. Engl., 35:99-102, 1996.
Truscott et al., J Cell Biol., 163(4):707-713, 2003.
Vermeer et al., Nature, 422(6929):322-6, 2003.
Wavrin and Viala, Synthesis, 326-330, 2002.
Weber, Advances Protein Chem., 41:1-36, 1991.
Wegenka et al., Mol. Cell Biol., 14(5):3186-96, 1994.
Wei et al., Cancer Cell, 7:167-178, 2005.
Wei et al., Cancer Res., 67(4):1853-8, 2007.
Wei et al., Mol. Cell., 21:295-305, 2006.
Weis, Cell, 112(4):441-51, 2003.
Wen et al., J. Biol. Chem., 278:38029-38039, 2003.
Wider, BioTechniques, 29:1278-1294, 2000.
Yamamoto et al., J. Biol. Chem., 272:12492-12494, 1997.
Yin et al., J. Biol. Chem., 278:35458-35464, 2003.
Yin et al., J. Biol. Chem., 279:45721-45727, 2004.
Yin et al., J. Biol. Chem., 282:257-266, 2007.
Young et al., Cell. 112(1):41-50, 2003.
Yu and Jove, Nat. Rev. Cancer, 4(2):97-105, 2004.
Zhang et al., Mol. Cell. Biol., 19:7138-7146, 1999.

1. A compound of the formula: 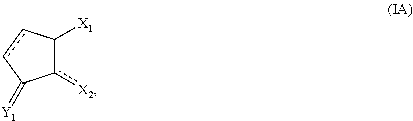
wherein:
Y₁ is O, NR₁, or N—OR₁;
wherein:
R₁ is hydrogen, alkyl_{(C≦6)}, or substituted alkyl_{(C≦6)};
X₁ is hydrogen, alkyl_{(C≦8)}, substituted alkyl_{(C≦8)}, 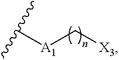
or taken together with X₂ as defined below;
wherein:
A₁ is alkanediyl_{(C≦8)}, alkenediyl_{(C≦8)}, alkynediyl_{(C≦8)}, arenediyl_{(C≦12)}, heteroarenediyl_{(C≦12)}, or a substituted version of any of these groups;
n is 0, 1, 2, 3, 4, 5, or 6;
X₃ is hydrogen, hydroxy, amino, cyano, or;
alkyl_{(C≦12)}, alkenyl_{(C≦12)}, alkynyl_{(C≦12)}, aryl_{(C≦12)}, heteroaryl_{(C≦12)}, heterocycloalkyl_{(C≦12)}, alkoxy_{(C≦12)}, alkenyloxy_{(C≦12)}, alkynyloxy_{(C≦12)}, aryloxy_{(C≦12)}, heteroaryloxy_{(C≦12)}, heterocycloalkyloxy_{(C≦12)}, acyloxy_{(C≦12)}, alkylamino_{(C≦12)}, dialkylamino_{(C≦12)}, alkenylamino_{(C≦12)}, alkynylamino_{(C≦12)}, arylamino_{(C≦12)}, heteroarylamino_{(C≦12)}, heterocycloalkylamino_{(C≦12)}, amido_{(C≦12)}, or a substituted version of any of these groups; or —C(O)NR₂R₃, —C(O)R₂; or —Y₂—R₄;
wherein:
R₂ and R₃ are each independently hydrogen, hydroxy, alkyl_{(C≦8)}, aryl_{(C≦8)}, alkoxy_{(C≦8)}, alkylsulfonyl_{(C≦8)}, arylsulfonyl_{(C≦8)}, or a substituted version of any of the last five groups; or R₂ is -alkoxydiyl_{(C≦6)}-S(O)₂-aryl_{(C≦12)} or a substituted version of this group; Y₂ is alkanediyl_{(C≦12)}, substituted alkanediyl_{(C≦12)}; alkoxydiyl_{(C≦12)}, or substituted alkoxydiyl_{(C≦12)}; and R₄ is hydrogen, —C(O)NR₂R₃, or —C(O)R₂; wherein R₂ and R₃ are as defined above and
X₂ is 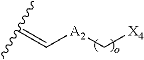
or taken together with X₁ as defined below;
wherein:
A₂ is alkanediyl_{(C≦8)}, alkenediyl_{(C≦8)}, alkynediyl_{(C≦8)} or a substituted version of any of these groups; or —CH₂CH(OR₄)—;
wherein:
R₄ is alkyl_{(C≦12)}; alkenyl_{(C≦12)}, alkynyl_{(C≦12)}, aryl_{(C≦12)}, heteroaryl_{(C≦12)}, acyl_{(C≦12)}, or a substituted version of any of these groups;
X₄ is hydrogen, hydroxy, or
alkyl_{(C≦12)}, alkenyl_{(C≦12)}, alkynyl_{(C≦12)}, aryl_{(C≦12)}, heteroaryl_{(C≦12)}, heterocycloalkyl_{(C≦12)}, aryloxy_{(C≦12)}, heteroaryloxy_{(C≦12)}, heterocycloalkoxy_{(C≦12)}, arylthio_{(C≦12)}, heteroarylthio_{(C≦12)}, heterocycloalkylthio_{(C≦12)}, arylsulfinyl_{(C≦12)}, heteroarylsulfinyl_{(C≦12)}, heterocycloalkylsulfinyl_{(C≦12)}, arylsulfonyl_{(C≦12)}, heteroarylsulfonyl_{(C≦12)}, heterocycloalkylsulfonyl_{(C≦12)}, or a substituted version of any of these groups; and
o is 1, 2, 3, 4, 5, or 6;
wherein:
X₁ and X₂ are taken together as shown in formula (II): 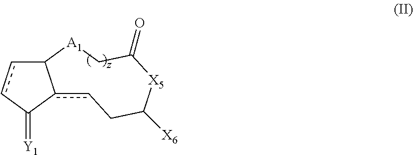
wherein:
Y₁ is O, NH, or N—OR₁;
wherein:
R₁ is hydrogen, alkyl_{(C≦6)}, or substituted alkyl_{(C≦6)};
A₁ is alkanediyl_{(C≦8)}, alkenediyl_{(C≦8)}, alkynediyl_{(C≦8)}, arenediyl_{(C≦12)}, heteroarenediyl_{(C≦12)}, or a substituted version of any of these groups;
z is 1, 2, 3, 4, 5, or 6;
X₅ is CR₄R₅, O, NH, NR₆, or S;
wherein:
R₄, R₅, and R₆ are each independently H, alkyl_{(C≦8)}, aryl_{(C≦8)}, aralkyl_{(C≦8)}; or a substituted version of any of the last three groups; and
X₆ is alkyl_{(C≦12)}; alkenyl_{(C≦12)}, alkynyl_{(C≦12)}, aryl_{(C≦12)}, heteroaryl_{(C≦12)} or a substituted version of any of these groups;
provided that the compound does not have the formula: 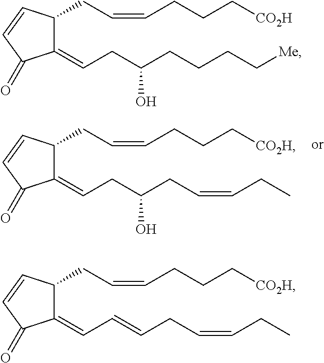
or a pharmaceutically acceptable salt thereof.

2. The compound of claim 1, further defined by the formula: 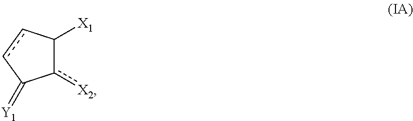
wherein:
Y₁ is O, NR₁, or N—OR₁;
wherein:
R₁ is hydrogen, alkyl_{(C≦6)}, or substituted alkyl_{(C≦6)};
X₁ is hydrogen, alkyl_{(C≦8)}, substituted alkyl_{(C≦8)}, 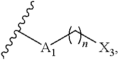
or taken together with X₂ as defined below;
wherein:
A₁ is alkanediyl_{(C≦8)}, alkenediyl_{(C≦8)}, alkynediyl_{(C≦8)}, arenediyl_{(C≦12)}, heteroarenediyl_{(C≦12)}, or a substituted version of any of these groups;
n is 0, 1, 2, 3, 4, 5, or 6;
X₃ is hydrogen, hydroxy, amino, cyano, or
alkyl_{(C≦12)}, alkenyl_{(C≦12)}, alkynyl_{(C≦12)}, aryl_{(C≦12)}, heteroaryl_{(C≦12)}, heterocycloalkyl_{(C≦12)}, or a substituted version of any of these groups; or —C(O)NR₂R₃ or —C(O)R₂;
wherein:
R₂ and R₃ are each independently hydrogen, hydroxy, alkyl_{(C≦6)}, aryl_{(C≦8)}, alkoxy_{(C≦6)}, alkylsulfonyl_{(C≦8)}, arylsulfonyl_{(C≦8)}, or a substituted version of any of the last five groups; or R₂ is -alkoxydiyl_{(C≦6)}-S(O)₂-aryl_{(C≦12)} or a substituted version of this group; and
X₂ is 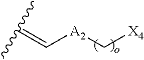
or taken together with X₁ as defined below;
wherein:
A₂ is alkanediyl_{(C≦8)}, alkenediyl_{(C≦8)}, alkynediyl_{(C≦8)} or a substituted version of any of these groups;
X₄ is hydrogen, hydroxy, or
alkyl_{(C≦12)}, alkenyl_{(C≦12)}, alkynyl_{(C≦12)}, aryl_{(C≦12)}, heteroaryl_{(C≦12)}, heterocycloalkyl_{(C≦12)}, aryloxy_{(C≦12)}, heteroaryloxy_{(C≦12)}, heterocycloalkoxy_{(C≦12)}, arylthio_{(C≦12)}, heteroarylthio_{(C≦12)}, heterocycloalkylthio_{(C≦12)}, arylsulfinyl_{(C≦12)}, heteroarylsulfinyl_{(C≦12)}, heterocycloalkylsulfinyl_{(C≦12)}, arylsulfonyl_{(C≦12)}, heteroarylsulfonyl_{(C≦12)}, heterocycloalkylsulfonyl_{(C≦12)}, or a substituted version of any of these groups; and
o is 0, 1, 2, 3, 4, 5, or 6;
wherein:
X₁ and X₂ are taken together as shown in formula (II): 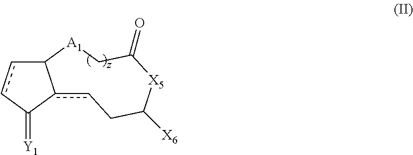
wherein:
Y₁ is O, NH, or N—OR₁;
wherein:
R₁ is hydrogen, alkyl_{(C≦6)}, or substituted alkyl_{(C≦6)};
A₁ is alkanediyl_{(C≦8)}, alkenediyl_{(C≦8)}, alkynediyl_{(C≦8)}, arenediyl_{(C≦12)}, heteroarenediyl_{(C≦12)}, or a substituted version of any of these groups;
z is 1, 2, 3, 4, 5, or 6;
X₅ is CR₄R₅, O, NH, NR₆, or S;
wherein:
R₄, R₅, and R₆ are each independently H, alkyl_{(C≦8)}, aryl_{(C≦8)}, aralkyl_{(C≦8)}; or a substituted version of any of the last three groups; and
X₆ is alkyl_{(C≦12)}; alkenyl_{(C≦12)}, alkynyl_{(C≦12)}, aryl_{(C≦12)}, heteroaryl_{(C≦12)} or a substituted version of any of these groups;
provided that the compound does not have the formula: 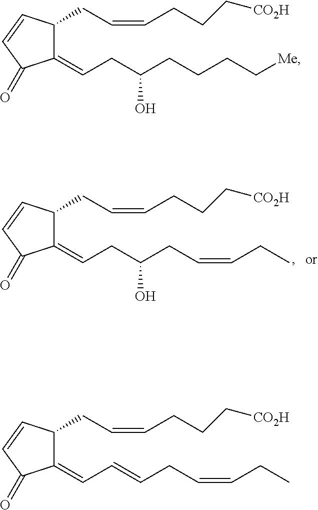 [SMILES: CCCCC[C@H](O)C/C=C1/C(=O)C=C[C@@H]1C/C=C\CCCC(=O)O ; CC/C=C\C[C@H](O)C/C=C1/C(=O)C=C[C@@H]1C/C=C\CCCC(=O)O ; CC/C=C\C/C=C/C=C1/C(=O)C=C[C@@H]1C/C=C\CCCC(=O)O]
or a pharmaceutically acceptable salt thereof.

3. The compound of claim 1, wherein the compound is further defined as:
V 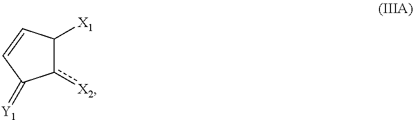
wherein:
Y₁ is O or N—OR₁;
wherein:
R₁ is hydrogen or alkyl_{(C≦6)};
X₁ is hydrogen or 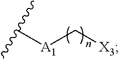
wherein:
A₁ is alkanediyl_{(C≦8)}, alkenediyl_{(C≦8)}, alkynediyl_{(C≦8)}, or heteroarenediyl_{(C≦12)};
n is 0, 1, 2, 3, or 4;
X₃ is hydrogen, hydroxy,
alkyl_{(C≦6)}, heteroaryl_{(C≦8)}, or a substituted version of any of these groups; or —C(O)NR₂R₃ or —C(O)R₂;
wherein:
R₂ and R₃ are each independently hydrogen, alkyl_{(C≦6)}, aryl_{(C≦8)}, alkoxy_{(C≦6)}, or a substituted version of any of the last three groups; and
X₂ is 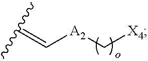
wherein:
A₂ is alkanediyl_{(C≦8)}, alkenediyl_{(C≦8)}, or a substituted version of any of these groups;
X₄ is hydrogen, hydroxy, or
alkyl_{(C≦12)}, alkenyl_{(C≦12)}, alkynyl_{(C≦12)}, aryl_{(C≦12)}, heteroaryl_{(C≦12)}, heterocycloalkyl_{(C≦12)}, aryloxy_{(C≦12)}, heteroaryloxy_{(C≦12)}, heterocycloalkoxy_{(C≦12)}, arylthio_{(C≦12)}, heteroarylthio_{(C≦12)}, heterocycloalkylthio_{(C≦12)}, arylsulfonyl_{(C≦12)}, heteroarylsulfonyl_{(C≦12)}, heterocycloalkylsulfonyl_{(C≦12)}, or a substituted version of any of these groups; and
o is 0, 1, 2, 3, 4, 5, or 6;
wherein:
X₁ and X₂ are taken together as shown in formula (IV): 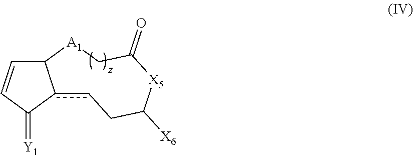
wherein:
Y₁ is O, NH, or N—OR₁;
wherein:
R₁ is hydrogen, alkyl_{(C≦6)}, or substituted alkyl_{(C≦6)};
A₁ is alkanediyl_{(C≦8)}, alkenediyl_{(C≦8)}, alkynediyl_{(C≦8)}, arenediyl_{(C≦12)}, heteroarenediyl_{(C≦12)}, or a substituted version of any of these groups;
z is 1, 2, 3, 4, 5, or 6;
X₅ is CR₄R₅, O, NH, NR₆, or S;
wherein:
R₄, R₅, and R₆ are each independently H, alkyl_{(C≦8)}, aryl_{(C≦8)}, aralkyl_{(C≦8)}; or a substituted version of any of the last three groups; and
X₆ is alkyl_{(C≦12)}; alkenyl_{(C≦12)}, alkynyl_{(C≦12)}, aryl_{(C≦12)}, heteroaryl_{(C≦12)} or a substituted version of any of these groups;
provided that the compound does not have the formula: 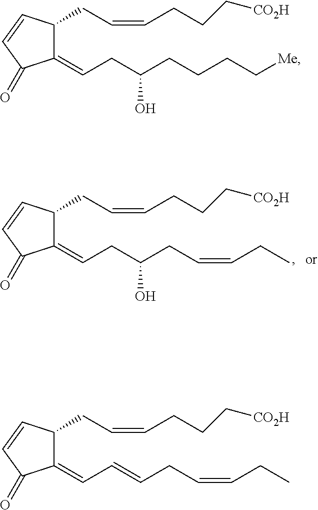 [SMILES: CCCCC[C@H](O)C/C=C1/C(=O)C=C[C@@H]1C/C=C\CCCC(=O)O ; CC/C=C\C[C@H](O)C/C=C1/C(=O)C=C[C@@H]1C/C=C\CCCC(=O)O ; CC/C=C\C/C=C/C=C1/C(=O)C=C[C@@H]1C/C=C\CCCC(=O)O]
or a pharmaceutically acceptable salt thereof.

4.-6. (canceled)

7. The compound of claim 1, wherein Y₁ is O.

8. The compound of claim 1, wherein Y₁ is N—OH or N—OMe.

9. The compound of claim 8, wherein Y₁ is N—OMe.

10.-95. (canceled)

96. The compound of claim 1, wherein X₁ and X₂ are taken together as defined by the formula: 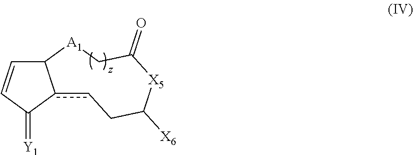
wherein:
Y₁ is O, NH, or N—OR₁;
wherein:
R₁ is hydrogen, alkyl_{(C≦6)}, or substituted alkyl_{(C≦6)};
A₁ is alkanediyl_{(C≦8)}, alkenediyl_{(C≦8)}, alkynediyl_{(C≦8)}, arenediyl_{(C≦12)}, heteroarenediyl_{(C≦12)}, or a substituted version of any of these groups;
z is 1, 2, 3, 4, 5, or 6;
X₅ is CR₄R₅, O, NH, NR₆, or S;
wherein:
R₄, R₅, and R₆ are each independently H, alkyl_{(C≦8)}, aryl_{(C≦8)}, aralkyl_{(C≦8)}; or a substituted version of any of the last three groups; and
X₆ is alkyl_{(C≦12)}; alkenyl_{(C≦12)}, alkynyl_{(C≦12)}, aryl_{(C≦12)}, heteroaryl_{(C≦12)} or a substituted version of any of these groups.

97. The compound of claim 96, wherein A₁ is alkenediyl_{(C≦6)}.

98. (canceled)

99. The compound of claim 96, wherein z is 1, 2, 3, or 4.

100.-101. (canceled)

102. The compound of claim 96, wherein X₅ is O.

103. The compound of claim 96, wherein X₆ is alkenyl_{(C≦12)}.

104. (canceled)

105. The compound of claim 1, wherein the compound is further defined as: 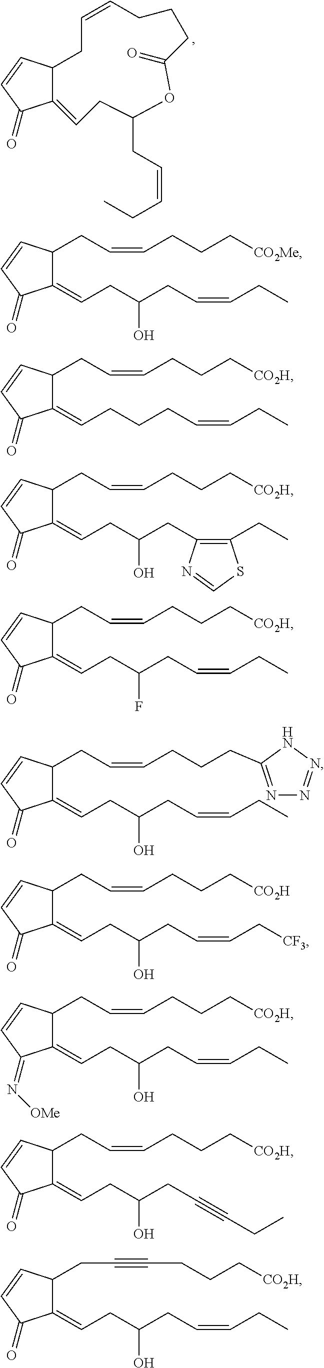 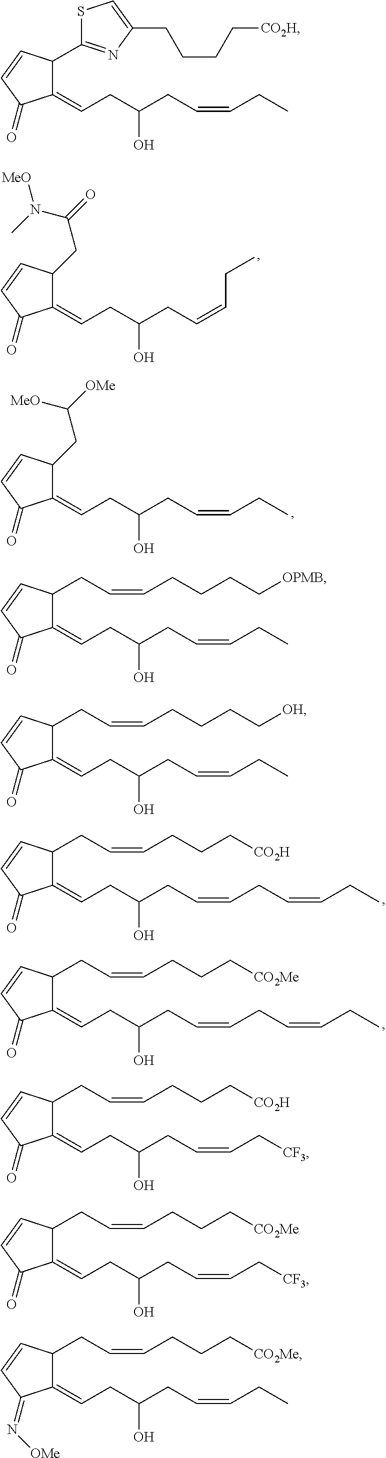 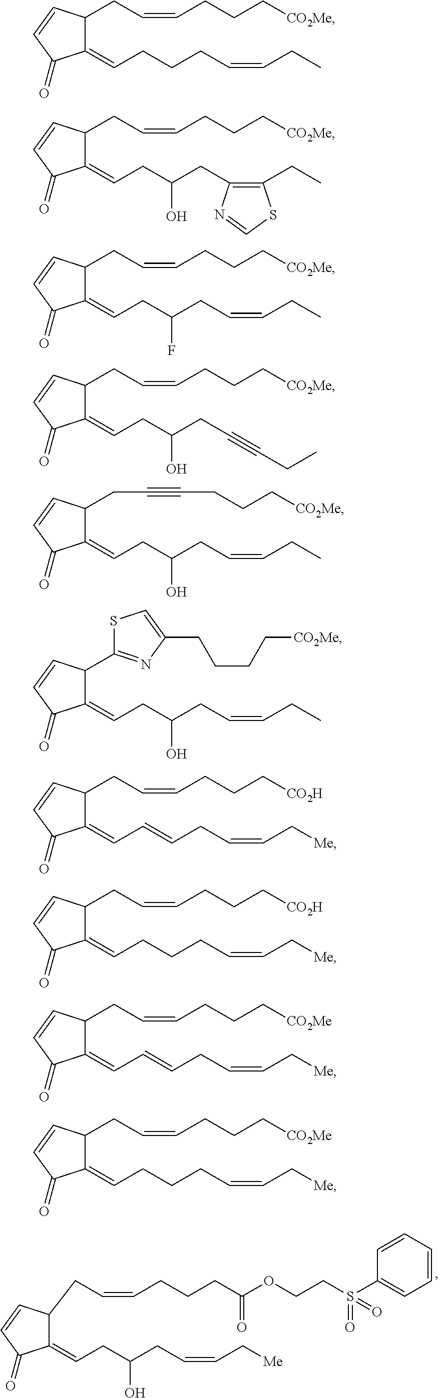 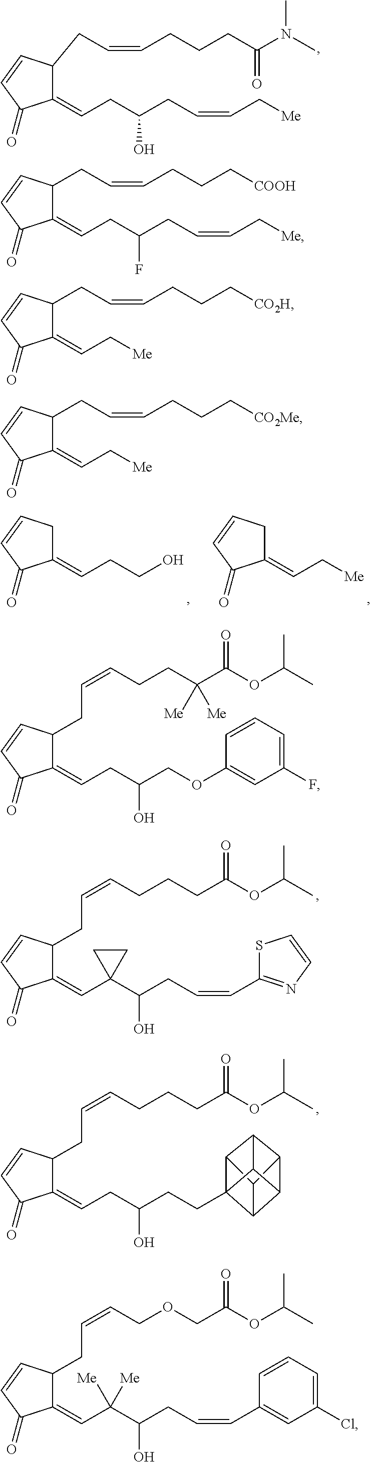 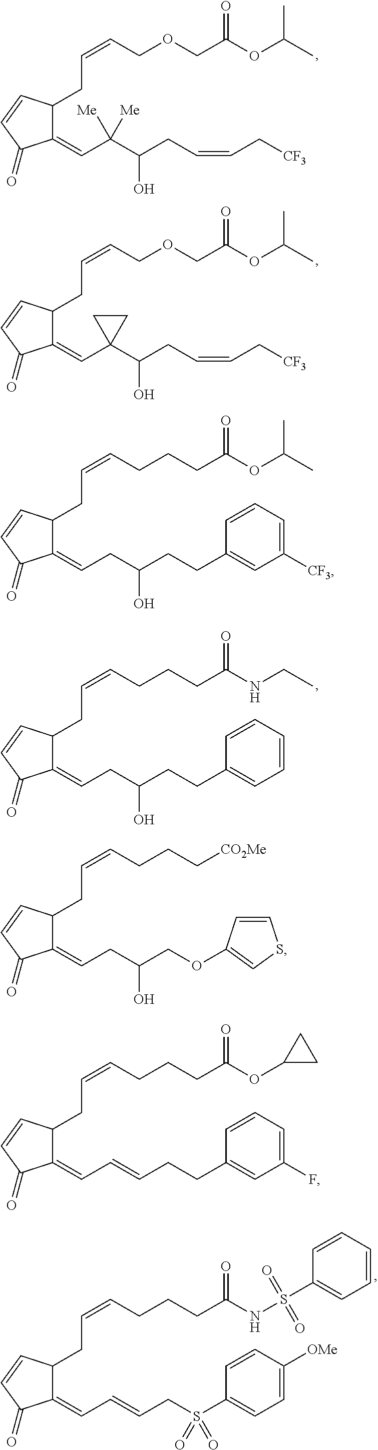 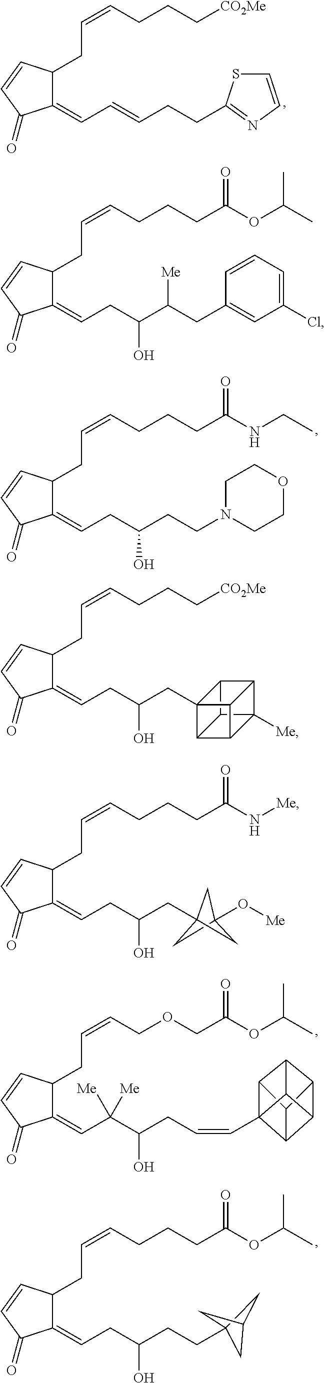 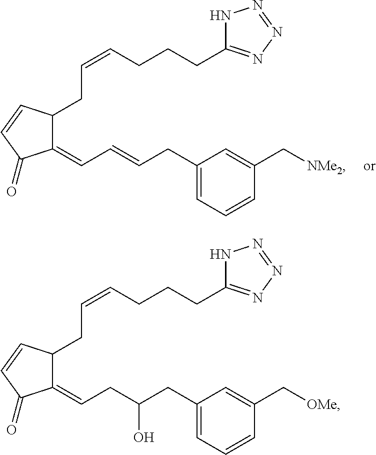
or an optical isomer or pharmaceutically acceptable salt thereof.

106. The compound of claim 105, further defined as: 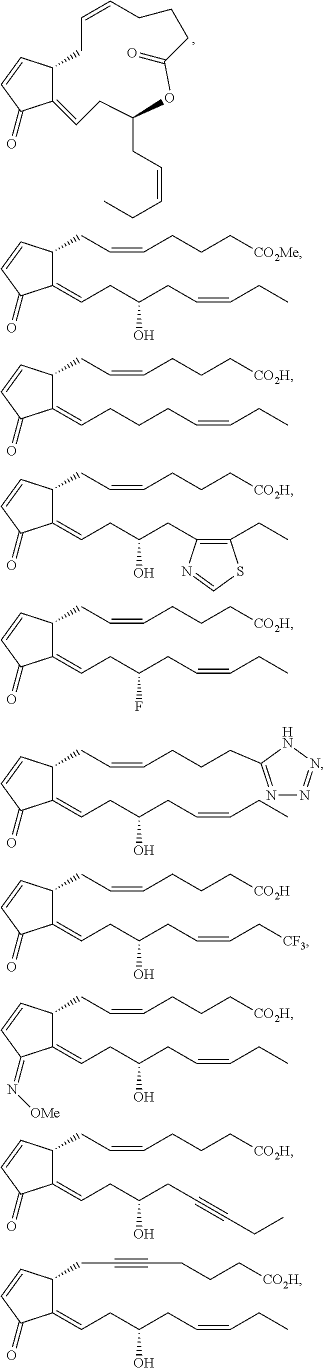 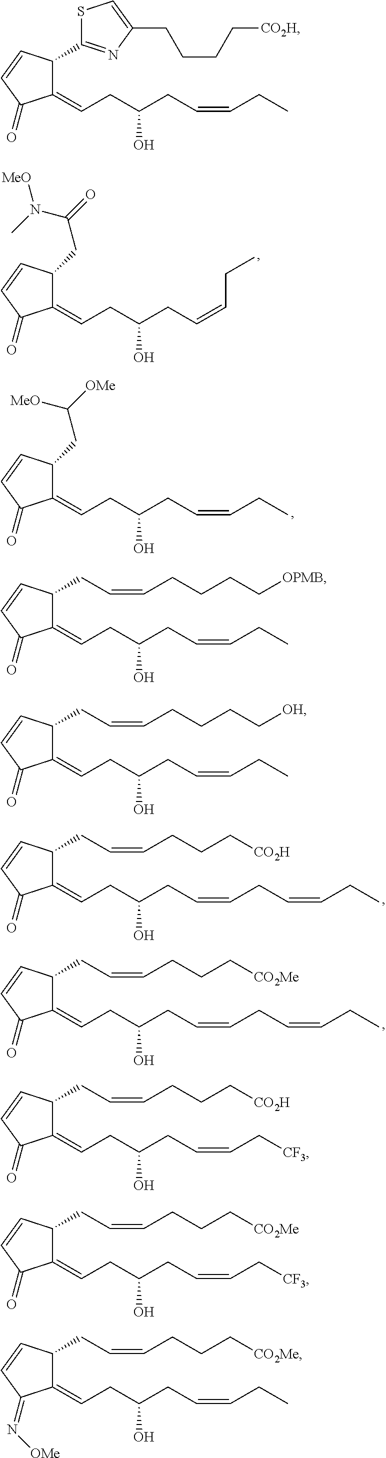 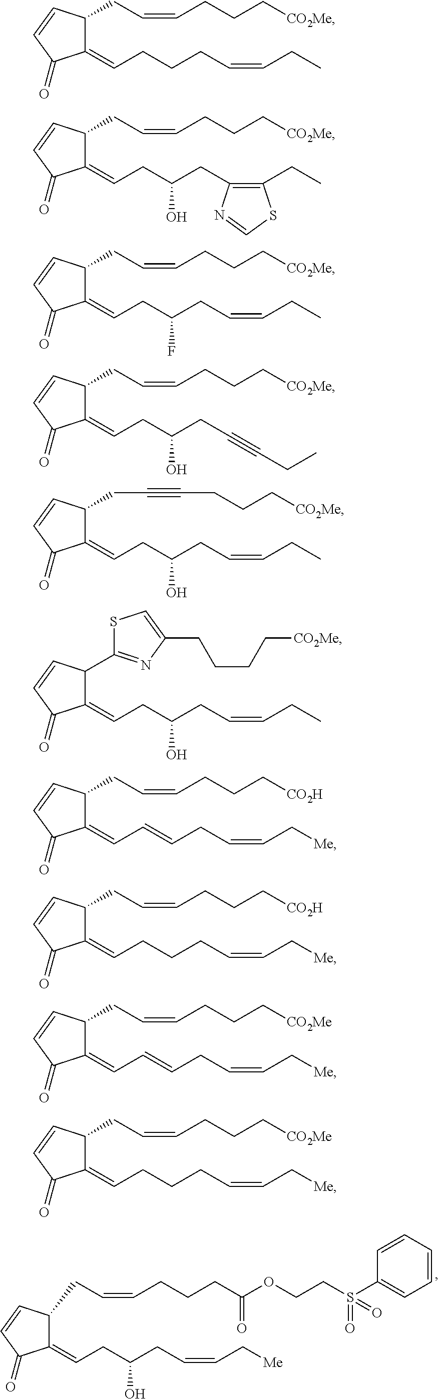 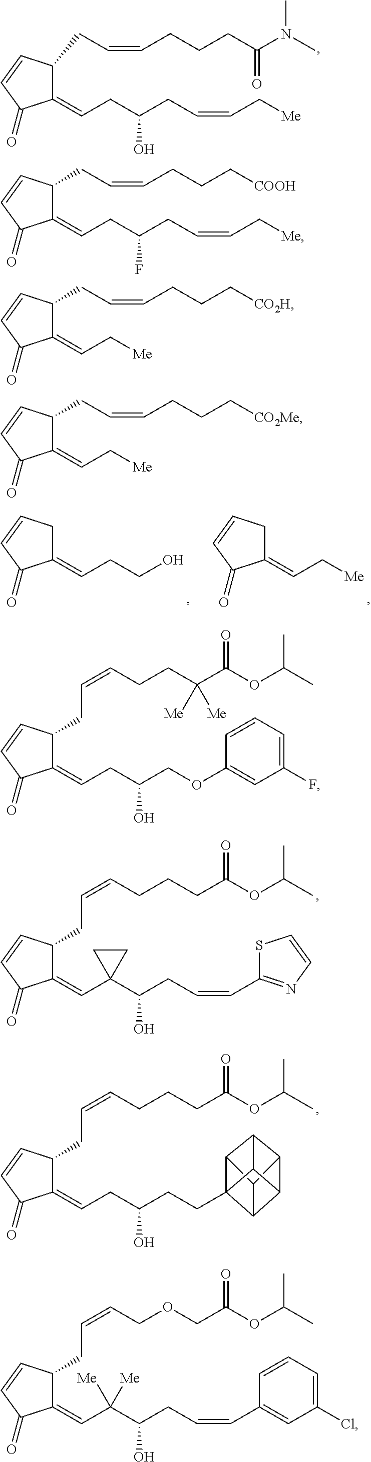 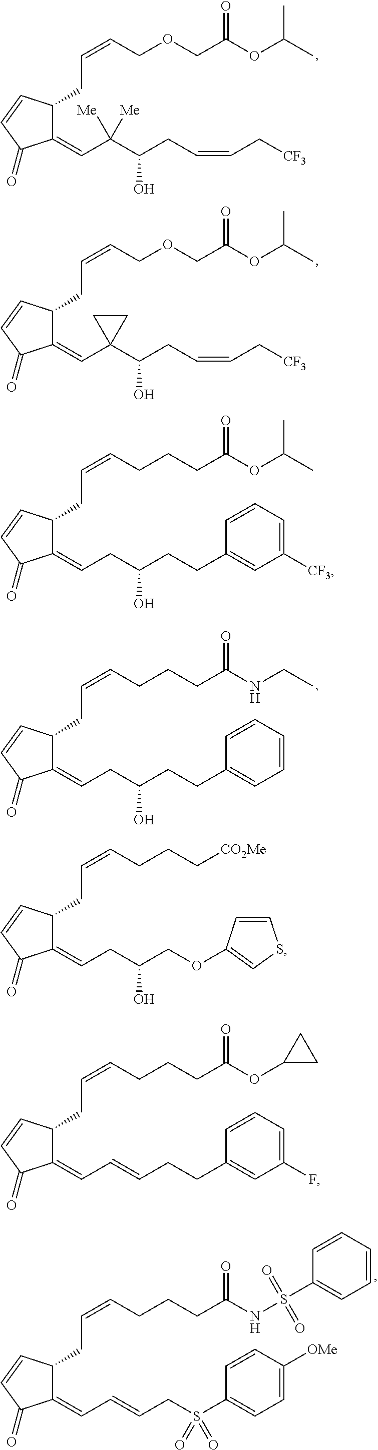 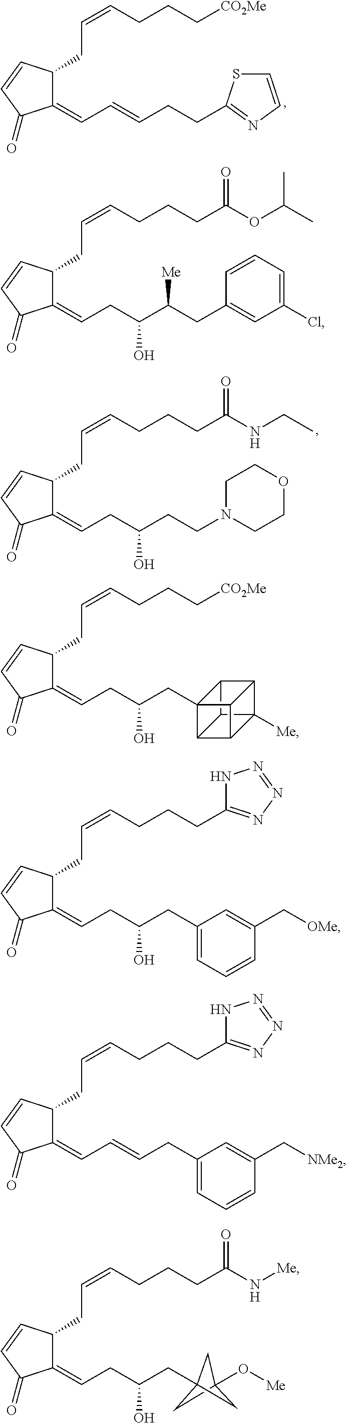 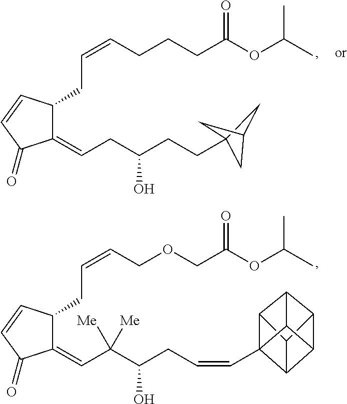 [SMILES: CC(C)OC(=O)CCC/C=C\C[C@H]1C=CC(=O)/C1=C/C[C@@H](O)CCC12CC(C1)C2 ; CC(C)OC(=O)COC/C=C\C[C@H]1C=CC(=O)/C1=C/C(C)(C)[C@@H](O)C/C=C\C12C3C4C5C3C1C5C42]
or a pharmaceutically acceptable salt thereof.

107. A pharmaceutical composition comprising a compound of claim 1 and an excipient.

108. (canceled)

109. The pharmaceutical composition of claim 107, wherein the composition is formulated for oral, topical, intraarterial, intraperitoneal, or intravenous administration.

110-114. (canceled)

115. A method of treating a disease or disorder in a patient in need thereof comprising administering to the patient a pharmaceutically effective amount of a compound or composition of claim 1 or a pharmaceutically acceptable salt or optical isomer thereof.

116. The method of claim 115, wherein the disease is cancer.

117.-118. (canceled)

119. The method of claim 116, wherein the cancer is leukemia.

120.-130. (canceled)

131. A method of preparing a compound comprised by reacting a base with a compound of the formula: 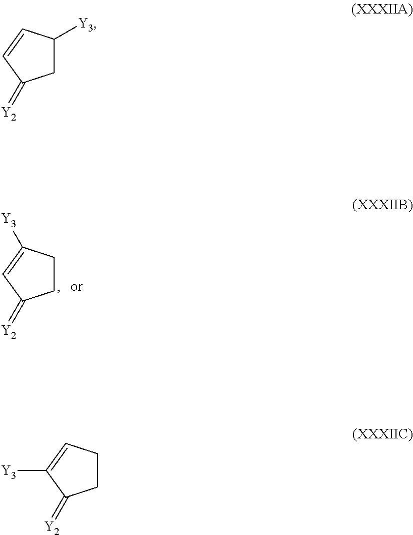
wherein:
Y₂ is O, S, NH, or NA₁;
wherein:
A₁ is alkyl_{(C≦6)}, alkoxy_{(C≦6)}, or a substituted version of any of these groups;
Y₃ is alkyl_{(C≦18)}, alkenyl_{(C≦18)}, alkynyl_{(C≦18)}, aryl_{(C≦18)}, aralkyl_{(C≦18)}, heteroaryl_{(C≦18)}, heteroaralkyl_{(C≦18)}, heterocycloalkyl_{(C≦18)}, or a substituted version of any these groups; or —X₁-A₂-R₁;
wherein:
X₁ is alkanediyl_{(C≦8)}, alkenediyl_{(C≦8)}, alkynediyl_{(C≦8)}, arenediyl_{(C≦12)}, heteroarenediyl_{(C≦12)}, or a substituted version of any of these groups;
A₂ is a covalent bond, O, S, S(O), S(O)₂, NH, or NR₂; wherein R₂ is alkyl_{(C≦6)} or substituted alkyl_{(C≦6)}; and
R₁ is alkyl_{(C≦12)}, alkenyl_{(C≦12)}, alkynyl_{(C≦12)}, aryl_{(C≦12)}, aralkyl_{(C≦12)}, heteroaryl_{(C≦12)}, heteroaralkyl_{(C≦12)}, heterocycloalkyl_{(C≦12)}, acyl_{(C≦12)}, or a substituted version of any of these groups;
and then adding a compound of the formula: 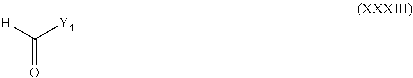
wherein:
Y₄ is alkyl_{(C≦18)}, alkenyl_{(C≦18)}, alkynyl_{(C≦18)}, aryl_{(C≦18)}, aralkyl_{(C≦18)}, heteroaryl_{(C≦18)}, heteroaralkyl_{(C≦18)}, heterocycloalkyl_{(C≦18)}, or a substituted version of any these groups; or —X₂-A₃-R₃;
wherein:
X₂ is alkanediyl_{(C≦8)}, alkenediyl_{(C≦8)}, alkynediyl_{(C≦8)}, arenediyl_{(C≦12)}, heteroarenediyl_{(C≦12)}, or a substituted version of any of these groups;
A₃ is a covalent bond, O, S, S(O), S(O)₂, NH, or NR₄; wherein R₄ is alkyl_{(C≦6)} or substituted alkyl_{(C≦6)}; and
R₃ is alkyl_{(C≦12)}, alkenyl_{(C≦12)}, alkynyl_{(C≦12)}, aryl_{(C≦12)}, aralkyl_{(C≦12)}, heteroaryl_{(C≦12)}, heteroaralkyl_{(C≦12)}, heterocycloalkyl_{(C≦12)}, acyl_{(C≦12)}, or a substituted version of any of these groups;
to form a compound of the formula: 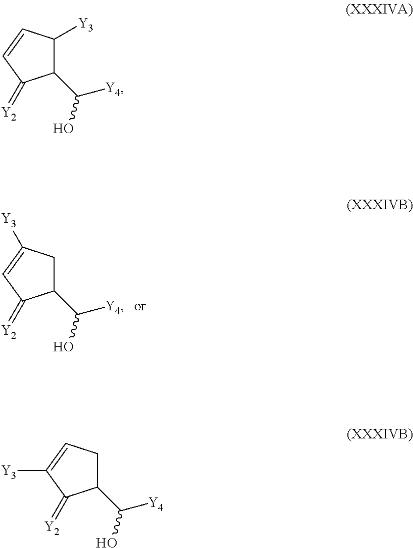
wherein Y₂, Y₃, and Y₄ are as defined above.

132.-293. (canceled)

294. A method of preparing an enone of the formula: 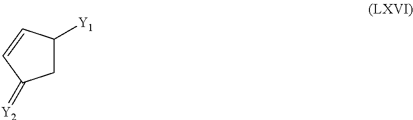
wherein:
Y₁ is alkyl_{(C≦18)}, alkenyl_{(C≦18)}, alkynyl_{(C≦18)}, aralkyl_{(C≦18)}, heteroaralkyl_{(C≦18)}, or a substituted version of any of these groups; and
Y₂ is O, S, and NR₁,
wherein R₁ is hydrogen, alkyl_{(C≦6)}, or substituted alkyl_{(C≦6)};
comprising
A) reacting a compound of the formula: 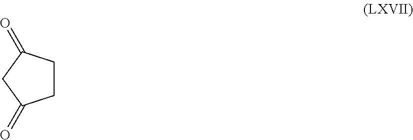
with an acid and a compound of the formula: 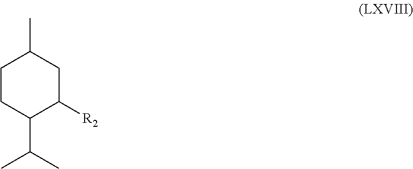
wherein:
R₂ is hydroxy, mercapto, or —NHR₁ wherein R₁ is as defined above;
to form a compound of the formula: 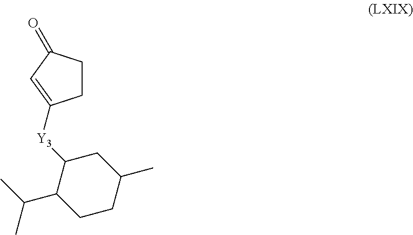
wherein:
Y₃ is —O—, —S—, or —NR₁—, wherein R₁ is as defined above;
B) reacting the compound of formula LXIX with a base and a compound of formula X₁—Y₁, wherein X₁ is halo or a group which enhances the ability of the hydroxyl group to be eliminated and Y₁ is as defined above; to form a compound of the formula: 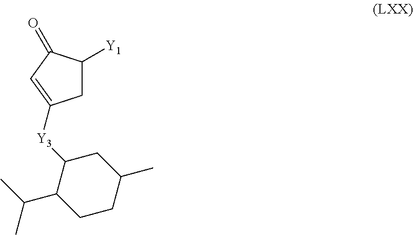
wherein: Y₁ and Y₃ are as defined above; and
C) reducing the compound of formula LXX with a reducing agent to form a compound of formula LXVI.

295.-434. (canceled)
